SEQUENCE LISTING (74 sequences)

SEQ ID NO: 1 - DNA, 1464 bp, Arabidopsis thaliana
  misc_feature (1)..(1464): sequence number 12707591
atgggtgaaaccgctgccgccaataaccaccgtcaccaccaccatcacggccaccaggtctttgacgtggccagccacgatttcgtccctccacaaccggcttttaaatgcttcgatgatgatggccgcctcaaaagaactgggactgtttggaccgcgagcgctcatataataactgcggttatcggatccggcgttttgtcattggcgtgggcgattgcacagctcggatggatcgctggccctgctgtgatgctattgttctctcttgttactctttactcctccacacttcttagcgactgctacagaaccggcgatgcagtgtctggcaagagaaactacacttacatggatgccgttcgatcaattctcggtgggttcaagttcaagatttgtgggttgattcaatacttgaatctctttggtatcgcaattggatacacgatacaggcgatcaagagatccaactgcttccacaagagtggaggaaaagacccatgtcacatgtccagtaatccttacatgatcgtatttggtgtggcagagatcttgctctctcaggttcctgatttcgatcagatttggtggatctccattgttgcagctgttatgtccttcacttactctgccattggtctagctcttggaatcgttcaagttgcagcgaatggagttttcaaaggaagtctcactggaataagcatcggaacagtgactcaaacacagaagatatggagaaccttccaagcacttggagacattgcctttgcgtactcatactctgttgtcctaatcgagattcaggatactgtaagatccccaccggcggaatcgaaaacgatgaagaaagcaacaaaaatcagtattgccgtcacaactatcttctacatgctatgtggctcaatgggttatgccgcttttggagatgcagcaccgggaaacctcctcaccggttttggattctacaacccgttttggctccttgacatagctaacgccgccattgttgtccacctcgttggagcttaccaagtctttgctcagcccatctttgcctttattgaaaaatcagtcgcagagagatatccagacaatgacttcctcagcaaggaatttgaaatcagaatccccggatttaagtctccttacaaagtaaacgttttcaggatggtttacaggagtggctttgtcgttacaaccaccgtgatatcgatgctgatgccgttttttaacgacgtggtcgggatcttaggggcgttagggttttggcccttgacggtttattttccggtggagatgtatattaagcagaggaaggttgagaaatggagcacgagatgggtgtgtttacagatgcttagtgttgcttgtcttgtgatctcggtggtcgccggggttggatcaatcgccggagtgatgcttgatcttaaggtctataagccattcaagtctacatattga

SEQ ID NO: 2 - protein, 487 aa, Arabidopsis thaliana
  misc_feature (1)..(487): sequence number 12707592
MGETAAANNHRHHHHHGHQVFDVASHDFVPPQPAFKCFDDDGRLKRTGTVWTASAHIITAVIGSGVLSLAWAIAQLGWIAGPAVMLLFSLVTLYSSTLLSDCYRTGDAVSGKRNYTYMDAVRSILGGFKFKICGLIQYLNLFGIAIGYTIQAIKRSNCFHKSGGKDPCHMSSNPYMIVFGVAEILLSQVPDFDQIWWISIVAAVMSFTYSAIGLALGIVQVAANGVFKGSLTGISIGTVTQTQKIWRTFQALGDIAFAYSYSVVLIEIQDTVRSPPAESKTMKKATKISIAVTTIFYMLCGSMGYAAFGDAAPGNLLTGFGFYNPFWLLDIANAAIVVHLVGAYQVFAQPIFAFIEKSVAERYPDNDFLSKEFEIRIPGFKSPYKVNVFRMVYRSGFVVTTTVISMLMPFFNDVVGILGALGFWPLTVYFPVEMYIKQRKVEKWSTRWVCLQMLSVACLVISVVAGVGSIAGVMLDLKVYKPFKSTY

SEQ ID NO: 3 - DNA, 1135 bp, Arabidopsis thaliana
  misc_feature (1)..(1135): sequence number 12385295
acctttaattttttcaccaattggatttggatctgtcaaaaatattggcctctttctctctttctctcttgctctctttctttgttgggttgatctcttcttccatggcgatccctacggagacacaacaccaggagaaggaggcttcagatgcttctgcagcagctgcacaaaagagatggactttaagcgatttcgacatcggtaagcctcttggcagaggcaaattcggtcacgtctatctcgccagagaaaaacggagcaatcacgttgtcgctctaaaggttcttttcaagagccagcttcaacaatcccaagttgaacatcagctcagaagagaagttgagattcagtctcatcttcgtcaccccaacatactccggctttatggctatttctatgatcaaaaaagagtttatttgatacttgagtatgctgctagaggcgaactttacaaggatcttcagaaatgcaaatacttcagcgaaagacgagctgctacttatgttgcctcattggcgagggctctcatctattgccatggcaagcatgtgatacacagagatattaaaccagagaatctgctaattggtgctcagggtgagctcaagattgcagactttggttggtcggtacacacatttaaccgaagaaggaccatgtgtggcacactagattaccttcctcctgagatggtcgaaagcgtagaacatgatgctagtgtagatatctggagccttgggattctctgttacgagtttctttatggtgtacctccttttgaagccatggagcactcagacacatacagaaggattgtgcaagtggatctcaagttccctcccaaaccaataatctctgcatctgcaaaggatcttattagccagatgcttgtcaaggagtcttcgcaacgtctgccattgcacaagcttctggagcatccgtggatcgtgcaaaacgctgatccttctggaatctacagagtttaaaaacaaaacgcttactgttctcgccaatctcgaccaaaactgttatctagaaacccaaaactgtattctttcttactattatttttcgctaattcatgttccaaataatgtgtattatgtagtatgtactaatactatatggcatt

SEQ ID NO: 4 - protein, 294 aa, Arabidopsis thaliana
  misc_feature (1)..(294): sequence number 12385296
MAIPTETQHQEKEASDASAAAAQKRWTLSDFDIGKPLGRGKFGHVYLAREKRSNHVVALKVLFKSQLQQSQVEHQLRREVEIQSHLRHPNILRLYGYFYDQKRVYLILEYAARGELYKDLQKCKYFSERRAATYVASLARALIYCHGKHVIHRDIKPENLLIGAQGELKIADFGWSVHTFNRRRTMCGTLDYLPPEMVESVEHDASVDIWSLGILCYEFLYGVPPFEAMEHSDTYRRIVQVDLKFPPKPIISASAKDLISQMLVKESSQRLPLHKLLEHPWIVQNADPSGIYRV

SEQ ID NO: 5 - DNA, 1135 bp, Arabidopsis thaliana; identical to SEQ ID NO: 3
  misc_feature (1)..(1135): sequence number 12385295

SEQ ID NO: 6 - protein, 294 aa, Arabidopsis thaliana; identical to SEQ ID NO: 4
  misc_feature (1)..(294): sequence number 12385296

SEQ ID NO: 7 - DNA, 966 bp, Arabidopsis thaliana
  misc_feature (1)..(966): sequence number 12688873
atgtcgtcgaagaaatccctagttcaaagtctcttcaacatttccaaaacctactccaggatttctggtcttacccgaatgcgtccgaccaaatccggcggcattcctcccgacgccggagattctggaatccgccgcagatttctccacaagagggcatttttctcgccggagatagttcctaaaggtggtaacttgatggagaaactcagggaattgactttgtccaataataatcgtattagactcgacgagatgttaccgccaccttcgccgaagaaatcatcaccggagtttttcccggcggtcacggtggaagacgtgaagaagctcatgagagcagcggaaatggagctggtgaaatcgaagctgagagagattggcaagaactgggttccttattcggagtttgttcgggtctgcggagaatacagttcggatcctgaacaaggtaaccgggtcgcgaatatgcttgacgaagctggaaacgtcatcgttttgggaaaactcgtctgccttaaacccgaagagctaacaagcgccatggctggtctgattccgacactcgaacccagtctcgacgccgagacaagacaagagttcgaacaacttgagatcataaaatcagatatcgacaaaagagccgatgatctggttcgaaaagaattatgggccggattaggcctaataatggcccaaacagttggattttttaggctgacgttttgggaactgtcgtgggacgtgatggaacccatatgcttctacgtaacttcgacatatttcatggctggttacgccttcttcctccgaacttcaaaggaaccttcctttgaaggtttttacaaaagccggttcgagacgaagcagaaacgtttgattaaaatgcttgatttcgatatcgatcgatttaccaagctacagaagatgcatcgtccaaatttgactaaatctggtcgttgttga

SEQ ID NO: 8 - protein, 321 aa, Arabidopsis thaliana
  misc_feature (1)..(321): sequence number 12688874
MSSKKSLVQSLFNISKTYSRISGLTRMRPTKSGGIPPDAGDSGIRRRFLHKRAFFSPEIVPKGGNLMEKLRELTLSNNNRIRLDEMLPPPSPKKSSPEFFPAVTVEDVKKLMRAAEMELVKSKLREIGKNWVPYSEFVRVCGEYSSDPEQGNRVANMLDEAGNVIVLGKLVCLKPEELTSAMAGLIPTLEPSLDAETRQEFEQLEIIKSDIDKRADDLVRKELWAGLGLIMAQTVGFFRLTFWELSWDVMEPICFYVTSTYFMAGYAFFLRTSKEPSFEGFYKSRFETKQKRLIKMLDFDIDRFTKLQKMHRPNLTKSGRC

SEQ ID NO: 9 - DNA, 1278 bp, Arabidopsis thaliana
  misc_feature (1)..(1278): sequence number 13617271
cacaaggctagggatcgaagaagcggcgatcactgatcgtatctcactacgatcacattaatggatagaatgtgtggtttccgctcgacggaagactattcggagaaagcgacgttgatgatgccgtccgattatcagtctttgatttgttcaaccaccggagacaatcaaagactgtttggatccgacgaactcgctaccgctttgtcctcggagttgcttccgcgtattcgaaaagctgaggataatttctctcttagtgtcatcaaatccaaaatcgcttctcatcctttgtatcctcgcttactccaaacctacatcgattgccaaaaggtgggagcgcctatggaaatagcgtgtatattggaagagattcagcgagagaaccatgtgtacaagagagatgttgctccattatcttgctttggagctgatcctgagcttgatgaattcatggaaacctactgtgatatattggttaaatacaaaaccgatcttgcgaggccgttcgacgaggctacaactttcataaacaagattgaaatgcagcttcagaacttgtgcactggtccagcgtctgctacagctctttcagatgatggtgcggtttcatctgacgaggaactgagagaagatgatgacatagcagcggatgacagccaacaaagaagcaatgaccgcgatctgaaggaccagctactacgcaaatttggtagccatatcagttcattgaaactcgagttctctaaaaagaagaagaaagggaagctaccaagagaagcaagacaagcgttgctcgattggtggaatgttcataataaatggccttaccctactgaaggcgacaaaatatctctggctgaagaaacaggtttggatcaaaaacaaatcaacaattggtttataaaccaaaggaaacgccattggaagccttcggagaacatgccgtttgatatgatggacgattctaatgaaacattctttaccgagtaatgaaaagagagacatgaaattgtgcattgtataatttttacactgtttttccaagaaaagaaaacagtaaaaagcttttggtaaatggggcatcatcgcgaatgaatggaacccgttagccaaaacggtcaagggcgtaacgagacattgtattggaaatagtggcaatattatgtcactaatcttccaatcatccaaattgatagatttcttatttgtattgaaccttacttagatagctgatgtgtcaactaaataatttattttcattatccattcgggtag

SEQ ID NO: 10 - protein, 310 aa, Arabidopsis thaliana
  misc_feature (1)..(310): sequence number 13617272
MDRMCGFRSTEDYSEKATLMMPSDYQSLICSTTGDNQRLFGSDELATALSSELLPRIRKAEDNFSLSVIKSKIASHPLYPRLLQTYIDCQKVGAPMEIACILEEIQRENHVYKRDVAPLSCFGADPELDEFMETYCDILVKYKTDLARPFDEATTFINKIEMQLQNLCTGPASATALSDDGAVSSDEELREDDDIAADDSQQRSNDRDLKDQLLRKFGSHISSLKLEFSKKKKKGKLPREARQALLDWWNVHNKWPYPTEGDKISLAEETGLDQKQINNWFINQRKRHWKPSENMPFDMMDDSNETFFTE

SEQ ID NO: 11 - DNA, 1313 bp, Arabidopsis thaliana
  misc_feature (1)..(1313): sequence number 13619728
aatcttgaaaagtgtttttgagagaaatataggttttacaaaatccaccgttgtgaattcatggaaatggtaaacgcagaagcaaaacagagtgtccctcttctcaccccttataagatgggaagattcaatctttctcatagggttgttctagcaccattgacgagacagaaatcgtacggaagcgttcctcagcctcacgctatcttgtattactctcagagaacgtcaccgggaggttttctcatcgctgaagccaccggggtttcagatacagctcaagggtatccagatacacctgggatatggactaaagagcatgtggaggcatggaagccaatcgttgatgctgtacatgccaaaggtggtatcttcttctgtcagatctggcatgttggccgcgtttctaatcgcggttttcagccaaggaggcaagctcctatctcttgtacggggaagccaattatgcctcaaatgcgtgctaatggcattgatgaagctcgctttacccctccaagacgtctaagtatcgaagaaatccccggcattgtcaatgattttaggcttgctgcaagaaatgctatggaagctggttttgatggagttgagattcatggagctcatggctatctgattgatcagttcatgaaggacaaagtgaatgacagaactgatgaatatggtggatcattgcaaaaccgttgcaaatttgctctggaagtagtcgatgcagtggctaaggagatcgggccagaccgtgttggaatcaggctctctccgtttgcagactatatggaatccggagacactaatccagaagcattagggctgtacatggtggaatctctgaacaaatatggaatcctctactgtcatatgattgaacccagaatgaaaacagtgggagaaatagcagcgtgttctcacacactaatgccaatgagggaagcctttaaggggactttcatctctgcaggaggtttcacgagggaagatgggaatgaggccgtggcaaagggacgaactgatcttgtggcttatggtcgatggtttctagccaacccagacctgccaaagcggttccaactggatgcaccgctgaataaatacaataggtcaacgttttacacttctgatcctgtcgtgggttacaccgattacccttcccttgaatcaacagcttaaaatcgtgttatcagtaatgtaatgtgtttccctaatgatgtaataagtttctggcttttgtttatactctaagtcattataccttcataataatttacatggatacattatcacaaaagagcttttat

SEQ ID NO: 12 - protein, 374 aa, Arabidopsis thaliana
  misc_feature (1)..(374): sequence number 13619729
MEMVNAEAKQSVPLLTPYKMGRFNLSHRVVLAPLTRQKSYGSVPQPHAILYYSQRTSPGGFLIAEATGVSDTAQGYPDTPGIWTKEHVEAWKPIVDAVHAKGGIFFCQIWHVGRVSNRGFQPRRQAPISCTGKPIMPQMRANGIDEARFTPPRRLSIEEIPGIVNDFRLAARNAMEAGFDGVEIHGAHGYLIDQFMKDKVNDRTDEYGGSLQNRCKFALEVVDAVAKEIGPDRVGIRLSPFADYMESGDTNPEALGLYMVESLNKYGILYCHMIEPRMKTVGEIAACSHTLMPMREAFKGTFISAGGFTREDGNEAVAKGRTDLVAYGRWFLANPDLPKRFQLDAPLNKYNRSTFYTSDPVVGYTDYPSLESTA

SEQ ID NO: 13 - DNA, 966 bp, Arabidopsis thaliana; identical to SEQ ID NO: 7
  misc_feature (1)..(966): sequence number 12688873

SEQ ID NO: 14 - protein, 321 aa, Arabidopsis thaliana; identical to SEQ ID NO: 8
  misc_feature (1)..(321): sequence number 12688874

SEQ ID NO: 15 - DNA, 1342 bp, Arabidopsis thaliana
  misc_feature (1)..(1342): sequence number 13603177
gtataaagacgacaaagtaaaccaaaaaaaaaaagagttctcctacaattttcctaaattcttggatttgagatttcactttttccgatttgaaacaatgatgataactcgcggtggagccaaggcggcgaaatcgctgttagtggcggctggaccacgtttgttctcgacggtccgtacggtttcgtctcacgaggctttatcagcaagccatattttgaagcctggtgttacatctgcttggatatggactagagctccgacgattggaggtatgagattcgctagcacgatcactctgggagagaaaactccgatgaaggaggaggacgcgaatcagaagaaaacagagaacgaatccaccggtggagacgccgccggaggtaataacaagggagataaaggaatcgcgagctattggggtgttgaacctaataagattactaaagaagatggttctgaatggaagtggaactgtttcaggccatgggaaacgtataaagctgatataacgatagatctgaagaagcatcatgttccaacgacgtttcttgatagaatagcttattggactgttaaatctcttcgttggcctaccgatttgttcttccagaggagatatggatgtcgagctatgatgcttgaaactgtagcagcagtacctggaatggttggaggaatgttactacactgcaaatcgcttcgacgttttgagcaaagtggaggatggattaaggctcttcttgaggaagcagagaatgagagaatgcatcttatgacattcatggaagtcgcgaaaccgaaatggtacgagagagcgctcgtgatcactgtgcaaggagtcttcttcaacgcttatttccttggttacttaatctctcccaagtttgctcatcgtatggttgggtaccttgaagaagaagcgatccattcttatactgagtttctcaaggaacttgacaaaggtaacattgagaatgttcctgctccggctattgctattgattactggaggcttcctgctgatgcgacacttcgtgatgttgtgatggttgttcgtgctgacgaggctcatcaccgtgatgtaaaccattttgcatctgatattcactaccaaggtcgtgaactaaaggaagctccagctccaattgggtatcattgattcgattaaaagaagagctttttctcaagtttaaaactttgttctaaagaatttaagttctttgacttgtatatacatcatcacctctgcttaagccatacttggattcggctttctttgaatgttgctacgaatgttctgatttcttctttacttttcctgtcaatgggcttttgggct

SEQ ID NO: 16 - protein, 354 aa, Arabidopsis thaliana
  misc_feature (1)..(354): sequence number 13603179
MMITRGGAKAAKSLLVAAGPRLFSTVRTVSSHEALSASHILKPGVTSAWIWTRAPTIGGMRFASTITLGEKTPMKEEDANQKKTENESTGGDAAGGNNKGDKGIASYWGVEPNKITKEDGSEWKWNCFRPWETYKADITIDLKKHHVPTTFLDRIAYWTVKSLRWPTDLFFQRRYGCRAMMLETVAAVPGMVGGMLLHCKSLRRFEQSGGWIKALLEEAENERMHLMTFMEVAKPKWYERALVITVQGVFFNAYFLGYLISPKFAHRMVGYLEEEAIHSYTEFLKELDKGNIENVPAPAIAIDYWRLPADATLRDVVMVVRADEAHHRDVNHFASDIHYQGRELKEAPAPIGYH

SEQ ID NO: 17 - DNA, 1418 bp, Arabidopsis thaliana
  misc_feature (1)..(1418): sequence number 12371508
attccactcccactaaacattccttctctcgctcactcttctccaatccttattttattttttgaaagtttaaaattttatacaacatatcaatttggggtagaaaaattcgaaagaaatgaaagagatgggagtgatagtgcttcttctccttcactcgttcttctacgttgccttttgcttcaatgatggactactaccaaacggtgacttcgaactcggtccacgacattcggacatgaaaggaacacaagttatcaacataacagcaatcccaaactgggaactctcaggctttgtcgagtacattccctcaggacacaaacaaggcgacatgatccttgtcgtgcctaaaggcgcattcgcagtacgtctaggcaacgaagcctcaatcaaacaaaaaatcagcgttaagaaagggtcgtactattcgataacgttcagtgctgctcgaacatgcgcacaagacgagcggttaaacgtttccgtggctcctcaccatgcagtgatgccgatacaaacagtgtatagtagctcaggttgggatttgtattcgtgggcttttaaggcccaaagtgactatgcagatatagtgatacataatccaggtgttgaggaagatcctgcttgtggacctctcattgatggtgttgctatgcgagcccttttccctcctcgtcccaccaataagaacattctaaagaacggaggattcgaagaaggtccttgggttttaccaaacatatcatctggtgttttgattccaccaaactccatcgacgatcactctccgttacctggttggatggtcgagtctcttaaagctgtcaaatacatagattccgatcatttctccgttcctcaaggccgtcgcgccgtcgaactcgtcgccgggaaagaaagcgccgtcgcacaagttgtccgcactatccctggaaaaacctacgtcctatccttctctgtcggagatgctagcaacgcttgcgccggatcaatgatcgtcgaagctttcgccggaaaagacacgatcaaggtcccgtatgaatcgaaagggaaaggaggattcaagcgatcgtcattgagattcgtcgctgtctcgagtcggactagagttatgttctacagtacgttttacgcgatgagaaacgacgatttctcgagcttatgtggaccggtgatcgacgacgttaagcttctcagtgctcggaggccgtgagcttgcggcgacgagttgattcacgggacaatgaatgatgacagtcactgtgggtttctcgcgtctagtgagaaattgggcttttaggcccagtggcccactgtttttgttgttgttttaaagcttaatgttatttgacaaagaaaaaagaaattactctggtcaatcatatcgaaccgtgaaattttatgatcttgtgatt

SEQ ID NO: 18 - protein, 365 aa, Arabidopsis thaliana
  misc_feature (1)..(365): sequence number 12371509
MKEMGVIVLLLLHSFFYVAFCFNDGLLPNGDFELGPRHSDMKGTQVINITAIPNWELSGFVEYIPSGHKQGDMILVVPKGAFAVRLGNEASIKQKISVKKGSYYSITFSAARTCAQDERLNVSVAPHHAVMPIQTVYSSSGWDLYSWAFKAQSDYADIVIHNPGVEEDPACGPLIDGVAMRALFPPRPTNKNILKNGGFEEGPWVLPNISSGVLIPPNSIDDHSPLPGWMVESLKAVKYIDSDHFSVPQGRRAVELVAGKESAVAQVVRTIPGKTYVLSFSVGDASNACAGSMIVEAFAGKDTIKVPYESKGKGGFKRSSLRFVAVSSRTRVMFYSTFYAMRNDDFSSLCGPVIDDVKLLSARRP

SEQ ID NO: 19 - DNA, 2880 bp, Arabidopsis thaliana
  misc_feature (1)..(2880): sequence number 12699286
atgggtaaagagaagtttcacatcaacattgtggtcattggccacgtcgattctggaaagtcgaccaccactgggcacttgatctacaagttgggtggtattgacaagcgtgtcattgagaggttcgagaaggaggctgctgagatgaacaagaggtccttcaagtacgcatgggttttggacaaacttaaggctgagcgtgagcgtggtatcaccattgacattgctctctggaagttcgagaccaccaagtactactgcactgtcattgatgctcctggccatcgtgatttcatcaagaacatgatcactggtacctcccaggctgattgtgctgtccttatcattgactccaccactggtggttttgaggctggtatctccaaggatggtcagacccgtgagcacgctctacttgctttcacccttggtgtcaagcagatgatctgctgttgtaacaagatggatgccactacccccaagtactccaaggccaggtacgatgaaatcatcaaggaggtgtcttcctacttgaagaaggttggttacaaccccgacaaaatcccatttgtgcccatctctggatttgagggtgacaacatgattgagaggtccaccaaccttgactggtacaagggaccaactctccttgaggctcttgaccagatcaacgagcccaagaggccgtcagacaagccccttcgtctcccacttcaggatgtctacaagattggtggtattggaacggtgccagtgggacgtgttgagactggtatgatcaagcctggtatggttgtgacctttgctcccacaggattgaccactgaggtcaagtctgttgagatgcaccacgagtctcttcttgaggcacttccaggtgacaacgttgggttcaatgttaagaatgttgctgtcaaggatcttaagagagggtacgtcgcatccaactccaaggatgaccctgccaagggtgctgctaacttcacctcccaggtcatcatcatgaaccaccctggtcagattggtaacggttacgccccagtcctggattgccacacctctcacattgcagtcaagttctctgagatcttgaccaagattgacaggcgttctggtaaggagattgagaaggagcccaagttcttgaagaatggtgatgctggtatggtgaagatgactccaaccaagcccatggttgtggagaccttctctgagtacccaccacttggacgtttcgctgtgagggacatgaggcagactgttgcagtcggtgttatcaagagtgttgacaagaaggacccaaccggagccaaggttaccaaggctgccgtcaagaagggtgaatcaaaggacagtgttagttttattacaatagtttggtatttggtcgcgtgtctgtgttcttgtttcgttttctccccgtcagagcgttgttctcgtaattgggttcttgatcggaggtggcggatctacacacacattcttcctccgcatcatccttctcccttgtgcgatatcgtttgcctaaccatgggtaaagagaagtttcacatcaacattgtggtcattggccacgtcgattctggaaagtcgacaaccactggacacttgatctacaagttgggtggtattgacaagcgtgtgatcgagaggttcgagaaggaggctgctgagatgaacaagaggtccttcaagtacgcatgggtgttggacaaacttaaggctgagcgtgagcgtggtatcaccattgacattgctctctggaagttcgagaccaccaagtactactgcactgtcattgatgctcctggtcatcgtgatttcatcaagaacatgatcactggtacctcccaggctgattgtgctgtccttatcattgactccaccactggtggttttgaggctggtatctccaaggatggtcagacccgtgagcacgctctccttgctttcacccttggtgtcaagcagatgatctgctgttgtaacaagatggatgccactacccccaagtactccaaggccaggtacgatgaaatcatcaaggaggtgtcttcctacttgaagaaggttggttacaaccccgacaaaatcccatttgtgcccatctctggatttgagggtgacaacatgattgagaggtccaccaaccttgactggtacaagggaccaactctccttgaggctcttgaccagatcaacgagcccaagaggccgtcagacaagccccttcgtctcccacttcaggatgtctacaagattggtggtattggaacggtgccagtgggacgtgttgagactggtatgatcaagcctggtatggttgtgacctttgctcccacaggattgaccactgaggtcaagtctgttgagatgcaccacgagtctcttcttgaggcacttccaggtgacaacgttgggttcaatgttaagaatgttgccgtgaaggatcttaagagagggtacgtcgcctccaactccaaggatgaccctgccaagggtgctgctaacttcacctcccaggtcatcatcatgaaccaccctggtcagattggtaacggttacgccccagtcttggattgccacacctctcacattgcagtcaagttctctgagatcttgaccaagattgacaggcgttctggtaaggagattgagaaggagcccaaattcttgaagaatggtgatgctggtatggtgaagatgactccaaccaagcccatggttgtggagaccttctctgagtacccaccacttggacgtttcgctgttagggacatgaggcagactgttgcagtcggtgttatcaagagtgttgacaagaaggacccaaccggagccaaggttaccaaggctgcagttaagaagggtgcaaagtga

SEQ ID NO: 20 - protein, 959 aa, Arabidopsis thaliana
  misc_feature (1)..(959): sequence number 12699287
MGKEKFHINIVVIGHVDSGKSTTTGHLIYKLGGIDKRVIERFEKEAAEMNKRSFKYAWVLDKLKAERERGITIDIALWKFETTKYYCTVIDAPGHRDFIKNMITGTSQADCAVLIIDSTTGGFEAGISKDGQTREHALLAFTLGVKQMICCCNKMDATTPKYSKARYDEIIKEVSSYLKKVGYNPDKIPFVPISGFEGDNMIERSTNLDWYKGPTLLEALDQINEPKRPSDKPLRLPLQDVYKIGGIGTVPVGRVETGMIKPGMVVTFAPTGLTTEVKSVEMHHESLLEALPGDNVGFNVKNVAVKDLKRGYVASNSKDDPAKGAANFTSQVIIMNHPGQIGNGYAPVLDCHTSHIAVKFSEILTKIDRRSGKEIEKEPKFLKNGDAGMVKMTPTKPMVVETFSEYPPLGRFAVRDMRQTVAVGVIKSVDKKDPTGAKVTKAAVKKGESKDSVSFITIVWYLVACLCSCFVFSPSERCSRNWVLDRRWRIYTHILPPHHPSPLCDIVCLTMGKEKFHINIVVIGHVDSGKSTTTGHLIYKLGGIDKRVIERFEKEAAEMNKRSFKYAWVLDKLKAERERGITIDIALWKFETTKYYCTVIDAPGHRDFIKNMITGTSQADCAVLIIDSTTGGFEAGISKDGQTREHALLAFTLGVKQMICCCNKMDATTPKYSKARYDEIIKEVSSYLKKVGYNPDKIPFVPISGFEGDNMIERSTNLDWYKGPTLLEALDQINEPKRPSDKPLRLPLQDVYKIGGIGTVPVGRVETGMIKPGMVVTFAPTGLTTEVKSVEMHHESLLEALPGDNVGFNVKNVAVKDLKRGYVASNSKDDPAKGAANFTSQVIIMNHPGQIGNGYAPVLDCHTSHIAVKFSEILTKIDRRSGKEIEKEPKFLKNGDAGMVKMTPTKPMVVETFSEYPPLGRFAVRDMRQTVAVGVIKSVDKKDPTGAKVTKAAVKKGAK

SEQ ID NO: 21 - DNA, 1500 bp, Arabidopsis thaliana
  misc_feature (1)..(1500): sequence number 6743209
atggcgagttccgttttctctcggttttctatatacttttgtgttcttctattatgccatggttctatggcccagctatttaatcccagcacaaacccatggcatagtcctcggcaaggaagttttagggagtgtagatttgatagactacaagcatttgagccacttcggaaagtgaggtcagaagctggggtgactgagtacttcgatgagaagaatgaattattccagtgcacgggtacttttgtgatccgacgtgtcattcagcctcaaggccttttggtacctcgatacacaaatactcctggcgtggtctacatcatccaagggagaggttctatgggtttaaccttccccggttgccctgcgacttaccagcaacaattccaacaattttcatctcaaggccaaagtcagagccaaaagtttagggatgagcaccaaaagattcatcaatttaggcaaggagacattgttgcactcccagctggtgttgcacattggttctacaatgatggtgatgcacctgttgttgccgtatatgtttatgacgtaaacaacaacgccaatcagcttgaacccaggcaaaaggagttcctattagccggcaacaacaatcgggctcagcaacaacaagtatatggtagctcaattgagcaacactctgggcaaaacatattcagcggattcggtgttgagatgctaagtgagtctttaggcatcaacgcagtagcagcaaagaggctacagagccaaaatgatcaaagaggagagatcatacatgtgaagaatggccttcaattgttgaaaccgactttgacacaacagcaagaacaagcacaagcacaagatcaatatcaacaagttcaatacagtgaacgacagcaaacatcttctcgatggaatggattggaggagaacttctgcacgatcaaggcgagagtaaacattgaaaatcctagtcgtgctgattcatacaacccacgtgccggaaggataacaagtgtcaatagtcagaagttccccatccttaacctcatccaaatgagcgctaccagagtaaacctataccagaatgctattctctcgccgttctggaacgtcaatgctcatagtttggtctatatgattcaagggcgatctcgagttcaagtcgttagtaactttggaaagactgtgtttgatggtgtccttcgcccaggacaattattgatcattccgcaacattatgctgtcttgaagaaagcagagcgtgaaggatgccaatatatcgcaatcaagacaaacgctaacgccttcgtcagccaccttgcagggaaaaactcagtattccgtgccttgccagttgatgtagtcgctaatgcgtatcgcatctcaagggagcaagcccgaagcctcaagaacaacaggggagaagagcacggtgccttcactcctagatttcaacaacaatactacccaggattatcgaatgagtccgaaagcgagacctcagagtaa

SEQ ID NO: 22 - protein, 499 aa, Arabidopsis thaliana
  misc_feature (1)..(499): sequence number 6743210
MASSVFSRFSIYFCVLLLCHGSMAQLFNPSTNPWHSPRQGSFRECRFDRLQAFEPLRKVRSEAGVTEYFDEKNELFQCTGTFVIRRVIQPQGLLVPRYTNTPGVVYIIQGRGSMGLTFPGCPATYQQQFQQFSSQGQSQSQKFRDEHQKIHQFRQGDIVALPAGVAHWFYNDGDAPVVAVYVYDVNNNANQLEPRQKEFLLAGNNNRAQQQQVYGSSIEQHSGQNIFSGFGVEMLSESLGINAVAAKRLQSQNDQRGEIIHVKNGLQLLKPTLTQQQEQAQAQDQYQQVQYSERQQTSSRWNGLEENFCTIKARVNIENPSRADSYNPRAGRITSVNSQKFPILNLIQMSATRVNLYQNAILSPFWNVNAHSLVYMIQGRSRVQVVSNFGKTVFDGVLRPGQLLIIPQHYAVLKKAEREGCQYIAIKTNANAFVSHLAGKNSVFRALPVDVVANAYRISREQARSLKNNRGEEHGAFTPRFQQQYYPGLSNESESETSE

SEQ ID NO: 23 - DNA, 743 bp, Arabidopsis thaliana
  misc_feature (1)..(743): sequence number 12673011
acatctcactgctcactactctcactgtaatcccttagatcttcttttcaaatttcaatggcgtccggtgatgttgagtatcggtgcttcgttggaggtctagcatgggccactgatgacagagctcttgagactgccttcgctcaatacggcgacgttattgattccaagatcattaacgatcgtgagactggaagatcaaggggattcggattcgtcaccttcaaggatgagaaagccatgaaggatgcgattgagggaatgaacggacaagatctcgatggccgtagcatcactgttaacgaggctcagtcacgaggaagcggtggcggcggaggccaccgtggaggtggcggcggtagacgcgagggtggaggaggatacagcggcggcggcggcggttactcctcaagaggtggtggtggcggaagctacggtggtggaagacgtgagggaggaggaggatacggtggtggtgaaggaggaggttacggaggaagcggtggtggtggaggatggtaattcctttaattaggtttgggattaccaatgaatgttctctctctcgcttgttatgcttctacttggttttgtgtgttctctattttgttctggttctgctttagatttgatgtaacagttcgtgattaggtattttggtatctggaaacgtaatgttaagtcacttgtcattctctaaataacaaatttcttcggagatattatctctgttgattgattct

SEQ ID NO: 24 - protein, 154 aa, Arabidopsis thaliana
  misc_feature (1)..(154): sequence number 12673012
MASGDVEYRCFVGGLAWATDDRALETAFAQYGDVIDSKIINDRETGRSRGFGFVTFKDEKAMKDAIEGMNGQDLDGRSITVNEAQSRGSGGGGGHRGGGGGRREGGGGYSGGGGGYSSRGGGGGSYGGGRREGGGGYGGGEGGGYGGSGGGGGW

SEQ ID NO: 25 - DNA, 2255 bp, Arabidopsis thaliana
  misc_feature (1)..(2255): sequence number 12712671
accaaatacaaaccctagccgccttattcgtcttcttcgttctctagttttttcctcagtctctgttcttagatcccttgtagtttccaaatcttccgataaaaatgtcgggtaaaggagaaggaccagctatcggtatcgatcttggtaccacttactcttgcgtcggagtatggcaacacgaccgtgttgagatcattgctaatgatcaaggaaacagaaccacgccatcttacgttgctttcaccgactccgagaggttgatcggtgacgcagctaagaatcaggtcgccatgaaccccgttaacaccgttttcgacgctaagaggttgatcggtcgtcgtttctctgacagctctgttcagagtgacatgaaattgtggccattcaagattcaagccggacctgccgataagccaatgatctacgtcgaatacaagggtgaagagaaagagttcgcagctgaggagatttcttccatggttcttattaagatgcgtgagattgctgaggcttaccttggtgtcacaatcaagaacgccgttgttaccgttccagcttacttcaacgactctcagcgtcaggctacaaaggatgctggtgtcatcgctggtttgaacgttatgcgaatcatcaacgagcctacagccgccgctattgcctacggtcttgacaaaaaggctaccagcgttggagagaagaatgttcttatcttcgatcttggtggtggcacttttgatgtctctcttcttaccattgaagagggtatctttgaggtgaaggcaactgctggtgacacccatcttggtggggaagattttgacaacagaatggttaaccactttgtccaagagttcaagaggaagagtaagaaggatatcaccggtaacccaagagctcttaggaggttgagaacttcctgtgagagagcgaagaggactctttcttccactgctcagaccaccatcgagattgactctctatacgagggtatcgacttctactccaccatcacccgtgctagatttgaggagctcaacatggatctcttcaggaagtgtatggagccagttgagaagtgtcttcgtgatgctaagatggacaagagcactgttcatgatgttgtccttgttggtggttctacccgtatccctaaggttcagcaattgctccaggacttcttcaacggcaaagagctttgcaagtctattaaccctgatgaggctgttgcctacggtgctgctgtccagggagctattctcagcggtgaaggaaacgagaaggttcaagatcttctattgctcgatgtcactcctctctcccttggtttggaaactgccggtggtgtcatgaccactttgatcccaaggaacacaaccatcccaaccaagaaggaacaagtcttctccacctactcagacaaccaacccggtgtgttgatccaggtgtacgaaggagagagagccagaaccaaggacaacaaccttcttggtaaatttgagctctccggaattcctccagctcctcgtggtgtcccccagatcacagtctgctttgacattgatgccaatggtatcctcaatgtctctgctgaggacaagaccaccggacagaagaacaagatcaccatcaccaatgacaagggtcgtctctccaaggatgagattgagaagatggttcaagaggctgagaagtacaagtccgaagacgaggagcacaagaagaaggttgaagccaagaacgctctcgagaactacgcttacaacatgaggaacaccatccaagacgagaagattggtgagaagctcccggctgcagacaagaagaagatcgaggattctattgagcaggcgattcaatggctcgagggtaaccagttggctgaggctgatgagttcgaagacaagatgaaggaattggagagcatctgcaacccaatcattgccaagatgtaccaaggagctggtggtgaagccggtggtccaggtgcctctggtatggacgatgatgctccccctgcttcaggcggtgctggacctaagatcgaggaggtcgactaatttgttggacattgacctctctctttctcctatctctatctcttttacttgcttttttttgatctgttaagactttttatgttgggcttttttaaagaagcccattttgtggtgttttttggttagtactattttgaacaatggttggttctataccagtttagctacgatgacggataaaattaaaagtttgcc

SEQ ID NO: 26 - protein, 651 aa, Arabidopsis thaliana
  misc_feature (1)..(651): sequence number 12712672
MSGKGEGPAIGIDLGTTYSCVGVWQHDRVEIIANDQGNRTTPSYVAFTDSERLIGDAAKNQVAMNPVNTVFDAKRLIGRRFSDSSVQSDMKLWPFKIQAGPADKPMIYVEYKGEEKEFAAEEISSMVLIKMREIAEAYLGVTIKNAVVTVPAYFNDSQRQATKDAGVIAGLNVMRIINEPTAAAIAYGLDKKATSVGEKNVLIFDLGGGTFDVSLLTIEEGIFEVKATAGDTHLGGEDFDNRMVNHFVQEFKRKSKKDITGNPRALRRLRTSCERAKRTLSSTAQTTIEIDSLYEGIDFYSTITRARFEELNMDLFRKCMEPVEKCLRDAKMDKSTVHDVVLVGGSTRIPKVQQLLQDFFNGKELCKSINPDEAVAYGAAVQGAILSGEGNEKVQDLLLLDVTPLSLGLETAGGVMTTLIPRNTTIPTKKEQVFSTYSDNQPGVLIQVYEGERARTKDNNLLGKFELSGIPPAPRGVPQITVCFDIDANGILNVSAEDKTTGQKNKITITNDKGRLSKDEIEKMVQEAEKYKSEDEEHKKKVEAKNALENYAYNMRNTIQDEKIGEKLPAADKKKIEDSIEQAIQWLEGNQLAEADEFEDKMKELESICNPIIAKMYQGAGGEAGGPGASGMDDDAPPASGGAGPKIEEVD

SEQ ID NO: 27 - DNA, 2085 bp, Arabidopsis thaliana
  misc_feature (1)..(2085): sequence number 13604752
aggagtaaagaagtaagaaacgaagccgttttgaagtcatctcttcagatatgtttgttctaattaaaatttccaagtgggaattagtttgtaattgaaggtatgcacgatttttagttacaattttaattcttcttcttcagatccaagaactctcagtctctgcgttcacactctttctttgaatccttcatcatcctaattcatctccaagaactgaatcagaagttgtatttcgctaattcaacttttccagtgtctgtcaattaggttttgattttggaagtagagaagttagaagaagaatggtgagaacgccgcgaagagggcagagatcaaagggaattaaagtgaagcattgtattcagttgactctattgcttggtgttgggatatggctgatttatcaaatgaagcattcacatgagaagaaagctgagtttgaagggacttcaaagattgttgttgatgatattgataatacagttgttaatcttggaaggaaagatcttagaccgcgtattgaggagacgaaagatgtgaaggacgaagtggaagatgaagaagggagcaagaatgaaggaggaggagacgtaagtactgataaggagaatggtgatgagattgtagagagggaggaggaggaaaaggctgtagaagagaataacgagaaggaagctgaaggtaccgggaatgaagagggaaacgaggattcaaacaatggagaaagtgagaaggttgttgatgagagtgaaggtggaaatgagataagtaatgaggaagctagggagatcaattacaagggagatgatgcgtcgagtgaggttatgcatgggacggaggagaagagcaatgaaaaggttgaagttgagggagaaagtaaatctaatagtactgaaaatgtcagtgtccatgaagatgagtcgggtccaaagaatgaagtattggagggttctgttattaaagaagtttctttgaacacaactgagaatggtagtgatgatggtgagcaacaagagacaaagagtgagttggattcaaagactggtgagaagggcttttctgattctaatggtgaattgcctgagactaacctgtcaacttccaatgcaactgaaactacagaatcttctgggagtgatgagtcaggatcgagcgggaaatccactggttatcaacaaacgaaaaacgaagaagatgagaaggaaaaggtacaatcatctgaagaggaaagcaaagtcaaagaatccgggaaaaatgagaaggacgcgtcctcgtcccaagacgaaagtaaagaggaaaaacccgagagaaagaagaaagaagagtcttcgtcccaaggggaaggtaaagaagaagaacccgagaaaagggagaaagaagactcttcatcccaagaggaaagtaaagaggaagaacctgagaacaaagagaaagaagcgtcttcctctcaggaggagaatgagattaaagaaactgagataaaggagaaagaagagtcttcgtcccaagaggggaatgagaacaaagaaacagaaaaaaagtcttccgaatctcagagaaaggaaaacaccaacagtgagaagaaaattgaacaggtggaatctactgattcttcaaacacacagaagggtgacgaacagaaaactgatgaaagcaagagagaatccggcaatgatacttcaaataaggaaacagaggatgatagttcaaaaacagagtcagagaagaaagaggaaaataacagaaatggtgaaacagaggagacccaaaacgaacaagaacagaccaagtccgctttggaaattagtcacactcaagatgttaaggatgctcgaactgatctagaaactcttcctgaaaccagcaatggattgatcagcgacaaagttgctgctgagtgatacttgttaaatgtgtgaagctgtcatgtattgtatgcatattctatacttctcaccaaatgacgggacttaagtcccaatcaaagtagtattgagtttttaatcgtaatcagtacatgtgtatgtatacttttatatctaattctctctgagtctctaataatgaagaagtttttttttt

SEQ ID NO: 28 - protein, 532 aa, Arabidopsis thaliana
  misc_feature (1)..(532): sequence number 13604753
MVRTPRRGQRSKGIKVKHCIQLTLLLGVGIWLIYQMKHSHEKKAEFEGTSKIVVDDIDNTVVNLGRKDLRPRIEETKDVKDEVEDEEGSKNEGGGDVSTDKENGDEIVEREEEEKAVEENNEKEAEGTGNEEGNEDSNNGESEKVVDESEGGNEISNEEAREINYKGDDASSEVMHGTEEKSNEKVEVEGESKSNSTENVSVHEDESGPKNEVLEGSVIKEVSLNTTENGSDDGEQQETKSELDSKTGEKGFSDSNGELPETNLSTSNATETTESSGSDESGSSGKSTGYQQTKNEEDEKEKVQSSEEESKVKESGKNEKDASSSQDESKEEKPERKKKEESSSQGEGKEEEPEKREKEDSSSQEESKEEEPENKEKEASSSQEENEIKETEIKEKEESSSQEGNENKETEKKSSESQRKENTNSEKKIEQVESTDSSNTQKGDEQKTDESKRESGNDTSNKETEDDSSKTESEKKEENNRNGETEETQNEQEQTKSALEISHTQDVKDARTDLETLPETSNGLISDKVAAE

SEQ ID NO: 29 - DNA, 967 bp, Arabidopsis thaliana
  misc_feature (1)..(967): sequence number 12679464
attcacaccggacattttgaaatctcaacaagaaccaaaccaaacaacaaaaaaacattcttaataattatctttctgttatgtcgatgacggcggattctcaatctgattatgcttttcttgagtccatacgacgacacttactaggaggaatcggagccgatactcagtgagtcgacagcgagttcggttactcaatcttgtgtaaccggtcagagcattaaaccggtgtacggacgaaaccctagctttagcaaactgtatccttgcttcaccgagagctggggagatttgccgttgaaagaaaacgattctgaggatatgttagtttacggtatcctcaacgacgcctttcacggcggttgggagccgtcttcttcgtcttccgacgaagatcgtagctctttcccgagtgttaagatcgagactccggagagtttcgcggcggtggattctgttccggtcaagaaggagaagacgagtcctgtttcggcggcggtgacggcggcgaagggaaagcattatagaggagtgagacaaaggccgtgggggaaatttgcggcggagattagagacccggcgaagaacggagctagggtttggttaggaacgtttgagacggcggaggacgcggcgttggcttacgacagagctgctttcaggatgcgtggttcccgcgctttgttgaattttccgttgagagttaattcaggagaacccgacccggttcgaatcaagtccaagagatcttctttttcttcttctaacgagaacggagctccgaagaagaggagaacggtggccgccggtggtggaatggataagggattgacggtgaagtgcgaggttgttgaagtggcacgtggcgatcgtttattggttttataattttgatttttctttgttggatgattatatgattcttcaaaaaagaagaacgttaataaaaaaattcgtttattattat

SEQ ID NO: 30 - protein, 188 aa, Arabidopsis thaliana
  misc_feature (1)..(188): sequence number 12679465
MLVYGILNDAFHGGWEPSSSSSDEDRSSFPSVKIETPESFAAVDSVPVKKEKTSPVSAAVTAAKGKHYRGVRQRPWGKFAAEIRDPAKNGARVWLGTFETAEDAALAYDRAAFRMRGSRALLNFPLRVNSGEPDPVRIKSKRSSFSSSNENGAPKKRRTVAAGGGMDKGLTVKCEVVEVARGDRLLVL

SEQ ID NO: 31 - DNA, 1114 bp, Arabidopsis thaliana
  misc_feature (1)..(1114): sequence number 12719868
aaatcaaatcttcttccttctctgttttcttaagctttttgaaaattttatcaatggcgactcctaacgaagtatctgcactttggttcatcgagaaacatctactcgacgaggcttctcctgtggctacagatccatggatgaagcacgaatcatcatcagcaacagaatctagctctgactcttcttctatcatcttcggatcatcgtcctcttctttcgccccaattgatttctctgaatccgtatgcaaacctgaaatcatcgatctcgatactcccagatctatggaatttctatcgattccatttgaatttgactcagaagtttctgtttctgatttcgattttaaaccttctaatcaaaatcaaaatcagtttgaaccggagcttaaatctcaaattcgtaaaccgccattgaagatttcgcttccagctaaaacagagtggattcaattcgcagctgaaaacaccaaaccggaagttactaaaccggtttcggaagaagagaagaagcattacagaggagtaagacaaagaccgtgggggaaattcgcggcggagattcgtgacccgaataaacgcggatctcgcgtttggcttgggacgtttgatacagcgattgaagcggctagagcttatgacgaagcagcgtttagactacgaggatcgaaagcgattttgaatttccctcttgaagttgggaagtggaaaccacgcgccgatgaaggtgagaagaaacggaagagagacgatgatgagaaagtgactgtggttgagaaagtgttgaagacggaacagagcgttgacgttaacggtggagagacgtttccgtttgtaacgtcgaatttaacggaattatgtgactgggatttaacggggtttcttaactttccgcttctgtcgccgttatctcctcatccaccgtttggttattcccagttgaccgttgtttgattagttttttttgagtttttgaacgatgtgtatgctgacgtggacgtacacgtaggtgcatgcgatgaaaaaaacatctatttgttcatatttttgcgtttttctatttgttcattctttttcacaattcacaatacattatttcagttaatgattac

SEQ ID NO: 32 - protein, 300 aa, Arabidopsis thaliana
  misc_feature (1)..(300): sequence number 12719870
MATPNEVSALWFIEKHLLDEASPVATDPWMKHESSSATESSSDSSSIIFGSSSSSFAPIDFSESVCKPEIIDLDTPRSMEFLSIPFEFDSEVSVSDFDFKPSNQNQNQFEPELKSQIRKPPLKISLPAKTEWIQFAAENTKPEVTKPVSEEEKKHYRGVRQRPWGKFAAEIRDPNKRGSRVWLGTFDTAIEAARAYDEAAFRLRGSKAILNFPLEVGKWKPRADEGEKKRKRDDDEKVTVVEKVLKTEQSVDVNGGETFPFVTSNLTELCDWDLTGFLNFPLLSPLSPHPPFGYSQLTVV

SEQ ID NO: 33 - DNA, 1025 bp, Arabidopsis thaliana
  misc_feature (1)..(1025): sequence number 12370148
attcccacttccacacatacacatatacaacagagcaagagagtcaatcaagtagagtgaagatggcaactaaacaagaagctttagccatcgatttcataagccaacaccttctcacagactttgtttccatggaaactgatcacccatctctttttaccaaccaacttcacaactttcactcagaaacaggccctagaaccatcaccaaccaatcccctaaaccgaattcgactcttaaccagcgtaaaccgcccttaccgaatctatccgtctcgagaacggtttcaacaaagacagagaaagaggaagaagagaggcactacaggggagtgagacgaagaccgtggggaaaatacgcggcggagattagggatccgaacaaaaagggttgtaggatctggcttgggacttacgacactgccgtggaagctggaagagcttatgaccaagcggcgtttcaattacgtggaagaaaagcaatcttgaatttccctctcgatgttagggttacgtcagaaacttgttctggggaaggagttatcggattagggaaacgaaagcgagataagggttctccgccggaagaggagaaggcggctagggttaaagtggaggaagaagagagtaatacgtcggagacgacggaggctgaggttgagccggtggtaccattgacgccgtcaagttggatggggttttgggatgtgggagcaggagatggtattttcagtattcctccgttatctccgacgtctcccaacttttccgttatctccgtcacttaaaacttcggaaaagtcaacgtacgatgacgttttcacttgcgtcactctcatgatttcatttattcttgtataatataaaggtagcggtagtgtgcaaatatcaaataagtagtttaattagtaccaatcattttattcattattttttttagtagaatatttggatgttgaaaatataaatttaattttgtatttgttgatgttataaatttattgattgtataaacattcttagtc

SEQ ID NO: 34 - protein, 241 aa, Arabidopsis thaliana
  misc_feature (1)..(241): sequence number 12370150
MATKQEALAIDFISQHLLTDFVSMETDHPSLFTNQLHNFHSETGPRTITNQSPKPNSTLNQRKPPLPNLSVSRTVSTKTEKEEEERHYRGVRRRPWGKYAAEIRDPNKKGCRIWLGTYDTAVEAGRAYDQAAFQLRGRKAILNFPLDVRVTSETCSGEGVIGLGKRKRDKGSPPEEEKAARVKVEEEESNTSETTEAEVEPVVPLTPSSWMGFWDVGAGDGIFSIPPLSPTSPNFSVISVT

SEQ ID NO: 35 - DNA, 2067 bp, Arabidopsis thaliana
  misc_feature (1)..(2067): sequence number 12560350
atggttaccttatgtgccacgttactgatccttctctcaatctttcttgcaactccgtcgaatgttcgaggaaatgcagagctgaaggctttaatggagctgaaatcgtcgcttgaccctgaaaacaagcttctccgttcatggacgtttaacggcgatccatgcgacggatctttcgaaggaattgcatgtaaccagcatctaaaagtcgcaaacatatcattacaagggaaacgtttggtcggaaaattgtctccggcggttgcagagctcaaatgtttgtcaggtctttacttacattacaatagtctctctggagagatacctcaagagatcacaaatcttactgaattatcagatctttatctcaatgttaataacttctctggtgagattccggcagatatcggctccatggctggcttgcaagttatggatctttgttgcaacagtttaacagggaagataccaaagaacattggatccttgaagaaacttaatgtgttgtctctgcaacacaacaaactaaccggagaggttccttggactttaggaaacttgagtatgttaagcaggcttgatttaagcttcaacaatctgttaggtttaatcccaaaaaccctagccaacattcctcagttggacactcttgacttgcgcaacaatactctctctggctttgttcctcctggtcttaagaagttgaatgggagcttccagtttgagaacaacaccgggttatgtggtatcgattttccttctttgagagcttgttctgctttcgataatgcgaataatatcgaacaatttaagcagcctcctggtgaaatagacactgataaatcagctcttcacaacattcccgagtctgtatatctccaaaagcattgcaaccaaacacattgcaagaaatcctcatcgaaactcccacaagttgctttgatttcaagcgtgattaccgtcactataacattgattggtgctggtatattgaccttctttcgctacagaagaaggaagcaaaagatcagtaacacacctgagttttccgagggaagactaagcacagatcaacaaaaagagttccgtgcatcgcctttagtgagtcttgcctacactaaagaatgggatccgctaggtgatagcagaaacggagctgagttttcacaagagcctcatctctttgttgtaaatagtagcttcaggtttaacttagaagacattgaatcagcaacccaatgcttctcagaagctaatctattgagcagaaacagcttcacctcagtgttcaaaggagtcctcagagatggttctccggtagctatcagaagcatcaacataagcagttgcaagaacgaagaagtcgaattcatgaacggtttaaagcttttatcttcgttgtcacacgaaaacttagtgaagctacgtggattctgctgttctagaggcagaggagagtgtttcctcatctatgattttgcttcaaaaggaaagctttcaaattttcttgacttacaagaacgcgaaactaatctggtccttgcttggtctgcaagaatctccatcatcaaagggattgcaaaaggtattgcttacttacatggaagtgatcaacagaagaagcctacaatagttcatcgaaacatctctgtcgaaaaaatcctacttgatgaacaatttaacccgttaatcgctgattcgggtcttcacaaccttctagcagacgatatggtcttctcagcactcaaaacaagtgcagcaatgggatatttagctccggaatacgtcacaaccggaaaattcaccgagaaaaccgatattttcgcctttggagtcatcattctccagatactctctggtaagctcatgcttacaagttcactgagaaatgcagctgaaaatggagaacataacgggttcatcgatgaagatcttcgtgaagagtttgataaaccagaggcgactgcaatggcgaggattgggataagctgtacacaggagataccaaacaataggcctaatatagagacattgcttgagaatataaactgtatgaagagtgaatga

SEQ ID NO: 36 - protein, 688 aa, Arabidopsis thaliana
  misc_feature (1)..(688): sequence number 12560351
MVTLCATLLILLSIFLATPSNVRGNAELKALMELKSSLDPENKLLRSWTFNGDPCDGSFEGIACNQHLKVANISLQGKRLVGKLSPAVAELKCLSGLYLHYNSLSGEIPQEITNLTELSDLYLNVNNFSGEIPADIGSMAGLQVMDLCCNSLTGKIPKNIGSLKKLNVLSLQHNKLTGEVPWTLGNLSMLSRLDLSFNNLLGLIPKTLANIPQLDTLDLRNNTLSGFVPPGLKKLNGSFQFENNTGLCGIDFPSLRACSAFDNANNIEQFKQPPGEIDTDKSALHNIPESVYLQKHCNQTHCKKSSSKLPQVALISSVITVTITLIGAGILTFFRYRRRKQKISNTPEFSEGRLSTDQQKEFRASPLVSLAYTKEWDPLGDSRNGAEFSQEPHLFVVNSSFRFNLEDIESATQCFSEANLLSRNSFTSVFKGVLRDGSPVAIRSINISSCKNEEVEFMNGLKLLSSLSHENLVKLRGFCCSRGRGECFLIYDFASKGKLSNFLDLQERETNLVLAWSARISIIKGIAKGIAYLHGSDQQKKPTIVHRNISVEKILLDEQFNPLIADSGLHNLLADDMVFSALKTSAAMGYLAPEYVTTGKFTEKTDIFAFGVIILQILSGKLMLTSSLRNAAENGEHNGFIDEDLREEFDKPEATAMARIGISCTQEIPNNRPNIETLLENINCMKSE

SEQ ID NO: 37 - DNA, 1089 bp, Arabidopsis thaliana
  misc_feature (1)..(1089): sequence number 13603142
atactcttcgactcagccgtcctttcgcagaaaccatttggagttggagctttggacgacgacaatggccccgaagaaaggagtgaaggtagctgctaagaagaagaccgcggagaaagtttcaaaccctctattcgagaggaggcctaagcaattcggtattggtggagctttacctcctaagaaggatctctctcgctacatcaaatggcccaaatccatccgtcttcaaaggcaaaagaggatcctgaagcagaggttgaaggtccctccagctcttaaccaattcaccaagactcttgacaagaatcttgctaccagcctcttcaaggtccttctgaagtacaggccagaagacaaagctgccaagaaggagcgtcttgtaaagaaggcccaagctgaagctgagggaaagccttctgagtctaagaagcccattgtagtcaaatacggcctcaaccatgtgacctacctcattgagcagaacaaggcccaacttgttgttattgctcatgatgtcgacccaattgagttggttgtctggttgcctgctctgtgcaggaagatggaagtcccgtactgcattgtcaagggcaaatctcgtcttggagcggttgttcaccagaagactgcttcttgcttgtgtttgaccactgtcaagaacgaggacaagctagagttcagcaaaatcctggaagctatcaaggccaacttcaatgacaagtacgaggagtacaggaagaaatggggaggaggcataatgggatctaagtctcaggcaaagaccaaggcaaaggaaagagttattgcaaaggaggctgcccaaaggatgaattaagaggctagcttcttttgtttgtggtttgctcggattcgtaaaacttaatagagctttttgtttagctggttcttgaagtacctcttttttagttgaaccctttattatggatgttttgcaatttcttgggacagtttcaatgttattcaagctgctggatcctctttggtctctcaaccacttaaaacttgacagtgtgaaattttagccaaatgcttattgcattctggaaagagttataagtttaattc

SEQ ID NO: 38 - protein, 257 aa, Arabidopsis thaliana
  misc_feature (1)..(257): sequence number 13603144
MAPKKGVKVAAKKKTAEKVSNPLFERRPKQFGIGGALPPKKDLSRYIKWPKSIRLQRQKRILKQRLKVPPALNQFTKTLDKNLATSLFKVLLKYRPEDKAAKKERLVKKAQAEAEGKPSESKKPIVVKYGLNHVTYLIEQNKAQLVVIAHDVDPIELVVWLPALCRKMEVPYCIVKGKSRLGAVVHQKTASCLCLTTVKNEDKLEFSKILEAIKANFNDKYEEYRKKWGGGIMGSKSQAKTKAKERVIAKEAAQRMN

SEQ ID NO: 39 - DNA, 1800 bp, Arabidopsis thaliana
  misc_feature (1)..(1800): sequence number 13608279
aatcatggcggagaaactcagtgatggcagcagcatcatctcagtccatcctagaccctccaagggtttctcctcgaagcttctcgatcttctcgagagacttgttgtcaagctcatgcacgatgcttctctccctctccactacctctcaggcaacttcgctcccatccgtgatgaaactcctcccgtcaaggatctccccgtccatggatttcttcccgaatgcttgaatggtgaatttgtgagggttggtccaaaccccaagtttgatgctgtcgctggatatcactggtttgatggagatgggatgattcatggggtacgcatcaaagatgggaaagctacttatgtttctcgatatgttaagacatcacgtcttaagcaggaagagttcttcggagctgccaaattcatgaagattggtgaccttaaggggtttttcggattgctaatggtcaatgtccaacagctgagaacgaagctcaaaatattggacaacacttatggaaatggaactgccaatacagcactcgtatatcaccatggaaaacttctagcattacaggaggcagataagccgtacgtcatcaaagttttggaagatggagacctgcaaactcttggtataatagattatgacaagagattgacccactccttcactgctcacccaaaagttgacccggttacgggtgaaatgtttacattcggctattcgcatacgccaccttatctcacatacagagttatctcgaaagatggcattatgcatgacccagtcccaattactatatcagagcctatcatgatgcatgattttgctattactgagacttatgcaatcttcatggatcttcctatgcacttcaggccaaaggaaatggtgaaagagaagaaaatgatatactcatttgatcccacaaaaaaggctcgttttggtgttcttccacgctatgccaaggatgaacttatgattagatggtttgagcttcccaactgctttattttccacaacgccaatgcttgggaagaagaggatgaagtcgtcctcatcacttgtcgtcttgagaatccagatcttgacatggtcagtgggaaagtgaaagaaaaactcgaaaattttggcaacgaactgtacgaaatgagattcaacatgaaaacgggctcagcttctcaaaaaaaactatccgcatctgcggttgatttccccagaatcaatgagtgctacaccggaaagaaacagagatacgtatatggaacaattctggacagtatcgcaaaggttaccggaatcatcaagtttgatctgcatgcagaagctgagacagggaaaagaatgctggaagtaggaggtaatatcaaaggaatatatgacctgggagaaggcagatatggttcagaggctatctatgttccgcgtgagacagcagaagaagacgacggttacttgatattctttgttcatgatgaaaacacagggaaatcatgcgtgactgtgatagacgcaaaaacaatgtcggctgaaccggtggcagtggtggagctgccgcacagggtcccatatggcttccatgccttgtttgttacagaggaacaactccaggaacaaactcttatataagccaagctgtgtgcatatacacataatacttctgagtcagagatgtgaaactcggtgatactgaattactgatattaccattacacacatattatggcgttatgtattcataatactgcctggtttctctttacatataattacatttcatcctccaatatcaacaaggtctctacttt

SEQ ID NO: 40 - protein, 538 aa, Arabidopsis thaliana
  misc_feature (1)..(538): sequence number 13608281
MAEKLSDGSSIISVHPRPSKGFSSKLLDLLERLVVKLMHDASLPLHYLSGNFAPIRDETPPVKDLPVHGFLPECLNGEFVRVGPNPKFDAVAGYHWFDGDGMIHGVRIKDGKATYVSRYVKTSRLKQEEFFGAAKFMKIGDLKGFFGLLMVNVQQLRTKLKILDNTYGNGTANTALVYHHGKLLALQEADKPYVIKVLEDGDLQTLGIIDYDKRLTHSFTAHPKVDPVTGEMFTFGYSHTPPYLTYRVISKDGIMHDPVPITISEPIMMHDFAITETYAIFMDLPMHFRPKEMVKEKKMIYSFDPTKKARFGVLPRYAKDELMIRWFELPNCFIFHNANAWEEEDEVVLITCRLENPDLDMVSGKVKEKLENFGNELYEMRFNMKTGSASQKKLSASAVDFPRINECYTGKKQRYVYGTILDSIAKVTGIIKFDLHAEAETGKRMLEVGGNIKGIYDLGEGRYGSEAIYVPRETAEEDDGYLIFFVHDENTGKSCVTVIDAKTMSAEPVAVVELPHRVPYGFHALFVTEEQLQEQTLI

SEQ ID NO: 41 - DNA, 691 bp, Arabidopsis thaliana
  misc_feature (1)..(691): sequence number 12704782
atcagaaatcgaaaaatcaaagttctcaagaagatatcaacaaaaaaaaaagtaaattctttaaaatgtcgatgattccaagtttcttcaacaacaacagacgaagcaacatctttgatccattctctcttgacgtatgggatccattcaaggaactaacatcatcatcactttctcgtgagaactcagcgatcgtgaacgcacgtgtggactggagagagacgcctgaggcccacgtgtttaaagctgacttgcctggattgaagaaggaggaagttaaagttgagattgaggaggatagtgttttgaagatcagtggagagagacacgtggagaaagaagataagaatgacacgtggcaccgtgtggagagatcgagtggacagtttacgaggaggtttaggttgccggagaatgtgaagatggatcaggttaaggctgcgatggagaatggtgtgttgactgttacggtgcctaaggctgagactaagaaggctgatgttaagtctattcagatctctggttgagtaatgggttcgagttttatcatcggagttgcttgtgtttttgtcatggttatggttcatgttttacttgagtgtgtgagtactctatctaaattataataatctccgattgagctatgaattatgatgtatcggatacatttgatcctaatgaagtatggaat

SEQ ID NO: 42 - protein, 153 aa, Arabidopsis thaliana
  misc_feature (1)..(153): sequence number 12704784
MSMIPSFFNNNRRSNIFDPFSLDVWDPFKELTSSSLSRENSAIVNARVDWRETPEAHVFKADLPGLKKEEVKVEIEEDSVLKISGERHVEKEDKNDTWHRVERSSGQFTRRFRLPENVKMDQVKAAMENGVLTVTVPKAETKKADVKSIQISG

SEQ ID NO: 43 - DNA, 911 bp, Arabidopsis thaliana
  misc_feature (1)..(911): sequence number 12420894
ctcttaaagcttctcgttttctctgccgtctctctcattcgcgcgacgcaaacgatcttcagccatggccaccgccgcagatgttgacgctgagattcagcaggcgctcactaacgaagtcaagctcttcaaccgctggacctatgacgacgttacggtcacagacatcagtcttgttgactacattggagttcaggcagctaaacatgctacctttgttccccacaccgctggaagatactctgtgaagagattcaggaaggctcagtgccccattgttgagaggctcaccaactctctcatgatgcacgggaggaacaacggtaagaaattgatggctgtcaggatcgtcaagcacgccatggagattatccacctcttgtctgacttgaacccaattcaggtcatcattgacgccattgtcaacagtggtccacgtgaagatgctaccagaattggatctgctggtgttgttaggagacaagctgttgatatctctcctctaagacgtgttaaccaggctatcttcttgattaccactggtgctcgtgaagctgctttcagaaacatcaagactatagctgagtgccttgctgatgaattgatcaacgcagccaagggctcttccaacagctatgccatcaagaagaaggatgagattgaaagagttgccaaggctaatcgttaaggattttccctttgccctgtgcgttacaatctcgtatcaatgagtttaatgttttatcttcatttagattgaaatatgtatctcagatgtttgctcttttgttttatgaagtttatttcgtctgaactactttgaatagataaatttttgatgctttaagctgtttcgcatcagtttattgtcaatttttgaacttatctaggccacctgaagctagaaattt

SEQ ID NO: 44 - protein, 207 aa, Arabidopsis thaliana [sequence omitted]

SEQ ID NO: 45 - DNA, 1075 bp, Arabidopsis thaliana
  misc_feature (1)..(1075): sequence number 13619634
gggtttttctctgaggaagaagcgtttcatttctctgaatttcatcgaaaatggcggaaagaggaggagaaggcggcgcagagcgtggcggtgaccgtggtgacttcggacgtggattcggcggtggacgtggaggtggccgtggccgtgatcgtggtccaagaggccgtggaagacgtggaggccgtgcctcggaagaaacgaaatgggttccagtgaccaaactaggtcgtctagtggctgacaataaaataacgaagctagagcagatctatctccattctctcccagtaaaggagtaccaaatcatagatcatcttgttggtcctacgttgaaagacgaggttatgaagatcatgcctgttcagaaacaaaccagagctggtcaaaggactagattcaaggcctttgttgttgttggtgatggtaatggtcatgttggtttgggtgtcaagtgttctaaggaagttgctactgccattagaggagctattattcttgctaagctctctgttgttccggtgaggagaggttactgggggaataagattgggaagccacacactgtgccttgtaaggttactgggaagtgtggctctgttactgtgagaatggttcctgctccgagaggttctggtattgttgctgctagggttcctaagaaggttcttcagttcgctggtattgatgatgtctttacttcttccagaggatctactaaaactcttggaaactttgtcaaggcgacgtttgactgcttacagaagacatatgggttccttacaccagagttctggaaagagactagattctccagatcgccctaccaagagcacactgatttcttgtcgactaaggctgtttctgcaaccaaggttatcacggagggtgaagaccaagcttaagaccttcatgagataagttttggttgttcttaatactaattcctactttgaaaaagagtatttttttttcttgattatctatgagttttgttattggtggtttcgttactgttttggattttgttagatgtttgccttaatgcaaatttcaatgaaatagcttttgcaat

SEQ ID NO: 46 - protein, 284 aa, Arabidopsis thaliana
  misc_feature (1)..(284): sequence number 13619635
MAERGGEGGAERGGDRGDFGRGFGGGRGGGRGRDRGPRGRGRRGGRASEETKWVPVTKLGRLVADNKITKLEQIYLHSLPVKEYQIIDHLVGPTLKDEVMKIMPVQKQTRAGQRTRFKAFVVVGDGNGHVGLGVKCSKEVATAIRGAIILAKLSVVPVRRGYWGNKIGKPHTVPCKVTGKCGSVTVRMVPAPRGSGIVAARVPKKVLQFAGIDDVFTSSRGSTKTLGNFVKATFDCLQKTYGFLTPEFWKETRFSRSPYQEHTDFLSTKAVSATKVITEGEDQA

SEQ ID NO: 47 - DNA, 667 bp, Arabidopsis thaliana
  misc_feature (1)..(667): sequence number 13616623
atttctcattcacttttcatttcaaaagtaaaacaagacaaacaaaaaatacacttaaccatttatttttctctcatgaagaataatactcaacctcaatcatctttcaagaaactttgccggaaactatcaccaaagagggaagattcagccggagagatacaacaacataacagtagcaatggtgaggacaagaacagagagttagaggctgttttttcttacatggatgcaaacagagacggtagaatctcaccagaagagcttcaaaagagtttcatgacattgggagaacaattgtctgatgaagaagccgtagctgctgttagattgtctgatacggacggagatgggatgttggattttgaggaattttctcagttaatcaaagtagatgacgaagaagagaagaagatggagctcaagggagcgtttagactgtatattgcagaaggtgaagattgtattacaccaagaagcttgaagatgatgctaaagaagctaggagaatcaagaaccactgatgattgtagagttatgattagtgcttttgatctcaatgctgatggagttttaagctttgatgagtttgctcttatgatgcgctaagcctccattgttgttgttgttgttgttgttgttgttcttttatttaatctcttattgt

SEQ ID NO: 48 - protein, 177 aa, Arabidopsis thaliana
  misc_feature (1)..(177): sequence number 13616625
MKNNTQPQSSFKKLCRKLSPKREDSAGEIQQHNSSNGEDKNRELEAVFSYMDANRDGRISPEELQKSFMTLGEQLSDEEAVAAVRLSDTDGDGMLDFEEFSQLIKVDDEEEKKMELKGAFRLYIAEGEDCITPRSLKMMLKKLGESRTTDDCRVMISAFDLNADGVLSFDEFALMMR

SEQ ID NO: 49 - DNA, 1765 bp, Arabidopsis thaliana
  misc_feature (1)..(1765): sequence number 13601536
aaaaatgtctcattgcttctctcgttctaaaaaaaatcttgctgctatctctctataagtccactcctccttcaagcaaagcaccttcctcttctttttgctcctctgagattggtttaagattaaaccagacccatctaagggatctggaacaagcttcgtctctggttccactctgatcatcagagtattaaaaatggagtctttcttctccagatccacctccatcgtctccaaattgagtttcttggccttatggatcgtcttcttgatttcttcatcttcttttacttcgacagaagcatatgatgcgcttgatccagaaggcaacattacaatgaaatgggatgttatgagctggactcctgatggctatgttgccgtggttacgatgttcaacttccagaaatacagacacattcaatctccaggatggacattaggttggaaatgggcaaagaaggaagttatatggagtatggttggagcacaaacaactgaacaaggtgattgttcaaagtacaaaggaaacataccacattgttgtaagaaggatccaacagttgtagacttgcttccagggactccttataatcagcagattgctaattgctgcaagggtggtgttatgaactcatgggttcaagaccctgccactgcggctagctccttccagattagtgttggtgctgctggaaccacaaacaaaaccgttagggtcccaagaaacttcactctcatgggacctggtccaggttacacttgtggtccagcaaagattgtcagaccaacaaaatttgtcacgactgacacacgcagaaccactcaagctatgatgacatggaacattacgtgcacatactcgcagttccttgctcaaagaactccaacttgctgtgtttctttatcttctttctacaatgaaaccattgttggatgtccaacttgtgcttgcggatgtcaaaacaacagaacagaatccggtgcctgcctcgacccggacacaccacacttagcctcggttgtgtcaccaccaacaaagaaaggaacggttttaccaccattagtgcaatgcacgagacacatgtgcccgatcagagtgcattggcatgtaaagcagaactacaaagagtattggcgtgtgaagatcacaatcacaaacttcaactatcgcttgaactacacacaatggaaccttgttgctcaacatccaaatctcgacaacatcactcaaatcttcagcttcaactacaaatctcttactccttacgctggactaaacgatacggcgatgttatggggagtgaagttctacaacgatttcttatcagaagcaggtcctcttgggaatgttcaatcagagattttgttccgtaaagaccaatcaaccttcacattcgagaaaggttgggcttttccacgaaggatttactttaatggagacaattgcgtcatgcctcctccagactcttacccttttcttcccaacggtggttcccggtcacaattctcattcgtcgccgccgtgctcctccctcttcttgtctttttcttcttctctgcctaatctcggatttacggttttgccactggtttgcttagggttacggcggagtggtataaacgtttatttatgattcttttgtgtcccacaaaaattataatcttttgatactttttaaaaatataaatagttttcaacttccttgtttttaaaagaaatttatatccttgtgttctgttggtccgtcgttgtagaatatcg

SEQ ID NO: 50 - protein, 456 aa, Arabidopsis thaliana
  misc_feature (1)..(456): sequence number 13601537
MESFFSRSTSIVSKLSFLALWIVFLISSSSFTSTEAYDALDPEGNITMKWDVMSWTPDGYVAVVTMFNFQKYRHIQSPGWTLGWKWAKKEVIWSMVGAQTTEQGDCSKYKGNIPHCCKKDPTVVDLLPGTPYNQQIANCCKGGVMNSWVQDPATAASSFQISVGAAGTTNKTVRVPRNFTLMGPGPGYTCGPAKIVRPTKFVTTDTRRTTQAMMTWNITCTYSQFLAQRTPTCCVSLSSFYNETIVGCPTCACGCQNNRTESGACLDPDTPHLASVVSPPTKKGTVLPPLVQCTRHMCPIRVHWHVKQNYKEYWRVKITITNFNYRLNYTQWNLVAQHPNLDNITQIFSFNYKSLTPYAGLNDTAMLWGVKFYNDFLSEAGPLGNVQSEILFRKDQSTFTFEKGWAFPRRIYFNGDNCVMPPPDSYPFLPNGGSRSQFSFVAAVLLPLLVFFFFSA

SEQ ID NO: 51 - DNA, 934 bp, Arabidopsis thaliana
  misc_feature (1)..(934): sequence number 13618061
ataaatacaagcctcctaactcataaaataagcataaccctaactctacaaagttcttctgattctttctctctctctctttctttcaagagcggttttcaatccattcgctaaagaccatgaacctagaagagaaaccaaccatgacggcttcaagggcttcccctcaagccgaacatctctactacgtccggtgtagcatctgcaacaccatcctcgcggttgggataccattgaagagaatgcttgacacggtaacggtgaaatgcggccattgtggtaacctctcgtttctcaccacaactcctcctcttcaaggccatgttagcctcacccttcagatgcagagctttggtggaagtgactataagaagggaagctcttcttcttcctcttcctccacctccagcgaccagcccccatctccctcacctccctttgtcgtcaaacctcctgagaagaagcagaggctcccatctgcatacaaccgcttcatgagggatgagatccaacgcatcaaaagtgccaatccggaaataccacaccgtgaagctttcagtgctgctgccaaaaattgggctaagtacatacccaactctcctacttccattacttccggaggccacaacatgatccatggcttgggattcggtgagaagaagtgaacaaaactcaggggaaaagaagcctaaaaataacaaacgcatgcacgtgtgcgagtggctgcgtcgtttttctcatcttgtgttgttcttctgtgtaattttcttatgtatgtcatgttgcagaaaatgatgttgccttagtttttatgactttatatttctgtctgtctttagatttgaaagtaacgtcacttgctatgtccctttggacgtttatgtctggtctttatttgtcttaatcctatcaaaattttatatgcgtattcctt

SEQ ID NO: 52 - protein, 181 aa, Arabidopsis thaliana
  misc_feature (1)..(181): sequence number 13618062
MNLEEKPTMTASRASPQAEHLYYVRCSICNTILAVGIPLKRMLDTVTVKCGHCGNLSFLTTTPPLQGHVSLTLQMQSFGGSDYKKGSSSSSSSSTSSDQPPSPSPPFVVKPPEKKQRLPSAYNRFMRDEIQRIKSANPEIPHREAFSAAAKNWAKYIPNSPTSITSGGHNMIHGLGFGEKK

SEQ ID NO: 53 - DNA, 1268 bp, Arabidopsis thaliana
  misc_feature (1)..(1268): sequence number 12705120
aaaactcttaaacagcttcctaacgagaggaaactgaggaacacaacaatggagtttcgtggagatgccaaccagaggattgctaggatttcagctcatctcactcctcagatggaggccaagaactctgtaatcggacgggaaaactgcagagctaaaggtggtaatccaggattcaaagtagcaattcttggagctgcaggtggaattggacaatctttatctttgctgatgaagatgaaccctcttgtctctttacttcatctctacgatgttgtcaatgctcctggcgtcactgctgacgtcagtcatatggacactggagctgttgtccgcgggttcttgggagcgaagcagcttgaggacgcgctaacgggtatggatcttgtgatcataccagccggtataccgaggaaaccagggatgacccgcgatgatctgtttaaaatcaatgctgggattgttaaaacactatgtgaaggtgtagcaaaatgttgtcctaatgctattgtcaacttgatcagcaaccctgtgaactctactgtccccattgccgctgaggttttcaagaaagctggaacttatgatcctaagaagctccttggagttactacactcgatgttgctcgtgccaacacatttgtggcagaagttcttggccttgatccaagagaagtcgatgtgccagtagttgggggacacgccggagtcacaatcttgccactactgtcacaggttaaacctcctagcagcttcacacctcaagaaattgagtacctgacaaaccggattcaaaatggtggaactgaagttgtggaggcaaaagctggagctggttctgcaacactttcaatggcatatgctgcagccaagtttgcagatgcttgccttcgcgggttaagaggagatgcgaatgtcgtagaatgctcttttgttgcttcacaggtgacagaattagctttctttgcaacaaaagtgcgccttggccgtacaggagcagaggaagtgtatcagcttggacccttaaacgaatacgaaaggattggtctggagaaagcaaaagatgaattagccggaagtattcagaaaggtgttgaattcatcagaaaatgaaactgagagataatcagagagatacaataagttatttcctcaactatatgatcatgtactcatcatcacatcatgcctatgtctcctctgcttctgatacaactttgtataaatccttatcagtttgtgtacgatatatgtgaccttttcaacgt

SEQ ID NO: 54 - protein, 354 aa, Arabidopsis thaliana
  misc_feature (1)..(354): sequence number 12705122
MEFRGDANQRIARISAHLTPQMEAKNSVIGRENCRAKGGNPGFKVAILGAAGGIGQSLSLLMKMNPLVSLLHLYDVVNAPGVTADVSHMDTGAVVRGFLGAKQLEDALTGMDLVIIPAGIPRKPGMTRDDLFKINAGIVKTLCEGVAKCCPNAIVNLISNPVNSTVPIAAEVFKKAGTYDPKKLLGVTTLDVARANTFVAEVLGLDPREVDVPVVGGHAGVTILPLLSQVKPPSSFTPQEIEYLTNRIQNGGTEVVEAKAGAGSATLSMAYAAAKFADACLRGLRGDANVVECSFVASQVTELAFFATKVRLGRTGAEEVYQLGPLNEYERIGLEKAKDELAGSIQKGVEFIRK

SEQ ID NO: 55 - DNA, 1225 bp, Arabidopsis thaliana
  misc_feature (1)..(1225): sequence number 13607229
attttaaaagtatcagtttacactgacacaatccttaactattttcctttgttcttcttcatctttattacacatttttttcaaggtctaccaaacgatgtcggttttcgaatcggagacttcgaacttccacgtctacaacaaccacgaaatccaaacgcaaccgcaaatgcaaacgtttctgtcggaggaggaaccggtagggagacagaactcgattttgtcactaactcttgacgaaattcagatgaaaagcggtaagagctttggagcgatgaacatggacgagttcctagcgaacttgtggacaaccgttgaagaaaacgacaacgaaggaggtggggctcacaacgacggagagaagccggcggtgctgccacgtcaagggtcgttgtccctccctgtgcctttatgcaagaaaacggtcgacgaggtttggctcgagatacaaaacggtgtacaacaacatccaccgtcgtcgaattccggtcaaaactccgccgaaaatattcgccggcaacaaacccttggtgagatcactctcgaggattttcttgttaaggctggtgttgtacaagaaccgttgaagacaacgatgaggatgtcgagttctgattttggttataaccccgagtttggagttggtttacattgtcagaaccaaaacaattatggtgataaccggtcggtttatagtgaaaaccgaccgttttactcggttttgggagaatcttcaagctgtatgaccgggaatgggaggagtaatcagtatctgaccggtttagatgcttttcggatcaagaaacggataattgatggtccacctgaaattttgatggagcggagacaacggcgaatgattaaaaaccgcgaatctgcggctcggtctcgagcccggagacaagcttatactgtggaactggagttggaattgaacaacctcacggaagaaaacacgaagctgaaggaaattgtggaggaaaatgagaagaaaagaagacaagagataataagtagaagcaaacaagtgactaaagagaagagcggagacaaattgagaaagattcggaggatggccagtgccgggtggtaaatagatatgagagtttgtgtaatttacatatgatattttattgatcgtttctatctatacacataaatatttgtatctatattatattttgagacaaacatggaatttgtgtatataggatcatttcttttt

SEQ ID NO: 56 - protein, 331 aa, Arabidopsis thaliana
  misc_feature (1)..(331): sequence number 13607230
MSVFESETSNFHVYNNHEIQTQPQMQTFLSEEEPVGRQNSILSLTLDEIQMKSGKSFGAMNMDEFLANLWTTVEENDNEGGGAHNDGEKPAVLPRQGSLSLPVPLCKKTVDEVWLEIQNGVQQHPPSSNSGQNSAENIRRQQTLGEITLEDFLVKAGVVQEPLKTTMRMSSSDFGYNPEFGVGLHCQNQNNYGDNRSVYSENRPFYSVLGESSSCMTGNGRSNQYLTGLDAFRIKKRIIDGPPEILMERRQRRMIKNRESAARSRARRQAYTVELELELNNLTEENTKLKEIVEENEKKRRQEIISRSKQVTKEKSGDKLRKIRRMASAGW

SEQ ID NO: 57 - DNA, 1267 bp, Arabidopsis thaliana
  misc_feature (1)..(1267): sequence number 12323871
aaaataagctctctttctactatttctctttctctttctactatttctctcctgtggagaaactcaggagatagagagagagagagagaagagaagagagcatgtatgtttggttttataatctctctactcataccaaagatttgtctcagacccaccacttggacagagagaacccaagctcctttctctctttttctcgatctactccttcttaatctccttttttgaaacttgaagccactttcaacatcatccttaaacttttgttcccttattcacaatctcctgccacctctcatttctctagctacatatatgggttctattagaggaaacattgaagagcctatatctcagtcattaacgaggcagaactctctctatagcttaaagctccatgaggttcaaacccacttaggaagttctggaaaaccactaggaagcatgaaccttgatgagcttctcaagactgtcttgccaccagctgaggaagggcttgttcgtcagggaagcttgacgttacctcgagatctcagtaaaaagacagttgatgaggtctggagagatatccaacaggacaagaatggaaacggtactagtactactactactcataagcagcctacactcggtgaaataacacttgaggatttgttgttgagagctggtgtagtgactgagacagtagtccctcaagaaaatgttgttaacatagcttcaaatgggcaatgggttgagtatcatcatcagcctcaacaacaacaagggtttatgacatatccggtttgcgagatgcaagatatggtgatgatgggtggattatcggatacaccacaagcgcctgggaggaaaagagtagctggagagattgtggagaagactgttgagaggagacagaagaggatgatcaagaacagagaatctgcagcacgttcacgagctaggaaacaggcttatacacatgaattagagatcaaggtttcaaggttagaagaagaaaacgaaaaacttcggaggctaaaggaggtggagaagatcctaccaagtgaaccaccaccagatcctaagtggaagctccggcgaacaaactctgcttctctctgatcctaaagactcttctttctttcttcttctttgtgttggtttatatcagaccgctttgttctttgtatattgtgtagactttattgactttgaacagcatgtctttataaacatttcttgagtgtattcctcttttgcagctagttcaaactttttgagt

SEQ ID NO: 58 - protein, 262 aa, Arabidopsis thaliana
  misc_feature (1)..(262): sequence number 12323872
MGSIRGNIEEPISQSLTRQNSLYSLKLHEVQTHLGSSGKPLGSMNLDELLKTVLPPAEEGLVRQGSLTLPRDLSKKTVDEVWRDIQQDKNGNGTSTTTTHKQPTLGEITLEDLLLRAGVVTETVVPQENVVNIASNGQWVEYHHQPQQQQGFMTYPVCEMQDMVMMGGLSDTPQAPGRKRVAGEIVEKTVERRQKRMIKNRESAARSRARKQAYTHELEIKVSRLEEENEKLRRLKEVEKILPSEPPPDPKWKLRRTNSASL

SEQ ID NO: 59 - DNA, 2353 bp, Arabidopsis thaliana
  misc_feature (1)..(2353): sequence number 13602983
ctgtgtctttatcttctctctcctcttcttgaaaaactgaacctttaattctttcttcacatctcctttagctttctgaagctgctatcttcaagctcaaaaccaaaccctagaaataaaatgacggcgacacaaatgctgcttcatcttcttcttcttccacttctcttccttcacttcagcaaccaagtcatggcagaaatcaccgacgagttagcgacattgatggaggttaaaacagagcttgacccagaagacaaacacttagcttcatggagtgttaatggagatctgtgtaaagactttgaaggtgttggttgtgactggaaaggacgagtttctaacatatctctacaagggaaaggcttatctggtaaaatctctccgaacattggaaagcttaaacatttgacgggtttgttcttgcactacaatgctcttgttggagatattcctagagaacttggtaacttgtcggaacttacggatctttatctcaatgttaataatctctccggtgagattccttcaaacattgggaagatgcaaggcttgcaagttttgcagctctgttacaacaatttgactggaagcattcctagggagcttagttcactgaggaagctgagtgttcttgctcttcaatctaacaaactcactggagctatacctgcgagtttaggagatttaagtgctctagagcggctagatttgagctacaatcatttgtttggttctgtaccaggcaagttagctagccctcctctgcttcgagttctcgacatccgcaacaactccctcactggcaatgtacctcctgtactgaagagattaaatgaaggtttttcgtttgaaaacaacttgggactatgtggagctgagttctcgccgttgaaatcttgcaatggcacagctcctgaggaacctaagccatacggtgcaaccgtgtttggttttccatcccgggatataccagaatcagctaatctacggtcaccttgtaatggaacaaactgtaatactcctccaaaatctcaccaaggtgcaatccttattggtttggttgtctcgactattgcattgtctgctattagcatcctcttgttcacgcattatcgcagacgcaaacagaagctttcaaccacttatgaaatgtcggacaaccgtctcaacaccgtaggaggaggttttaggaagaacaatggttctccattggccagtcttgaatatactaatggttgggatccactttcagacaataggaatctcagtgtctttgctcaagaagttatccagagctttcgattcaatcttgaagaggttgaaactgctacacagtatttctcagaagtgaatttgcttggaagaagcaacttctctgcgacttacaaaggaatcttgagagatggttctgctgtagcgatcaaacggtttagtaaaacaagttgcaaatctgaagaacctgagttcttgaaaggactcaacatgttggcgtctctgaaacatgaaaacttgtcaaaacttagaggcttctgctgttcaagaggccgtggagaatgttttctcatctatgattttgctcctaatggaaatttactgagctatcttgatctcaaagatggtgatgcgcatgttcttgattggtccacgagagtatccattgctaaaggcatcgcaaaagggattgcttatctacattcatacaaaggaagcaagccagcattggttcaccaaaacatctcagcggagaaagttctaatcgaccagcgatacagtcctctactctcaaactcaggcctccacacacttctcacaaacgacattgtcttctctgcactcaaagacagtgcagcaatgggctacctcgctccagaatacaccacaaccggacgtttcaccgagaaaaccgatgtctacgctttcgggattcttgtgtttcagataatctccgggaaacagaaggttaggcatcttgttaagcttggaactgaagcttgtaggttcaatgattacatagatccaaatcttcaaggaagattctttgaatatgaagccacaaagctagctagaatcgcgtggctatgcactcatgaatctcccattgaaagaccatccgtggaagctgtagttcatgagctaggtaactgtagtagctgtctctgagaactctctcgaggcaaatgtgtaatgtgtaccttaggaatcaattaaggaagggtttgttagtcaccttttcctgtgtttagggttaagaaaaaggtgccttttcccttcccattaacgtttaaaaactgttcaacgtgttgtactttattggtttgtatgatttgtgtttatgttaagctcttcaacc

SEQ ID NO: 60 - protein, 680 aa, Arabidopsis thaliana
  misc_feature (1)..(680): sequence number 13602984
MTATQMLLHLLLLPLLFLHFSNQVMAEITDELATLMEVKTELDPEDKHLASWSVNGDLCKDFEGVGCDWKGRVSNISLQGKGLSGKISPNIGKLKHLTGLFLHYNALVGDIPRELGNLSELTDLYLNVNNLSGEIPSNIGKMQGLQVLQLCYNNLTGSIPRELSSLRKLSVLALQSNKLTGAIPASLGDLSALERLDLSYNHLFGSVPGKLASPPLLRVLDIRNNSLTGNVPPVLKRLNEGFSFENNLGLCGAEFSPLKSCNGTAPEEPKPYGATVFGFPSRDIPESANLRSPCNGTNCNTPPKSHQGAILIGLVVSTIALSAISILLFTHYRRRKQKLSTTYEMSDNRLNTVGGGFRKNNGSPLASLEYTNGWDPLSDNRNLSVFAQEVIQSFRFNLEEVETATQYFSEVNLLGRSNFSATYKGILRDGSAVAIKRFSKTSCKSEEPEFLKGLNMLASLKHENLSKLRGFCCSRGRGECFLIYDFAPNGNLLSYLDLKDGDAHVLDWSTRVSIAKGIAKGIAYLHSYKGSKPALVHQNISAEKVLIDQRYSPLLSNSGLHTLLTNDIVFSALKDSAAMGYLAPEYTTTGRFTEKTDVYAFGILVFQIISGKQKVRHLVKLGTEACRFNDYIDPNLQGRFFEYEATKLARIAWLCTHESPIERPSVEAVVHELGNCSSCL

SEQ ID NO: 61 - DNA, 1209 bp, Arabidopsis thaliana
  misc_feature (1)..(1209): sequence number 13607852
gttagatgatctgtccatttttttcttttttcttctgtgtataaatatatttgagcacaaagaaaaactaataaccttctgttttcagcaagtagggtcttataaccttcaaagaaatattccttcaattgaaaacccataaaccaaaatagatattacaaaaggaaagagagatattttcaagaacaacataattagaaaagcagaagcagcagttaagtggtactgagataaatgatatagtttctcttcaagaacagtttctcattacccaccttctcctttttgctgatctatcgtaatcttgagaactcaggatcaatggaggaaggtgggagtagtcacgacgcagagagtagcaagaaactagggagagggaaaatagagataaagaggatagagaacacaacaaatcgtcaagttactttctgcaaacgacgcaatggtcttctcaagaaagcttatgaactctctgtcttgtgtgatgccgaagttgccctcgtcatcttctccactcgtggccgtctctatgagtacgccaacaacagtgtgaggggtacaattgaaaggtacaagaaagcttgttccgatgccgtcaaccctccttccgtcaccgaagctaatactcagtactatcagcaagaagcctctaagcttcggaggcagattcgagatattcagaattcaaataggcatattgttggggaatcacttggttccttgaacttcaaggaactcaaaaacctagaaggacgtcttgaaaaaggaatcagccgtgtccgctccaaaaagaatgagctgttagtggcagagatagagtatatgcagaagagggaaatggagttgcaacacaataacatgtacctgcgagcaaagatagccgaaggcgccagattgaatccggaccagcaggaatcgagtgtgatacaagggacgacagtttacgaatccggtgtatcttctcatgaccagtcgcagcattataatcggaactatattccggtgaaccttcttgaaccgaatcagcaattctccggccaagaccaacctcctcttcaacttgtgtaactcaaaacatgataacttgtttcttctcctcataacgattaagagagagacgagagagttcattttatatttataacgcgactgtgtattcatagtttaggttctaataatgataataacaaaactgttgtttctttgctt

SEQ ID NO: 62 - protein, 248 aa, Arabidopsis thaliana
  misc_feature (1)..(248): sequence number 13607853
MEEGGSSHDAESSKKLGRGKIEIKRIENTTNRQVTFCKRRNGLLKKAYELSVLCDAEVALVIFSTRGRLYEYANNSVRGTIERYKKACSDAVNPPSVTEANTQYYQQEASKLRRQIRDIQNSNRHIVGESLGSLNFKELKNLEGRLEKGISRVRSKKNELLVAEIEYMQKREMELQHNNMYLRAKIAEGARLNPDQQESSVIQGTTVYESGVSSHDQSQHYNRNYIPVNLLEPNQQFSGQDQPPLQLV

SEQ ID NO: 63 - DNA, 943 bp, Arabidopsis thaliana
  misc_feature (1)..(943): sequence number 12677570
gtgacttaaaaagctcctaggcaccgaaatcgagacactctcgacaaccatgctctctgacgccgggggtggttccgattgccggcgtcgggacttatcaactcccatcaaccttcatgtgttctatataagctttatcttcattgaatcttcctccgttatctcaaatctctcaaaatatctaaatctccttttttatgtgagcttcttcactgaaagttttctttgtgacggaagagtatatcgatgctctattggctccgatctgactcagatcttggatgcatctctgtcttcgaacccaaaacaagaaaattcacaacagtccaacagctcctcttctcaaacatcagagcaagacttcatcaacttatcaaaaagctctagatctggactagcaccaacaccacctttggtttcttctcaccggttttcgttgatggcaggagtatctcttggaccatcagatgtgcttcttccgctgggaacgtcgacggcacacgacgagctcaaacgctggcaatggtcaccctatatgattcacagtcgcccatcattccaattcttcagaatgacggaggcgctttccttatcccgacaacatcaaccctagggtttgattggatgagcctcatctttcattaaagcccaatatctcccatgtggcccatatccaggtctgatttccctatcaatttgcatagtctctctttgctttgtttctcgacaaaggatggtcgtcgactccattcagctttaggaacaatgcatctagaggggatttgcttattcaattatcgaaaccttatgattaaccaattggttcttaaccttgcatcttcacaattgtttaaattaaaattttgaaacatgacatgtaagaagcaaatcttagtttttaattcataaaaacttcttagatatgaagcaaatcttagttttt

SEQ ID NO: 64 - protein, 187 aa, Arabidopsis thaliana
  misc_feature (1)..(187): sequence number 12677571
MLSDAGGGSDCRRRDLSTPINLHVFYISFIFIESSSVISNLSKYLNLLFYVSFFTESFLCDGRVYRCSIGSDLTQILDASLSSNPKQENSQQSNSSSSQTSEQDFINLSKSSRSGLAPTPPLVSSHRFSLMAGVSLGPSDVLLPLGTSTAHDELKRWQWSPYMIHSRPSFQFFRMTEALSLSRQHQP

SEQ ID NO: 65 - DNA, 1024 bp, Arabidopsis thaliana
  misc_feature (1)..(1024): sequence number 13504706
acaactcgattaccaaaaagcagagccatccaaccataaaactcaaaacacacagattccactggcgtgtgctctcctcacttcactcgtccttgaaacttgagcttcaattacaaagaatggctgcttcaagtgctgtcaccgcaaactacgtcctcaagccacctccattcgcactggatgctttggagccgcatatgagcaaacaaactctggagtttcactggggaaaacatcacagagcttacgtggacaacctcaagaaacaggttcttggaaccgagcttgaaggcaagcccttagagcacattatccacagcacttacaacaatggtgatctcctccctgctttcaacaacgctgctcaggcgtggaaccacgagttcttctgggagtcaatgaaaccaggtggtggaggaaaaccatcaggagagcttcttgctttgcttgaaagagatttcacttcttatgagaagttctatgaagagttcaatgctgctgcagccactcagtttggagctggctgggcctggcttgcttattcaaatgaaaaactcaaagtagtgaaaactcccaatgctgtgaatccccttgtgctcggctctttcccattgcttaccattgatgtctgggagcatgcttactaccttgacttccagaaccgaagaccagattacataaagacattcatgaccaatcttgtgtcttgggaagctgtaagtgccagacttgaggccgccaaggctgcttctgcttaagcaaatttctgaacaatttgacttcagtgacagtgagttctgcatcaccgaagtctcttataaaatattggtcgctgtaataaggacacagctctcttgttgtgtatgtgtcacagagttcttcattttgcttgtgtaatgaacaattaaacatgctcttttctgagtgtgtgtgcgttttgtgtgtgtcaagtttttcatcgtctcctttattaaactcaaattggcacctaccatcagtaattcatagtttggcatggcccatt

SEQ ID NO: 66 - protein, 212 aa, Arabidopsis thaliana
  misc_feature (1)..(212): sequence number 13504708
MAASSAVTANYVLKPPPFALDALEPHMSKQTLEFHWGKHHRAYVDNLKKQVLGTELEGKPLEHIIHSTYNNGDLLPAFNNAAQAWNHEFFWESMKPGGGGKPSGELLALLERDFTSYEKFYEEFNAAAATQFGAGWAWLAYSNEKLKVVKTPNAVNPLVLGSFPLLTIDVWEHAYYLDFQNRRPDYIKTFMTNLVSWEAVSARLEAAKAASA

SEQ ID NO: 67 - DNA, 1785 bp, Arabidopsis thaliana
  misc_feature (1)..(1785): sequence number 12728113
atgagtgactcggtgtcgatctcggttccgtataggaatttgaggaaggaaattgaacttgagacggtcaccaagcatcgtcaaaacgaatctggttcttcgtcgttctctgaatctgcttctccttcgaatcattctgattcggctgatggtgaatctgtgtcgaagaattgtagtttagtgacgttggttcttagttgtacagttgccgctggagttcaatttggttgggcattgcaactttctcttcttactccttatattcagacccttggaatatcgcatgctttttcttcgtttatttggctgtgcggcccaattacaggccttgtggtccagccttttgttggcatttggagtgataaatgtacttcaaagtatggaagaagacgaccatttattctagttggatcattcatgatctcaatagcagtgataataatcggattttctgcagacattgggtatctgttaggagattcaaaggaacattgcagtactttcaaaggcacacgaaccagggcagctgttgtctttatcattgggttttggttgttggatctagcaaacaatacagtacagggacctgctcgtgctcttctagctgatctatcaggtcctgatcagcggaatactgcaaatgctgtgttctgcttgtggatggctattgggaacatccttgggttttctgccggtgctagcggaaaatggcaagaatggttcccttttttaactagtagagcatgttgtgctgcatgtggaaatctcaaagcagcgtttcttcttgcagtggtctttctcactatatgtactcttgtcacaatctattttgctaaagagattccttttacaagcaacaagcccacccgcatacaagattctgcacctttgttggatgatctccagtccaaaggccttgagcattcaaaattaaataatggtactgccaatggaatcaagtatgagagagtggaacgtgatacggatgaacagtttggcaattcagagaatgagcatcaagatgagacctacgttgatggccctggatctgttttagtgaatttgctaactagtttaaggcatttgccaccggctatgcactcagttcttatcgtcatggctcttacatggttatcctggttccccttctttctgttcgatacagattggatgggaagagaagtttaccatggggatccaacaggagatagtttgcatatggaactctatgatcaaggtgtacgtgaaggtgcacttggtttgctactaaactctgttgttcttgggatcagctcatttctcattgaaccaatgtgtcagcggatgggtgctcgggttgtatgggctttgagcaattttactgtatttgcctgcatggcgggaacagctgtaatcagcttgatgtctctcagtgatgacaaaaatggaattgaatacataatgcgtggaaacgaaacaacaagaaccgcagccgtaatcgtttttgcactccttggttttcccctagctatcacatacagtgtccctttctctgtcacagcagaagtcactgctgattccggtggcggtcaaggtttggctataggagtgttgaatctcgcaatcgttattccccagatgatagtatcacttggagcgggtccatgggatcaattgtttggaggaggaaacttaccggcgtttgttttggcgtctgttgctgctttcgctgctggagttattgcattgcaaaggcttcccacgctatcgagttctttcaagtccaccggtttccacatcggctaa

SEQ ID NO: 68 - protein, 594 aa, Arabidopsis thaliana
  misc_feature (1)..(594): sequence number 12728114
MSDSVSISVPYRNLRKEIELETVTKHRQNESGSSSFSESASPSNHSDSADGESVSKNCSLVTLVLSCTVAAGVQFGWALQLSLLTPYIQTLGISHAFSSFIWLCGPITGLVVQPFVGIWSDKCTSKYGRRRPFILVGSFMISIAVIIIGFSADIGYLLGDSKEHCSTFKGTRTRAAVVFIIGFWLLDLANNTVQGPARALLADLSGPDQRNTANAVFCLWMAIGNILGFSAGASGKWQEWFPFLTSRACCAACGNLKAAFLLAVVFLTICTLVTIYFAKEIPFTSNKPTRIQDSAPLLDDLQSKGLEHSKLNNGTANGIKYERVERDTDEQFGNSENEHQDETYVDGPGSVLVNLLTSLRHLPPAMHSVLIVMALTWLSWFPFFLFDTDWMGREVYHGDPTGDSLHMELYDQGVREGALGLLLNSVVLGISSFLIEPMCQRMGARVVWALSNFTVFACMAGTAVISLMSLSDDKNGIEYIMRGNETTRTAAVIVFALLGFPLAITYSVPFSVTAEVTADSGGGQGLAIGVLNLAIVIPQMIVSLGAGPWDQLFGGGNLPAFVLASVAAFAAGVIALQRLPTLSSSFKSTGFHIG

SEQ ID NO: 69 - DNA, 1472 bp, Arabidopsis thaliana
  misc_feature (1)..(1472): sequence number 12705056
aaatacacctaacttgtttagtacacaacagcaacatcaaactctaataaacccaagttggtgtatactataatggtgatggctggtgcttcttctttggatgagatcagacaggctcagagagctgatggacctgcaggcatcttggctattggcactgctaaccctgagaaccatgtgcttcaggcggagtatcctgactactacttccgcatcaccaacagtgaacacatgaccgacctcaaggagaagttcaagcgcatgtgcgacaagtcgacaattcggaaacgtcacatgcatctgacggaggaattcctcaaggaaaacccacacatgtgtgcttacatggctccttctctggacaccagacaggacatcgtggtggtcgaagtccctaagctaggcaaagaagcggcagtgaaggccatcaaggagtggggccagcccaagtcaaagatcactcatgtcgtcttctgcactacctccggcgtcgacatgcctggtgctgactaccagctcaccaagcttcttggtctccgtccttccgtcaagcgtctcatgatgtaccagcaaggttgcttcgccggcggtactgtcctccgtatcgctaaggatctcgccgagaacaatcgtggagcacgtgtcctcgttgtctgctctgagatcacagccgttaccttccgtggtccctctgacacccaccttgactccctcgtcggtcaggctcttttcagtgatggcgccgccgcactcattgtggggtcggaccctgacacatctgtcggagagaaacccatctttgagatggtgtctgccgctcagaccatccttccagactctgatggtgccatagacggacatttgagggaagttggtctcaccttccatctcctcaaggatgttcccggcctcatctccaagaacattgtgaagagtctagacgaagcgtttaaacctttggggataagtgactggaactccctcttctggatagcccaccctggaggtccagcgatcctagaccaggtggagataaagctaggactaaaggaagagaagatgagggcgacacgtcacgtgttgagcgagtatggaaacatgtcgagcgcgtgcgttctcttcatactagacgagatgaggaggaagtcagctaaggatggtgtggccacgacaggagaagggttggagtggggtgtcttgtttggtttcggaccaggtctcactgttgagacagtcgtcttgcacagcgttcctctctaaacagaacgcttgccttctatctgcctacctacctacgcaaaactttaatcctgtcttatgttttatataatataatcattatatgtttacgcaataattaaggaagaattcatttgatgtgatatgtgatatgtgctggacaggtctattcgactgtttttgtactctcttttttgtgtctttttacaatattaaatctatgggtcttgaat

SEQ ID NO: 70 - protein, 395 aa, Arabidopsis thaliana
  misc_feature (1)..(395): sequence number 12705057
MVMAGASSLDEIRQAQRADGPAGILAIGTANPENHVLQAEYPDYYFRITNSEHMTDLKEKFKRMCDKSTIRKRHMHLTEEFLKENPHMCAYMAPSLDTRQDIVVVEVPKLGKEAAVKAIKEWGQPKSKITHVVFCTTSGVDMPGADYQLTKLLGLRPSVKRLMMYQQGCFAGGTVLRIAKDLAENNRGARVLVVCSEITAVTFRGPSDTHLDSLVGQALFSDGAAALIVGSDPDTSVGEKPIFEMVSAAQTILPDSDGAIDGHLREVGLTFHLLKDVPGLISKNIVKSLDEAFKPLGISDWNSLFWIAHPGGPAILDQVEIKLGLKEEKMRATRHVLSEYGNMSSACVLFILDEMRRKSAKDGVATTGEGLEWGVLFGFGPGLTVETVVLHSVPL

SEQ ID NO: 71 - DNA, 1473 bp, Arabidopsis thaliana
  misc_feature (1)..(1473): sequence number 12371852
aaatacacctaacttgtttagtacacaacagcaacatcaaactctaataaacccaagttggtgtatactataatggtgatggctggtgcttcttctttggatgagatcagacaggctcagagagctgatggacctgcaggcatcttggctattggcactgctaaccctgagaaccatgtgcttcaggcggagtatcctgactactacttccgcatcaccaacagtgaacacatgaccgacctcaaggagaagttcaagcgcatgtgcgacaagtcgacaattcggaaacgtcacatgcatctgacggaggaattcctcaaggaaaacccacacatgtgtgcttacatggctccttctctggacaccagacaggacatcgtggtggtcgaagtccctaagctaggcaaagaagcggcagtgaaggccatcaaggagtggggccagcccaagtcaaagatcactcatgtcgtcttctgcactacctccggcgtcgacatgcctggtgctgactaccagctcaccaagcttcttggtctccgtccttccgtcaagcgtctcatgatgtaccagcaaggttgcttcgccggcggtactgtcctccgtatcgctaaggatctcgccgagaacaatcgtggagcacgtgtcctcgttgtctgctctgagatcacagccgttaccttccgtggtccctctgacacccaccttgactccctcgtcggtcaggctcttttcagtgatggcgccgccgcactcattgtggggtcggaccctgacacatctgtcggagagaaacccatctttgagatggtgtctgccgctcagaccatccttccagactctgatggtgccatagacggacatttgagggaagttggtctcaccttccatctcctcaaggatgttcccggcctcatctccaagaacattgtgaagagtctagacgaagcgtttaaacctttggggataagtgactggaactccctcttctggatagcccaccctggaggtccagcgatcctagaccaggtggagataaagctaggactaaaggaagagaagatgagggcgacacgtcacgtgttgagcgagtatggaaacatgtcgagcgcgtgcgttctcttcatactagacgagatgaggaggaagtcagctaaggatggtgtggccacgacaggagaagggttggagtggggtgtcttgtttggtttcggaccaggtctcactgttgagacagtcgtcttgcacagcgttcctctctaaacagaacgcttgccttctatctgcctacctacctacgcaaaactttaatcctgtcttatgttttatataatataatcattatatgtttacgcaataattaaggaagaattcatttgatgtgatatgtgatatgtgctggacaggtctattcgactgtttttgtactctcttttttgtgtctttttacaatattaaatctatgggtcttgaatc

SEQ ID NO: 72 - protein, 395 aa, Arabidopsis thaliana; identical to SEQ ID NO: 70
  misc_feature (1)..(395): sequence number 12371853

SEQ ID NO: 73 - DNA, 19 bp, Artificial Sequence
  oligo(dT)18 primer
ttttttttttttttttttv

SEQ ID NO: 74 - DNA, 19 bp, Artificial Sequence
  oligo dTV primer
ttttttttttttttttttn

(19) United States
(12) Patent Application Publication
(10) Pub. No.: US 2005/0223434 A1
(43) Pub. Date: Oct. 6, 2005

(54) Nucleotide sequences and polypeptides encoded thereby useful for modifying plant characteristics

(75) Inventors: Nickolai Alexandrov, Thousand Oaks, CA (US); Cook Zhihong, Woodland Hills, CA (US); Yiwen Fang, Los Angeles, CA (US); Kenneth Feldmann, Newbury Park, CA (US); Edward A. Kiegle, Chester, VT (US); Shing Kwok, Woodland Hills, CA (US); Yu-Ping Lu, Oak Park, CA (US); Roger Penell, Malibu, CA (US); Richard Schneeberger, Van Nuys, CA (US); Chuan-Yin Wu, Newbury Park, CA (US)

Correspondence Address: BIRCH STEWART KOLASCH & BIRCH, PO BOX 747, FALLS CHURCH, VA, 22040-0747, US

(73) Assignee: Ceres, Inc., Thousand Oaks, CA (US)

(21) Appl. No.: 10/950,095

(22) Filed: Sep. 23, 2004

Related U.S. Application Data
(60) Provisional application No. 60/505,420, filed on Sep. 23, 2003.

(51) Int. Cl. A01H 1/00; C12N 15/29

(52) U.S. Cl. 800/298; 536/23.6; 435/419; 435/320.1; 435/6; 800/278

(57) ABSTRACT

Isolated polynucleotides and polypeptides encoded thereby are described, together with the use of those products for making transgenic plants.

### FIELD OF THE INVENTION

The present invention relates to isolated polynucleotides, polypeptides encoded thereby, and the use of those products for making transgenic plants.

### BACKGROUND OF THE INVENTION

There are more than 300,000 species of plants. They show a wide diversity of forms, ranging from delicate liverworts, adapted for life in a damp habitat, to cacti, capable of surviving in the desert. The plant kingdom includes herbaceous plants, such as corn, whose life cycle is measured in months, to the giant redwood tree, which can live for thousands of years. This diversity reflects the adaptations of plants to survive in a wide range of habitats. This is seen most clearly in the flowering plants (phylum Angiospermophyta), which are the most numerous, with over 250,000 species. They are also the most widespread, being found from the tropics to the arctic.

The process of plant breeding involving man's intervention in natural breeding and selection is some 20,000 years old. It has produced remarkable advances in adapting existing species to serve new purposes. The world's economics was largely based on the successes of agriculture for most of these 20,000 years.

Plant breeding involves choosing parents, making crosses to allow recombination of gene (alleles) and searching for and selecting improved forms. Success depends on the genes/alleles available, the combinations required and the ability to create and find the correct combinations necessary to give the desired properties to the plant. Molecular genetics technologies are now capable of providing new genes, new alleles and the means of creating and selecting plants with the new, desired characteristics.

Plants specifically improved for agriculture, horticulture and other industries can be obtained using molecular technologies. As an example, great agronomic value can result from modulating the size of a plant as a whole or of any of its organs. The green revolution came about as a result of creating dwarf wheat plants, which produced a higher seed yield than taller plants because they could withstand higher levels and inputs of fertilizer and water.

Similarly, modulation of the size and stature of an entire plant, or a particular portion of a plant, allows production of plants better suited for a particular industry. For example, reductions in the height of specific ornamentals, crops and tree species can be beneficial by allowing easier harvesting. Alternatively, increasing height may be beneficial by providing more biomass. Other examples of commercially desirable traits include increasing the length of the floral stems of cut flowers, increasing or altering leaf size and shape, enhancing the size of seeds and/or fruits, enhancing yields by specifically stimulating hormone (e.g. Brassinolide) synthesis and stimulating early flowering or evoking late flowering by altering levels of gibberellic acid or other hormones in specific cells. Changes in organ size and biomass also result in changes in the mass of constituent molecules such as secondary products.

To summarize, molecular genetic technologies provide the ability to modulate and manipulate growth, development and biochemistry of the entire plant as well as at the cell, tissue and organ levels. Thus, plant morphology, development and biochemistry are altered to maximize or minimize the desired plant trait.

### SUMMARY OF THE INVENTION

The present invention, therefore, relates to isolated polynucleotides, polypeptides encoded thereby, and the use of those products for making transgenic plants.

The present invention also relates to processes for increasing the yield in plants, recombinant nucleic acid molecules and polypeptides used for these processes, their uses as well as to plants with an increased yield.

In the field of agriculture and forestry efforts are constantly being made to produce plants with an increased overall yield or increased yeild of biomass or chemical components, in particular in order to guarantee the supply of the constantly increasing world population with food and to guarantee the supply of reproducible raw materials. Conventionally, people try to obtain plants with an increased yield by breeding, but this is time-consuming and labor-intensive. Furthermore, appropriate breeding programs must be performed for each relevant plant species.

Recently, progress has been made by the genetic manipulation of plants. That is, by introducing into and expressing recombinant nucleic acid molecules in plants. Such approaches have the advantage of not usually being limited to one plant species, but being transferable to other plant species as well. EP-A 0 511 979, for example, discloses that the expression of a prokaryotic asparagine synthetase in plant cells inter alia leads to an increase in biomass production. Similarly, WO 96/21737 describes the production of plants with increased yield from the expression of deregulated or unregulated fructose-1,6-bisphosphatase due to an increased rate of the photosynthesis. Nevertheless, there still is a need for generally applicable processes that improve yield in plants interesting for agriculture or forestry purposes. The present invention addresses this need and relates to a process for increasing yield and/or biomass in plants, characterized in that recombinant DNA molecules stably integrated into the genome of plants are expressed and alter or modulate plant growth, development and/or biochemistry.

It was surprisingly found that the expression of the proteins according to the invention specifically lead to an alteration or modulation of yield.

The term “alteration or modulation of yield” preferably relates to an alteration or modulation in biomass production, in particular when determined as the fresh weight of the plant. Such an alteration or modulation in yield preferably refers to the so-called “sink” organs of the plant, which are the organs that take up the photoassimilates produced during photosynthesis, but can also refer to the “source” organs that produce the photoassimilates. Particularly preferred are parts of plants which can be harvested, such as seeds, fruits, storage roots, roots, tubers, flowers, buds, shoots, stems or wood and leaves. The alteration or modulation in yield according to the invention is at least 3% with regard to the biomass in comparison to non-transformed plants of the same genotype when cultivated under the same conditions, preferably at least 10% and particularly preferred at least 20%.

### BRIEF DESCRIPTION OF THE INDIVIDUAL TABLES

Table 1—Polynucleotide and Polypeptide Sequences

Table 1 sets forth the specific polynucleotide and polypeptide sequence of the invention. Each sequence is provided a number that directly follows a “>” symbol, and the description of the sequence directly follows on the next line in Table 1. It will be noted that a polynuceotide sequence is directly followed by the encoded polypeptide sequence.

Table 2—Microarray Results

Table 2 presents the results of the differential expression experiments for the mRNAs, as reported by their corresponding cDNA ID number, that were differentially transcribed under a particular set of conditions as compared to a control sample.

The “cDNA_ID” provides the identifier number for the cDNA tracked in the experiment. The column headed “EXPT_REP_ID” provides an identifier number for the particular experiment conducted. The column headed “SHORT_NAME” (e.g. At_{—}0.001%_MeJA_cDNAP) provides a short description of the experimental conditions used. The values in the column headed “Differential (±)” indicate whether expression of the cDNA was increased (+) or decreased (−) compared to the control.

Table 3—Microarray Experimental Parameters

Table 3 provides the experimental parameters used in conducting the microarray experiments. The first column, “Utility Section” idicates in which section a discussion of the utility can be found. The second column, “EXPT_REP_ID,” indicates the individual experiment. (e.g. 108569) detailed. The third column again uses the “SHORT_NAME” heading to identifiy the experiment (e.g. At_{—}0.001%_MeJA_cDNA_P). The fourth column, “PARAM_NAME,” identifies the parameter used or measured (e.g. Timepoint (hr)), while the fifth column, “VALUE” provides the descriptor for the particular parameter (e.g. “6”). As an example, when read together one understands that the “Methyl Jasmonate” section of the Specification provides information pertinent to the 0.001% MeJA (methyl jasmonate) experiment 108569, which contains data taken from a 6 hr Timepoint.

### DETAILED DESCRIPTION OF THE INVENTION

### 1. DEFINITIONS

The following terms are utilized throughout this application:

Allelic variant: An “allelic variant” is an alternative form of the same SDF, which resides at the same chromosomal locus in the organism. Allelic variations can occur in any portion of the gene sequence, including regulatory regions. Allelic variants can arise by normal genetic variation in a population. Allelic variants can also be produced by genetic engineering methods. An allelic variant can be one that is found in a naturally occurring plant, including a cultivar or ecotype. An allelic variant may or may not give rise to a phenotypic change, and may or may not be expressed. An allele can result in a detectable change in the phenotype of the trait represented by the locus. A phenotypically silent allele can give rise to a product.

Chimeric: The term “chimeric” is used to describe genes, as defined supra, or contructs wherein at least two of the elements of the gene or construct, such as the promoter and the coding sequence and/or other regulatory sequences and/or filler sequences and/or complements thereof, are heterologous to each other.

Constitutive Promoter: Promoters referred to herein as “constitutive promoters” actively promote transcription under most, but not necessarily all, environmental conditions and states of development or cell differentiation. Examples of constitutive promoters include the cauliflower mosaic virus (CaMV) 35S transcript initiation region and the 1′ or 2′ promoter derived from T-DNA of Agrobacterium tumefaciens, and other transcription initiation regions from various plant genes, such as the maize ubiquitin-1 promoter, known to those of skill.

Coordinately Expressed: The term “coordinately expressed,” as used in the current invention, refers to genes that are expressed at the same or a similar time and/or stage and/or under the same or similar environmental conditions.

Domain: Domains are fingerprints or signatures that can be used to characterize protein families and/or parts of proteins. Such fingerprints or signatures can comprise conserved (1) primary sequence, (2) secondary structure, and/or (3) three-dimensional conformation. Generally, each domain has been associated with either a family of proteins or motifs. Typically, these families and/or motifs have been correlated with specific in-vitro and/or in-vivo activities. A domain can be any length, including the entirety of the sequence of a protein. Detailed descriptions of the domains, associated families and motifs, and correlated activities of the polypeptides of the instant invention are described below. Usually, the polypeptides with designated domain(s) can exhibit at least one activity that is exhibited by any polypeptide that comprises the same domain(s).

Endogenous: The term “endogenous,” within the context of the current invention refers to any polynucleotide, polypeptide or protein sequence which is a natural part of a cell or organisms regenerated from said cell.

Exogenous: “Exogenous,” as referred to within, is any polynucleotide, polypeptide or protein sequence, whether chimeric or not, that is initially or subsequently introduced into the genome of an individual host cell or the organism regenerated from said host cell by any means other than by a sexual cross. Examples of means by which this can be accomplished are described below, and include Agrobacterium-mediated transformation (of dicots—e.g. Salomon et al. EMBO J. 3:141 (1984); Herrera-Estrella et al. EMBO J. 2:987 (1983); of monocots, representative papers are those by Escudero et al., Plant J. 10:355 (1996), Ishida et al., Nature Biotechnology 14:745 (1996), May et al., Bio/Technology 13:486 (1995)), biolistic methods (Armaleo et al., Current Genetics 17:97 1990)), electroporation, in planta techniques, and the like. Such a plant containing the exogenous nucleic acid is referred to here as a T₀ for the primary transgenic plant and T₁ for the first generation. The term “exogenous” as used herein is also intended to encompass inserting a naturally found element into a non-naturally found location.

Gene: The term “gene,” as used in the context of the current invention, encompasses all regulatory and coding sequence contiguously associated with a single hereditary unit with a genetic function. Genes can include non-coding sequences that modulate the genetic function that include, but are not limited to, those that specify polyadenylation, transcriptional regulation, DNA conformation, chromatin conformation, extent and position of base methylation and binding sites of proteins that control all of these. Genes comprised of “exons” (coding sequences), which may be interrupted by “introns” (non-coding sequences), encode proteins. A gene's genetic function may require only RNA expression or protein production, or may only require binding of proteins and/or nucleic acids without associated expression. In certain cases, genes adjacent to one another may share sequence in such a way that one gene will overlap the other. A gene can be found within the genome of an organism, artificial chromosome, plasmid, vector, etc., or as a separate isolated entity.

Heterologous sequences: “Heterologous sequences” are those that are not operatively linked or are not contiguous to each other in nature. For example, a promoter from corn is considered heterologous to an Arabidopsis coding region sequence. Also, a promoter from a gene encoding a growth factor from corn is considered heterologous to a sequence encoding the corn receptor for the growth factor. Regulatory element sequences, such as UTRs or 3′ end termination sequences that do not originate in nature from the same gene as the coding sequence originates from, are considered heterologous to said coding sequence. Elements operatively linked in nature and contiguous to each other are not heterologous to each other. On the other hand, these same elements remain operatively linked but become heterologous if other filler sequence is placed between them. Thus, the promoter and coding sequences of a corn gene expressing an amino acid transporter are not heterologous to each other, but the promoter and coding sequence of a corn gene operatively linked in a novel manner are heterologous.

Homologous gene: In the current invention, “homologous gene” refers to a gene that shares sequence similarity with the gene of interest. This similarity may be in only a fragment of the sequence and often represents a functional domain such as, examples including without limitation a DNA binding domain, a domain with tyrosine kinase activity, or the like. The functional activities of homologous genes are not necessarily the same.

Inducible Promoter: An “inducible promoter” in the context of the current invention refers to a promoter which is regulated under certain conditions, such as light, chemical concentration, protein concentration, conditions in an organism, cell, or organelle, etc. A typical example of an inducible promoter, which can be utilized with the polynucleotides of the present invention, is PARSK1, the promoter from the Arabidopsis gene encoding a serine-threonine kinase enzyme, and which promoter is induced by dehydration, abscissic acid and sodium chloride (Wang and Goodman, Plant J. 8:37 (1995)). Examples of environmental conditions that may affect transcription by inducible promoters include anaerobic conditions, elevated temperature, or the presence of light.

Misexpression: The term “misexpression” refers to an increase or a decrease in the transcription of a coding region into a complementary RNA sequence as compared to the wild-type. This term also encompasses expression of a gene or coding region for a different time period as compared. to the wild-type and/or from a non-natural location within the plant genome.

Orthologous Gene: In the current invention “orthologous gene” refers to a second gene that encodes a gene product that performs a similar function as the product of a first gene. The orthologous gene may also have a degree of sequence similarity to the first gene. The orthologous gene may encode a polypeptide that exhibits a degree of sequence similarity to a polypeptide corresponding to a first gene. The sequence similarity can be found within a functional domain or along the entire length of the coding sequence of the genes and/or their corresponding polypeptides.

Percentage of sequence identity: “Percentage of sequence identity,” as used herein, is determined by comparing two optimally aligned sequences over a comparison window, where the fragment of the polynucleotide or amino acid sequence in the comparison window may comprise additions or deletions (e.g., gaps or overhangs) as compared to the reference sequence (which does not comprise additions or deletions) for optimal alignment of the two sequences. The percentage is calculated by determning the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (USA) 85: 2444 (1988), by computerized implementations of these algorithms (GAP, BESTFIT, BLAST, PASTA, and TFASTA in the Wisconsin Genetics Software Package, Genetics Computer Group (GCG), 575 Science Dr., Madison, Wis.), or by inspection. Given that two sequences have been identified for comparison, GAP and BESTFIT are preferably employed to determine their optimal alignment. Typically, the default values of 5.00 for gap weight and 0.30 for gap weight length are used. The term “substantial sequence identity” between polynucleotide or polypeptide sequences refers to polynucleotide or polypeptide comprising a sequence that has at least 80% sequence identity, preferably at least 85%, more preferably at least 90% and most preferably at least 95%, even more preferably, at least 96%, 97%, 98% or 99% sequence identity compared to a reference sequence using the programs.

Plant Promoter: A “plant promoter” is a promoter capable of initiating transcription in plant cells and can drive or facilitate transcription of a fragment of the SDF of the instant invention or a coding sequence of the SDF of the instant invention. Such promoters need not be of plant origin. For example, promoters derived from plant viruses, such as the CaMV35S promoter or from Agrobacterium tumefaciens such as the T-DNA promoters, can be plant promoters. A typical example of a plant promoter of plant origin is the maize ubiquitin-1 (ubi-1)promoter known to those of skill.

Promoter: The term “promoter,” as used herein, refers to a region of sequence determiinants located upstream from the start of transcription of a gene and which are involved in recognition and binding of RNA polymerase and other proteins to initiate and modulate transcription. A basal promoter is the minimal sequence necessary for assembly of a transcription complex required for transcription initiation. Basal promoters frequently include a “TATA box” element usually located between 15 and 35 nucleotides upstream from the site of initiation of transcription. Basal promoters also sometimes include a “CCAAT box” element (typically a sequence CCAAT) and/or a GGGCG sequence, usually located between 40 and 200 nucleotides, preferably 60 to 120 nucleotides, upstream from the start site of transcription.

Regulatory Sequence: The term “regulatory sequence,” as used in the current invention, refers to any nucleotide sequence that influences transcription or translation initiation and rate, and stability and/or mobility of the transcript or polypeptide product. Regulatory sequences include, but are not limited to, promoters, promoter control elements, protein binding sequences, 5′ and 3′ UTRs, transcriptional start site, termination sequence, polyadenylation sequence, introns, certain sequences within a coding sequence, etc.

Signal Peptide: A “signal peptide” as used in the current invention is an amino acid sequence that targets the protein for secretion, for transport to an intracellular compartment or organelle or for incorporation into a membrane. Signal peptides are indicated in the tables and a more detailed description located below.

Specific Promoter: In the context of the current invention, “specific promoters” refers to a subset of inducible promoters that have a high preference for being induced in a specific tissue or cell and/or at a specific time during development of an organism. By “high preference” is meant at least 3-fold, preferably 5-fold, more preferably at least 10-fold still more preferably at least 20-fold, 50-fold or 100-fold increase in transcription in the desired tissue over the transcription in any other tissue. Typical examples of temporal and/or tissue specific promoters of plant origin that can be used with the polynucleotides of the present invention, are: PTA29, a promoter which is capable of driving gene transcription specifically in tapetum and only during anther development (Koltonow et al., Plant Cell 2:1201 (1990); RCc2 and RCc3, promoters that direct root-specific gene transcription in rice (Xu et al., Plant Mol. Biol. 27:237 (1995); TobRB27, a root-specific promoter from tobacco (Yamamoto et al., Plant Cell 3:371 (1991)). Examples of tissue-specific promoters under developmental control include promoters that initiate transcription only in certain tissues or organs, such as root, ovule, fruit, seeds, or flowers. Other suitable promoters include those from genes encoding storage proteins or the lipid body membrane protein, oleosin. A few root-specific promoters are noted above.

Stringency: “Stringency” as used herein is a function of probe length, probe composition (G+C content), and salt concentration, organic solvent concentration, and temperature of hybridization or wash conditions. Stringency is typically compared by the parameter Tₘ, which is the temperature at which 50% of the complementary molecules in the hybridization are hybridized, in terms of a temperature differential from Tₘ. High stringency conditions are those providing a condition of Tₘ-5° C. to Tₘ-10° C. Medium or moderate stringency conditions are those providing Tₘ-20° C. to Tₘ-29° C. Low stringency conditions are those providing a condition of Tₘ-40° C. to Tₘ-48° C. The relationship of hybridization conditions to Tₘ (in ° C.) is expressed in the mathematical equation
Tₘ=81.5 −16.6(log₁₀[Na⁺])+0.41(%G+C)−(600/N) (1)
where N is the length of the probe. This equation works well for probes 14 to 70 nucleotides in length that are identical to the target sequence. The equation below for Tₘ of DNA-DNA hybrids is useful for probes in the range of 50 to greater than 500 nucleotides, and for conditions that include an organic solvent (formamide).
Tₘ=81.5+16.6 log {[Na⁺]/(1+0.7[Na⁺])}+0.41(% G+C)−500/L 0.63(%formamide) (2)
where L is the length of the probe in the hybrid. (P. Tijessen, “Hybridization with Nucleic Acid Probes” in Laboratory Techniques in Biochemistry and Molecular Biology, P. C. vand der Vliet, ed., c. 1993 by Elsevier, Amsterdam.) The Tₘ of equation (2) is affected by the nature of the hybrid; for DNA-RNA hybrids Tₘ is 10-15° C. higher than calculated, for RNA-RNA hybrids Tₘ is 20-25° C. higher. Because the Tₘ decreases about 1° C. for each 1% decrease in homology when a long probe is used (Bonner et al., J. Mol. Biol. 81:123 (1973)), stringency conditions can be adjusted to favor detection of identical genes or related family members.

Equation (2) is derived assuming equilibrium and therefore, hybridizations according to the present invention are most preferably performed under conditions of probe excess and for sufficient time to achieve equilibrium. The time required to reach equilibrium can be shortened by inclusion of a hybridization accelerator such as dextran sulfate or another high volume polymer in the hybridization buffer.

Stringency can be controlled during the hybridization reaction or after hybridization has occurred by altering the salt and temperature conditions of the wash solutions used. The formulas shown above are equally valid when used to compute the stringency of a wash solution. Preferred wash solution stringencies lie within the ranges stated above; high stringency is 5-8° C. below Tₘ, medium or moderate stringency is 26-29° C. below Tₘ and low stringency is 45-48° C. below Tₘ.

Substantially free of: A composition containing A is “substantially free of” B when at least 85% by weight of the total A+B in the composition is A. Preferably, A comprises at least about 90% by weight of the total of A+B in the composition, more preferably at least about 95% or even 99% by weight. For example, a plant gene or DNA sequence can be considered substantially free of other plant genes or DNA sequences.

T₁: As used in the current application, the term T₁ refers to the cell or plant that is the direct result of a transformation experiment

T₂: As used in the current application, the term T2 refers to the progeny of the cell or plant that is the direct result of a transformation experiment.

T₃: As used in the current application, the term T₃ refers to second generation progeny of the cell or plant that is the direct result of a transformation experiment.

Translational start site: In the context of the current invention, a “translational start site” is usually an ATG in the cDNA transcript, more usually the first ATG. A single cDNA, however, may have multiple translational start sites.

Transcription start site: “Transcription start site” is used in the current invention to describe the point at which transcription is initiated. This point is typically located about 25 nucleotides downstream from a TFIID binding site, such as a TATA box. Transcription can initiate at one or more sites within the gene, and a single gene may have multiple transcriptional start sites, some of which may be specific for transcription in a particular cell-type or tissue.

Untranslated region (UTR): A “UTR” is any contiguous series of nucleotide bases that is transcribed, but is not translated. These untranslated regions may be associated with particular functions such as increasing mRNA message stability. Examples of UTRs include, but are not limited to polyadenylation signals, terminations sequences, sequences located between the transcriptional start site and the first exon (5′ UTR) and sequences located between the last exon and the end of the mRNA (3′ UTR).

Variant: The term “variant” is used herein to denote a polypeptide or protein or polynucleotide molecule that differs from others of its kind in some way. For example, polypeptide and protein variants can consist of changes in amino acid sequence and/or charge and/or post-translational modifications (such as glycosylation, etc).

### 2. IMPORTANT CHARACTERISTICS OF THE POLYNUCEOTIDES OF THE INVENTION

The genes and polynucleotides of the present invention are of interest because when they are misexpressed (i.e. when expressed at a non-natural location or in an increased amount) they produce plants with modified characteristics as discussed below and as evidenced by the results of differential expression experiments. These traits can be used to exploit or maximize plant products or characteristics. For example, an increase in plant height is beneficial in species grown or harvested for their main stem or trunk, such as ornamental cut flowers, fiber crops (e.g. flax, kenaf, hesperaloe, hemp) and wood producing trees. Increase in inflorescence thickness is also desirable for some ornamentals, while increases in the number, shape and size of leaves can lead to increased production/harvest from leaf crops such as lettuce, spinach, cabbage and tobacco

### 3. THE GENES OF THE INVENTION

The sequences of the invention were isolated from Arabidopsis thaliana.

### 4. USE OF THE GENES TO MAKE TRANSGENIC PLANTS

To use the sequences of the present invention or a combination of them or parts and/or mutants and/or fusions and/or variants of them, recombinant DNA constructs are prepared which comprise the polynucleotide sequences of the invention inserted into a vector, and which are suitable for transformation of plant cells. The construct is made using standard recombinant DNA techniques (Sambrook et al. 1989) and is introduced into the species of interest by Agrobacterium-mediated transformation or by other means of transformation as referenced below.

The vector backbone is any of those typical in the art such as plasmids, viruses, artificial chromosomes, BACs, YACs and PACs and vectors of the sort described by
(a) BAC: Shizuya et al., Proc. Natl. Acad. Sci. USA 89: 8794-8797 (1992); Hamilton et al., Proc. Natl. Acad. Sci. USA 93: 9975-9979 (1996);
(b) YAC: Burke et al., Science 236:806-812 (1987);
(c) PAC: Sternberg N. et al., Proc Natl Acad Sci USA. January;87(1):103-7 (1990);
(d) Bacteria-Yeast Shuttle Vectors: Bradshaw et al., Nucl Acids Res 23: 4850-4856 (1995);
(e) Lambda Phage Vectors: Replacement Vector, e.g., Frischauf et al., J. Mol Biol 170: 827-842 (1983); or Insertion vector, e.g., Huynh et al., In: Glover NM (ed) DNA Cloning: A practical Approach, Vol. 1 Oxford: IRL Press (1985); T-DNA gene fusion vectors :Walden et al., Mol Cell Biol 1: 175-194 (1990); and
(g) Plasmid vectors: Sambrook et al., infra.

Typically, the construct comprises a vector containing a sequence of the present invention with any desired transcriptional and/or translational regulatory sequences, such as promoters, UTRs, and 3′ end termination sequences. Vectors also typically include one or more of the following: origins of replication, scaffold attachment regions (SARs), markers, homologous sequences, introns, etc. The vector may also comprise a marker gene that confers a selectable phenotype on plant cells. The marker may encode biocide resistance, particularly antibiotic resistance, such as resistance to kanamycin, G418, bleomycin, hygromycin, or herbicide resistance, such as resistance to chlorosulfuron or phosphinotricin.

A plant promoter fragment is used that directs transcription of the gene in all tissues of a regenerated plant and may be a constitutive promoter, such as 355. Alternatively, the plant promoter directs transcription of a sequence of the invention in a specific tissue (tissue-specific promoters) or is otherwise under more precise environmental control (inducible promoters).

If proper polypeptide production is desired, a polyadenylation region at the 3′-end of the coding region is typically included. Thee polyadenylation region is derived from the natural gene, from a variety of other plant genes, or from T-DNA.

Knock-In Constructs

Ectopic expression of the sequences of the invention can also be accomplished using a “knock-in” approach. Here, the first component, an “activator line,” is created by generating a transgenic plant comprising a transcriptional activator operatively linked to a promoter. The second component comprises the desired cDNA sequence operatively linked to the target binding sequence/region of the transcriptional activator. The second component is transformed into the “activator line” or is used to transform a host plant to produce a “target” line that is crossed with the “activator line” by ordinary breeding methods. In either case, the result is the same. That is, the promoter drives production of the transcriptional activator protein that then binds to the target binding region to facilitate expression of the desired cDNA.

Any promoter that functions in plants is used in the first component., such as the 35S Cauliflower Mosaic Virus promoter or a tissue or organ specific promoter. Suitable transcriptional activator polypeptides include, but are not limited to, those encoding HAP1 and GAL4. The binding sequence recognized and targeted by the selected transcriptional activator protein is used in the second component.

Transformation

Techniques for transforming a wide variety of higher plant species are well known and described in the technical and scientific literature. See, e.g. Weising et al., Ann. Rev. Genet. 22:421 (1988); and Christou, Euphytica, v. 85, n.1-3:13-27, (1995).

The person skilled in the art knows processes for the transformation of monocotyledonous and dicotyledonous plants. A variety of techniques are available for introducing DNA into a plant host cell. These techniques comprise transformation of plant cells by DNA injection, DNA electroporation, use of bolistics methods, protoplast fusion and via T-DNA using Agrobacterium tumefaciens or Agrobacterium rhizogenes, as well as further possibilities.

DNA constructs of the invention are introduced into the genome of the desired plant host by a variety of conventional techniques. For example, the DNA construct is introduced directly into the genomic DNA of the plant cell using techniques such as electroporation, microinjection and polyethylene glycol precipitation of plant cell protoplasts or protoplast fusion. Electroporation techniques are described in Fromm et al. Proc. Natl Acad. Sci. USA 82:5824 (1985). Microinjection techniques are known in the art and well described in the scientific and patent literature. The plasmids do not have to fulfill specific requirements for use in DNA electroporation or DNA injection into plant cells. Simple plasmids such as pUC derivatives can be used.

The introduction of DNA constructs using polyethylene glycol precipitation is described in Paszkowski et al. EMBO J. 3:2717 (1984). Introduction of foreign DNA using protoplast fusion is described by Willmitzer (Willmitzer, L., 1993 Transgenic plants. In: Biotechnology, A Multi-Volume Comprehensive Treatise (H. J. Rehm, G. Reed, A. Pühler, P. Stadler, eds.), Vol. 2, 627-659, VCH Weinheim-New York-Basel-Cambridge).

Alternatively, the DNA constructs of the invention are introduced directly into plant tissue using ballistic methods, such as DNA particle bombardment. Ballistic transformation techniques are described in Klein et al. Nature 327:773 (1987). Introduction of foreign DNA using ballistics is described by Willmitzer (Willmitzer, L., 1993 Transgenic plants. In: Biotechnology, A Multi-Volume Comprehensive Treatise (H. J. Rehm, G. Reed, A. Pühler, P. Stadler, eds.), Vol. 2, 627-659, VCH Weinheim-New York-Basel-Cambridge).

DNA constructs are also introduced with the help of Agrobacteria. The use of Agrobacteria for plant cell transformation is extensively examined and sufficiently disclosed in the specification of EP-A 120 516, and in Hoekema (In: The Binary Plant Vector System Offsetdrukkerij Kanters B. V., Alblasserdam (1985), Chapter V), Fraley et al. (Crit. Rev. Plant. Sci. 4, 1-46) and An et al. (EMBO J. 4 (1985), 277-287). Using this technique, the DNA constructs of the invention are combined with suitable T-DNA flanking regions and introduced into a conventional Agrobacterium tumefaciens host vector. The virulence functions of the Agrobacterium tumefaciens host direct the insertion of the construct and adjacent marker(s) into the plant cell DNA when the cell is infected by the bacteria (McCormac et al., 1997, Mol. Biotechnol. 8 199; Hamilton, 1997, Gene 200:107; Salomon et al., 1984 EMBO J. 3:141; Herrera-Estrella et al., 1983 EMBO J. 2:987). Agrobacterium tumefaciens-mediated transformation techniques, including disarming and use of binary or co-integrate vectors, are well described in the scientific literature. See, for example Hamilton, C M., Gene 200:107 (1997); Müller et al. Mol. Gen. Genet. 207:171 (1987); Komari et al. Plant J. 10:165 (1996); Venkateswarlu et al. Biotechnology 9:1103 (1991) and Gleave, A P., Plant Mol. Biol. 20:1203 (1992); Graves and Goldman, Plant Mol. Biol. 7:34 (1986) and Gould et al., Plant Physiology 95:426 (1991).

For plant cell T-DNA transfer of DNA, plant explants, plant cells that have been cultured in suspension or protoplasts are co-cultivated with Agrobacterium tumefaciens or Agrobacterium rhizogenes. Whole plants are regenerated from the infected plant material using a suitable medium that contains antibiotics or biocides for the selection of transformed cells. Plants obtained in this way are then examnined for the presence of the DNA introduced. The transformation of dicotyledonous plants via Ti-plasmid-vector systems and Agrobacterium tumefaciens is well established.

Monocotyledonous plants are also transformed by means of Agrobacterium based vectors (See Chan et al., Plant Mol. Biol. 22 (1993), 491-506; Hiei et al., Plant J. 6 (1994), 271-282; Deng et al., Science in China 33 (1990), 28-34; Wilmink et al., Plant Cell Reports 11 (1992), 76-80; May et al., Bio/Technology 13 (1995), 486-492; Conner and Domisse; Int. J. Plant Sci. 153 (1992), 550-555; Ritchie et al., Transgenic Res. 2 (1993), 252-265). Maize transformation in particular is described in the literature (see, for example, WO95/06128, EP 0 513 849; EP 0 465 875; Fromm et al., Biotechnology 8 (1990), 833-844; Gordon-Kamm et al., Plant Cell 2 (1990), 603-618; Koziel et al., Biotechnology 11 (1993), 194-200). In EP 292 435 and in Shillito et al. (1989, Bio/Technology 7, 581) fertile plants are obtained from a mucus-free, soft (friable) maize callus. Prioli and Söndahl (1989, Bio/Technology 7, 589) also report regenerating fertile plants from maize protoplasts of the maize Cateto inbred line, Cat 100-1.

Other cereal species have also been successfully transformed, such as barley (Wan and Lemaux, see above; Ritala et al., see above) and wheat (Nehra et al., 1994, Plant J. 5, 285-297).

Alternatives to Agrobacterium transformation for monocotyledonous plants are ballistics, protoplast fusion, electroporation of partially permeabilized cells and use of glass fibers (See Wan and Lemaux, Plant Physiol. 104 (1994), 37-48; Vasil et al., Bio/Technology 11 (1993), 1553-1558; Ritala et al., Plant Mol. Biol. 24 (1994), 317-325; Spencer et al., Theor. Appl. Genet. 79 (1990), 625-631)).

Introduced DNA is usually stable after integration into the plant genome and is transmitted to the progeny of the transformed cell or plant. Generally the transformed plant cell contains a selectable marker that makes the transformed cells resistant to a biocide or an antibiotic such as kanamycin, G 418, bleomycin, hygromycin, phosphinotricin or others. Therefore, the individually chosen marker should allow the selection of transformed cells from cells lacking the introduced DNA.

The transformed cells grow within the plant in the usual way (McCormick et al., 1986, Plant Cell Reports 5 , 81-84) and the resulting plants are cultured normally. Transformed plant cells obtained by any of the above transformation techniques are cultured to regenerate a whole plant that possesses the transformed genotype and thus the desired phenotype. Such regeneration techniques rely on manipulation of certain phytohormones in a tissue culture growth medium, typically relying on a biocide and/or herbicide marker that has been introduced together with the desired nucleotide sequences.

Plant regeneration from cultured protoplasts is described in Evans et al., Protoplasts Isolation and Culture in “Handbook of Plant Cell Culture,” pp. 124-176, MacMillan Publishing Company, New York, 1983; and Binding, Regeneration of Plants, Plant Protoplasts, pp. 21-73, CRC Press, Boca Raton, 1988. Regeneration also occurs from plant callus, explants, organs, or parts thereof. Such regeneration techniques are described generally in Klee et al. Ann. Rev. of plant Phys. 38:467 (1987). Regeneration of monocots (rice) is described by Hosoyama et al. (Biosci. Biotechnol. Biochem. 58:1500 (1994)) and by Ghosh et al. (J. Biotechnol. 32:1 (1994)).

Seeds are obtained from the plants and used for testing stability and inheritance. Generally, two or more generations are cultivated to ensure that the phenotypic feature is stably maintained and transmitted.

One of skill will recognize that after the expression cassette is stably incorporated in transgenic plants and confirmed to be operable, it can be introduced into other plants by sexual crossing. Any of a number of standard breeding techniques can be used, depending upon the species to be crossed.

The nucleic acids of the invention are used to confer the trait of increased yeild, on essentially any plant.

The nucleotide sequences according to the invention generally encode an appropriate protein from any organism, in particular from plants, fungi, bacteria or animals. The sequences preferably encode proteins from plants or fungi. Preferably, the plants are higher plants, in particular starch or oil storing useful plants, such as potato or cereals such as rice, maize, wheat, barley, rye, triticale, oat, millet, etc., as well as spinach, tobacco, sugar beet, soya, cotton etc.

In principle, the process according to the invention can be applied to any plant. Therefore, monocotyledonous as well as dicotyledonous plant species are particularly suitable. The process is preferably used with plants that are interesting for agriculture, horticulture and/or forestry. Examples are vegetable plants such as cucumber, melon, pumpkin, eggplant, zucchini, tomato, spinach, cabbage species, peas, beans, etc., as well as fruits such as pears, apples, etc.

Thus, the invention has use over a broad range of plants, including species from the genera Anacardium, Arachis, Asparagus, Atropa, Avena, Brassica, Citrus, Citrullus, Capsicum, Carthamus, Cocos, Coffea, Cucumis, Cucurbita, Daucus, Elaeis, Fragaria, Glycine, Gossypium, Helianthus, Heterocallis, Hordeum, Hyoscyamus, Lactuca, Linum, Lolium,Lupinus, Lycopersicon, Malus, Manihot, Majorana, Medicago, Nicotiana, Olea, Oryza, Panieum, Pannesetum, Persea, Phaseolus, Pistachia, Pisum, Pyrus, Prunus, Raphanus, Ricinus, Secale, Senecio, Sinapis, Solanum, Sorghum, Theobromus, Trigonella, Triticum, Vicia, Vitis, Vigna, and, Zea.

Microaray Analysis

A major way that a cell controls its response to internal or external stimuli is by regulating the rate of transcription of specific genes. For example, the differentiation of cells during organogenensis into forms characteristic of the organ is associated with the selective activation and repression of large numbers of genes. Thus, specific organs, tissues and cells are functionally distinct due to the different populations of rnRNAs and protein products they possess. Internal signals program the selective activation and repression programs. For example, internally synthesized hormones produce such signals. The level of hormone is raised by increasing the level of transcription of genes encoding proteins concerned with hormone synthesis.

To measure how a cell reacts to internal and/or external stimuli, individual mRNA levels are measured and used as an indicator for the extent of transcription of the gene. Cells are exposed to a stimulus, and mRNA isolated and assayed at different time points after stimulation. The mRNA from the stimulated cells is compared to control cells that are not stimulated. The mRNA levels that are higher in the stimulated cell versus the control indicate a stimulus-specific response of the cell. The same is true of mRNA levels that are lower in stimulated cells versus the control condition.

Similar studies are performed with cells taken from an organism with a defined mutation in its genome as compared with cells without the mutation. Altered mRNA levels in the mutated cells indicate how the mutation causes transcriptional changes. These transcriptional changes are associated with the phenotype that the mutated cells exhibit that is different from the phenotype exhibited by the control cells.

Applicants use microarray techniques to measure the levels of mRNAs in cells from mutant plants, stimulated plants, and/or cells selected from specific organs. Microarray techniques are also used to measure the levels of mRNAs in cells from plants transformed with the polynucleotides of the invention. In this case, transformants with the genes of the invention are grown to an appropriate stage, and tissue samples prepared for the microarray differential expression analysis.

Microarray Experimental Procedures and Results

Procedures

1. Sample Tissue Preparation

Tissue samples for each of the expression analysis experiments were prepared as follows:

(a) Roots

Seeds of Arabidopsis thaliana (Ws) are sterilized in full strength bleach for less than 5 min., washed more than 3 times in sterile distilled deionized water and plated on MS agar plates. The plates are placed at 4° C. for 3 nights and then placed vertically into a growth chamber having 16 hr light/8 hr dark cycles, 23° C., 70% relative humidity and ˜11,000 LUX. After 2 weeks, the roots are cut from the agar, flash frozen in liquid nitrogen and stored at −80° C.

(b) Rosette Leaves Stems, and Siliques

Arabidopsis thaliana (Ws) seed is vernalized at 4° C. for 3 days before sowing in Metro-mix soil type 350. Flats are placed in a growth chamber having 16 hr light/8 hr dark, 80% relative humidity, 23° C. and 13,000 LUX for germination and growth. After 3 weeks, rosette leaves, stems, and siliques are harvested, flash frozen in liquid nitrogen and stored at −80° C. until use. After 4 weeks, siliques (<5 mm, 5-10 mm and >10 mm) are harvested, flash frozen in liquid nitrogen and stored at −80° C. until use. Five week old whole plants (used as controls) are harvested, flash frozen in liquid nitrogen and kept at −80° C. until RNA is isolated.

(c) Germination

Arabidopsis thaliana seeds (ecotype Ws) are sterilized in bleach and rinsed with sterile water. The seeds are placed in 100 mm petri plates containing soaked autoclaved filter paper. Plates are foil-wrapped and left at 4° C. for 3 nights to vernalize. After cold treatment, the foil is removed and plates are placed into a growth chamber having 16 hr light/8 hr dark cycles, 23° C., 70% relative humidity and ˜11,000 lux. Seeds are collected 1 d, 2 d, 3 d and 4 d later, flash frozen in liquid nitrogen and stored at ˜80° C. until RNA is isolated.

(d) Abscissic Acid (ABA)

Seeds of Arabidopsis thaliana (ecotype Wassilewskija) are sown in trays and left at 4° C. for 4 days to vernalize. They are then transferred to a growth chamber having grown 16 hr light/8 hr dark, 13,000 LUX, 70% humidity, and 20° C. and watered twice a week with 1 L of 1× Hoagland's solution. Approximately 1,000 fourteen day old plants are sprayed with 200-250 mls of 100 μM ABA in a 0.02% solution of the detergent Silwet L-77. Whole seedlings, including roots, are harvested within a 15 to 20 minute time period at 1 hr and 6 hr after treatment, flash-frozen in liquid nitrogen and stored at −80° C.

Seeds of maize hybrid 35A (Pioneer) are sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats are watered every three days for 7 days. Seedlings are carefully removed from the sand and placed in 1-liter beakers with 100 μM ABA for treatment. Control plants are treated with water. After 6 hr and 24 hr, aerial and root tissues are separated and flash frozen in liquid nitrogen prior to storage at −80° C.

(e) Brassinosteroid Responsive

Two separate experiments are performed, one with epi-brassinolide and one with the brassinosteroid biosynthetic inhibitor brassinazole. In the epi-brassinolide experiments, seeds of wild-type Arabidopsis thaliana (ecotype Wassilewskija) and the brassinosteroid biosynthetic mutant dwf4-1 are sown in trays and left at 4° C. for 4 days to vernalize. They are then transferred to a growth chamber having 16 hr light/8 hr dark, 11,000 LUX, 70% humidity and 22° C. temperature. Four week old plants are sprayed with a 1 μM solution of epi-brassinolide and shoot parts (unopened floral primordia and shoot apical meristems) are harvested three hours later. Tissue is flash-frozen in liquid nitrogen and stored at −80° C.

In the brassinazole experiments, seeds of wild-type Arabidopsis thaliana (ecotype Wassilewskija) are grown as described above. Four week old plants are sprayed with a 1 μM solution of brassinazole and shoot parts (unopened floral primordia and shoot apical meristems) are harvested three hours later. Tissue is flash-frozen in liquid nitrogen and stored at −80° C.

In addition to the spray experiments, tissue is prepared from two different mutants; (1) a dwf4-1 knock out mutant and (2) a mutant overexpressing the dwf4-1 gene.

Seeds of wild-type Arabidopsis thaliana (ecotype Wassilewskija) and of the dwf4-1 knock out and overexpressor mutants are sown in trays and left at 4° C. for 4 days to vernalize. They are then transferred to a growth chamber having 16 hr light/8 hr dark, 11,000 LUX, 70% humidity and 22° C. temperature. Tissue from shoot parts (unopened floral primordia and shoot apical meristems) is flash-frozen in liquid nitrogen and stored at −80° C.

Another experiment is completed with seeds of Arabidopsis thaliana (ecotype Wassilewskija), which are sown in trays and left at 4° C. for 4 days to vernalize. They are then transferred to a growth chamber. Plants are grown under long-day (16 hr light: 8 hr. dark) conditions, 13,000 LUX light intensity, 70% humidity, 20° C. temperature and watered twice a week with 1 L 1× Hoagland's solution (recipe recited in Feldmann et al., (1987) Mol. Gen. Genet. 208: 1-9 and described as complete nutrient solution). Approximately 1,000 14 day old plants are sprayed with 200-250 mls of 0.1 μM Epi-Brassinolite in 0.02% solution of the detergent Silwet L-77. At 1 hr. and 6 hrs. after treatment aerial tissues are harvested within a 15 to 20 minute time period and flash-frozen in liquid nitrogen.

Seeds of maize hybrid 35A (Pioneer) are sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats are watered every three days for 7 days. Seedlings are carefully removed from the sand and placed in 1-liter beakers with 0.1 μM epi-brassinolide for treatment. Control plants are treated with distilled deionized water. After 24 hr, aerial and root tissues are separated and flash frozen in liquid nitrogen prior to storage at −80° C.

(f) Nitrogen: High to Low

Wild type Arabidopsis thaliana seeds (ecotpye Ws) are surface sterilized with 30% Clorox, 0.1% Triton X-100 for 5 minutes. Seeds are then rinsed with 4-5 exchanges of sterile double distilled deionized water. Seeds are vernalized at 4° C. for 2-4 days in darkness. After cold treatment, seeds are plated on modified 1× MS media (without NH₄NO₃ or KNO₃), 0.5% sucrose, 0.5 g/L MES pH5.7, 1% phytagar and supplemented with KNO₃ to a final concentration of 60 mM (high nitrate modified 1× MS media). Plates are then grown for 7 days in a Percival growth chamber at 22° C. with 16 hr. light/8 hr dark.

Germinated seedlings are then transferred to a sterile flask containing 50 mL of high nitrate modified 1× MS liquid media. Seedlings are grown with mild shaking for 3 additional days at 22° C. in 16 hr. light/8 hr dark (in a Percival growth chamber) on the high nitrate modified 1× MS liquid media.

After three days of growth on high nitrate modified 1× MS liquid media, seedlings are transferred either to a new sterile flask containing 50 mL of high nitrate modified 1× MS liquid media or to low nitrate modified 1× MS liquid media (containing 20 □M KNO₃). Seedlings are grown in these media conditions with mild shaking at 22° C. in 16 hr light/8 hr dark for the appropriate time points and whole seedlings harvested for total RNA isolation via the Trizol method (LifeTech.). The time points used for the microarray experiments are 10 min. and 1 hour time points for both the high and low nitrate modified 1× MS media.

Alternatively, seeds that are surface sterilized in 30% bleach containing 0.1% Triton X-100 and further rinsed in sterile water, are planted on MS agar, (0.5% sucrose) plates containing 50 mM KNO₃ (potassium nitrate). The seedlings are grown under constant light (3500 LUX) at 22° C. After 12 days, seedlings are transferred to MS agar plates containing either 1 mM KNO₃ or 50 mM KNO₃. Seedlings transferred to agar plates containing 50 mM KNO₃ are treated as controls in the experiment. Seedlings transferred to plates with 1 mM KNO₃ are rinsed thoroughly with sterile MS solution containing 1 mM KNO₃. There are ten plates per transfer. Root tissue is collected and frozen in 15 mL Falcon tubes at various time points which include 1 hour, 2 hours, 3 hours, 4 hours, 6 hours, 9 hours, 12 hours, 16 hours, and 24 hours.

Maize 35A19 Pioneer hybrid seeds are sown on flats containing sand and grown in a Conviron growth chamber at 25° C., 16 hr light/8 hr dark, ˜13,000 LUX and 80% relative humidity. Plants are watered every three days with double distilled deionized water. Germinated seedlings are allowed to grow for 10 days and re watered with high nitrate modified 1× MS liquid media (see above). On day 11, young corn seedlings are removed from the sand (with their roots intact) and rinsed briefly in high nitrate modified 1× MS liquid media. The equivalent of half a flat of seedlings are then submerged (up to their roots) in a beaker containing either 500 mL of high or low nitrate modified 1× MS liquid media (see above for details).

At appropriate time points, seedlings are removed from their respective liquid media, the roots separated from the shoots and each tissue type flash frozen in liquid nitrogen and stored at −80° C. This is repeated for each time point. Total RNA is isolated using the Trizol method (see above) with root tissues only.

Corn root tissues isolated at the 4 hr and 16 hr time points are used for the microarray experiments. Both the high and low nitrate modified 1× MS media are used.

(g) Nitrogen: Low to High

Arabidopsis thaliana ecotype Ws seeds are sown on flats containing 4 L of a 1:2 mixture of Grace Zonolite vermiculite and soil. Flats are watered with 3 L of water and vernalized at 4° C. for five days. Flats are placed in a Conviron growth chamber having 16 hr light/8 hr dark at 20° C., 80% humidity and 17,450 LUX. Flats are watered with approximately 1.5 L of water every four days. Mature, bolting plants (24 days after germination) are bottom treated with 2 L of either a control (100 mM mannitol pH 5.5) or an experimental (50 mM ammonium nitrate, pH 5.5) solution. Roots, leaves and siliques are harvested separately 30, 120 and 240 minutes after treatment, flash frozen in liquid nitrogen and stored at −80° C.

Hybrid maize seed (Pioneer hybrid 35A19) are aerated overnight in deionized water. Thirty seeds are plated in each flat, which contained 4 liters of Grace zonolite vermiculite. Two liters of water are bottom fed and flats are kept in a Conviron growth chamber with 16 hr light/8 hr dark at 20° C. and 80% humidity. Flats are watered with 1 L of tap water every three days. Five day old seedlings are treated as described above with 2 L of either a control (100 mM mannitol pH 6.5) solution or 1 L of an experimental (50 mM ammonium nitrate, pH 6.8) solution. Fifteen shoots per time point per treatment are harvested 10, 90 and 180 minutes after treatment, flash frozen in liquid nitrogen and stored at −80° C.

Alternatively, seeds of Arabidopsis thaliana (ecotype Wassilewskija) are left at 4° C. for 3 days to vernalize. They are then sown on vermiculite in a growth chamber having 16 hours light/8 hours dark, 12,000-14,000 LUX, 70% humidity, and 20° C. They are bottom-watered with tap water, twice weekly. Twenty-four days old plants are sprayed with either water (control) or 0.6% ammonium nitrate at 4 μL/cm² of tray surface. Total shoots and some primary roots are cleaned of vermiculite, flash-frozen in liquid nitrogen and stored at −80° C.

(h) Methyl Jasmonate

Seeds of Arabidopsis thaliana (ecotype Wassilewskija) are sown in trays and left at 4° C. for 4 days to vernalize before being transferred to a growth chamber having 16 hr light/8 hr. dark, 13,000 LUX, 70% humidity, 20° C. temperature and watered twice a week with 1 L of a 1× Hoagland's solution. Approximately 1,000 14 day old plants are sprayed with 200-250 mls of 0.001% methyl jasmonate in a 0.02% solution of the detergent Silwet L-77. At 1 hr and 6 hrs after treatment, whole seedlings, including roots, are harvested within a 15 to 20 minute time period, flash-frozen in liquid nitrogen and stored at −80° C.

Seeds of maize hybrid 35A (Pioneer) are sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats are watered every three days for 7 days. Seedlings are carefully removed from the sand and placed in 1-liter beakers with 0.001% methyl jasmonate for treatment. Control plants are treated with water. After 24 hr, aerial and root tissues are separated and flash frozen in liquid nitrogen prior to storage at −80° C.

(i) Salicylic Acid

Seeds of Arabidopsis thaliana (ecotype Wassilewskija) are sown in trays and left at 4° C. for 4 days to vernalize before being transferred to a growth chamber having 16 hr light/8 hr. dark, 13,000 LUX, 70% humidity, 20° C. temperature and watered twice a week with 1 L of a 1× Hoagland's solution. Approximately 1,000 14 day old plants are sprayed with 200-250 mls of 5 mM salicylic acid (solubilized in 70% ethanol) in a 0.02% solution of the detergent Silwet L-77. At 1 hr and 6 hrs after treatment, whole seedlings, including roots, are harvested within a 15 to 20 minute time period flash-frozen in liquid nitrogen and stored at −80° C.

Alternatively, seeds of wild-type Arabidopsis thaliana (ecotype Columbia) and mutant CS3726 are sown in soil type 200 mixed with osmocote fertilizer and Marathon insecticide and left at 4° C. for 3 days to vernalize. Flats are incubated at room temperature with continuous light. Sixteen days post germination plants were sprayed with 2 mM SA, 0.02% SilwettL-77 or control solution (0.02% SilwettL-77. Aerial parts or flowers are harvested 1 hr, 4 hr, 6 hr, 24 hr and 3 weeks post-treatment flash frozen and stored at −80° C.

Seeds of maize hybrid 35A (Pioneer) are sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats are watered every three days for 7 days. Seedlings are carefully removed from the sand and placed in 1-liter beakers with 2 mM SA for treatment. Control plants are treated with water. After 12 hr and 24 hr, aerial and root tissues are separated and flash frozen in liquid nitrogen prior to storage at −80° C.

(j) Drought Stress

Seeds of Arabidopsis thaliana (Wassilewskija) are sown in pots and left at 4° C. for three days to vernalize before being transferred to a growth chamber having 16 hr light/8 hr dark, 150,000-160,000 LUX, 20° C. and 70% humidity. After 14 days, aerial tissues are cut and left to dry on 3MM Whatman paper in a petri-plate for 1 hour and 6 hours. Aerial tissues exposed for 1 hour and 6 hours to 3 MM Whatman paper wetted with 1× Hoagland's solution serve as controls. Tissues are harvested, flash-frozen in liquid nitrogen and stored at −80° C.

Alternatively, Arabidopsis thaliana (Ws) seed is vernalized at 4° C. for 3 days before sowing in Metromix soil type 350. Flats are placed in a growth chamber with 23° C., 16 hr light/8 hr. dark, 80% relative humidity, ˜13,000 LUX for germination and growth. Plants are watered with 1-1.5 L of water every four days. Watering is stopped 16 days after germination for the treated samples, but continues for the control samples. Rosette leaves and stems, flowers and siliques are harvested 2 d, 3 d, 4 d, 5 d, 6 d and 7 d after watering is stopped. Tissue is flash frozen in liquid nitrogen and kept at −80° C. until RNA is isolated. Flowers and siliques are also harvested on day 8 from plants that have undergone a 7 d drought treatment followed by 1 day of watering. Control plants (whole plants) are harvested after 5 weeks, flash frozen in liquid nitrogen and stored as above.

Seeds of maize hybrid 35A (Pioneer) are sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats are watered every three days for 7 days. Seedlings are carefully removed from the sand and placed in empty 1-liter beakers at room temperature for treatment. Control plants are placed in water. After 1 hr, 6 hr, 12 hr and 24 hr aerial and root tissues are separated and flash frozen in liquid nitrogen prior to storage at −80° C.

(k) Osmotic Stress

Seeds of Arabidopsis thaliana (Wassilewskija) are sown in trays and left at 4° C. for three days to vernalize before being transferred to a growth chamber having 16 hr light/8 hr dark, 12,000-14,000 LUX, 20° C., and 70% humidity. After 14 days, the aerial tissues are cut and placed on 3 MM Whatman paper in a petri-plate wetted with 20% PEG (polyethylene glycol-Mᵣ8,000) in 1× Hoagland's solution. Aerial tissues on 3 MM Whatman paper containing 1× Hoagland's solution alone serve as the control. Aerial tissues are harvested at 1 hour and 6 hours after treatment, flash-frozen in liquid nitrogen and stored at −80° C.

Seeds of maize hybrid 35A (Pioneer) are sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats are watered every three days for 7 days. Seedlings are carefully removed from the sand and placed in 1-liter beakers with 10% PEG (polyethylene glycol-Mᵣ8,000) for treatment. Control plants are treated with water. After 1 hr and 6 hr aerial and root tissues are separated and flash frozen in liquid nitrogen prior to storage at −80° C.

Seeds of maize hybrid 35A (Pioneer) are sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats are watered every three days for 7 days. Seedlings are carefully removed from the sand and placed in 1-liter beakers with 150 mM NaCl for treatment. Control plants are treated with water. After 1 hr, 6 hr, and 24 hr aerial and root tissues are separated and flash frozen in liquid nitrogen prior to storage at −80° C.

(l) Heat Shock Treatment

Seeds of Arabidopsis Thaliana (Wassilewskija) are sown in trays and left at 4° C. for three days to vernalize before being transferred to a growth chamber with 16 hr light/8 hr dark, 12,000-14,000 Lux, 70% humidity and 20° C., fourteen day old plants were transferred to a 42° C. growth chamber and aerial tissues are harvested 1 hr and 6 hr after transfer. Control plants are left at 20° C. and aerial tissues are harvested. Tissues are flash-frozen in liquid nitrogen and stored at −80° C.

Seeds of maize hybrid 35A (Pioneer) are sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats are watered every three days for 7 days. Seedlings are carefully removed from the sand and placed in 1-liter beakers containing 42° C. water for treatment. Control plants are treated with water at 25° C. After 1 hr and 6 hr aerial and root tissues are separated and flash frozen in liquid nitrogen prior to storage at −80° C.

(m) Cold Shock Treatment

Seeds of Arabidopsis thaliana (Wassilewskija) are sown in trays and left at 4° C. for three days to vernalize before being transferred to a growth chamber having 16 hr light/8 hr dark, 12,000-14,000 LUX, 20° C. and 70% humidity. Fourteen day old plants are transferred to a 4° C. dark growth chamber and aerial tissues are harvested 1 hour and 6 hours later. Control plants are maintained at 20° C. and covered with foil to avoid exposure to light. Tissues are flash-frozen in liquid nitrogen and stored at −80° C.

Seeds of maize hybrid 35A (Pioneer) are sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats are watered every three days for 7 days. Seedlings are carefully removed from the sand and placed in 1-liter beakers containing 4° C. water for treatment. Control plants are treated with water at 25° C. After 1 hr and 6 hr aerial and root tissues are separated and flash frozen in liquid nitrogen prior to storage at −80° C.

(n) Arabidopsis Seeds

Fruits (pod+seed) 0-5 mm

Seeds of Arabidopsis thaliana (ecotype Wassilewskija) are sown in pots and left at 4° C. for two to three days to vernalize. They are then transferred to a growth chamber. Plants are grown under long-day (16 hr light: 8 hr dark) conditions, 7000-8000 LUX light intensity, 70% humidity, and 22° C. temperature. 3-4 siliques (fruits) bearing developing seeds are selected from at least 3 plants and are hand-dissected to determine what developmental stage(s) are represented by the enclosed embryos. Description of the stages of Arabidopsis embryogenesis used in this determination are summarized by Bowman (1994). Silique lengths are then determined and used as an approximate determinant for embryonic stage. Siliques 0-5 mm in length containing post fertilization through pre-heart stage [0-72 hours after fertilization (HAF)] embryos are harvested and flash frozen in liquid nitrogen.

Fruits (pod+seed) 5-10 mm

Seeds of Arabidopsis thaliana (ecotype Wassilewskija) are sown in pots and left at 4° C. for two to three days to vernalize. They are then transferred to a growth chamber. Plants are grown under long-day (16 hr light: 8 hr dark) conditions, 7000-8000 LUX light intensity, 70% humidity, and 22° C. temperature. 3-4 siliques (fruits) bearing developing seeds are selected from at least 3 plants and are hand-dissected to determine what developmental stage(s) are represented by the enclosed embryos. Description of the stages of Arabidopsis embryogenesis used in this determination are summarized by Bowman (1994). Silique lengths are then determined and used as an approximate determinant for embryonic stage. Siliques 5-10 mm in length containing heart-through early upturned-U-stage [72-120 hours after fertilization (HAF)] embryos are harvested and flash frozen in liquid nitrogen.

Fruits (pod +seed) >10 mm

Seeds of Arabidopsis thaliana (ecotype Wassilewskija) are sown in pots and left at 4° C. for two to three days to vernalize. They are then transferred to a growth chamber. Plants are grown under long-day (16 hr light: 8 hr dark) conditions, 7000-8000 LUX light intensity, 70% humidity, and 22° C. temperature. 3-4 siliques (fruits) bearing developing seeds are selected from at least 3 plants and are hand-dissected to determine what developmental stage(s) are represented by the enclosed embryos. Description of the stages of Arabidopsis embryogenesis used in this determination are summarized by Bowman (1994). Silique lengths are then determined and used as an approximate determinant for embryonic stage. Siliques>10 mm in length containing green, late upturned-U-stage [>120 hours after fertilization (HAF)-9 days after flowering (DAF)] embryos are harvested and flash frozen in liquid nitrogen.

Green Pods 5-10 mm (Control Tissue for Samples 72-74)

Seeds of Arabidopsis thaliana (ecotype Wassilewskija) are sown in pots and left at 4° C. for two to three days to vernalize. They are then transferred to a growth chamber. Plants are grown under long-day (16 hr light: 8 hr dark) conditions, 7000-8000 LUX light intensity, 70% humidity, and 22° C. temperature. 3-4 siliques (fruits) bearing developing seeds are selected from at least 3 plants and are hand-dissected to determine what developmental stage(s) are represented by the enclosed embryos. Description of the stages of Arabidopsis embryogenesis used in this determination are summarized by Bowman (1994). Silique lengths are then determined and used as an approximate determinant for embryonic stage. Green siliques 5-10 mm in length containing developing seeds 72-120 hours after fertilization (HAF)] are opened and the seeds removed. The remaining tissues (green pods minus seed) are harvested and flash frozen in liquid nitrogen.

Green Seeds from Fruits>10 mm

Seeds of Arabidopsis thaliana (ecotype Wassilewskija) are sown in pots and left at 4° C. for two to three days to vernalize. They are then transferred to a growth chamber. Plants are grown under long-day (16 hr light: 8 hr dark) conditions, 7000-8000 LUX light intensity, 70% humidity, and 22° C. temperature. 3-4 siliques (fruits) bearing developing seeds are selected from at least 3 plants and are hand-dissected to determine what developmental stage(s) are represented by the enclosed embryos. Description of the stages of Arabidopsis embryogenesis used in this determination are summarized by Bowman (1994). Silique lengths are then determined and used as an approximate determinant for embryonic stage. Green siliques>10 mm in length containing developing seeds up to 9 days after flowering (DAF)] are opened and the seeds removed and harvested and flash frozen in liquid nitrogen.

Brown Seeds from Fruits>10 mm

Seeds of Arabidopsis thaliana (ecotype Wassilewskija) are sown in pots and left at 4° C. for two to three days to vernalize. They are then transferred to a growth chamber. Plants are grown under long-day (16 hr light: 8 hr dark) conditions, 7000-8000 LUX light intensity, 70% humidity, and 22° C. temperature. 3-4 siliques (fruits) bearing developing seeds are selected from at least 3 plants and are hand-dissected to determine what developmental stage(s) are represented by the enclosed embryos. Description of the stages of Arabidopsis embryogenesis used in this determination are summarized by Bowman (1994). Silique lengths are then determined and used as an approximate determinant for embryonic stage. Yellowing siliques>10 mm in length containing brown, dessicating seeds>11 days after flowering (DAF)] are opened and the seeds removed and harvested and flash frozen in liquid nitrogen.

Green/Brown Seeds from Fruits>10 mm

Seeds of Arabidopsis thaliana (ecotype Wassilewskija) are sown in pots and left at 4° C. for two to three days to vernalize. They are then transferred to a growth chamber. Plants are grown under long-day (16 hr light: 8 hr dark) conditions, 7000-8000 LUX light intensity, 70% humidity, and 22° C. temperature. 3-4 siliques (fruits) bearing developing seeds are selected from at least 3 plants and are hand-dissected to determine what developmental stage(s) are represented by the enclosed embryos. Description of the stages of Arabidopsis embryogenesis used in this determination are summarized by Bowman (1994). Silique lengths are then determined and used as an approximate determinant for embryonic stage. Green siliques>10 mm in length containing both green and brown seeds>9 days after flowering (DAF)] are opened and the seeds removed and harvested and flash frozen in liquid nitrogen.

Mature Seeds (24 hours After Imbibition)

Mature dry seeds of Arabidopsis thaliana (ecotype Wassilewskija) are sown onto moistened filter paper and left at 4° C. for two to three days to vernalize. Imbibed seeds are then transferred to a growth chamber [16 hr light: 8 hr dark conditions, 7000-8000 LUX light intensity, 70% humidity, and 22° C. temperature], the emerging seedlings are harvested after 48 hours and flash frozen in liquid nitrogen.

Mature Seeds (Dry)

Seeds of Arabidopsis thaliana (ecotype Wassilewskija) are sown in pots and left at 4° C. for two to three days to vernalize. They are then transferred to a growth chamber. Plants are grown under long-day (16 hr light: 8 hr dark) conditions, 7000-8000 LUX light intensity, 70% humidity, and 22° C. temperature and taken to maturity. Mature dry seeds are collected, dried for one week at 28° C., and vernalized for one week at 4° C. before being used as a source of RNA.

(o) Herbicide Treament

Arabidopsis thaliana (Ws) seeds are sterilized for 5 min. with 30% bleach, 50 μl Triton in a total volume of 50 ml. Seeds are vernalized at 4° C. for 3 days before being plated onto GM agar plates at a density of about 144 seeds per plate. Plates are incubated in a Percival growth chamber having 16 hr light/8 hr dark, 80% relative humidity, 22° C. and 11,000 LUX for 14 days.

Plates are sprayed (˜0.5 mls/plate) with water, Finale (1.128 g/L), Glean (1.88 g/L), RoundUp (0.01 g/L) or Trimec (0.08 g/L). Tissue is collected and flash frozen in liquid nitrogen at the following time points: 0, 1, 2, 4, 8, 12 and 24 hours. Frozen tissue is stored at −80° C. prior to RNA isolation.

(p) Root Tips

Seeds of Arabidopsis thaliana (ecotye Ws) are placed on MS plates and vernalized at 4° C. for 3 days before being placed in a 25° C. growth chamber having 16 hr light/8 hr dark, 70% relative humidty and about 3 W/m². After 6 days, young seedlings are transferred to flasks containing B5 liquid medium, 1% sucrose and 0.05 mg/l indole-3-butyric acid. Flasks are incubated at room temperature with 100 rpm agitation. Media is replaced weekly. After three weeks, roots re harvested and incubated for 1 hr with 2% pectinase, 0.2% cellulase, pH 7 before straining through a #80 (Sigma) sieve. The root body material remaining on the sieve (used as the control) is flash frozen and stored at −80° C. until use. The material that passed through the #80 sieve is strained through a #200 (Sigma) sieve and the material remaining on the sieve (root tips) is flash frozen and stored at −80° C. until use. Approximately 10 mg of root tips are collected from one flask of root culture.

Seeds of maize hybrid 35A (Pioneer) are sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats are watered every three days for 8 days. Seedlings are carefully removed from the sand and the root tips (˜2 mm long) are removed and flash frozen in liquid nitrogen prior to storage at −80° C. The tissues above the root tips (˜1 cm long) are cut, treated as above and used as control tissue.

(q) Imbibed Seed

Seeds of maize hybrid 35A (Pioneer) are sown in water-moistened sand in covered flats (10 rows, 5-6 seed/row) and covered with clear, plastic lids before being placed in a growth chamber having 16 hr light (25° C.)/8 hr dark (20° C.), 75% relative humidity and 13,000-14,000 LUX. One day after sowing, whole seeds are flash frozen in liquid nitrogen prior to storage at −80° C. Two days after sowing, embryos and endosperm are isolated and flash frozen in liquid nitrogen prior to storage at −80° C. On days 3-6, aerial tissues, roots and endosperm are isolated and flash frozen in liquid nitrogen prior to storage at −80° C.

(r) Flowers (Green, White or Buds)

Approximately 10 μl of Arabidopsis thaliana seeds (ecotype Ws) are sown on 350 soil (containing 0.03% marathon) and vernalized at 4° C. for 3 days. Plants are then grown at room temperature under fluorescent lighting until flowering. Flowers are harvested after 28 days in three different categories. Buds that have not opened at all and are completely green are categorized as “flower buds” (also referred to as green buds by the investigator). Buds that have started to open, with white petals emerging slightly are categorized as “green flowers” (also referred to as white buds by the investigator). Flowers that have mostly opened (with no silique elongation) with white petals completely visible are categorized as “white flowers” (also referred to as open flowers by the investigator). Buds and flowers are harvested with forceps, flash frozen in liquid nitrogen and stored at −80° C. until RNA is isolated.

2. Microarray Hybridization Procedures

Microarray technology provides the ability to monitor mRNA transcript levels of thousands of genes in a single experiment. These experiments simultaneously hybridize two differentially labeled fluorescent cDNA pools to glass slides that have been previously spotted with cDNA clones of the same species. Each arrayed cDNA spot has a corresponding ratio of fluorescence that represents the level of disparity between the respective mRNA species in the two sample pools. Thousands of polynucleotides are spotted on one slide, and each experiment generates a global expression pattern.

Coating Slides

The microarray consists of a chemically coated microscope slide, referred to herein as a “chip” with numerous polynucleotide samples arrayed at a high density. The poly-L-lysine coating allows for this spotting at high density by providing a hydrophobic surface, reducing the spreading of spots of DNA solution arrayed on the slides. Glass microscope slides (Gold Seal #3010 manufactured by Gold Seal Products, Portsmouth, N.H., USA) are coated with a 0.1% W/V solution of Poly-L-lysine (Sigma, St. Louis, Mo.) using the following protocol:
1. Slides are placed in slide racks (Shandon Lipshaw #121). The racks are then put in chambers (Shandon Lipshaw #121).
2. Cleaning solution is prepared:
   70 g NaOH is dissolved in 280 mL ddH2O.
   420 mL 95% ethanol is added. The total volume is 700 mL (=2×350 mL); it is stirred until completely mixed. If the solution remains cloudy, ddH₂O is added until clear.
3. The solution is poured into chambers with slides; the chambers are covered with glass lids. The solution is mixed on an orbital shaker for 2 hr.
4. The racks are quickly transferred to fresh chambers filled with ddH₂O. They are rinsed vigorously by plunging racks up and down. Rinses are repeated 4× with fresh ddH₂O each time, to remove all traces of NaOH-ethanol.
5. Polylysine solution is prepared:
   0 mL poly-L-lysine+70 mL tissue culture PBS in 560 mL water, using plastic graduated cylinder and beaker.
6. Slides are transferred to polylysine solution and shaken for 1 hr.
7. The rack is transferred to a fresh chambers filled with ddH₂O. It is plunged up and down 5× to rinse.
8. The slides are centrifuged on microtiter plate carriers (paper towels are placed below the rack to absorb liquid) for 5 min. @ 500 rpm. The slide racks are transferred to empty chambers with covers.
9. Slide racks are dried in a 45 C oven for 10 min.
10. The slides are stored in a closed plastic slide box.
11. Normally, the surface of lysine coated slides is not very hydrophobic immediately after this process, but becomes increasingly hydrophobic with storage. A hydrophobic surface helps ensure that spots do not run together while printing at high densities. After they ag for 10 days to a month ,the slides are ready to use. However, coated slides that have been sitting around for long periods of time are usually too old to be used. This is because they develop opaque patches, visible when held to the light, and these result in high background hybridization from the fluorescent probe.

Alternatively, pre-coated glass slides are purchased from TeleChem International, Inc. (Sunnyvale, Calif. 94089; catalog number SMM-25, Superamine substrates).

PCR Amplification OfcDNA Clone Inserts

Polynucleotides are amplified from Arabidopsis cDNA clones using insert specific probes. The resulting 100 uL PCR reactions are purified with Qiaquick 96 PCR purification columns (Qiagen, Valencia, Calif., USA) and eluted in 30 uL of 5 mM Tris. 8.5uL of the elution are mixed with 1.5 uL of 20×SSC to give a final spotting solution of DNA in 3×SSC. The concentrations of DNA generated from each clone vary between 10-100 ng/ul, but are usually about 50 ng/ul.

Arraying of PCR Products on Glass Slides

PCR products from cDNA clones are spotted onto the poly-L-Lysine coated glass slides using an arrangement of quill-tip pins (ChipMaker 3 spotting pins; Telechem, International, Inc., Sunnyvale, Calif., USA) and a robotic arrayer (PixSys 3500, Cartesian Technologies, Irvine, Calif., USA). Around 0.5 nl of a prepared PCR product is spotted at each location to produce spots with approximately 100 um diameters. Spot center-to-center spacing is from 180 um to 210 um depending on the array. Printing is conducted in a chamber with relative humidity set at 50%.

Slides containing maize sequences are purchased from Agilent Technology (Palo Alto, Calif. 94304).

Post-Processing of Slides

After arraying, slides are processed through a series of steps—rehydration, UV cross-linking, blocking and denaturation—required prior to hybridization. Slides are rehydrated by placing them over a beaker of warm water (DNA face down), for 2-3 sec, to distribute the DNA more evenly within the spots, and then snap dried on a hot plate (DNA side, face up). The DNA is then cross-linked to the slides by UV irradiation (60-65 mJ; 2400 Stratalinker, Stratagene, La Jolla, Calif., USA).

Following this, a blocking step is performed to modify remaining free lysine groups, and hence minimize their ability to bind labeled probe DNA. To achieve this the arrays are placed in a slide rack. An empty slide chamber is left ready on an orbital shaker. The rack is bent slightly inwards in the middle, to ensure the slides do not run into each other while shaking. The blocking solution is prepared as follows: 3×350-ml glass chambers (with metal tops) are set to one side, and a large round Pyrex dish with dH₂O is placed ready in the microwave. At this time, 15 ml sodium borate is prepared in a 50 ml conical tube.

6-g succinic anhydride is dissolved in approx. 325-350 mL 1-methyl-2-pyrrolidinone. Rapid addition of reagent is crucial.
a. Immediately after the last flake of the succinic anhydride dissolves, the 15-mL sodium borate is added.
b. Immediately after the sodium borate solution is mixed in, the solution is poured into an empty slide chamber.
c. The slide rack is plunged rapidly and evenly in the solution. It is vigorously shaken up and down for a few seconds, making sure slides never leave the solution.
d. It is mixed on an orbital shaker for 15-20 min. Meanwhile, the water in the Pyrex dish (enough to cover slide rack) is heated to boiling.

Following this, the slide rack is gently plunged in the 95 C water Oust stopped boiling) for 2 min. Then the slide rack is plunged 5× in 95% ethanol. The slides and rack are centrifuged for 5 min. @ 500 rpm. The slides are loaded quickly and evenly onto the carriers to avoid streaking. The arrays are used immediately or stored in a slide box.

The Hybridization process begins with the isolation of mRNA from the two tissues in question (see “Isolation of total RNA” and “Isolation of mRNA”, below) followed by their conversion to single stranded cDNA (see “Generation of probes for hybridization”, below). The cDNA from each tissue is independently labeled with a different fluorescent dye and then both samples are pooled together. This final differentially labeled cDNA pool is then placed on a processed microarray and allowed to hybridize (see “Hybridization and wash conditions”, below).

Isolation of Total RNA

Approximately 1 g of plant tissue is ground in liquid nitrogen to a fine powder and transferred into a 50-ml centrifuge tube containing 10 ml of Trizol reagent. The tube is vigorously vortexed for 1 rmin and then incubated at room temperature for 10-20 min. on an orbital shaker at 220 rpm. Two ml of chloroform are added to the tube and the solution vortexed vigorously for at least 30-sec before again incubating at room temperature with shaking. The sample is then centrifuged at 12,000×g (10,000 rpm) for 15-20 min at 4° C. The aqueous layer is removed and mixed by inversion with 2.5 ml of 1.2 M NaCl/0.8 M Sodium Citrate and 2.5 ml of isopropyl alcohol added. After a 10 min. incubation at room temperature, the sample is centrifuged at 12,000×g (10,000 rpm) for 15 min at 4° C. The pellet is washed with 70% ethanol, re-centrifuged at 8,000 rpm for 5 min and then air dried at room temperature for 10 min. The resulting total RNA is dissolved in either TE (10 mM Tris-HCl, 1 mM EDTA, pH 8.0) or DEPC (diethylpyrocarbonate) treated deionized water (RNAse-free water). For subsequent isolation of mRNA using the Qiagen kit, the total RNA pellet is dissolved in RNAse-free water.

Isolation of mRNA

mRNA is isolated using the Qiagen Oligotex mRNA Spin-Column protocol (Qiagen, Valencia, Calif.). Briefly, 500 μl OBB buffer (20 mM Tris-Cl, pH 7.5, 1 M NaCl, 2 mM EDTA, 0.2% SDS) is added to 500 μl of total RNA (0.5-0.75 mg) and mixed thoroughly. The sample is first incubated at 70° C. for 3 min, then at room temperature for 10 minutes and finally centrifuged for 2 min at 14,000-18,000×g. The pellet is resuspended in 400 μl OW2 buffer (10 mM Tris-Cl, pH 7.5, 150 mM NaCl, 1 mM EDTA) by vortexing, the resulting solution placed on a small spin column in a 1.5 ml RNase-free microcentrifuge tube and centrifuged for 1 min at 14,000-18,000×g. The spin column is transferred to a new 1.5 ml RNase-free microcentrifuge tube and washed with 400 μl of OW2 buffer. To release the isolated mRNA from the resin, the spin column is again transferred to a new RNase-free 1.5 ml microcentrifuge tube, 20-100 μl 70° C. OEB buffer (5 mM Tris-Cl, pH 7.5) added and the resin resuspended in the resulting solution via pipeting. The mRNA solution is collected after centrifuging for 1 min at 14,000-18,000×g.

Alternatively, mRNA is isolated using the Stratagene Poly(A) Quik mRNA Isolation Kit (Startagene, La Jolla, Calif.). Here, up to 0.5 mg of total RNA (maximum volume of 1 ml) are incubated at 65° C. for 5 minutes, snap cooled on ice and 0.1× volumes of 10× sample buffer (10 mM Tris-HCl (pH 7.5), 1 mM EDTA (pH 8.0) 5 M NaCl) added. The RNA sample is applied to a prepared push column and passed through the column at a rate of ˜1 drop every 2 sec. The solution collected is reapplied to the column and collected as above. 200 μl of high salt buffer (10 mM Tris-HCl (pH 7.5), 1 mM EDTA, 0.5 NaCl) are applied to the column and passed through the column at a rate of ˜1 drop every 2 sec. This step is repeated and followed by three low salt buffer (10 mM Tris-HCl (pH 7.5), 1 mM EDTA, 0.1 M NaCl) washes preforned in a similar manner. mRNA is eluted by applying to the column four separate 200 μl aliquots of elution buffer (10 mM Tris-HCl (pH 7.5), 1 mM EDTA) preheated to 65° C. Here, the elution buffer is passed through the column at a rate of 1 drop/sec. The resulting mRNA solution is precipitated by adding 0.1× volumes of 10× sample buffer, 2.5 volumes of ice-cold 100% ethanol, incubating overnight at −20° C. and centrifuging at 14,000-18,000×g for 20-30 min at 4° C. The pellet is washed with 70% ethanol and air dried for 10 min. at room temperature before resuspension in RNase-free deionized water.

Preparation of Yeast Controls

Plasmid DNA is isolated from the following yeast clones using Qiagen filtered maxiprep kits (Qiagen, Valencia, Calif.): YAL022c(Fun26), YAL031 c(Fun21), YBR032w, YDL131w, YDL182w, YDL194w, YDL196w, YDR050c and YDR116c. Plasmid DNA is linearized with either BsrBI (YAL022c(Fun26), YAL031 c(Fun21), YDL131w, YDL182w, YDL194w, YDL196w, YDR050c) or AflIII (YBR032w, YDR116c) and isolated.

In Vitro Transcription of Yeast Clones

The following solution is incubated at 37° C. for 2 hours: 17 μl of isolated yeast insert DNA (1 μg), 20 μl 5× buffer, 10 μl 100 mM DTT, 2.5 μl (100 U) RNasin, 20 μl 2.5 mM (ea.) rNTPs, 2.7 μl (40 U) SP6 polymerase and 27.8 μl RNase-free deionized water. 2 μl (2 U) Ampli DNase I is added and the incubation continued for another 15 min. 10 μl 5M NH₄OAC and 100 μl phenol:chloroform:isoamyl alcohol (25:24:1) are added, the solution vortexed and then centrifuged to separate the phases. To precipitate the RNA, 250 μl ethanol is added and the solution incubated at −20° C. for at least one hour. The sample is then centrifuged for 20 min at 4° C. at 14,000-18,000×g, the pellet washed with 500 μl of 70% ethanol, air dried at room temperature for 10 min and resuspended in 100 μl of RNase-free deionized water. The precipitation procedure is then repeated.

Alternatively, after the two-hour incubation, the solution is extracted with phenol/chloroform once before adding 0.1 volume 3M sodium acetate and 2.5 volumes of 100% ethanol. The solution is centrifuged at 15,000rpm, 4° C. for 20 minutes and the pellet resuspended in RNase-free deionized water. The DNase I treatment is carried out at 37° C. for 30 minutes using 2 U of Ampli DNase I in the following reaction condition: 50 mM Tris-HCl (pH 7.5), 10 mM MgCl₂. The DNase I reaction is then stopped with the addition of NH₄OAC and phenol:chloroform:isoamyl alcohol (25:24:1), and RNA isolated as described above.

0. 15-2.5 ng of the in vitro transcript RNA from each yeast clone are added to each plant mRNA sample prior to labeling to serve as positive (internal) probe controls.

Generation of Probes for Hybridization

Generation of Labeled Probes for Hybridization from First-Strand cDNA

Hybridization probes are generated from isolated mRNA using an Atlas™ Glass Fluorescent Labeling Kit (Clontech Laboratories, Inc., Palo Alto, Calif., USA). This entails a two step labeling procedure that first incorporates primary aliphatic amino groups during cDNA synthesis and then couples fluorescent dye to the cDNA by reaction with the amino functional groups. Briefly, 5 μg of oligo(dT)₁₈ primer d(TTTTTTTTTTTTTTTTTTV) is mixed with Poly A+ mRNA (1.5-2 μg mRNA isolated using the Qiagen Oligotex mRNA Spin-Column protocol or-the Stratagene Poly(A) Quik mRNA Isolation protocol (Stratagene, La Jolla, Calif., USA)) in a total volume of 25 μl. The sample is incubated in a thermocycler at 70° C. for 5 min, cooled to 48° C. and 10 μl of 5× cDNA Synthesis Buffer (kit supplied), 5 μl 10× dNTP mix (dATP, dCTP, dGTP, dTTP and aminoallyl-dUTP; kit supplied), 7.5 μl deionized water and 2.5 μl MMLV Reverse Transcriptase (500 U) added. The reaction is then incubated at 48° C. for 30 minutes, followed by 1 hr incubation at 42° C. At the end of the incubation the reaction is heated to 70° C. for 10 min, cooled to 37° C. and 0.5 μl (5 U) RNase H added, before incubating for 15 min at 37° C. The solution is vortexed for 1 min after the addition of 0.5 μl 0.5 M EDTA and 5 μl of QuickClean Resin (kit supplied) then centrifuged at 14,000-18,000×g for 1 min. After removing the supernatant to a 0.45 μm spin filter (kit supplied), the sample is again centrifuged at 14,000-18,000×g for 1 min, and 5.5 μl 3 M sodium acetate and 137.5 μl of 100% ethanol added to the sample before incubating at −20° C. for at least 1 hr. The sample is then centrifuged at 14,000-18,000×g at 4° C. for 20 min, the resulting pellet washed with 500 μl 70% ethanol, air-dried at room temperature for 10 min and resuspended in 10 μl of 2× fluorescent labeling buffer (kit provided). 10 μl each of the fluorescent dyes Cy3 and Cy5 (Amersham Pharmacia (Piscataway, N.J., USA); prepared according to Atlas™ kit directions of Clontech) are added and the sample incubated in the dark at room temperature for 30 min.

The fluorescently labeled first strand cDNA is precipitated by adding 2 μl 3M sodium acetate and 50 μl 100% ethanol, incubated at −20° C. for at least 2 hrs, centrifuged at 14,000-18,000×g for 20 min, washed with 70% ethanol, air-dried for 10 min and dissolved in 100 μl of water.

Alternatively, 3-4 μg mRNA, 2.5 (˜8.9 ng of in vitro translated mRNA) μl yeast control and 3 μg oligo dTV (TTTTTTTTTTTTTTTTTT(A/C/G) are mixed in a total volume of 24.7 μl. The sample is incubated in a thermocycler at 70° C. for 10 min. before chilling on ice. To this, 8 μl of 5× first strand buffer (SuperScript II RNase H-Reverse Transcriptase kit from Invitrogen (Carlsbad, Calif. 92008); cat no. 18064022), 0.8° C. of aa-dUTP/dNTP mix (50×; 25 mM dATP, 25 mM dGTP, 25 mM dCTP, 15 mM dTTP, 10 mM aminoallyl-dUTP), 4 μl of 0.1 M DTT and 2.5 μl (500 units) of Superscript R.T.II enzyme (Stratagene) are added. The sample is incubated at 42° C. for 2 hours before a mixture of 10° C. of 1M NaOH and 10° C. of 0.5 M EDTA are added. After a 15 minute incubation at 65° C., 25 μi of 1 M Tris pH 7.4 is added. This is mixed with 450 μl of water in a Microcon 30 column before centrifugation at 11,000×g for 12 min. The column is washed twicewith 450 μl (centrifugation at 11,000 g, 12 min.) before eluting the sample by inverting the Microcon column and centrifuging at 11,000×g for 20 seconds. Sample is dehydrated by centrifugation under vacuum and stored at −20° C.

Each reaction pellet is dissolved in 9 μl of 0.1 M carbonate buffer (0.1M sodium carbonate and sodium bicarbonate, pH=8.5-9) and 4.5 μl of this placed in two microfuge tubes. 4.5 μl of each dye (in DMSO) are added and the mixture incubated in the dark for 1 hour. 4.5 μl of 4 M hydroxylamine is added and again incubated in the dark for 15 minutes.

Regardless of the method used for probe generation, the probe is purified using a Qiagen PCR cleanup kit (Qiagen, Valencia, Calif., USA), and eluted with 100 ul EB (kit provided). The sample is loaded on a Microcon YM-30 (Millipore, Bedford, Mass., USA) spin column and concentrated to 4-5 ul in volume.

Probes for maize microarrays are generated using the Fluorescent Linear Amplification Kit (cat. No. G2556A) from Agilent Technologies (Palo Alto, Calif.).

Hybridization and Wash Conditions

The following Hybridization and Washing Condition are used:

Hybridization Conditions:

Labeled probe is heated at 95° C. for 3 min and chilled on ice. Then 25 μL of the hybridization buffer which is warmed at 42 C is added to the probe, mixing by pipeting, to give a final concentration of:
50% formamide
   4×SSC
   0.03% SDS
5× Denhardt's solution
0.1 μg/ml single-stranded salmon sperm DNA

The probe is kept at 42 C. Prior to the hybridization, the probe is heated for 1 more min., added to the array, and then covered with a glass cover slip. Slides are placed in hybridization chambers (Telechem, Sunnyvale, Calif.) and incubated at 42° C. overnight.

Washing Conditions:

A. Slides are washed in 1×SSC+0.03% SDS solution at room temperature for 5 minutes,
B. Slides are washed in 0.2×SSC at room temperature for 5 minutes,
C. Slides are washed in 0.05×SSC at room temperature for 5 minutes.

After A, B, and C, slides are spun at 800×g for 2 min. to dry. They are then scanned.

Maize microarrays are hybridized according to the instructions included Fluorescent Linear Amplification Kit (cat. No. G2556A) from Agilent Technologies (Palo Alto, Calif.).

Scanning of Slides

The chips are scanned using a ScanArray 3000 or 5000 (General Scanning, Watertown, Mass., USA). The chips are scanned at 543 and 633nm, at 10 um resolution to measure the intensity of the two fluorescent dyes incorporated into the samples hybridized to the chips.

Data Extraction and Analysis

The images generated by scanning slides consiste of two 16-bit TIFF images representing the fluorescent emissions of the two samples at each arrayed spot. These images are then quantified and processed for expression analysis using the data extraction software Imagene™ (Biodiscovery, Los Angeles, Calif., USA). Imagene output is subsequently analyzed using the analysis program Genespring™ (Silicon Genetics, San Carlos, Calif., USA). In Genespring, the data is imported using median pixel intensity measurements derived from Imagene output. Background subtraction, ratio calculation and normalization are all conducted in Genespring. Normalization is achieved by breaking the data in to 32 groups, each of which represented one of the 32 pin printing regions on the microarray. Groups consist of 360 to 550 spots. Each group is independently normalized by setting the median of ratios to one and multiplying ratios by the appropriate factor.

Results

TABLE 2 presents the results of the differential expression experiments for the mRNAs, as reported by their corresponding cDNA ID number, that are differentially transcribed under a particular set of conditions as compared to a control sample. The cDNA ID numbers correspond to those utilized in the Reference and Sequence Tables. Increases in mRNA abundance levels in experimental plants versus the controls are denoted with the plus sign (+). Likewise, reductions in mRNA abundance levels in the experimental plants are denoted with the minus (−) sign.

The Table is organized according to the clone number with each set of experimental conditions being denoted by the term “Expt Rep ID:” followed by a “short name”. TABLE 3 links each Expt Rep ID with a short description of the experiment and the parameters. The experiment numbers are referenced in the appropriate utility/functions sections herein.

The sequences showing differential expression in a particular experiment (denoted by either a “+” or “−” in the Table) thereby shows utility for a function in a plant, and these functions/utilities are described in detail below, where the title of each section (i.e. a “utlity section”) is correlated with the particular differential expression experiment in TABLE 3.

Organ-Affecting Genes, Gene Components, Products (Including Differentiation and Function)

Root Genes

The economic values of roots arise not only from harvested adventitious roots or tubers, but also from the ability of roots to funnel nutrients to support growth of all plants and increase their vegetative material, seeds, fruits, etc. Roots have four main functions. First, they anchor the plant in the soil. Second, they facilitate and regulate the molecular signals and molecular traffic between the plant, soil, and soil fauna. Third, the root provides a plant with nutrients gained from the soil or growth medium. Fourth, they condition local soil chemical and physical properties.

Root genes are active or potentially active to a greater extent in roots than in most other organs of the plant. These genes and gene products regulate many plant traits from yield to stress tolerance. Root genes are used to modulate root growth and development.

Differential Expression of the Sequences in Roots

The relative levels of mRNA product in the root versus the aerial portion of the plant is measured. Specifically, mRNA is isolated from roots and root tips of Arabidopsis plants and compared to mRNA isolated from the aerial portion of the plants utilizing microarray procedures. Results are presented in TABLE 2.

Reproduction Genes, Gene Components and Products

Reproduction genes are defined as genes or components of genes capable of modulating any aspect of sexual reproduction from flowering time and inflorescence development to fertilization and finally seed and fruit development. These genes are of great economic interest as well as biological importance. The fruit and vegetable industry grosses over $1 billion USD a year. The seed market, valued at approximately $15 billion USD annually, is even more lucrative.

Inflorescence and Floral Development Genes, Gene Components and Products

During reproductive growth the plant enters a program of floral development that culminates in fertilization, followed by the production of seeds. Senescence may or may not follow. The flower formation is a precondition for the sexual propagation of plants and is therefore essential for the propagation of plants that cannot be propagated vegetatively, as well as for the formation of seeds and fruits. The point of time at which the merely vegetative growth of plants changes into flower formation is of vital importance, for example in agriculture, horticulture and plant breeding. Also the number of flowers is often of economic importance, for example in the case of various useful plants (tomato, cucumber, zucchini, cotton etc.) with which an increased number of flowers may lead to an increased yield, or in the case of growing ornamental plants and cut flowers.

Flowering plants exhibit one of two types of inflorescence architecture: indeterminate, in which the inflorescence grows indefinitely, or determinate, in which a terminal flower is produced. Adult organs of flowering plants develop from groups of stem cells called meristems. The identity of a meristem is inferred from structures it produces: vegetative meristems give rise to roots and leaves, inflorescence meristems give rise to flower meristems, and flower meristems give rise to floral organs such as sepals and petals. Not only are meristems capable of generating new meristems of different identity, but their own identity can change during development. For example, a vegetative shoot meristem can be transformed into an inflorescence meristem upon floral induction, and in some species, the inflorescence meristem itself will eventually become a flower meristem. Despite the importance of meristem transitions in plant development, little is known about the underlying mechanisms.

Following germination, the shoot meristem produces a series of leaf meristems on its flanks. However, once floral induction has occurred, the shoot meristem switches to the production of flower meristems. Flower meristems produce floral organ primordia, which develop individually into sepals, petals, stamens or carpels. Thus, flower formation can be thought of as a series of distinct developmental steps, i.e. floral induction, the formation of flower primordia and the production of flower organs. Mutations disrupting each of the steps have been isolated in a variety of species, suggesting that a genetic hierarchy directs the flowering process (see for review, Weigel and Meyerowitz, In Molecular Basis of Morphogenesis (ed. M. Bernfield). 51st Annual Symposium of the Society for Developmental Biology, pp. 93-107, New York, 1993).

Expression of many reproduction genes and gene products is orchestrated by internal programs or the surrounding environment of a plant. These genes are used to modulate traits such as fruit and seed yield

Seed and Fruit Development Genes, Gene Components and Products

The ovule is the primary female sexual reproductive organ of flowering plants. At maturity it contains the egg cell and one large central cell containing two polar nuclei encased by two integuments that, after fertilization, develops into the embryo, endosperm, and seed coat of the mature seed, respectively. As the ovule develops into the seed, the ovary matures into the fruit or silique. As such, seed and fruit development requires the orchestrated transcription of numerous polynucleotides, some of which are ubiquitous, others that are embryo-specific and still others that are expressed only in the endosperm, seed coat, or fruit. Such genes are termed fruit development responsive genes and are used to modulate seed and fruit growth and development such as seed size, seed yield, seed composition and seed dormancy.

Differential Expression of the Sequences in Siliques, Inflorescences and Flowers

The relative levels of mRNA product in the siliques relative to the plant as a whole is measured. The results are presented in TABLE 2.

Differential Expression of the Sequences in Hybrid Seed Development

The levels of mRNA product in the seeds relative to those in a leaf and floral stems is measured. The results are presented TABLE 2.

Development Genes, Gene Components and Products

Imbibition and Germination Responsive Genes, Gene Components and Products

Seeds are a vital component of the world's diet. Cereal grains alone, which comprise ˜90% of all cultivated seeds, contribute up to half of the global per capita energy intake. The primary organ system for seed production in flowering plants is the ovule. At maturity, the ovule consists of a haploid female gametophyte or embryo sac surrounded by several layers of maternal tissue including the nucleus and the integuments. The embryo sac typically contains seven cells including the egg cell, two synergids, a large central cell containing two polar nuclei, and three antipodal cells. That pollination results in the fertilization of both egg and central cell. The fertilized egg develops into the embryo. The fertilized central cell develops into the endosperm. And the integuments mature into the seed coat. As the ovule develops into the seed, the ovary matures into the fruit or silique. Late in development, the developing seed ends a period of extensive biosynthetic and cellular activity and begins to desiccate to complete its development and enter a dormant, metabolically quiescent state. Seed dormancy is generally an undesirable characteristic in agricultural crops, where rapid germination and growth are required. However, some degree of dormancy is advantageous, at least during seed development. This is particularly true for cereal crops because it prevents germination of grains while still on the ear of the parent plant (preharvest sprouting), a phenomenon that results in major losses to the agricultural industry. Extensive domestication and breeding of crop species have ostensibly reduced the level of dormancy mechanisms present in the seeds of their wild ancestors, although under some adverse environmental conditions, dormancy may reappear. By contrast, weed seeds frequently mature with inherent dormancy mechanisms that allow some seeds to persist in the soil for many years before completing germination.

Germination commences with imbibition, the uptake of water by the dry seed, and the activation of the quiescent embryo and endosperm. The result is a burst of intense metabolic activity. At the cellular level, the genome is transformed from an inactive state to one of intense transcriptional activity. Stored lipids, carbohydrates and proteins are catabolized fueling seedling growth and development. DNA and organelles are repaired, replicated and begin functioning. Cell expansion and cell division are triggered. The shoot and root apical meristem are activated and begin growth and organogenesis. Schematic 4 summarizes some of the metabolic and cellular processes that occur during imbibition. Germination is complete when a part of the embryo, the radicle, extends to penetrate the structures that surround it. In Arabidopsis, seed germination takes place within twenty-four (24) hours after imbibition. As such, germination requires the rapid and orchestrated transcription of numerous polynucleotides. Germination is followed by expansion of the hypocotyl and opening of the cotyledons. Meristem development continues to promote root growth and shoot growth, which is followed by early leaf formation.

Imbibition and Germination Genes

Imbibition and germination includes those events that commence with the uptake of water by the quiescent dry seed and terminate with the expansion and elongation of the shoots and roots. The germination period exists from imbibition to when part of the embryo, usually the radicle, extends to penetrate the seed coat that surrounds it. Imbibition and germination genes are defined as genes, gene components and products capable of modulating one or more processes of imbibition and germination described above. They are useful to modulate many plant traits from early vigor to yield to stress tolerance.

Differential Exipression of the Sequences in Germinating Seeds and Imbibed Embryos

The levels of mRNA product in the seeds versus the plant as a whole is measured. The results are presented in TABLE 2.

Hormone Responsive Genes, Gene Components and Products

Abscissic Acid Responsive Genes, Gene Components and Products

Plant hormones are naturally occurring substances, effective in very small amounts, which act as signals to stimulate or inhibit growth or regulate developmental processes in plants. Abscisic acid (ABA) is a ubiquitous hormone in vascular plants that has been detected in every major organ or living tissue from the root to the apical bud. The major physiological responses affected by ABA are dormancy, stress stomatal closure, water uptake, abscission and senescence. In contrast to Auxins, cytokinins and gibberellins, which are principally growth promoters, ABA primarily acts as an inhibitor of growth and metabolic processes.

Changes in ABA concentration internally or in the surrounding environment in contact with a plant results in modulation of many genes and gene products. These genes and/or products are responsible for effects on traits such as plant vigor and seed yield.

While ABA responsive polynucleotides and gene products can act alone, combinations of these polynucleotides also affect growth and development. Useful combinations include different ABA responsive polynucleotides and/or gene products that have similar transcription profiles or similar biological activities, and members of the same or similar biochemical pathways. Whole pathways or segments of pathways are controlled by transcription factor proteins and proteins controlling the activity of signal transduction pathways. Therefore, manipulation of such protein levels is especially useful for altering phenotypes and biochemical activities of plants. In addition, the combination of an ABA responsive polynucleotide and/or gene product with another environmentally responsive polynucleotide is also useful because of the interactions that exist between hormone-regulated pathways, stress and defence induced pathways, nutritional pathways and development.

Differential Expression of the Sequences in ABA Treated Plants

The relative levels of mRNA product in plants treated with ABA versus controls treated with water are measured. . Results are presented in TABLE 2.

Brassinosteroid Responsive Genes, Gene Components And Products

Plant hormones are naturally occuring substances, effective in very small amounts, which act as signals to stimulate or inhibit growth or regulate developmental processes in plants. Brassinosteroids (BRs) are the most recently discovered, and least studied, class of plant hormones. The major physiological response affected by BRs is the longitudinal growth of young tissue via cell elongation and possibly cell division. Consequently, disruptions in BR metabolism, perception and activity frequently result in a dwarf phenotype. In addition, because BRs are derived from the sterol metabolic pathway, any perturbations to the sterol pathway can affect the BR pathway. In the same way, perturbations in the BR pathway can have effects on the later part of the sterol pathway and thus the sterol composition of membranes.

Changes in BR concentration in the surrounding environment or in contact with a plant result in modulation of many genes and gene products. These genes and/or products are responsible for effects on traits such as plant biomass and seed yield. These genes were discovered and characterized from a much larger set of genes by experiments designed to find genes whose mRNA abundance changed in response to application of BRs to plants.

While BR responsive polynucleotides and gene products can act alone, combinations of these polynucleotides also affect growth and development. Useful combinations include different BR responsive polynucleotides and/or gene products that have similar transcription profiles or similar biological activities, and members of the same or functionally related biochemical pathways. Whole pathways or segments of pathways are controlled by transcription factors and proteins controlling the activity of signal transduction pathways. Therefore, manipulation of such protein levels is especially useful for altering phenotypes and biochemical activities of plants. In addition, the combination of a BR responsive polynucleotide and/or gene product with another environmentally responsive polynucleotide is useful because of the interactions that exist between hormone-regulated pathways, stress pathways, nutritional pathways and development. Here, in addition to polynucleotides having similar transcription profiles and/or biological activities, useful combinations include polynucleotides that may have different transcription profiles but which participate in common or overlapping pathways.

Differential Expression of the Sequences in Epi-brassinolide or Brassinozole Plants

The relative levels of mRNA product in plants treated with either epi-brassinolide or brassinozole are measured. Results are presented in TABLE 2.

Metabolism Affecting Genes, Gene Components and Products

Nitrogen Responsive Genes, Gene Components and Products

Nitrogen is often the rate-limiting element in plant growth, and all field crops have a fundamental dependence on exogenous nitrogen sources. Nitrogenous fertilizer, which is usually supplied as ammonium nitrate, potassium nitrate, or urea, typically accounts for 40% of the costs associated with crops, such as corn and wheat in intensive agriculture. Increased efficiency of nitrogen use by plants enables the production of higher yields with existing fertilizer inputs and/or enable existing yields of crops to be obtained with lower fertilizer input, or better yields on soils of poorer quality. Also, higher amounts of proteins in the crops are also produced more cost-effectively. “Nitrogen responsive” genes and gene products are used to alter or modulate plant growth and development.

Differential Expression of the Sequences in Whole Seedlings Shoots and Roots

The relative levels of mRNA product in whole seedlings, shoots and roots treated with either high or low nitrogen media are compared to controls. Results are presented in TABLE 2.

Viability Genes, Gene Components and Products

Plants contain many proteins and pathways that when blocked or induced lead to cell, organ or whole plant death. Gene variants that influence these pathways have profound effects on plant survival, vigor and performance. The critical pathways include those concerned with metabolism and development or protection against stresses, diseases and pests. They also include those involved in apoptosis and necrosis. Viability genes can be modulated to affect cell or plant death. Herbicides are, by definition, chemicals that cause death of tissues, organs and whole plants. The genes and pathways that are activated or inactivated by herbicides include those that cause cell death as well as those that function to provide protection.

Differential Expression of the Sequences in Herbicide Treated Plants and Herbicide Resistant Mutants

The relative levels of mRNA product in plants treated with heribicide and mutants resistant to heribicides are compared to control plants. Results are presented in TABLE 2.

Stress Responsive Genes, Gene Components and Products

Cold Responsive Genes, Gene Components and Products

The ability to endure low temperatures and freezing is a major determinant of the geographical distribution and productivity of agricultural crops. Even in areas considered suitable for the cultivation of a given species or cultivar, can give rise to yield decreases and crop failures as a result of aberrant, freezing temperatures. Even modest increases (1-2° C.) in the freezing tolerance of certain crop species would have a dramatic impact on agricultural productivity in some areas. The development of genotypes with increased freezing tolerance provide a more reliable means to minimize crop losses and diminish the use of energy-costly practices to modify the microclimate.

Sudden cold temperatures result in modulation of many genes and gene products, including promoters. These genes and/or products are responsible for effects on traits such as plant vigor and seed yield.

Manipulation of one or more cold responsive gene activities is useful to modulate growth and development.

Differential Expression of the Sequences in Cold Treated Plants

The relative levels of mRNA product in cold treated plants are compared to control plants. Results are presented in TABLE 2.

Heat Responsive Genes, Gene Components and Products

The ability to endure high temperatures is a major determinant of the geographical distribution and productivity of agricultural crops. Decreases in yield and crop failure frequently occur as a result of aberrant, hot conditions even in areas considered suitable for the cultivation of a given species or cultivar. Only modest increases in the heat tolerance of crop species would have a dramatic impact on agricultural productivity. The development of genotypes with increased heat tolerance provide a more reliable means to minimize crop losses and diminish the use of energy-costly practices to modify the microclimate.

Changes in temperature in the surrounding environment or in a plant microclimate results in modulation of many genes and gene products.

Differential Expression of the Sequences in Heat Treated Plants

The relative levels of mRNA product in heat treated plants are compared to control plants. Results are presented in TABLE 2.

Drought Responsive Genes, Gene Components and Products

The ability to endure drought conditions is a major determinant of the geographical distribution and productivity of agricultural crops. Decreases in yield and crop failure frequently occur as a result of aberrant, drought conditions even in areas considered suitable for the cultivation of a given species or cultivar. Only modest increases in the drought tolerance of crop species would have a dramatic impact on agricultural productivity. The development of genotypes with increased drought tolerance provide a more reliable means to minimize crop losses and diminish the use of energy-costly practices to modify the microclimate.

Drought conditions in the surrounding environment or within a plant, results in modulation of many genes and gene products.

Differential Expression of the Sequences in Drought Treated Plants and Drought Mutants

The relative levels of mRNA product in drought treated plants and drought mutants are compared to control plants. Results are presented in TABLE 2.

Methyl Jasmonate (Jasmonate) Responsive Genes, Gene Components and Products

Jasmonic acid and its derivatives, collectively referred to as jasmonates, are naturally occurring derivatives of plant lipids. These substances are synthesized from linolenic acid in a lipoxygenase-dependent biosynthetic pathway. Jasmonates are signalling molecules which have been shown to be growth regulators as well as regulators of defense and stress responses. As such, jasmonates represent a separate class of plant hormones. Jasmonate responsive genes are used to modulate plant growth and development.

Differential Expression of the Sequences in Methyl Jasmonate Treated Plants

The relative levels of mRNA product in methyl jasmonate treated plants are compared to control plants. Results are presented in TABLE 2.

Salicylic Acid Responsive Genes, Gene Components and Products

Plant defense responses can be divided into two groups: constitutive and induced. Salicylic acid (SA) is a signaling molecule necessary for activation of the plant induced defense system known as systemic acquired resistance or SAR. This response, which is triggered by prior exposure to avirulent pathogens, is long lasting and provides protection against a broad spectrum of pathogens. Another induced defense system is the hypersensitive response (HR). HR is far more rapid, occurs at the sites of pathogen (avirulent pathogens) entry and precedes SAR. SA is also the key signaling molecule for this defense pathway.

Differential Expression of the Sequences in Salicylic Acid Treated Plants

The relative levels of mRNA product in salicylic acid treated plants are compared to control plants. Results are presented in TABLE 2.

Osmotic Stress Responsive Genes, Gene Components and Products

The ability to endure and recover from osmotic and salt related stress is a major determinant of the geographical distribution and productivity of agricultural crops. Osmotic stress is a major component of stress imposed by saline soil and water deficit. Decreases in yield and crop failure frequently occur as a result of aberrant or transient environmental stress conditions even in areas considered suitable for the cultivation of a given species or cultivar. Only modest increases in the osmotic and salt tolerance of a crop species would have a dramatic impact on agricultural productivity. The development of genotypes with increased osmotic tolerance provide a more reliable means to minimize crop losses and diminish the use of energy-costly practices to modify the soil environment. Thus, osmotic stress responsive genes are used to modulate plant growth and development.

Differential Expression of the Sequences in PEG Treated Plants

The relative levels of mRNA product in PEG treated plants are compared to control plants. Results are presented in TABLE 2.

Shade Responsive Genes, Gene Components and Products

Plants sense the ratio of Red (R):Far Red (FR) light in their environment and respond differently to particular ratios. A low R:FR ratio, for example, enhances cell elongation and favors flowering over leaf production. The changes in R:FR ratios mimic and cause the shading response effects in plants. The response of a plant to shade in the canopy structures of agricultural crop fields influences crop yields significantly. Therefore manipulation of genes regulating the shade avoidance responses improve crop yields. While phytochromes mediate the shade avoidance response, the down-stream factors participating in this pathway are largely unknown. One potential downstream participant, ATHB-2, is a member of the HD-Zip class of transcription factors and shows a strong and rapid response to changes in the R:FR ratio. ATHB-2 overexpressors have a thinner root mass, smaller and fewer leaves and longer hypocotyls and petioles. This elongation arises from longer epidermal and cortical cells, and a decrease in secondary vascular tissues, paralleling the changes observed in wild-type seedlings grown under conditions simulating canopy shade. On the other hand, plants with reduced ATHB-2 expression have a thick root mass and many larger leaves and shorter hypocotyls and petioles. Here, the changes in the hypocotyl result from shorter epidermal and cortical cells and increased proliferation of vascular tissue. Interestingly, application of Auxin is able to reverse the root phenotypic consequences of high ATHB-2 levels, restoring the wild-type phenotype. Consequently, given that ATHB-2 is tightly regulated by phytochrome, these data suggest that ATHB-2 may link the Auxin and phytochrome pathways in the shade avoidance response pathway.

Shade responsive genes are used to modulate plant growth and development.

Differential Expression of the Sequences in Far-Red Light Treated Plants

The relative levels of mRNA product in far-red light treated plants are compared to control plants. Results are presented in TABLE 2.

Viability Genes, Gene Components and Products

Plants contain many proteins and pathways that when blocked or induced lead to cell, organ or whole plant death. Gene variants that influence these pathways can have profound effects on plant survival, vigor and performance. The critical pathways include those concerned with metabolism and development or protection against stresses, diseases and pests. They also include those involved in apoptosis and necrosis. The applicants have elucidated many such genes and pathways by discovering genes that, when inactivated, lead to cell or plant death.

Herbicides are, by definition, chemicals that cause death of tissues, organs and whole plants. The genes and pathways that are activated or inactivated by herbicides include those that cause cell death as well as those that function to provide protection. The applicants have elucidated these genes.

The genes defined in this section have many uses including manipulating which cells, tissues and organs are selectively killed, which are protected, making plants resistant to herbicides, discovering new herbicides and making plants resistant to various stresses.

Viability genes are also identified from a much larger set of genes by experiments designed to find genes whose mRNA products changed in concentration in response to applications of different herbicides to plants. Viability genes are characteristically differentially transcribed in response to fluctuating herbicide levels or concentrations, whether internal or external to an organism or cell. Table 2 reports the changes in transcript levels of various viability genes.

Early Seedling-Phase Specific Responsive Genes, Gene Components and Products

One of the more active stages of the plant life cycle is a few days after germination is complete, also referred to as the early seedling phase. During this period the plant begins development and growth of the first leaves, roots, and other organs not found in the embryo. Generally this stage begins when germination ends. The first sign that germination has been completed is usually that there is an increase in length and fresh weight of the radicle. Such genes and gene products can regulate a number of plant traits to modulate yield. For example, these genes are active or potentially active to a greater extent in developing and rapidly growing cells, tissues and organs, as exemplified by development and growth of a seedling 3 or 4 days after planting a seed.

Rapid, efficient establishment of a seedling is very important in commercial agriculture and horticulture. It is also vital that resources are approximately partitioned between shoot and root to facilitate adaptive growth. Phototropism and geotropism need to be established. All these require post-germination process to be sustained to ensure that vigorous seedlings are produced. Early seedling phase genes, gene components and products are useful to manipulate these and other processes.

Guard Cell Genes, Gene Components and Products

Scattered throughout the epidermis of the shoot are minute pores called stomata. Each stomal pore is surrounded by two guard cells. The guard cells control the size of the stomal pore, which is critical since the stomata control the exchange of carbon dioxide, oxygen, and water vapor between the interior of the plant and the outside atmosphere. Stomata open and close through turgor changes driven by ion fluxes, which occur mainly through the guard cell plasma membrane and tonoplast. Guard cells are known to respond to a number of external stimuli such as changes in light intensity, carbon dioxide and water vapor, for example. Guard cells also sense and rapidly respond to internal stimuli including changes in ABA, auxin and calcium ion flux.

Thus, genes, gene products, and fragments thereof differentially transcribed and/or translated in guard cells can be useful to modulate ABA responses, drought tolerance, respiration, water potential, and water management as examples. All of which can in turn affect plant yield including seed yield, harvest index, fruit yield, etc.

To identify such guard cell genes, gene products, and fragments thereof, Applicants perform a microarray experiment comparing the transcript levels of genes in guard cells versus leaves. Experimental data is shown below.

Nitric Oxide Responsive Genes, Gene Components and Products

The rate-limiting element in plant growth and yield is often its ability to tolerate suboptimal or stress conditions, including pathogen attack conditions, wounding and the presence of various other factors. To combat such conditions, plant cells deploy a battery of inducible defense responses, including synergistic interactions between nitric oxide (NO), reactive oxygen intermediates (ROS), and salicylic acid (SA). NO has been shown to play a critical role in the activation of innate immune and inflammatory responses in animals. At least part of this mammalian signaling pathway is present in plants, where NO is known to potentiate the hypersensitive response (HR). In addition, NO is a stimulator molecule in plant photomorphogenesis.

Changes in nitric oxide concentration in the internal or surrounding environment, or in contact with a plant, results in modulation of many genes and gene products.

In addition, the combination of a nitric oxide responsive polynucleotide and/or gene product with other environmentally responsive polynucleotides is also useful because of the interactions that exist between hormone regulated pathways, stress pathways, pathogen stimulated pathways, nutritional pathways and development.

Nitric oxide responsive genes and gene products function either to increase or dampen the above phenotypes or activities either in response to changes in nitric oxide concentration or in the absence of nitric oxide fluctuations. More specifically, these genes and gene products modulate stress responses in an organism. In plants, these genes and gene products are useful for modulating yield under stress conditions. Measurments of yield include seed yield, seed size, fruit yield, fruit size, etc.

Shoot-Apical Meristem Genes, Gene Components and Products

New organs, stems, leaves, branches and inflorescences develop from the stem apical meristem (SAM). The growth structure and architecture of the plant therefore depends on the behavior of SAMs. Shoot apical meristems (SAMs) are comprised of a number of morphologically undifferentiated, dividing cells located at the tips of shoots. SAM genes elucidated here are capable of modifying the activity of SAMs and thereby many traits of economic interest from ornamental leaf shape to organ number to responses to plant density.

In addition, a key attribute of the SAM is its capacity for self-renewal. Thus, SAM genes of the instant invention are useful for modulating one or more processes of SAM structure and/or function including (I) cell size and division; (II) cell differentiation and organ primordia. The genes and gene components of this invention are useful for modulating any one or all of these cell division processes generally, as in timing and rate, for example. In addition, the polynucleotides and polypeptides of the invention can control the response of these processes to the internal plant programs associated with embryogenesis, and hormone responses, for example.

Because SAMs determine the architecture of the plant, modified plants will be useful in many agricultural, horticultural, forestry and other industrial sectors. Plants with a different shape, numbers of flowers and seed and fruits will have altered yields of plant parts. For example, plants with more branches can produce more flowers, seed or fruits. Trees without lateral branches will produce long lengths of clean timber. Plants with greater yields of specific plant parts will be useful sources of constituent chemicals.

The invention being thus described, it will be apparent to one of ordinary skill in the art that various modifications of the materials and methods for practicing the invention can be made. Such modifications are to be considered within the scope of the invention as defined by the following claims.

Each of the references from the patent and periodical literature cited herein is hereby expressly incorporated in its entirety by such citation.

TABLE 1

>12707591
atgggtgaaaccgctgccgccaataaccaccgtcaccaccaccatcacggccaccaggtc
tttgacgtggccagccacgatttcgtccctccacaaccggcttttaaatgcttcgatgat
gatggccgcctcaaaagaactgggactgtttggaccgcgagcgctcatataataactgcg
gttatcggatccggcgttttgtcattggcgtgggcgattgcacagctcggatggatcgct
ggccctgctgtgatgctattgttctctcttgttactctttactcctccacacttcttagc
gactgctacagaaccggcgatgcagtgtctggcaagagaaactacacttacatggatgcc
gttcgatcaattctcggtgggttcaagttcaagatttgtgggttgattcaatacttgaat
ctctttggtatcgcaattggatacacgatacaggcgatcaagagatccaactgcttccac
aagagtggaggaaaagacccatgtcacatgtccagtaatccttacatgatcgtatttggt
gtggcagagatcttgctctctcaggttcctgatttcgatcagatttggtggatctccatt
gttgcagctgttatgtccttcacttactctgccattggtctagctcttggaatcgttcaa
gttgcagcgaatggagttttcaaaggaagtctcactggaataagcatcggaacagtgact
caaacacagaagatatggagaaccttccaagcacttggagacattgcctttgcgtactca
tactctgttgtcctaatcgagattcaggatactgtaagatccccaccggcggaatcgaaa
acgatgaagaaagcaacaaaaatcagtattgccgtcacaactatcttctacatgctatgt
ggctcaatgggttatgccgcttttggagatgcagcaccgggaaacctcctcaccggtttt
ggattctacaacccgttttggctccttgacatagctaacgccgccattgttgtccacctc
gttggagcttaccaagtctttgctcagcccatctttgcctttattgaaaaatcagtcgca
gagagatatccagacaatgacttcctcagcaaggaatttgaaatcagaatccccggattt
aagtctccttacaaagtaaacgttttcaggatggtttacaggagtggctttgtcgttaca
accaccgtgatatcgatgctgatgccgttttttaacgacgtggtcgggatcttaggggcg
ttagggttttggcccttgacggtttattttccggtggagatgtatattaagcagaggaag
gttgagaaatggagcacgagatgggtgtgtttacagatgcttagtgttgcttgtcttgtg
atctcggtggtcgccggggttggatcaatcgccggagtgatgcttgatcttaaggtctat
aagccattcaagtctacatattga
>12707592
MGETAAANNHRHHHHHGHQVFDVASHDFVPPQPAFKCFDDDGRLKRTGTVWTASAHIITA
VIGSGVLSLAWAIAQLGWIAGPAVMLLFSLVTLYSSTLLSDCYRTGDAVSGKRNYTYMDA
VRSILGGFKFKICGLIQYLNLFGIAIGYTIQAIKRSNCFHKSGGKDPCHMSSNPYMIVFG
VAEILLSQVPDFDQIWWISIVAAVMSFTYSAIGLALGIVQVAANGVFKGSLTGISTGTVT
QTQKIWRTFQALGDIAFAYSYSVVLIEIQDTVRSPPAESKTMKKATKISIAVTTIFYMLC
GSMGYAAFGDAAPGNLLTGFGFYNPFWLLDIANAAIVVHLVGAYQVFAQPIFAFIEKSVA
ERYPDNDFLSKEFEIRIPGFKSPYKVNVFRMVYRSGFVVTTTVISMLMPFFNDVVGILGA
LGFWPLTVYFPVEMYIKQRKVEKWSTRWVCLQMLSVACLVISVVAGVGSIAGVMLDLKVY
KPFKSTY*
>12385295
ACCTTTAATTTTTTCACCAATTGGATTTGGATCTGTCAAAAATATTGGCCTCTTTCTCTC
TTTCTCTCTTGCTCTCTTTCTTTGTTGGGTTGATCTCTTCTTCCATGGCGATGCCTACGG
AGACACAACACCAGGAGAAGGAGGCTTCAGATGCTTCTGCAGCAGCTGCACAAAAGAGAT
GGACTTTAAGCGATTTCGACATCGGTAAGCCTCTTGGCAGAGGCAAATTCGGTCACGTCT
ATCTCGCCAGAGAAAAACGGAGCAATCACGTTGTCGCTCTAAAGGTTCTTTTCAAGAGCC
AGCTTCAACAATCCCAAGTTGAACATCAGCTCAGAAGAGAAGTTGAGATTCAGTCTCATC
TTCGTCACCCCAACATACTCCGGCTTTATGGCTATTTCTATGATCAAAAAAGAGTTTATT
TGATACTTGAGTATGCTGCTAGAGGCGAACTTTACAAGGATCTTCAGAAATGCAAATACT
TCAGCGAAAGACGAGCTGCTACTTATGTTGCCTCATTGGCGAGGGCTCTCATCTATTGCC
ATGGCAAGCATGTGATACACAGAGATATTAAACCAGAGAATCTGCTAATTGGTGCTCAGG
GTGAGCTCAAGATTGCAGACTTTGGTTGGTCGGTACACACATTTAACGGAAGAAGGACCA
TGTGTGGCACACTAGATTACCTTCCTCCTGAGATGGTCGAAAGCGTAGAACATGATGCTA
GTGTAGATATCTGGAGGCTTGGGATTCTCTGTTACGAGTTTCTTTATGGTGTACCTCCTT
TTGAAGCCATGGAGCACTCAGACACATACAGAAGGATTGTGCAAGTGGATGTCAAGTTCC
CTCCCAAACCAATAATCTCTGCATCTGCAAAGGATCTTATTAGCCAGATGCTTGTCAAGG
AGTCTTCGCAACGTCTGCCATTGCACAAGCTTCTGGAGCATCCGTGGATCGTGCAAAACG
CTGATCCTTCTGGAATCTACAGAGTTTAAAAACAAAACGCTTACTGTTCTCGCCAATCTC
GACCAAAACTGTTATCTAGAAACCCAAAACTGTATTCTTTCTTACTATTATTTTTCGCTA
ATTCATGTTCCAAATAATGTGTATTATGTAGTATGTACTAATACTATATGGCATT
>12385296
MAIPTETQHQEKEASDASAAAAQKRWTLSDFDIGKPLGRGKFGHVYLAREKRSNHVVALK
VLFKSQLQQSQVEHQLRREVEIQSHLRHPNILRLYGYFYDQKRVYLTLEYAARGELYKDL
QKCKYFSERRAATYVASLARALIYCHGKHVIHRDIKPENLLIGAQGELKIADFGWSVHTF
NRRRTMCGTLDYLPPEMVESVEHDASVDIWSLGILCYEFLYGVPPFEAMEHSDTYRRIVQ
VDLKFPPKPIISASAKDLISQMLVKESSQRLPLHKLLEHPWIVQNADPSGIYRV*
>12385295
ACCTTTAATTTTTTCACCAATTGGATTTGGATCTGTCAAAAATATTGGCCTCTTTCTCTC
TTTCTCTCTTGCTCTCTTTCTTTGTTGGGTTGATCTCTTCTTCCATGGCGATCCCTACGG
AGACACAACACCAGGAGAAGGAGGCTTCAGATGCTTCTGCAGCAGCTGCACAAAAGAGAT
GGACTTTAAGCGATTTCGACATCGGTAAGCCTCTTGGCAGAGGCAAATTCGGTCACGTCT
ATCTCGCCAGAGAAAAACGGAGCAATCACGTTGTCGCTCTAAAGGTTCTTTTCAAGAGCC
AGCTTCAACAATCCCAAGTTGAACATCAGCTCAGAAGAGAAGTTGAGATTCAGTCTCATC
TTCGTCACCCCAACATACTCCGGCTTTATGGCTATTTCTATGATCAAAAAAGAGTTTATT
TGATACTTGAGTATGCTGCTAGAGGCGAACTTTACAAGGATCTTCAGAAATGCAAATACT
TCAGCGAAAGACGAGCTGCTACTTATGTTGCCTCATTGGCGAGGGCTCTCATCTATTGCC
ATGGCAAGCATGTGATACACAGAGATATTAAACCAGAGAATCTGCTAATTGGTGCTCAGG
GTGAGCTCAAGATTGCAGACTTTGGTTGGTCGGTACACACATTTAACCGAAGAAGGACCA
TGTGTGGCACACTAGATTACCTTCCTCCTGAGATGGTCGAAAGCGTAGAACATGATGCTA
GTGTAGATATCTGGAGCCTTGGGATTCTCTGTTACGAGTTTCTTTATGGTGTACCTCCTT
TTGAAGCCATGGAGCACTCAGACACATACAGAAGGATTGTGCAAGTGGATCTCAAGTTCC
CTCCCAAACCAATAATCTCTGCATCTGCAAAGGATCTTATTAGCCAGATGCTTGTCAAGG
AGTCTTCGCAACGTCTGCCATTGCACAAGCTTCTGGAGCATCCGTGGATCGTGCAAAACG
CTGATCCTTCTGGAATCTACAGAGTTTAAAAACAAAACGCTTACTGTTCTCGCCAATCTC
GACCAAAACTGTTATCTAGAAACCCAAAACTGTATTCTTTCTTACTATTATTTTTCGCTA
ATTCATGTTCCAAATAATGTGTATTATGTAGTATGTACTAATACTATATGGCATT
>12385296
MAIPTETQHQEKEASDASAAAAQKRWTLSDFDIGKPLGRGKFGHVYLAREKRSNHVVALK
VLFKSQLQQSQVEHQLRREVEIQSHLRHPNILRLYGYFYDQKRVYLILEYAARGELYKDL
QKCKYFSERRAATYVASLARALIYCHGKHVIHRDIKPENLLIGAQGELKIADFGWSVHTF
NRRRTMCGTLDYLPPEMVESVEHDASVDIWSLGILCYEFLYGVPPFEAMEHSDTYRRIVQ
VDLKFPPKPIISASAKDLISQMLVKESSQRLPLHKLLEHPWIVQNADPSGIYRV*
>12688873
ATGTCGTCGAAGAAATCCCTAGTTCAAAGTCTCTTCAACATTTCCAAAACCTACTCCAGG
ATTTCTGGTCTTACCCGAATGCGTCCGACCAAATCCGGCGGCATTCCTCCCGACGCCGGA
GATTCTGGAATCCGCCGCAGATTTCTCCACAAGAGGGCATTTTTCTCGCCGGAGATAGTT
CCTAAAGGTGGTAACTTGATGGAGAAACTCAGGGAATTGACTTTGTCCAATAATAATCGT
ATTAGACTCGACGAGATGTTACCGCCACCTTCGCCGAAGAAATCATCACCGGAGTTTTTC
CCGGCGGTCACGGTGGAAGACGTGAAGAAGCTCATGAGAGCAGCGGAAATGGAGCTGGTG
AAATCGAAGCTGAGAGAGATTGGCAAGAACTGGGTTCCTTATTCGGAGTTTGTTCGGGTC
TGCGGAGAATACAGTTCGGATCCTGAACAAGGTAACCGGGTCGCGAATATGCTTGACGAA
GCTGGAAACGTCATCGTTTTGGGAAAACTCGTCTGCCTTAAACCCGAAGAGCTAACAAGC
GCCATGGCTGGTCTGATTCCGACACTCGAACCCAGTCTCGACGCCGAGACAAGACAAGAG
TTCGAACAACTTGAGATCATAAAATCAGATATCGACAAAAGAGCCGATGATCTGGTTCGA
AAAGAATTATGGGCCGGATTAGGCCTAATAATGGCCCAAACAGTTGGATTTTTTAGGCTG
ACGTTTTGGGAACTGTCGTGGGACGTGATGGAACCCATATGCTTCTACGTAACTTCGACA
TATTTCATGGCTGGTTACGCCTTCTTCCTCCGAACTTCAAAGGAACCTTCCTTTGAAGGT
TTTTAGAAAAGCGGGTTCGAGACGAAGCAGAAACGTTTGATTAAAATGCTTGATTTCGAT
ATCGATCGATTTACCAAGCTACAGAAGATGCATCGTCCAAATTTGACTAAATCTGGTCGT
TGTTGA
>12688874
MSSKKSLVQSLFNISKTYSRISGLTRMRPTKSGGIPPDAGDSGIRRRFLHKRAFFSPEIV
PKGGNLMEKLRELTLSNNNRIRLDEMLPPPSPKKSSPEFFPAVTVEDVKKLMRAAEMELV
KSKLREIGKNWVPYSEFVRVCGEYSSDPEQGNRVANMLDEAGNVIVLGKLVGLKPEELTS
AMAGLIPTLEPSLDAETRQEFEQLEIIKSDIDKRADDLVRKELWAGLGLIMAQTVGFFRL
TFWELSWDVMEPICFYVTSTYFMAGYAFFLRTSKEPSFEGFYKSRFETKQKRLIKMLDFD
IDRFTKLQKMHRPNLTKSGRC*
>13617271
CACAAGGCTAGGGATCGAAGAAGCGGCGATCACTGATCGTATCTCACTACGATCACATTA
ATGGATAGAATGTGTGGTTTCCGCTCGACGGAAGACTATTCGGAGAAAGCGACGTTGATG
ATGCCGTCCGATTATCAGTCTTTGATTTGTTCAACCACCGGAGACAATCAAAGACTGTTT
GGATCCGACGAACTCGCTACCGCTTTGTCCTCGGAGTTGCTTCCGCGTATTCGAAAAGCT
GAGGATAATTTCTCTCTTAGTGTCATCAAATCCAAAATCGCTTCTCATCCTTTGTATCCT
CGCTTACTCCAAACCTACATCGATTGCCAAAAGGTGGGAGCGCCTATGGAAATAGCGTGT
ATATTGGAAGAGATTCAGCGAGAGAACCATGTGTACAAGAGAGATGTTGCTCCATTATCT
TGCTTTGGAGCTGATCCTGAGCTTGATGAATTCATGGAAACCTACTGTGATATATTGGTT
AAATACAAAACCGATCTTGCGAGGCCGTTCGACGAGGCTACAACTTTCATAAACAAGATT
GAAATGCAGCTTCAGAACTTGTGCACTGGTCCAGCGTCTGCTACAGCTCTTTCAGATGAT
GGTGCGGTTTCATCTGACGAGGAACTGAGAGAAGATGATGACATAGCAGCGGATGACAGC
CAACAAAGAAGCAATGACCGCGATCTGAAGGACCAGCTACTACGCAAATTTGGTAGCCAT
ATCAGTTCATTGAAACTCGAGTTCTCTAAAAAGAAGAAGAAAGGGAAGCTACCAAGAGAA
GCAAGACAAGCGTTGCTCGATTGGTGGAATGTTCATAATAAATGGCCTTACCCTACTGAA
GGCGACAAAATATCTCTGGCTGAAGAAACAGGTTTGGATCAAAAACAAATCAACAATTGG
TTTATAAACCAAAGGAAACGCCATTGGAAGCCTTCGGAGAACATGCCGTTTGATATGATG
GACGATTCTAATGAAACATTCTTTACCGAGTAATGAAAAGAGAGACATGAAATTGTGCAT
TGTATAATTTTTACACTGTTTTTCCAAGAAAAGAAAACAGTAAAAAGCTTTTGGTAAATG
GGGCATCATCGCGAATGAATGGAACCCGTTAGCCAAAACGGTCAAGGGCGTAACGAGACA
TTGTATTGGAAATAGTGGCAATATTATGTCACTAATCTTCCAATCATCCAAATTGATAGA
TTTCTTATTTGTATTGAACCTTACTTAGATAGCTGATGTGTCAACTAAATAATTTATTTT
CATTATCCATTCGGGTAG
>13617272
MDRMCGFRSTEDYSEKATLMMPSDYQSLICSTTGDNQRLFGSDELATALSSELLPRIRKA
EDNFSLSVIKSKIASHPLYPRLLQTYIDCQKVGAPMEIACILEEIQRENHVYKRDVAPLS
CFGADPELDEFMETYCDILVKYKTDLARPFDEATTFINKIEMQLQNLCTGPASATALSDD
GAVSSDEELREDDDIAADDSQQRSNDRDLKDQLLRKFGSHISSLKLEFSKKKKKGKLPRE
ARQALLDWWNVHNKWPYPTEGDKISLAEETGLDQKQINNWFINQRKRHWKPSENMPFDMM
DDSNETFFTE*
>13619728
AATCTTGAAAAGTGTTTTTGAGAGAAATATAGGTTTTACAAAATCCACCGTTGTGAATTC
ATGGAAATGGTAAACGCAGAAGCAAAACAGAGTGTCCCTCTTCTCACCCCTTATAAGATG
GGAAGATTCAATCTTTCTCATAGGGTTGTTCTAGCACCATTGACGAGACAGAAATCGTAC
GGAAGCGTTCCTCAGCCTCACGCTATCTTGTATTACTCTCAGAGAACGTCACCGGGAGGT
TTTCTCATCGCTGAAGCCACCGGGGTTTCAGATACAGCTCAAGGGTATCCAGATACACCT
GGGATATGGACTAAAGAGCATGTGGAGGCATGGAAGCCAATCGTTGATGCTGTACATGCC
AAAGGTGGTATCTTCTTCTGTCAGATCTGGCATGTTGGCCGCGTTTCTAATCGCGGTTTT
CAGCCAAGGAGGCAAGCTCCTATCTCTTGTACGGGGAAGCCAATTATGCCTCAAATGCGT
GCTAATGGCATTGATGAAGCTCGCTTTACCCCTCCAAGACGTCTAAGTATCGAAGAAATC
CCCGGCATTGTCAATGATTTTAGGCTTGCTGCAAGAAATGCTATGGAAGCTGGTTTTGAT
GGAGTTGAGATTCATGGAGCTCATGGCTATCTGATTGATCAGTTCATGAAGGACAAAGTG
AATGACAGAACTGATGAATATGGTGGATCATTGCAAAACCGTTGCAAATTTGCTCTGGAA
GTAGTCGATGCAGTGGCTAAGGAGATCGGGCCAGACCGTGTTGGAATCAGGCTCTCTCCG
TTTGCAGACTATATGGAATCCGGAGACACTAATCCAGAAGCATTAGGGCTGTACATGGTG
GAATCTCTGAACAAATATGGAATCCTCTACTGTCATATGATTGAACCCAGAATGAAAACA
GTGGGAGAAATAGCAGCGTGTTCTCACACACTAATGCCAATGAGGGAAGCCTTTAAGGGG
ACTTTCATCTCTGCAGGAGGTTTCACGAGGGAAGATGGGAATGAGGCCGTGGCAAAGGGA
CGAACTGATCTTGTGGCTTATGGTCGATGGTTTCTAGCCAACCCAGACCTGCCAAAGCGG
TTCCAACTGGATGGACCGCTGAATAAATACAATAGGTCAACGTTTTACACTTCTGATCCT
GTCGTGGGTTACACCGATTACCCTTCCCTTGAATCAACAGCTTAAAATCGTGTTATCAGT
AATGTAATGTGTTTCCCTAATGATGTAATAAGTTTCTGGCTTTTGTTTATACTCTAAGTC
ATTATACCTTCATAATAATTTACATGGATACATTATCACAAAAGAGCTTTTAT
>13619729
MEMVNAEAKQSVPLLTPYKMGRFNLSHRVVLAPLTRQKSYGSVPQPHAILYYSQRTSPGG
FLIAEATGVSDTAQGYPDTPGIWTKEHVEAWKPIVDAVHAKGGIFFCQIWHVGRVSNRGF
QPRRQAPISCTGKPIMPQMRANGIDEARFTPPRRLSIEEIPGIVNDFRLAARNANEAGFD
GVEIHGAHGYLIDQFMKDKVNDRTDEYGGSLQNRCKFALEVVDAVAKEIGPDRVGIRLSP
FADYMESGDTNPEALGLYMVESLNKYGILYCHMIEPRMKTVGEIAACSHTLMPMREAFKG
TFISAGGFTREDGNEAVAKGRTDLVAYGRWFLANPDLPKRFQLDAPLNKYNRSTFYTSDP
VVGYTDYPSLESTA*
>12688873
ATGTCGTCGAAGAAATCCCTAGTTCAAAGTCTCTTCAACATTTCCAAAACCTACTCCAGG
ATTTCTGGTCTTACCCGAATGCGTCCGACCAAATCCGGCGGCATTCCTCCCGACGCCGGA
GATTCTGGAATCCGCCGCAGATTTCTCCACAAGAGGGCATTTTTCTCGCCGGAGATAGTT
CCTAAAGGTGGTAACTTGATGGAGAAACTCAGGGAATTGACTTTGTCCAATAATAATCGT
ATTAGACTCGACGAGATGTTACCGCCACCTTCGCCGAAGAAATCATCACCGGAGTTTTTC
CCGGCGGTCACGGTGGAAGACGTGAAGAAGCTCATGAGAGCAGCGGAAATGGAGCTGGTG
AAATCGAAGCTGAGAGAGATTGGCAAGAACTGGGTTCCTTATTCGGAGTTTGTTCGGGTC
TGCGGAGAATACAGTTCGGATCCTGAACAAGGTAACCGGGTCGCGAATATGCTTGACGAA
GCTGGAAACGTCATCGTTTTGGGAAAACTCGTCTGCCTTAAACCCGAAGAGCTAACAAGC
GCCATGGCTGGTCTGATTCCGACACTCGAACCCAGTCTCGACGCCGAGACAAGACAAGAG
TTCGAACAACTTGAGATCATAAAATCAGATATCGACAAAAGAGCCGATGATCTGGTTCGA
AAAGAATTATGGGCCGGATTAGGCCTAATAATGGCCCAAACAGTTGGATTTTTTAGGCTG
ACGTTTTGGGAACTGTCGTGGGACGTGATGGAACCCATATGCTTCTACGTAACTTCGACA
TATTTCATGGCTGGTTACGCCTTCTTCCTCCGAACTTCAAAGGAACCTTCCTTTGAAGGT
TTTTACAAAAGCCGGTTCGAGACGAAGCAGAAACGTTTGATTAAAATGCTTGATTTCGAT
ATCGATCGATTTACCAAGCTACAGAAGATGCATCGTCCAAATTTGACTAAATCTGGTCGT
TGTTGA
>12688874
MSSKKSLVQSLFNISKTYSRISGLTRMRPTKSGGIPPDAGDSGIRRRFLHKRAFFSPEIV
PKGGNLMEKLRELTLSNNNRIRLDEMLPPPSPKKSSPEFFPAVTVEDVKKLMRAAEMELV
KSKLREIGKNWVPYSEFVRVCGEYSSDPEQGNRVANMLDEAGNVIVLGKLVCLKPEELTS
AMAGLIPTLEPSLDAETRQEFEQLEIIKSDIDKRADDLVRKELWAGLGLIMAQTVGFFRL
TFWELSWDVMEPICFYVTSTYFMAGYAFFLRTSKEPSFEGFYKSRFETKQKRLIKMLDFD
IDRFTKLQKMHRPNLTKSGRC*
>13603177
GTATAAAGACGACAAAGTAAACCAAAAAAAAAAAGAGTTCTCCTACAATTTTCCTAAATT
CTTGGATTTGAGATTTCACTTTTTCCGATTTGAAACAATGATGATAACTCGCGGTGGAGC
CAAGGCGGCGAAATCGCTGTTAGTGGCGGCTGGACCACGTTTGTTCTCGACGGTCCGTAC
GGTTTCGTCTCACGAGGCTTTATCAGCAAGCCATATTTTGAAGCCTGGTGTTACATCTGC
TTGGATATGGACTAGAGCTCCGACGATTGGAGGTATGAGATTCGCTAGCAGGATCACTCT
GGGAGAGAAAACTCCGATGAAGGAGGAGGACGCGAATCAGAAGAAAACAGAGAACGAATC
CACCGGTGGAGACGCCGCCGGAGGTAATAACAAGGGAGATAAAGGAATCGCGAGCTATTG
GGGTGTTGAACCTAATAAGATTACTAAAGAAGATGGTTCTGAATGGAAGTGGAACTGTTT
CAGGCCATGGGAAACGTATAAAGCTGATATAACGATAGATCTGAAGAAGCATCATGTTCC
AACGACGTTTCTTGATAGAATAGCTTATTGGACTGTTAAATCTCTTCGTTGGCCTAGCGA
TTTGTTCTTCCAGAGGAGATATGGATGTCGAGCTATGATGCTTGAAACTGTAGCAGCAGT
ACCTGGAATGGTTGGAGGAATGTTACTACACTGCAAATCGCTTCGACGTTTTGAGCAAAG
TGGAGGATGGATTAAGGCTCTTCTTGAGGAAGCAGAGAATGAGAGAATGCATCTTATGAC
ATTCATGGAAGTCGCGAAACCGAAATGGTACGAGAGAGCGCTGGTGATCACTGTGCAAGG
AGTCTTCTTCAAGGCTTATTTCCTTGGTTACTTAATCTCTCCCAAGTTTGCTCATCGTAT
GGTTGGGTACCTTGAAGAAGAAGCGATCCATTCTTATACTGAGTTTCTCAAGGAACTTGA
CAAAGGTAACATTGAGAATGTTCCTGCTCCGGCTATTGCTATTGATTACTGGAGGCTTCC
TGCTGATGCGACACTTCGTGATGTTGTGATGGTTGTTCGTGCTGACGAGGCTCATCACCG
TGATGTAAACCATTTTGCATCTGATATTCACTACGAAGGTCGTGAACTAAAGGAAGCTCC
AGCTCCAATTGGGTATCATTGATTCGATTAAAAGAAGAGCTTTTTCTCAAGTTTAAAACT
TTGTTCTAAAGAATTTAAGTTCTTTGACTTGTATATACATCATCACCTCTGCTTAAGCCA
TACTTGGATTCGGCTTTCTTTGAATGTTGCTACGAATGTTCTGATTTCTTCTTTACTTTT
CCTGTCAATGGGCTTTTGGGCT
>13603179
MMITRGGAKAAKSLLVAAGPRLFSTVRTVSSHEALSASHILKPGVTSAWIWTRAPTIGGM
RFASTITLGEKTPMKEEDANQKKTENESTGGDAAGGNNKGDKGIASYWGVEPNKITKEDG
SEWKWNCFRPWETYKADITIDLKKHHVPTTFLDRIAYWTVKSLRWPTDLFFQRRYGCRAM
MLETVAAVPGMVGGMLLHCKSLRRFEQSGGWIKALLEEAENERMHLMTFMEVAKPKWYER
ALVITVQGVFFNAYFLGYLISPKFAHRMVGYLEEEAIHSYTEFLKELDKGNIENVPAPAI
AIDYWRLPADATLRDVVMVVRADEAHHRDVNHFASDIHYQGRELKEAPAPIGYH*
>12371508
ATTCCACTCCCACTAAACATTCCTTCTCTCGCTCACTCTTCTCCAATCCTTATTTTATTT
TTTGAAAGTTTAAAATTTTATACAACATATCAATTTGGGGTAGAAAAATTCGAAAGAAAT
GAAAGAGATGGGAGTGATAGTGCTTCTTCTCCTTCACTCGTTCTTCTACGTTGCCTTTTG
CTTCAATGATGGACTACTACCAAACGGTGACTTCGAACTCGGTCCACGACATTCGGACAT
GAAAGGAACACAAGTTATCAACATAACAGCAATCCCAAACTGGGAACTCTCAGGCTTTGT
CGAGTACATTCCCTCAGGACACAAACAAGGCGACATGATCCTTGTCGTGCCTAAAGGCGC
ATTCGCAGTACGTCTAGGCAACGAAGCCTCAATCAAACAAAAAATCAGCGTTAAGAAAGG
GTCGTACTATTCGATAACGTTCAGTGCTGCTCGAACATGCGCACAAGACGAGCGGTTAAA
CGTTTCCGTGGCTCCTCACCATGCAGTGATGCCGATACAAACAGTGTATAGTAGCTCAGG
TTGGGATTTGTATTCGTGGGCTTTTAAGGCCCAAAGTGACTATGCAGATATAGTGATACA
TAATCCAGGTGTTGAGGAAGATCCTGCTTGTGGACCTCTCATTGATGGTGTTGCTATGCG
AGCCCTTTTCCCTCCTCGTCCCACCAATAAGAACATTCTAAAGAACGGAGGATTCGAAGA
AGGTCCTTGGGTTTTACCAAACATATCATCTGGTGTTTTGATTCCACCAAACTCCATCGA
CGATCACTCTCCGTTACCTGGTTGGATGGTCGAGTCTCTTAAAGCTGTCAAATACATAGA
TTCCGATCATTTCTCCGTTCCTCAAGGCCGTCGCGCCGTCGAACTCGTCGCCGGGAAAGA
AAGCGCCGTCGCACAAGTTGTCCGCACTATCCCTGGAAAAACCTACGTCCTATCCTTCTC
TGTCGGAGATGCTAGCAACGCTTGCGCCGGATCAATGATCGTCGAAGCTTTCGCCGGAAA
AGACACGATCAAGGTCCCGTATGAATCGAAAGGGAAAGGAGGATTCAAGCGATCGTCATT
GAGATTCGTCGCTGTCTCGAGTCGGACTAGAGTTATGTTCTACAGTACGTTTTACGCGAT
GAGAAACGACGATTTCTCGAGCTTATGTGGACCGGTGATCGACGACGTTAAGCTTCTCAG
TGCTCGGAGGCCGTGAGCTTGCGGCGACGAGTTGATTCACGGGACAATGAATGATGACAG
TCACTGTGGGTTTCTCGCGTCTAGTGAGAAATTGGGCTTTTAGGCCCAGTGGCCCACTGT
TTTTGTTGTTGTTTTAAAGCTTAATGTTATTTGACAAAGAAAAAAGAAATTACTCTGGTC
AATCATATCGAACCGTGAAATTTTATGATCTTGTGATT
>12371509
MKEMGVIVLLLLHSFFYVAFCFNDGLLPNGDFELGPRHSDMKGTQVINITAIPNWELSGF
VEYIPSGHKQGDMILVVPKGAFAVRLGNEASIKQKISVKKGSYYSITFSAARTCAQDERL
NVSVAPHHAVMPIQTVYSSSGWDLYSWAFKAQSDYADTVIHNPGVEEDPACGPLIDGVAM
RALFPPRPTNKNILKNGGFEEGPWVLPNISSGVLIPPNSIDDHSPLPGWMVESLKAVKYI
DSDHFSVPQGRRAVELVAGKESAVAQVVRTIPGKTYVLSFSVGDASNACAGSMIVEAFAG
KDTIKVPYESKGKGGFKRSSLRFVAVSSRTRVMFYSTFYAMRNDDFSSLCGPVIDDVKLL
SARRP*
>12699286
atgggtaaagagaagtttcacatcaacattgtggtcattggccacgtcgattctggaaag
tcgaccaccactgggcacttgatctacaagttgggtggtattgacaagcgtgtcattgag
aggttcgagaaggaggctgctgagatgaacaagaggtccttcaagtacgcatgggttttg
gacaaacttaaggctgagcgtgagcgtggtatcaccattgacattgctctctggaagttc
gagaccaccaagtactactgcactgtcattgatgctcctggccatcgtgatttcatcaag
aacatgatcactggtacctcccaggctgattgtgctgtccttatcattgactccaccact
ggtggttttgaggctggtatctccaaggatggtcagacccgtgagcacgctctacttgct
ttcacccttggtgtcaagcagatgatctgctgttgtaacaagatggatgccactaccccc
aagtactccaaggccaggtacgatgaaatcatcaaggaggtgtcttcctacttgaagaag
gttggttacaaccccgacaaaatcccatttgtgcccatctctggatttgagggtgacaac
atgattgagaggtccaccaaccttgactggtacaagggaccaactctccttgaggctctt
gaccagatcaacgagcccaagaggccgtcagacaagccccttcgtctcccacttcaggat
gtctacaagattggtggtattggaacggtgccagtgggacgtgttgagactggtatgatc
aagcctggtatggttgtgacctttgctcccacaggattgaccactgaggtcaagtctgtt
gagatgcaccacgagtctcttcttgaggcacttccaggtgacaacgttgggttcaatgtt
aagaatgttgctgtcaaggatcttaagagagggtacgtcgcatccaactccaaggatgac
cctgccaagggtgctgctaacttcacctcccaggtcatcatcatgaaccaccctggtcag
attggtaacggttacgccccagtcctggattgccacacctctcacattgcagtcaagttc
tctgagatcttgaccaagattgacaggcgttctggtaaggagattgagaaggagcccaag
ttcttgaagaatggtgatgctggtatggtgaagatgactccaaccaagcccatggttgtg
gagaccttctctgagtacccaccacttggacgtttcgctgtgagggacatgaggcagact
gttgcagtcggtgttatcaagagtgttgacaagaaggacccaaccggagccaaggttacc
aaggctgccgtcaagaagggtgaatcaaaggacagtgttagttttattacaatagtttgg
tatttggtcgcgtgtctgtgttcttgtttcgttttctccccgtcagagcgttgttctcgt
aattgggttcttgatcggaggtggcggatctacacacacattcttcctccgcatcatcct
tctcccttgtgcgatatcgtttgcctaaccatgggtaaagagaagtttcacatcaacatt
gtggtcattggccacgtcgattctggaaagtcgacaaccactggacacttgatctacaag
ttgggtggtattgacaagcgtgtgatcgagaggttcgagaaggaggctgctgagatgaac
aagaggtccttcaagtacgcatgggtgttggacaaacttaaggctgagcgtgagcgtggt
atcaccattgacattgctctctggaagttcgagaccaccaagtactactgcactgtcatt
gatgctcctggtcatcgtgatttcatcaagaacatgatcactggtacctcccaggctgat
tgtgctgtccttatcattgactccaccactggtggttttgaggctggtatctccaaggat
ggtcagacccgtgagcacgctctccttgctttcacccttggtgtcaagcagatgatctgc
tgttgtaacaagatggatgccactacccccaagtactccaaggccaggtacgatgaaatc
atcaaggaggtgtcttcctacttgaagaaggttggttacaaccccgacaaaatcccattt
gtgcccatctctggatttgagggtgacaacatgattgagaggtccaccaaccttgactgg
tacaagggaccaactctccttgaggctcttgaccagatcaacgagcccaagaggccgtca
gacaagccccttcgtctcccacttcaggatgtctacaagattggtggtattggaacggtg
ccagtgggacgtgttgagactggtatgatcaagcctggtatggttgtgacctttgctccc
acaggattgaccactgaggtcaagtctgttgagatgcaccacgagtctcttcttgaggca
cttccaggtgacaacgttgggttcaatgttaagaatgttgccgtgaaggatcttaagaga
gggtacgtcgcctccaactccaaggatgaccctgccaagggtgctgctaacttcacctcc
caggtcatcatcatgaaccaccctggtcagattggtaacggttacgccccagtcttggat
tgccacacctctcacattgcagtcaagttctctgagatcttgaccaagattgacaggcgt
tctggtaaggagattgagaaggagcccaaattcttgaagaatggtgatgctggtatggtg
aagatgactccaaccaagcccatggttgtggagaccttctctgagtacccaccacttgga
cgtttcgctgttagggacatgaggcagactgttgcagtcggtgttatcaagagtgttgac
aagaaggacccaaccggagccaaggttaccaaggctgcagttaagaagggtgcaaagtga
>12699287
MGKEKFHINIVVIGHVDSGKSTTTGHLIYKLGGIDKRVIERFEKEAAEMNKRSFKYAWVL
DKLKAERERGITIDIALWKFETTKYYCTVIDAPGHRDFIKNMITGTSQADCAVLIIDSTT
GGFEAGISKDGQTREHALLAFTLGVKQMICCCNKMDATTPKYSKARYDEIIKEVSSYLKK
VGYNPDKIPFVPISGFEGDNMIERSTNLDWYKGPTLLEALDQINEPKRPSDKPLRLPLQD
VYKIGGIGTVPVGRVETGMIKPGMVVTFAPTGLTTEVKSVEMHHESLLEALPGDNVGFNV
KNVAVKDLKRGYVASNSKDDPAKGAANFTSQVIIMNHPGQIGNGYAPVLDCHTSHIAVKF
SEILTKIDRRSGKEIEKEPKFLKNGDAGMVKMTPTKPMVVETFSEYPPLGRFAVRDMRQT
VAVGVIKSVDKKDPTGAKVTKAAVKKGESKDSVSFITIVWYLVACLCSCFVFSPSERCSR
NWVLDRRWRIYTHILPPHHPSPLCDIVCLTMGKEKFHINIVVIGHVDSGKSTTTGHLIYK
LGGIDKRVIERFEKEAAEMNKRSFKYAWVLDKLKAERERGITIDIALWKFETTKYYCTVI
DAPGHRDFIKNMITGTSQADGAVLIIDSTTGGFEAGISKDGQTREHALLAFTLGVKQMIC
CCNKMDATTPKYSKARYDEIIKEVSSYLKKVGYNPDKIPFVPISGFEGDNMIERSTNLDW
YKGPTLLEALDQINEPKRPSDKPLRLPLQDVYKTGGIGTVPVGRVETGMIKPGMVVTFAP
TGLTTEVKSVEMHHESLLEALPGDNVGFNVKNVAVKDLKRGYVASNSKDDPAKGAANFTS
QVIIMNHPGQIGNGYAPVLDCHTSHIAVKFSETLTKIDRRSGKEIEKEPKFLKNGDAGMV
KMTPTKPMVVETFSEYPPLGRFAVRDMRQTVAVGVIKSVDKKDPTGAKVTKAAVKKGAK*
>6743209
atggcgagttccgttttctctcggttttctatatacttttgtgttcttctattatgccat
ggttctatggcccagctatttaatcccagcacaaacccatggcatagtcctcggcaagga
agttttagggagtgtagatttgatagactacaagcatttgagccacttcggaaagtgagg
tcagaagctggggtgactgagtacttcgatgagaagaatgaattattccagtgcacgggt
acttttgtgatccgacgtgtcattcagcctcaaggccttttggtacctcgatacacaaat
actcctggcgtggtctacatcatccaagggagaggttctatgggtttaaccttccccggt
tgccctgcgacttaccagcaacaattccaacaattttcatctcaaggccaaagtcagagc
caaaagtttagggatgagcaccaaaagattcatcaatttaggcaaggagacattgttgca
ctcccagctggtgttgcacattggttctacaatgatggtgatgcacctgttgttgccgta
tatgtttatgacgtaaacaacaacgccaatcagcttgaacccaggcaaaaggagttccta
ttagccggcaacaacaatcgggctcagcaacaacaagtatatggtagctcaattgagcaa
cactctgggcaaaacatattcagcggattcggtgttgagatgctaagtgagtctttaggc
atcaacgcagtagcagcaaagaggctacagagccaaaatgatcaaagaggagagatcata
catgtgaagaatggccttcaattgttgaaaccgactttgacacaacagcaagaacaagca
caagcacaagatcaatatcaacaagttcaatacagtgaacgacagcaaacatcttctcga
tggaatggattggaggagaacttctgcacgatcaaggcgagagtaaacattgaaaatcct
agtcgtgctgattcatacaacccacgtgccggaaggataacaagtgtcaatagtcagaag
ttccccatccttaacctcatccaaatgagcgctaccagagtaaacctataccagaatgct
attctctcgccgttctggaacgtcaatgctcatagtttggtctatatgattcaagggcga
tctcgagttcaagtcgttagtaactttggaaagactgtgtttgatggtgtccttcgccca
ggacaattattgatcattccgcaacattatgctgtcttgaagaaagcagagcgtgaagga
tgccaatatatcgcaatcaagacaaacgctaacgccttcgtcagccaccttgcagggaaa
aactcagtattccgtgccttgccagttgatgtagtcgctaatgcgtatcgcatctcaagg
gagcaagcccgaagcctcaagaacaacaggggagaagagcacggtgccttcactcctaga
tttcaacaacaatactacccaggattatcgaatgagtccgaaagcgagacctcagagtaa
>6743210
MASSVFSRFSIYFCVLLLCHGSMAQLFNPSTNPWHSPRQGSFRECRFDRLQAFEPLRKVR
SEAGVTEYFDEKNELFQCTGTFVIRRVIQPQGLLVPRYTNTPGVVYIIQGRGSMGLTFPG
CPATYQQQFQQFSSQGQSQSQKFRDEHQKIHQFRQGDIVALPAGVAHWFYNDGDAPVVAV
YVYDVNNNANQLEPRQKEFLLAGNNNRAQQQQVYGSSIEQHSGQNIFSGFGVEMLSESLG
INAVAAKRLQSQNDQRGEIIHVKNGLQLLKPTLTQQQEQAQAQDQYQQVQYSERQQTSSR
WNGLEENFCTIKARVNIENPSRADSYNPRAGRITSVNSQKFPILNLIQMSATRVNLYQNA
ILSPFWNVNAHSLVYMIQGRSRVQVVSNFGKTVFDGVLRPGQLLIIPQHYAVLKKAEREG
CQYIAIKTNANAFVSHLAGKNSVFRALPVDVVANAYRISREQARSLKNNRGEEHGAFTPR
FQQQYYPGLSNESESETSE*
>12673011
ACATCTCACTGCTCACTACTCTCACTGTAATCCCTTAGATCTTCTTTTCAAATTTCAATG
GCGTCCGGTGATGTTGAGTATCGGTGCTTCGTTGGAGGTCTAGCATGGGCCACTGATGAC
AGAGCTCTTGAGACTGCCTTCGCTCAATACGGCGACGTTATTGATTCCAAGATCATTAAC
GATCGTGAGACTGGAAGATCAAGGGGATTCGGATTCGTCACCTTCAAGGATGAGAAAGCC
ATGAAGGATGCGATTGAGGGAATGAACGGACAAGATCTCGATGGCCGTAGCATCACTGTT
AACGAGGCTCAGTCACGAGGAAGCGGTGGCGGCGGAGGCCACCGTGGAGGTGGCGGCGGT
AGACGCGAGGGTGGAGGAGGATACAGCGGCGGCGGCGGCGGTTACTCCTCAAGAGGTGGT
GGTGGCGGAAGCTACGGTGGTGGAAGACGTGAGGGAGGAGGAGGATACGGTGGTGGTGAA
GGAGGAGGTTACGGAGGAAGCGGTGGTGGTGGAGGATGGTAATTCCTTTAATTAGGTTTG
GGATTACCAATGAATGTTCTCTCTCTCGCTTGTTATGCTTCTACTTGGTTTTGTGTGTTC
TCTATTTTGTTCTGGTTCTGCTTTAGATTTGATGTAACAGTTCGTGATTAGGTATTTTGG
TATCTGGAAACGTAATGTTAAGTGACTTGTCATTCTCTAAATAACAAATTTCTTCGGAGA
TATTATCTCTGTTGATTGATTCT
>12673012
MASGDVEYRCFVGGLAWATDDRALETAFAQYGDVIDSKIINDRETGRSRGFGFVTFKDEK
AMKDAIEGMNGQDLDGRSITVNEAQSRGSGGGGGHRGGGGGRREGGGGYSGGGGGYSSRG
GGGGSYGGGRREGGGGYGGGEGGGYGGSGGGGGW*
>12712671
ACCAAATACAAACCCTAGCCGCCTTATTCGTCTTCTTCGTTCTCTAGTTTTTTCCTCAGT
CTCTGTTCTTAGATCCCTTGTAGTTTCGAAATCTTCCGATAAAAATGTCGGGTAAAGGAG
AAGGACCAGCTATCGGTATCGATCTTGGTACCACTTACTCTTGCGTCGGAGTATGGCAAC
ACGACCGTGTTGAGATCATTGCTAATGATCAAGGAAACAGAACCACGCCATCTTACGTTG
CTTTCACCGACTCCGAGAGGTTGATCGGTGACGCAGCTAAGAATCAGGTCGCCATGAACC
CCGTTAACACCGTTTTCGACGCTAAGAGGTTGATCGGTCGTCGTTTCTCTGACAGCTCTG
TTCAGAGTGACATGAAATTGTGGCCATTCAAGATTCAAGCCGGACCTGCCGATAAGCCAA
TGATCTACGTCGAATACAAGGGTGAAGAGAAAGAGTTCGCAGCTGAGGAGATTTCTTCCA
TGGTTCTTATTAAGATGCGTGAGATTGCTGAGGCTTACCTTGGTGTCACAATCAAGAACG
CCGTTGTTACCGTTCCAGCTTACTTCAACGACTCTCAGCGTCAGGCTACAAAGGATGCTG
GTGTCATCGCTGGTTTGAACGTTATGCGAATCATCAACGAGCCTACAGCCGCCGCTATTG
CCTACGGTCTTGACAAAAAGGCTACCAGCGTTGGAGAGAAGAATGTTCTTATCTTCGATC
TTGGTGGTGGCACTTTTGATGTCTCTCTTCTTACCATTGAAGAGGGTATCTTTGAGGTGA
AGGCAACTGCTGGTGACACCCATCTTGGTGGGGAAGATTTTGACAACAGAATGGTTAACC
ACTTTGTCCAAGAGTTCAAGAGGAAGAGTAAGAAGGATATCACCGGTAACCCAAGAGCTC
TTAGGAGGTTGAGAACTTCCTGTGAGAGAGCGAAGAGGACTCTTTCTTCCACTGCTCAGA
CCACCATCGAGATTGACTCTCTATACGAGGGTATCGACTTCTACTCCACCATCACCCGTG
CTAGATTTGAGGAGCTCAACATGGATCTCTTCAGGAAGTGTATGGAGCCAGTTGAGAAGT
GTCTTCGTGATGCTAAGATGGACAAGAGCACTGTTCATGATGTTGTCCTTGTTGGTGGTT
CTACCCGTATCCCTAAGGTTCAGCAATTGCTCCAGGACTTCTTCAACGGCAAAGAGCTTT
GCAAGTCTATTAACCCTGATGAGGCTGTTGCCTACGGTGCTGCTGTCCAGGGAGCTATTC
TCAGCGGTGAAGGAAACGAGAAGGTTCAAGATCTTCTATTGCTCGATGTCAGTCCTCTCT
CCCTTGGTTTGGAAACTGCCGGTGGTGTCATGACCACTTTGATCCCAAGGAACACAACCA
TCCCAACCAAGAAGGAACAAGTCTTCTCCACCTACTCAGACAACCAACCCGGTGTGTTGA
TCCAGGTGTACGAAGGAGAGAGAGCCAGAACCAAGGACAACAACCTTCTTGGTAAATTTG
AGCTCTCCGGAATTCCTCCAGCTCCTCGTGGTGTCCCCCAGATCACAGTCTGCTTTGACA
TTGATGCCAATGGTATCCTCAATGTCTCTGCTGAGGACAAGACCACCGGACAGAAGAACA
AGATCACCATCACCAATGACAAGGGTCGTCTCTCCAAGGATGAGATTGAGAAGATGGTTC
AAGAGGCTGAGAAGTACAAGTCCGAAGACGAGGAGCACAAGAAGAAGGTTGAAGCCAAGA
ACGCTCTCGAGAACTACGCTTACAACATGAGGAACACCATCCAAGACGAGAAGATTGGTG
AGAAGCTCCCGGCTGCAGACAAGAAGAAGATCGAGGATTCTATTGAGCAGGCGATTCAAT
GGCTCGAGGGTAACCAGTTGGCTGAGGCTGATGAGTTCGAAGACAAGATGAAGGAATTGG
AGAGCATCTGCAACCCAATCATTGCCAAGATGTACCAAGGAGCTGGTGGTGAAGCCGGTG
GTCCAGGTGCCTCTGGTATGGACGATGATGCTCCCCCTGCTTCAGGCGGTGCTGGACCTA
AGATCGAGGAGGTCGACTAATTTGTTGGACATTGACCTCTCTCTTTCTCCTATCTCTATC
TCTTTTACTTGCTTTTTTTTGATCTGTTAAGACTTTTTATGTTGGGCTTTTTTAAAGAAG
CCCATTTTGTGGTGTTTTTTGGTTAGTACTATTTTGAACAATGGTTGGTTCTATACCAGT
TTAGCTACGATGACGGATAAAATTAAAAGTTTGCC
>12712672
MSGKGEGPAIGIDLGTTYSCVGVWQHDRVEIIANDQGNRTTPSYVAFTDSERLIGDAAKN
QVAMNPVNTVFDAKRLIGRRFSDSSVQSDMKLWPFKIQAGPADKPMIYVEYKGEEKEFAA
EEISSMVLIKMREIAEAYLGVTIKNAVVTVPAYFNDSQRQATKDAGVIAGLNVMRIINEP
TAAAIAYGLDKKATSVGEKNVLIFDLGGGTFDVSLLTIEEGIFEVKATAGDTHLGGEDFD
NRNVNHFVQEFKRKSKKDITGNPRALRRLRTSCERAKRTLSSTAQTTIEIDSLYEGIDFY
STITRARFEELNMDLFRKCMEPVEKCLRDAKMDKSTVHDVVLVGGSTRIPKVQQLLQDFF
NGKELCKSINPDEAVAYGAAVQGAILSGEGNEKVQDLLLLDVTPLSLGLETAGGVMTTLI
PRNTTIPTKKEQVFSTYSDNQPGVLIQVYEGERARTKDNNLLGKFELSGIPPAPRGVPQI
TVGFDIDANGILNVSAEDKTTGQKNKITITNDKGRLSKDEIEKMVQEAEKYKSEDEEHKK
KVEAKNALENYAYNMRNTIQDEKIGEKLPAADKKKIEDSIEQAIQWLEGNQLAEADEFED
KMKELESICNPIIAKMYQGAGGEAGGPGASGMDDDAPPASGGAGPKIEEVD*
>13604752
AGGAGTAAAGAAGTAAGAAACGAAGCGGTTTTGAAGTCATCTCTTCAGATATGTTTGTTC
TAATTAAAATTTGCAAGTGGGAATTAGTTTGTAATTGAAGGTATGCACGATTTTTAGTTA
CAATTTTAATTCTTCTTCTTCAGATCCAAGAACTCTCAGTCTCTGCGTTCACACTCTTTC
TTTGAATCCTTCATCATCGTAATTCATCTCCAAGAACTGAATCAGAAGTTGTATTTCGCT
AATTCAACTTTTCCAGTGTCTGTCAATTAGGTTTTGATTTTGGAAGTAGAGAAGTTAGAA
GAAGAATGGTGAGAACGCGGCGAAGAGGGCAGAGATCAAAGGGAATTAAAGTGAAGCATT
GTATTCAGTTGACTCTATTGCTTGGTGTTGGGATATGGCTGATTTATCAAATGAAGCATT
CACATGAGAAGAAAGCTGAGTTTGAAGGGACTTCAAAGATTGTTGTTGATGATATTGATA
ATACAGTTGTTAATCTTGGAAGGAAAGATCTTAGACCGCGTATTGAGGAGACGAAAGATG
TGAAGGACGAAGTGGAAGATGAAGAAGGGAGCAAGAATGAAGGAGGAGGAGACGTAAGTA
CTGATAAGGAGAATGGTGATGAGATTGTAGAGAGGGAGGAGGAGGAAAAGGCTGTAGAAG
AGAATAACGAGAAGGAAGCTGAAGGTACCGGGAATGAAGAGGGAAACGAGGATTCAAACA
ATGGAGAAAGTGAGAAGGTTGTTGATGAGAGTGAAGGTGGAAATGAGATPAGTAATGAGG
AAGCTAGGGAGATCAATTACAAGGGAGATGATGCGTCGAGTGAGGTTATGCATGGGACGG
AGGAGAAGAGCAATGAATAGGTTGAAGTTGAGGGAGAAGTAAATCTAAGTAGTACTGAAA
ATGTCAGTGTCCATGAAGATGAGTCGGGTCCAAAGAATGAAGTATTGGAGGGTTCTGTTA
TTAAAGAAGTTTCTTTGAACACAACTGAGAATGGTAGTGATGATGGTGAGCAACAAGAGA
CAAAGAGTGAGTTGGATTCAAAGACTGGTGAGAAGGGCTTTTCTGATTCTAATGGTGAAT
TGCCTGAGACTAACCTGTCAACTTCCAATGCAACTGAAACTACAGAATCTTCTGCGAGTG
ATGAGTCAGGATCGAGCGGGAAATCCACTGGTTATCAACAAACGAAAAACGAAGAAGATG
AGAAGGAAAAGGTACAATCATCTGAAGAGGAAAGCAAAGTCAAAGAATCCGGGAAAAATG
AGAAGGACGCGTCCTCGTCCCAAGACGAAAGTAAAGAGGAAAAACCCGAGAGAAAGAAGA
AAGAAGAGTCTTCGTCCCAAGGGGAAGGTAAAGAAGAAGAACCCGAGAAAAGGGAGAAAG
AAGACTCTTCATCCCAAGAGGAAAGTAAAGAGGAAGAACCTGAGAACAAAGAGAAAGAAG
CGTCTTCCTCTCAGGAGGAGAATGAGATTAAAGAAACTGAGATAAAGGAGAAAGAAGAGT
CTTCGTCCCAAGAGGGGAATGAGAACAAAGAAACAGAAAAAAAGTCTTCCGAATCTCAGA
GAAAGGAAAACACCAACAGTGAGAAGAAAATTGAACAGGTGGAATCTACTGATTCTTCAA
ACACACAGAAGGGTGACGAACAGAAAACTGATGAAAGCAAGAGAGAATCCGGCAATGATA
CTTCAAATAAGGAAACAGAGGATGATAGTTCAAAAACAGAGTCAGAGAAGAAAGAGGAAA
ATAACAGAAATGGTGAAACAGAGGAGACCCAAAACGAACAAGAACAGACCAAGTCCGCTT
TGGAAATTAGTCACACTCAAGATGTTAAGGATGCTCGAACTGATCTAGAAACTCTTCCTG
AAACCAGCAATGGATTGATCAGCGACAAAGTTGCTGCTGAGTGATACTTGTTAAATGTGT
GAAGCTGTCATGTATTGTATGCATATTCTATACTTCTCACCAAATGACGGGACTTAAGTC
CCAATCAAAGTAGTATTGAGTTTTTAATCGTAATCAGTACATGTGTATGTATACTTTTAT
ATCTAATTCTCTCTGAGTCTCTAATAATGAAGAAGTTTTTTTTTT
>13604753
MVRTPRRGQRSKGIKVKHCIQLTLLLGVGIWLIYQMKHSHEKKAEFEGTSKIVVDDIDNT
VVNLGRKDLRPRIEETKDVKDEVEDEEGSKNEGGGDVSTDKENGDEIVEREEEEKAVEEN
NEKEAEGTGNEEGNEDSNNGESEKVVDESEGGNEISNEEAREINYKGDDASSEVMHGTEE
KSNEKVEVEGESKSNSTENVSVHEDESGPKNEVLEGSVIKEVSLNTTENGSDDGEQQETK
SELDSKTGEKGFSDSNGELPETNLSTSNATETTESSGSDESGSSGKSTGYQQTKNEEDEK
EKVQSSEEESKVKESGKNEKDASSSQDESKEEKPERKKKEESSSQGEGKEEEPEKREKED
SSSQEESKEEEPENKEKEASSSQEENEIKETEIKEKEESSSQEGNENKETEKKSSESQRK
ENTNSEKKIEQVESTDSSNTQKGDEQKTDESKRESGNDTSNKETEDDSSKTESEKKEENN
RNGETEETQNEQEQTKSALEISHTQDVKDARTDLETLPETSNGLISDKVAAE*
>12679464
ATTCACACCGGACATTTTGAAATCTCAACAAGAACCAAACCAAACAACAAAAAAACATTC
TTAATAATTATCTTTCTGTTATGTCGATGACGGCGGATTCTCAATCTGATTATGCTTTTG
TTGAGTCCATACGACGACACTTACTAGGAGGAATCGGAGCCGATACTCAGTGAGTCGACA
GCGAGTTCGGTTACTCAATCTTGTGTAACGGGTCAGAGCATTAAACCGGTGTACGGACGA
AACCCTAGCTTTAGCAAACTGTATCCTTGCTTCACCGAGAGCTGGGGAGATTTGCCGTTG
AAAGAAAACGATTCTGAGGATATGTTAGTTTACGGTATCCTCAACGACGCCTTTCACGGC
GGTTGGGAGCCGTCTTCTTCGTCTTCCGACGAAGATCGTAGCTCTTTGCCGAGTGTTAAG
ATCGAGACTCCGGAGAGTTTGGCGGCGGTGGATTGTGTTCCGGTQAAGAAGGAGAAGACG
AGTCCTGTTTGGGCGGCGGTGACGGCGGCGAAGGGAAAGCATTATAGAGGAGTGAGAGAA
AGGCCGTGGGGGAAATTTGCGGCGGAGATTAGAGACCCGGCGAAGAACGGAGCTAGGGTT
TGGTTAGGAACGTTTGAGACGGCGGAGGACGCGGCGTTGGCTTACGACAGAGCTGCTTTC
AGGATGCGTGGTTCGCGCGCTTTGTTGAATTTTCGGTTGAGAGTTAATTCAGGAGAACCC
GACCCGGTTCGAATCAAGTCGAAGAGATCTTCTTTTTCTTCTTCTAACGAGAACGGAGCT
CCGAAGAAGAGGAGAACGGTGGCCGCCGGTGGTGGAATGGATAAGGGATTGACGGTGAAG
TGCGAGGTTGTTGAAGTGGGAGGTGGCGATCGTTTATTGGTTTTATAATTTTGATTTTTC
TTTGTTGGATGATTATATGATTCTTCAAAAAAGAAGAACGTTAATAAAAAAATTCGTTTA
TTATTAT
>12679465
MLVYGILNDAFHGGWEPSSSSSDEDRSSPPSVKTETPESFAAVDSVPVKKEKTSPVSAAV
TAAKGKHYRGVRQRPWGKFAAEIRDPAKNGARVWLGTFETAEDAALAYDRAAFRMRGSRA
LLNFPLRVNSGEPDPVRIKSKRSSFSSSNENGAPKKRRTVAAGGGMDKGLTVKCEVVEVA
RGDRLLVL*
>12719868
AAATCAAATCTTCTTCCTTCTCTGTTTTCTTAAGCTTTTTGAAAATTTTATCAATGGCGA
CTCCTAACGAAGTATCTGCACTTTGGTTCATCGAGAAACATCTACTCGACGAGGCTTCTC
CTGTGGCTACAGATCCATGGATGAAGCACGAATCATCATCAGCAACAGAATCTAGCTCTG
ACTCTTCTTCTATCATCTTCGGATCATCGTCCTCTTCTTTCGCCCCAATTGATTTCTCTG
AATCCGTATGCAAACCTGAAATCATCGATCTCGATACTCCCAGATCTATGGAATTTCTAT
CGATTCGATTTGAATTTGACTCAGAAGTTTCTGTTTCTGATTTCGATTTTAAACCTTCTA
ATCAAAATCAAAATCAGTTTGAACCGGAGCTTAAATCTCAAATTCGTAAACCGCCATTGA
AGATTTCGCTTCCAGCTAAAACAGAGTGGATTCAATTCGCAGCTGAAAACACCAAACCGG
AAGTTACTAAACCGGTTTCGGAAGAAGAGAAGAAGCATTACAGAGGAGTAAGACAAAGAC
CGTGGGGGAAATTCGCGGCGGAGATTCGTGACCCGAATAAACGCGGATCTCGCGTTTGGC
TTGGGACGTTTGATACAGCGATTGAAGCGGCTAGAGCTTATGACGAAGCAGCGTTTAGAC
TACGAGGATCGAAAGCGATTTTGAATTTCCCTCTTGAAGTTGGGAAGTGGAAACCACGCG
CCGATGAAGGTGAGAAGAAACGGAAGAGAGACGATGATGAGAAAGTGACTGTGGTTGAGA
AAGTGTTGAAGACGGAACAGAGCGTTGACGTTAACGGTGGAGAGACGTTTCCGTTTGTAA
CGTCGAATTTAACGGAATTATGTGACTGGGATTTAACGGGGTTTCTTAACTTTCCGCTTC
TGTCGCCGTTATCTCCTCATCCACCGTTTGGTTATTCCCAGTTGACCGTTGTTTGATTAG
TTTTTTTTGAGTTTTTGAACGATGTGTATGCTGACGTGGACGTACACGTAGGTGCATGCG
ATGAAAPAAACATCTATTTGTTCATATTTTTGCGTTTTTCTATTTGTTCATTCTTTTTCA
CAATTCACAATACATTATTTCAGTTAATGATTAC
>12719870
MATPNEVSALWFIEKHLLDEASPVATDPWMKHESSSATESSSDSSSIIFGSSSSSFAPID
FSESVCKPEIIDLDTPRSMEFLSIPFEFDSEVSVSDFDFKPSNQNQNQFEPELKSQIRKP
PLKISLPAKTEWIQFAAENTKPEVTKPVSEEEKKHYRGVRQRPWGKFAAEIRDPNKRGSR
VWLGTFDTAIEAARAYDEAAFRLRGSKATLNFPLEVGKWKPRADEGEKKRKRDDDEKVTV
VEKVLKTEQSVDVNGGETFPFVTSNLTELCDWDLTGFLNFPLLSPLSPHPPFGYSQLTVV
*
>12370148
ATTCCCACTTCCACACATACACATATACAACAGAGCAAGAGAGTCAATCAAGTAGAGTGA
AGATGGCAACTAAACAAGAAGCTTTAGCCATCGATTTCATAAGCCAACACCTTCTCACAG
ACTTTGTTTCCATGGAAACTGATCACCCATCTCTTTTTACCAACCAACTTCACAACTTTC
ACTCAGAAACAGGCCCTAGAACCATCACCAACCAATCCCCTAAACCGAATTCGACTCTTA
ACCAGCGTAAACCGCCCTTACCGAATCTATCCGTCTCGAGAACGGTTTCAAGAAAGACAG
AGAAAGAGGAAGAAGAGAGGCACTACAGGGGAGTGAGACGAAGACCGTGGGGAAAATACG
CGGCGGAGATTAGGGATCCGAACAAAAAGGGTTGTAGGATCTGGCTTGGGACTTACGACA
CTGCGGTGGAAGCTGGAAGAGCTTATGACCAAGCGGCGTTTCAATTACGTGGAAGAAAAG
CAATCTTGAATTTCCCTCTCGATGTTAGGGTTACGTCAGAAACTTGTTCTGGGGAAGGAG
TTATCGGATTAGGGAAACGAAAGCGAGATAAGGGTTCTCCGCCGGAAGAGGAGAAGGCGG
CTAGGGTTAAAGTGGAGGAAGAAGAGAGTAATACGTCGGAGACGACGGAGGCTGAGGTTG
AGCCGGTGGTAGCATTGACGCCGTCAAGTTGGATGGGGTTTTGGGATGTGGGAGGAGGAG
ATGGTATTTTCAGTATTCCTCCGTTATCTCCGACGTCTCCCAACTTTTCCGTTATCTGGG
TCACTTAAAACTTCGGAAAAGTCAACGTACGATGACGTTTTCACTTGCGTCACTCTCATG
ATTTCATTTATTCTTGTATAATATAAAGGTAGCGGTAGTGTGCAAATATCAAATAAGTAG
TTTAATTAGTACCAATCATTTTATTCATTATTTTTTTTAGTAGAATATTTGGATGTTGAA
AATATAAATTTAATTTTGTATTTGTTGATGTTATAAATTTATTGATTGTATAAACATTCT
TAGTC
>12370150
MATKQEALAIDFISQHLLTDFVSMETDHPSLFTNQLHNFHSETGPRTITNQSPKPNSTLN
QRKPPLPNLSVSRTVSTKTEKEEEERHYRGVRRRPWGKYAAEIRDPNKKGCRIWLGTYDT
AVEAGRAYDQAAFQLRGRKAILNFPLDVRVTSETCSGEGVIGLGKRKRDKGSPPEEEKAA
RVKVEEEESNTSETTEAEVEPVVPLTPSSWMGFWDVGAGDGIFSIPPLSPTSPNFSVISV
>12560350
ATGGTTACCTTATGTGCCACGTTACTGATCCTTCTCTCAATCTTTCTTGCAACTCCGTCG
AATGTTCGAGGAAATGCAGAGCTGAAGGCTTTAATGGAGCTGAAATCGTCGCTTGACCCT
GAAAACAAGCTTCTCCGTTCATGGACGTTTAACGGCGATCCATGCGACGGATCTTTCGAA
GGAATTGCATGTAACCAGCATCTAAAAGTCGCAAACATATCATTACAAGGGAAACGTTTG
GTCGGAAAATTGTCTCCGGCGGTTGCAGAGCTCAAATGTTTGTCAGGTCTTTACTTACAT
TACAATAGTCTCTCTGGAGAGATACCTCAAGAGATCACAAATCTTACTGAATTATCAGAT
CTTTATCTCAATGTTAATAAGTTCTCTGGTGAGATTCCGGCAGATATCGGCTCCATGGCT
GGCTTGCAAGTTATGGATCTTTGTTGCAACAGTTTAACAGGGAAGATACCAAAGAACATT
GGATCCTTGAAGAAACTTAATGTGTTGTCTCTGCAACACAACAAACTAACCGGAGAGGTT
CCTTGGACTTTAGGAAACTTGAGTATGTTAAGCAGGCTTGATTTAAGCTTCAACAATCTG
TTAGGTTTAATCCCAAAAACCCTAGCCAACATTCCTCAGTTGGACACTCTTGACTTGCGC
AACAATACTCTCTCTGGCTTTGTTCCTCCTGGTCTTAAGAAGTTGAATGGGAGCTTCCAG
TTTGAGAACAACACCGGGTTATGTGGTATCGATTTTCCTTCTTTGAGAGCTTGTTCTGCT
TTCGATAATGCGAATAATATCGAACAATTTAAGCAGCCTCCTGGTGAAATAGACACTGAT
AAATCAGCTCTTCACAACATTCCCGAGTCTGTATATCTCCAAAAGCATTGCAACCAAACA
CATTGCAAGAAATCCTCATCGAAACTCCCACAAGTTGCTTTGATTTCAAGCGTGATTACC
GTCACTATAACATTGATTGGTGCTGGTATATTGACCTTCTTTCGCTACAGAAGAAGGAAG
CAAAAGATCAGTAACACACCTGAGTTTTCCGAGGGAAGACTAAGCACAGATCAACAAAAA
GAGTTCCGTGCATCGCCTTTAGTGAGTCTTGCCTACACTAAAGAATGGGATCCGCTAGGT
GATAGCAGAAACGGAGCTGAGTTTTCACAAGAGCCTCATCTCTTTGTTGTAAATAGTAGC
TTCAGGTTTAACTTAGAAGACATTGAATCAGCAACCCAATGCTTCTCAGAAGCTAATCTA
TTGAGCAGAAACAGCTTCACCTCAGTGTTCAAAGGAGTCCTCAGAGATGGTTCTCCGGTA
GCTATCAGAAGCATCAACATAAGCAGTTGCAAGAACGAAGAAGTCGAATTCATGAACGGT
TTAAAGCTTTTATCTTCGTTGTCACACGAAAACTTAGTGAAGCTACGTGGATTCTGCTGT
TCTAGAGGCAGAGGAGAGTGTTTCCTCATCTATGATTTTGCTTCAAAAGGAAAGCTTTCA
AATTTTCTTGACTTACAAGAACGCGAAACTAATCTGGTCCTTGCTTGGTCTGCAAGAATC
TCCATCATCAAAGGGATTGCAAAAGGTATTGCTTACTTACATGGAAGTGATCAACAGAAG
AAGCCTACAATAGTTCATCGAAACATCTCTGTCGAAAAAATCCTACTTGATGAACAATTT
AACCCGTTAATCGCTGATTCGGGTCTTCACAACCTTCTAGCAGACGATATGGTCTTCTCA
GCACTCAAAACAAGTGCAGCAATGGGATATTTAGCTCCGGAATACGTCACAACCGGAAAA
TTCACCGAGAAAACCGATATTTTCGCCTTTGGAGTCATCATTCTCCAGATACTCTCTGGT
AAGCTCATGCTTACAAGTTCACTGAGAAATGCAGCTGAAAATGGAGAACATAACGGGTTC
ATCGATGAAGATCTTCGTGAAGAGTTTGATAAACCAGAGGCGACTGCAATGGCGAGGATT
GGGATAAGCTGTACACAGGAGATACCAAACAATAGGCCTAATATAGAGACATTGCTTGAG
AATATAAACTGTATGAAGAGTGAATGA
>12560351
MVTLCATLLILLSIFLATPSNVRGNAELKALMELKSSLDPENKLLRSWTFNGDPCDGSFE
GIACNQHLKVANISLQGKRLVGKLSPAVAELKCLSGLYLHYNSLSGEIPQEITNLTELSD
LYLNVNNFSGEIPADIGSMAGLQVMDLCCNSLTGKIPKNIGSLKKLNVLSLQHNKLTGEV
PWTLGNLSMLSRLDLSFNNLLGLIPKTLANIPQLDTLDLRNNTLSGEVPPGLKKLNGSFQ
FENNTGLCGIDFPSLRACSAFDNANNIEQFKQPPGEIDTDKSALHNIPESVYLQKHCNQT
HCKKSSSKLPQVALISSVITVTITLIGAGILTFFRYRRRKQKISNTPEFSEGRLSTDQQK
EFRASPLVSLAYTKEWDPLGDSRNGAEFSQEPHLFVVNSSFRFNLEDIESATQCFSEANL
LSRNSFTSVFKGVLRDGSPVAIRSINISSCKNEEVEPMNGLKLLSSLSHENLVKLRGFCC
SRGRGECFLIYDFASKGKLSNFLDLQERETNLVLAWSARISIIKGIAKGIAYLHGSDQQK
KPTIVHRNISVEKILLDEQFNPLIADSGLHNLLADDMVFSALKTSAAMGYLAPEYVTTGK
FTEKTDIFAFGVIILQILSGKLMLTSSLRNAAENGEHNGFIDEDLREEFDKPEATAMARI
GISCTQETPNNRPNIETLLENINCMKSE*
>13603142
ATACTCTTCGACTGAGCCGTCCTTTCGCAGAAAGCATTTGGAGTTGGAGCTTTGGAGGAG
GACAATGGCCCCGAAGAAAGGAGTGAAGGTAGCTGCTAAGAAGAAGACCGCGGAGAAAGT
TTCAAACCCTCTATTCGAGAGGAGGCCTAAGCAATTCGGTATTGGTGGAGCTTTACCTCC
TAAGAAGGATCTCTCTCGCTACATCAAATGGCCCAAATCCATCCGTCTTCAAAGGCAAAA
GAGGATCCTGAAGCAGAGGTTGAAGGTCCCTCCAGCTCTTAACCAATTCACCAAGACTCT
TGACAAGAATCTTGCTACCAGCCTCTTCAAGGTCCTTCTGAAGTACAGGCCAGAAGACAA
AGCTGCCAAGAAGGAGCGTCTTGTAAAGAAGGCCCAAGCTGAAGCTGAGGGAAAGCCTTC
TGAGTCTAAGAAGCCCATTGTAGTCAAATACGGCCTCAACCATGTGACCTACCTCATTGA
GCAGAACAAGGCCCAACTTGTTGTTATTGCTCATGATGTCGACCCAATTGAGTTGGTTGT
CTGGTTGCCTGCTCTGTGCAGGAAGATGGAAGTCCCGTACTGCATTGTCAAGGGCAAATC
TCGTCTTGGAGCGGTTGTTCACCAGAAGACTGCTTCTTGCTTGTGTTTGACCACTGTCAA
GAACGAGGACAAGCTAGAGTTCAGCAAAATCCTGGAAGCTATCAAGGCCAACTTCAATGA
CAAGTACGAGGAGTACAGGAAGAAATGGGGAGGAGGCATAATGGGATCTAAGTCTCAGGC
AAAGACCAAGGCAAAGGAAAGAGTTATTGCAAAGGAGGCTGCCCAAAGGATGAATTAAGA
GGCTAGCTTCTTTTGTTTGTGGTTTGCTCGGATTCGTAAAACTTAATAGAGCTTTTTGTT
TAGCTGGTTCTTGAAGTACCTCTTTTTTAGTTGAACCCTTTATTATGGATGTTTTGCAAT
TTCTTGGGACAGTTTCAATGTTATTCAAGCTGCTGGATCCTCTTTGGTCTCTCAACCACT
TAAAACTTGACAGTGTGAAATTTTAGCCAAATGCTTATTGCATTCTGGAAAGAGTTATAA
GTTTAATTC
>13603144
MAPKKGVKVAAKKKTAEKVSNPLFERRPKQFGIGGALPPKKDLSRYIKWPKSIRLQRQKR
ILKQRLKVPPALNQFTKTLDKNLATSLFKVLLKYRPEDKAAKKERLVKKAQAEAEGKPSE
SKKPIVVKYGLNHVTYLIEQNKAQLVVIAHDVDPIELVVWLPALCRKMEVPYCIVKGKSR
LGAVVHQKTASCLCLTTVKNEDKLEFSKILEAIKANFNDKYEEYRKKWGGGIMGSKSQAK
TKAKERVIAKEAAQRMN*
>13 608279
AATCATGGCGGAGAAACTCAGTGATGGCAGCAGCATCATCTCAGTCCATCCTAGACCCTC
CAAGGGTTTCTCCTCGAAGCTTCTCGATCTTCTCGAGAGACTTGTTGTCAAGCTCATGCA
CGATGCTTCTCTCCCTCTCCACTACCTCTCAGGCAACTTCGCTCCCATCCGTGATGAAAC
TCCTCCCGTCAAGGATCTCCCCGTCCATGGATTTCTTCCCGAATGCTTGAATGGTGAATT
TGTGAGGGTTGGTCCAAACCCCAAGTTTGATGCTGTCGCTGGATATCACTGGTTTGATGG
AGATGGGATGATTCATGGGGTACGCATCAAAGATGGGAAAGCTACTTATGTTTCTCGATA
TGTTAAGACATCACGTCTTAAGCAGGAAGAGTTCTTCGGAGCTGCCAAATTCATGAAGAT
TGGTGACCTTAAGGGGTTTTTCGGATTGCTAATGGTCAATGTCCAACAGCTGAGAACGAA
GCTCAAAATATTGGACAACACTTATGGAAATGGAACTGCCAATACAGCACTCGTATATCA
CCATGGAAAACTTCTAGCATTACAGGAGGCAGATAAGCCGTACGTCATCAAAGTTTTGGA
AGATGGAGACCTGCAAACTCTTGGTATAATAGATTATGACAAGAGATTGACCCACTCCTT
CACTGCTCACCCAAAAGTTGACCCGGTTACGGGTGAAATGTTTACATTCGGCTATTCGCA
TACGCCACCTTATCTCACATACAGAGTTATCTCGAAAGATGGCATTATGCATGACCCAGT
CCCAATTACTATATCAGAGCCTATCATGATGCATGATTTTGCTATTACTGAGACTTATGC
AATCTTCATGGATCTTCCTATGCACTTCAGGCCAAAGGAAATGGTGAAAGAGAAGAAAAT
GATATACTCATTTGATCCCACAAAAAAGGCTCGTTTTGGTGTTCTTCCAGGCTATGCCAA
GGATGAACTTATGATTAGATGGTTTGAGCTTCCCAACTGCTTTATTTTCCACAACGCCAA
TGCTTGGGAAGAAGAGGATGAAGTCGTCCTCATCACTTGTCGTCTTGAGAATCCAGATCT
TGACATGGTCAGTGGGAAAGTGAAAGAAAAACTCGAAAATTTTGGCAACGAACTGTACGA
AATGAGATTCAACATGAAAACGGGCTCAGCTTCTCAAAAAAAACTATCCGCATCTGCGGT
TGATTTCCCCAGAATCAATGAGTGCTACACCGGAAAGAAACAGAGATACGTATATGGAAC
AATTCTGGACAGTATCGCAAAGGTTACCGGAATCATCAAGTTTGATCTGCATGCAGAAGC
TGAGACAGGGAAAAGAATGCTGGAAGTAGGAGGTAATATCAAAGGAATATATGACCTGGG
AGAAGGCAGATATGGTTCAGAGGCTATGTATGTTGCGCGTGAGAGAGCAGAAGAAGACGA
CGGTTACTTGATATTCTTTGTTCATGATGAAAACACAGGGAAATCATGCGTGACTGTGAT
AGACGCAAAAACAATGTCGGCTGAACCGGTGGCAGTGGTGGAGCTGCCGCACAGGGTCCC
ATATGGCTTCCATGCCTTGTTTGTTACAGAGGAACAACTCCAGGAACAAACTCTTATATA
AGCCAAGCTGTGTGCATATACACATAATACTTGTGAGTCAGAGATGTGAAACTCGGTGAT
ACTGAATTACTGATATTACCATTACACACATATTATGGCGTTATGTATTCATAATACTGC
CTGGTTTCTCTTTACATATAATTACATTTCATCCTCCAATATCAACAAGGTCTCTACTTT
>13608281
MAEKLSDGSSIISVHPRPSKGFSSKLLDLLERLVVKLMHDASLPLHYLSGNFAPIRDETP
PVKDLPVHGFLPECLNGEFVRVGPNPKFDAVAGYHWFDGDGMIHGVRIKDGKATYVSRYV
KTSRLKQEEFFGAAKFMKIGDLKGFFGLLMVNVQQLRTKLKILDNTYGNGTANTALVYHH
GKLLALQEADKPYVIKVLEDGDLQTLGIIDYDKRLTHSFTAHPKVDPVTGEMFTFGYSHT
PPYLTYRVISKDGIMHDPVPITISEPIMMHDFAITETYAIFMDLPMHFRPKEMVKEKKMI
YSFDPTKKARFGVLPRYAKDELMIRWFELPNCFIFHNANAWEEEDEVVLITCRLENPDLD
MVSGKVKEKLENFGNELYEMRFNMKTGSASQKKLSASAVDFPRTNECYTGKKQRYVYGTI
LDSIAKVTGIIKFDLHAEAETGKRMLEVGGNIKGIYDLGEGRYGSEAIYVPRETAEEDDG
YLIFFVHDENTGKSCVTVIDAKTMSAEPVAVVELPHRVPYGFHALFVTEEQLQEQTLI*
>12704782
ATCAGAAATCGAAAAATCAAAGTTCTCAAGAAGATATCAACAAAAAAAAAAGTAAATTCT
TTAAAATGTCGATGATTCCAAGTTTCTTCAACAACAACAGACGAAGCAACATCTTTGATC
CATTCTCTCTTGACGTATGGGATCCATTCAAGGAACTAACATCATCATCACTTTCTCGTG
AGAACTCAGCGATCGTGAACGCACGTGTGGACTGGAGAGAGACGCCTGAGGCCCACGTGT
TTAAAGCTGACTTGCCTGGATTGAAGAAGGAGGAAGTTAAAGTTGAGATTGAGGAGGATA
GTGTTTTGAAGATCAGTGGAGAGAGACACGTGGAGAAAGAAGATAAGAATGACACGTGGC
ACCGTGTGGAGAGATCGAGTGGACAGTTTACGAGGAGGTTTAGGTTGCCGGAGAATGTGA
AGATGGATCAGGTTAAGGCTGCGATGGAGAATGGTGTGTTGACTGTTACGGTGCCTAAGG
CTGAGACTAAGAAGGCTGATGTTAAGTCTATTCAGATCTCTGGTTGAGTAATGGGTTCGA
GTTTTATCATCGGAGTTGCTTGTGTTTTTGTCATGGTTATGGTTCATGTTTTACTTGAGT
GTGTGAGTACTCTATCTAAATTATAATAATCTCCGATTGAGCTATGAATTATGATGTATC
GGATACATTTGATCCTAATGAAGTATGGAAT
>12704784
MSMIPSFFNNNRRSNIFDPFSLDVWDPFKELTSSSLSRENSAIVNARVDWRETPEAHVFK
ADLPGLKKEEVKVEIEEDSVLKISGERHVEKEDKNDTWHRVERSSGQFTRRFRLPENVKM
DQVKAAMENGVLTVTVPKAETKKADVKSIQISG*
>12420894
CTCTTAAAGCTTCTCGTTTTCTCTGCCGTCTCTCTCATTCGCGCGACGCAAACGATCTTC
AGCCATGGCCACCGCCGCAGATGTTGACGCTGAGATTCAGCAGGCGCTCACTAACGAAGT
CAAGCTCTTCAACCGCTGGACCTATGACGACGTTACGGTCACAGACATCAGTCTTGTTGA
CTACATTGGAGTTCAGGCAGCTAAACATGCTACCTTTGTTCCCCACACCGCTGGAAGATA
CTCTGTGAAGAGATTCAGGAAGGCTCAGTGCCCCATTGTTGAGAGGCTCACCAACTCTCT
CATGATGCACGGGAGGAACAACGGTAAGAAATTGATGGCTGTCAGGATCGTCAAGCACGC
CATGGAGATTATCCACCTCTTGTCTGACTTGAACCCAATTCAGGTCATCATTGACGCCAT
TGTCAACAGTGGTCCACGTGAAGATGCTACCAGAATTGGATCTGCTGGTGTTGTTAGGAG
ACAAGCTGTTGATATCTCTCCTCTAAGACGTGTTAACCAGGCTATCTTCTTGATTACCAC
TGGTGCTCGTGAAGCTGCTTTCAGAAACATCAAGACTATAGCTGAGTGCCTTGCTGATGA
ATTGATCAACGCAGCCAAGGGCTCTTCCAACAGCTATGCCATCAAGAAGAAGGATGAGAT
TGAAAGAGTTGCCAAGGCTAATCGTTAAGGATTTTCCCTTTGCCCTGTGCGTTACAATCT
CGTATCAATGAGTTTAATGTTTTATCTTCATTTAGATTGAAATATGTATCTCAGATGTTT
GCTCTTTTGTTTTATGAAGTTTATTTCGTCTGAACTACTTTGAATAGATAAATTTTTGAT
GCTTTAAGCTGTTTCGCATCAGTTTATTGTCAATTTTTGAACTTATCTAGGCCACCTGAA
GCTAGAAATTT
>12420895
MATAADVDAEIQQALTNEVKLFNRWTYDDVTVTDISLVDYIGVQAAKHATFVPHTAGRYS
VKRFRKAQCPIVERLTNSLMMHGRNNGKKLMAVRIVKHANEIIHLLSDLNPIQVIIDAIV
NSGPREDATRIGSAGVVRRQAVDISPLRRVNQAIFLITTGAREAAFRNIKTIAECLADEL
INAAKGSSNSYAIKKKDEIERVAKANR*
>13619634
GGGTTTTTCTCTGAGGAAGAAGCGTTTCATTTCTGTGAATTTCATCGAAAATGGCGGAAA
GAGGAGGAGAAGGCGGCGCAGAGCGTGGCGGTGACCGTGGTGACTTCGGACGTGGATTCG
GCGGTGGACGTGGAGGTGGCCGTGGCCGTGATCGTGGTCCAAGAGGCCGTGGAAGACGTG
GAGGCCGTGCCTCGGAAGAAACGAAATGGGTTCCAGTGACCAAACTAGGTCGTCTAGTGG
CTGACAATAAAATAACGAAGCTAGAGCAGATCTATCTCCATTCTCTCCCAGTAAAGGAGT
ACCAAATCATAGATCATCTTGTTGGTCCTACGTTGAAAGACGAGGTTATGAAGATCATGC
CTGTTCAGAAACAAACCAGAGCTGGTCAAAGGACTAGATTCAAGGCCTTTGTTGTTGTTG
GTGATGGTAATGGTCATGTTGGTTTGGGTGTCAAGTGTTCTAAGGAAGTTGCTACTGCCA
TTAGAGGAGCTATTATTCTTGCTAAGCTCTCTGTTGTTCCGGTGAGGAGAGGTTACTGGG
GGAATAAGATTGGGAAGCCACACACTGTGCCTTGTAAGGTTACTGGGAAGTGTGGCTCTG
TTACTGTGAGAATGGTTCCTGCTCCGAGAGGTTCTGGTATTGTTGCTGCTAGGGTTCCTA
AGAAGGTTCTTCAGTTCGCTGGTATTGATGATGTCTTTACTTCTTCCAGAGGATCTACTA
AAACTCTTGGAAACTTTGTGAAGGCGACGTTTGACTGCTTACAGAAGACATATGGGTTCC
TTACACCAGAGTTCTGGAAAGAGACTAGATTCTCCAGATCGCCCTACCAAGAGCACACTG
ATTTCTTGTCGACTAAGGCTGTTTCTGCAACCAAGGTTATCACGGAGGGTGAAGACCAAG
CTTAAGACCTTCATGAGATAAGTTTTGGTTGTTCTTAATACTAATTCCTACTTTGAAAAA
GAGTATTTTTTTTTCTTGATTATCTATGAGTTTTGTTATTGGTGGTTTCGTTACTGTTTT
GGATTTTGTTAGATGTTTGCCTTAATGCAAATTTCAATGAAATAGCTTTTGCAAT
>13619635
MAERGGEGGAERGGDRGDFGRGFGGGRGGGRGRDRGPRGRGRRGGRASEETKWVPVTKLG
RLVADNKITKLEQIYLHSLPVKEYQIIDHLVGPTLKDEVMKIMPVQKQTRAGQRTRFKAF
VVVGDGNGHVGLGVKCSKEVATAIRGAIILAKLSVVPVRRGYWGNKIGKPHTVPCKVTGK
CGSVTVRMVPAPRGSGIVAARVPKKVLQFAGIDDVFTSSRGSTKTLGNFVKATFDCLQKT
YGFLTPEFWKETRFSRSPYQEHTDFLSTKAVSATKVITEGEDQA*
>13616623
ATTTCTCATTCACTTTTCATTTCAAAAGTAAAACAAGACAAACAAAAAATACACTTAACC
ATTTATTTTTCTCTCATGAAGAATAATACTCAACCTCAATCATCTTTCAAGAAACTTTGC
CGGAAACTATCACCAAAGAGGGAAGATTCAGCCGGAGAGATACAACAACATAACAGTAGC
AATGGTGAGGACAAGAACAGAGAGTTAGAGGCTGTTTTTTCTTACATGGATGCAAACAGA
GACGGTAGAATCTCACCAGAAGAGCTTCAAAAGAGTTTCATGACATTGGGAGAACAATTG
TCTGATGAAGAAGCCGTAGCTGCTGTTAGATTGTCTGATACGGACGGAGATGGGATGTTG
GATTTTGAGGAATTTTCTCAGTTAATCALAGTAGATGACGAAGAAGAGAAGAAGATGGAG
CTCAAGGGAGCGTTTAGACTGTATATTGCAGAAGGTGAAGATTGTATTACACCAAGAAGC
TTGAAGATGATGCTAAAGAAGCTAGGAGAATCAAGAACCACTGATGATTGTAGAGTTATG
ATTAGTGCTTTTGATCTCAATGCTGATGGAGTTTTAAGCTTTGATGAGTTTGCTCTTATG
ATGCGCTAAGCCTCCATTGTTGTTGTTGTTGTTGTTGTTGTTGTTCTTTTATTTAATCTC
TTATTGT
>13616625
MKNNTQPQSSFKKLCRKLSPKREDSAGEIQQHNSSNGEDKNRELEAVFSYMDANRDGRIS
PEELQKSFMTLGEQLSDEEAVAAVRLSDTDGDGMLDFEEFSQLIKVDDEEEKKMELKGAF
RLYIAEGEDCITPRSLKMMLKKLGESRTTDDCRVMISAFDLNADGVLSFDEFALMMR*
>13601536
AAAAATGTCTCATTGCTTCTCTCGTTCTAAAAAAAATCTTGCTGCTATCTCTCTATAAGT
CCACTCCTCCTTCAAGCAAAGCACCTTCCTCTTCTTTTTGCTCCTCTGAGATTGGTTTAA
GATTAAACCAGAGCCATCTAAGGGATCTGGAACAAGCTTCGTCTCTGGTTCCACTCTGAT
CATCAGAGTATTAAAAATGGAGTCTTTCTTCTCCAGATCCACCTCCATCGTCTCCAAATT
GAGTTTCTTGGCCTTATGGATCGTCTTCTTGATTTCTTCATCTTCTTTTACTTCGACAGA
AGCATATGATGCGCTTGATCCAGAAGGCAACATTACAATGAAATGGGATGTTATCAGCTG
GACTCCTGATGGCTATGTTGCCGTGGTTACGATGTTCAACTTCCAGAAATACAGACAGAT
TCAATCTCCAGGATGGACATTAGGTTGGAAATGGGCAAAGAAGGAAGTTATATGGAGTAT
GGTTGGAGCACAAACAACTGAACAAGGTGATTGTTCAAAGTACAAAGGAAACATACCACA
TTGTTGTAAGAAGGATCCAACAGTTGTAGACTTGCTTCCAGGGACTCCTTATAATCAGCA
GATTGCTAATTGCTGCAAGGGTGGTGTTATGAACTCATGGGTTCAAGACCCTGCGACTGC
GGCTAGCTCCTTCCAGATTAGTGTTGGTGCTGCTGGAACCACAAACAAAACCGTTAGGGT
CCCAAGAAACTTCACTCTCATGGGACCTGGTCCAGGTTACACTTGTGGTCCAGCAAAGAT
TGTCAGACCAACAAAATTTGTCACGACTGACACACGCAGAACCACTCAAGCTATGATGAC
ATGGAACATTACGTGCACATACTCGCAGTTCCTTGCTCAAAGAACTCCAACTTGCTGTGT
TTCTTTATCTTCTTTCTACAATGAAACCATTGTTGGATGTCCAACTTGTGCTTGCGGATG
TCAAAAGAACAGAACAGAATCCGGTGCCTGCCTCGACCCGGACACACCACACTTAGCCTC
GGTTGTGTGACCACCAACAAAGAAAGGAACGGTTTTACCACCATTAGTGCAATGCACGAG
ACACATGTGCCCGATCAGAGTGCATTGGCATGTAAAGCAGAACTACAAAGAGTATTGGCG
TGTGAAGATCACAATCACAAACTTCAACTATCGCTTGAACTACACACAATGGAACCTTGT
TGCTCAACATCCAAATCTCGACAACATCACTCAAATCTTCAGCTTCAACTACAAATCTCT
TACTCCTTACGCTGGACTAAACGATACGGCGATGTTATGGGGAGTGAAGTTCTACAACGA
TTTCTTATGAGAAGCAGGTCCTCTTGGGAATGTTCAATCAGAGATTTTGTTCCGTAAAGA
CCAATCAACCTTCACATTCGAGAAAGGTTGGGCTTTTCCACGAAGGATTTACTTTAATGG
AGACAATTGCGTCATGCCTCCTCCAGACTCTTACCCTTTTCTTCCCAACGGTGGTTCCCG
GTCACAATTCTCATTCGTCGCCGCCGTGCTCCTCCCTCTTCTTGTCTTTTTCTTCTTCTC
TGCCTAATCTCGGATTTACGGTTTTGCCACTGGTTTGCTTAGGGTTACGGCGGAGTGGTA
TAAACGTTTATTTATGATTCTTTTGTGTCCCACAAAAATTATAATCTTTTGATACTTTTT
AAAAATATAAATAGTTTTCAACTTCCTTGTTTTTAAAAGAAATTTATATCCTTGTGTTCT
GTTGGTCCGTCGTTGTAGAATATCG
>13601537
MESFFSRSTSIVSKLSFLALWIVFLISSSSFTSTEAYDALDPEGNITMKWDVMSWTPDGY
VAVVTMFNFQKYRHIQSPGWTLGWKWAKKEVIWSMVGAQTTEQGDCSKYKGNIPHCCKKD
PTVVDLLPGTPYNQQIANCCKGGVMNSWVQDPATAASSFQISVGAAGTTNKTVRVPRNFT
LMGPGPGYTCGPAKIVRPTKFVTTDTRRTTQAMMTWNITCTYSQFLAQRTPTCCVSLSSF
YNETIVGCPTCACGCQNNRTESGACLDPDTPHLASVVSPPTKKGTVLPPLVQCTRHMCPI
RVHWHVKQNYKEYWRVKITITNFNYRLNYTQWNLVAQHPNLDNITQIFSFNYKSLTPYAG
LNDTAMLWGVKFYNDFLSEAGPLGNVQSEILFRKDQSTFTFEKGWAFPRRIYFNGDNCVM
PPPDSYPFLPNGGSRSQFSFVAAVLLPLLVFFFFSA*
>13618061
ATAAATACAAGCCTCCTAACTCATAAAATAAGCATAACCCTAACTCTACAAAGTTCTTCT
GATTCTTTCTCTCTCTCTCTTTCTTTCAAGAGCGGTTTTCAATCCATTCGCTAAAGACCA
TGAACCTAGAAGAGAAACCAACCATGACGGCTTCAAGGGCTTCCCCTCAAGCCGAACATC
TCTACTACGTCCGGTGTAGCATCTGCAACACCATCCTCGCGGTTGGGATACCATTGAAGA
GAATGCTTGACACGGTAACGGTGAAATGCGGCCATTGTGGTAACCTCTCGTTTCTCACCA
CAACTCCTCCTCTTCAAGGCCATGTTAGCCTCACCCTTCAGATGCAGAGCTTTGGTGGAA
GTGACTATAAGAAGGGAAGCTCTTCTTCTTCCTCTTCCTCCACCTCCAGCGACCAGCCCC
CATCTCCCTCACCTCCCTTTGTCGTCAAACCTCCTGAGAAGAAGCAGAGGCTCCCATCTG
CATACAACCGCTTCATGAGGGATGAGATCCAACGCATCAAAAGTGCCAATCCGGAACTAC
CACACCGTGAAGGTTTCAGTGCTGCTGCCAAAAATTGGGCTAAGTACATACCCAACTCTC
CTACTTCCATTACTTCCGGAGGCCACAACATGATCCATGGCTTGGGATTCGGTGAGAAGA
AGTGAACAAAACTCAGGGGAAAAGAAGCCTAAAAATAACAAACGCATGCACGTGTGCGAG
TGGCTGCGTCGTTTTTCTCATCTTGTGTTGTTCTTCTGTGTAATTTTCTTATGTATGTCA
TGTTGCAGAAAATGATGTTGCCTTAGTTTTTATGACTTTATATTTCTGTCTGTCTTTAGA
TTTGAAAGTAACGTCACTTGCTATGTCCCTTTGGACGTTTATGTCTGGTCTTTATTTGTC
TTAATCCTATCAAAATTTTATATGCGTATTCCTT
>13618062
MNLEEKPTMTASRASPQAEHLYYVRCSICNTILAVGIPLKRMLDTVTVKCGHCGNLSFLT
TTPPLQGHVSLTLQMQSFGGSDYKKGSSSSSSSSTSSDQPPSPSPPFVVKPPEKKQRLPS
AYNRFMRDEIQRIKSANPEIPHREAFSAAAKNWAKYIPNSPTSITSGGHNMIHGLGFGEK
K*
>12705120
AAAACTCTTAAACAGCTTCCTAACGAGAGGAAACTGAGGAACACAACAATGGAGTTTCGT
GGAGATGCCAACCAGAGGATTGCTAGGATTTCAGCTCATGTCACTGCTCAGATGGAGGCC
AAGAACTCTGTAATCGGACGGGAAAACTGCAGAGCTAAAGGTGGTAATCCAGGATTCAAA
GTAGCAATTCTTGGAGCTGCAGGTGGAATTGGACAATCTTTATCTTTGCTGATGAAGATG
AACGCTCTTGTCTCTTTACTTCATCTCTACGATGTTGTCAATGCTCCTGGCGTCACTGCT
GACGTCAGTCATATGGACACTGGAGCTGTTGTCCGCGGGTTCTTGGGAGCGAAGCAGCTT
GAGGACGCGCTAACGGGTATGGATCTTGTGATCATACCAGCCGGTATACCGAGGAAACCA
GGGATGACCCGCGATGATCTGTTTAAAATCAATGCTGGGATTGTTAAAACACTATGTGAA
GGTGTAGCAAAATGTTGTCCTAATGCTATTGTCAACTTGATCAGCAACCCTGTGAACTCT
ACTGTCCCCATTGCCGCTGAGGTTTTCAAGAAAGCTGGAACTTATGATCCTAAGAAGCTC
CTTGGAGTTACTACACTCGATGTTGCTCGTGCCAACACATTTGTGGCAGAAGTTCTTGGC
CTTGATCCAAGAGAAGTCGATGTGCCAGTAGTTGGGGGACACGCCGGAGTCACAATCTTG
CCACTACTGTCACAGGTTAAACCTCCTAGCAGCTTCACACCTCAAGAAATTGAGTACCTG
ACAAACCGGATTCAAAATGGTGGAACTGAAGTTGTGGAGGCAAAAGCTGGAGCTGGTTCT
GCAACACTTTCAATGGCATATGCTGCAGCCAAGTTTGCAGATGCTTGCCTTCGCGGGTTA
AGAGGAGATGCGAATGTCGTAGAATGCTCTTTTGTTGCTTCACAGGTGACAGAATTAGCT
TTCTTTGCAACAAAAGTGCGCCTTGGCCGTACAGGAGCAGAGGAAGTGTATCAGCTTGGA
CCCTTAAACGAATACGAAAGGATTGGTCTGGAGAAAGCAAAAGATGAATTAGCCGGAAGT
ATTCAGAAAGGTGTTGAATTCATCAGAAAATGAAACTGAGAGATAATCAGAGAGATACAA
TAAGTTATTTCCTCAACTATATGATCATGTACTCATCATCACATCATGCCTATGTCTCCT
CTGCTTCTGATACAACTTTGTATAAATCCTTATCAGTTTGTGTACGATATATGTGACCTT
TTCAACGT
>12705122
MEFRGDANQRIARISAHLTPQMEAKNSVIGRENCRAKGGNPGFKVAILGAAGGIGQSLSL
LMKMNPLVSLLHLYDVVNAPGVTADVSHMDTGAVVRGFLGAKQLEDALTGMDLVIIPAGI
PRKPGMTRDDLFKINAGIVKTLCEGVAKCCPNAIVNLISNPVNSTVPIAAEVFKKAGTYD
PKKLLGVTTLDVARANTFVAEVLGLDPREVDVPVVGGHAGVTILPLLSQVKPPSSFTPQE
IEYLTNRIQNGGTEVVEAKAGAGSATLSMAYAAAKFADACLRGLRGDANVVEGSFVASQV
TELAFFATKVRLGRTGAEEVYQLGPLNEYERIGLEKAKDELAGSIQKGVEFIRK*
>13607229
ATTTTAAAAGTATCAGTTTACACTGACACAATCCTTAACTATTTTCCTTTGTTCTTCTTC
ATCTTTATTACACATTTTTTTCAAGGTCTACCAAACGATGTCGGTTTTCGAATCGGAGAC
TTCGAACTTCCACGTCTACAACAACCACGAAATCCAAACGCAACCGCAAATGCAAACGTT
TCTGTCGGAGGAGGAACCGGTAGGGAGACAGAACTCGATTTTGTCACTAACTCTTGACGA
AATTCAGATGAAAAGCGGTAAGAGCTTTGGAGCGATGAACATGGACGAGTTCCTAGCGAA
CTTGTGGACAACCGTTGAAGAAAACGACAACGAAGGAGGTGGGGCTCACAACGACGGAGA
GAAGCCGGCGGTGCTGCCACGTCAAGGGTCGTTGTCCCTCCCTGTGCCTTTATGCAAGAA
AACGGTCGACGAGGTTTGGCTCGAGATACAAAACGGTGTACAACAACATCCACCGTCGTC
GAATTCCGGTCAAAACTCCGCCGAAAATATTCGCCGGCAACAAACCCTTGGTGAGATCAC
TCTCGAGGATTTTCTTGTTAAGGCTGGTGTTGTACAAGAACCGTTGAAGACAACGATGAG
GATGTCGAGTTCTGATTTTGGTTATAACCCCGAGTTTGGAGTTGGTTTACATTGTCAGAA
CCAAAACAATTATGGTGATAACCGGTCGGTTTATAGTGAAAACCGACCGTTTTACTCGGT
TTTGGGAGAATCTTCAAGCTGTATGACCGGGAATGGGAGGAGTAATCAGTATCTGACCGG
TTTAGATGCTTTTCGGATCAAGAAACGGATAATTGATGGTCCACCTGAAATTTTGATGGA
GCGGAGACAACGGCGAATGATTAAAAACCGCGAATCTGCGGCTCGGTCTCGAGCCCGGAG
ACAAGCTTATACTGTGGAACTGGAGTTGGAATTGAACAACCTCACGGAAGAAAACACGAA
GCTGAAGGAAATTGTGGAGGAAAATGAGAAGAAAAGAAGACAAGAGATAATAAGTAGAAG
CAAACAAGTGACTAAAGAGAAGAGCGGAGACAAATTGAGAAAGATTCGGAGGATGGCCAG
TGCCGGGTGGTAAATAGATATGAGAGTTTGTGTAATTTACATATGATATTTTATTGATCG
TTTCTATCTATACACATAAATATTTGTATCTATATTATATTTTGAGACAAACATGGAATT
TGTGTATATAGGATCATTTCTTTTT
>13607230
MSVFESETSNFHVYNNHEIQTQPQMQTFLSEEEPVGRQNSILSLTLDEIQMKSGKSFGAM
NMDEFLANLWTTVEENDNEGGGAHNDGEKPAVLPRQGSLSLPVPLCKKTVDEVWLEIQNG
VQQHPPSSNSGQNSAENIRRQQTLGEITLEDFLVKAGVVQEPLKTTMRMSSSDFGYNPEF
GVGLHGQNQNNYGDNRSVYSENRPFYSVLGESSSCMTGNGRSNQYLTGLDAFRIKKRIID
GPPEILMERRQRRMIKNRESAARSRARRQAYTVELELELNNLTEENTKLKEIVEENEKKR
RQEIISRSKQVTKEKSGDKLRKIRRIVIASAGW*
>12323871
AAAATAAGCTCTCTTTCTACTATTTCTCTTTCTCTTTCTACTATTTCTCTCCTGTGGAGA
AACTCAGGAGATAGAGAGAGAGAGAGAGAAGAGAAGAGAGCATGTATGTTTGGTTTTATA
ATCTCTCTACTCATACCAAAGATTTGTCTCAGACCCACCACTTGGACAGAGAGAACCCAA
GCTCCTTTCTCTCTTTTTCTGGATCTACTCCTTCTTAATCTCCTTTTTTGAAACTTGAAG
CCACTTTCAACATCATCCTTAAACTTTTGTTCCCTTATTCACAATCTCCTGCCACCTCTC
ATTTCTCTAGCTACATATATGGGTTCTATTAGAGGAAACATTGAAGAGCCTATATCTCAG
TCATTAACGAGGCAGAACTCTCTCTATAGCTTAAAGCTCCATGAGGTTCAAACCCACTTA
GGAAGTTCTGGAAAACCACTAGGAAGCATGAACCTTGATGAGCTTCTCAAGACTGTCTTG
CCACCAGCTGAGGAAGGGCTTGTTCGTCAGGGAAGCTTGACGTTACCTCGAGATCTCAGT
AAAAAGACAGTTGATGAGGTCTGGAGAGATATCCAACAGGACAAGAATGGAAACGGTACT
AGTACTACTACTACTCATAAGCAGCCTACACTCGGTGAAATAACACTTGAGGATTTGTTG
TTGAGAGGTGGTGTAGTGACTGAGACAGTAGTCCCTCAAGAAAATGTTGTTAACATAGCT
TCAAATGGGCAATGGGTTGAGTATCATCATCAGCCTCAACAACAACAAGGGTTTATGACA
TATCCGGTTTGCGAGATGCAAGATATGGTGATGATGGGTGGATTATCGGATACACCACAA
GCGCCTGGGAGGAAAAGAGTAGCTGGAGAGATTGTGGAGAAGACTGTTGAGAGGAGACAG
AAGAGGATGATCAAGAACAGAGAATCTGCAGCACGTTCACGAGCTAGGAAACAGGCTTAT
ACACATGAATTAGAGATCAAGGTTTCAAGGTTAGAAGAAGAAAACGAAAAACTTCGGAGG
CTAAAGGAGGTGGAGAAGATCCTACCAAGTGAACCACCACCAGATCCTAAGTGGAAGCTC
CGGCGAACAAACTCTGCTTCTCTCTGATCCTAAAGACTCTTCTTTCTTTCTTCTTCTTTG
TGTTGGTTTATATCAGACCGCTTTGTTCTTTGTATATTGTGTAGACTTTATTGACTTTGA
ACAGCATGTCTTTATAAACATTTCTTGAGTGTATTCCTCTTTTGCAGCTAGTTCAAACTT
TTTGAGT
>12323872
MGSIRGNIEEPISQSLTRQNSLYSLKLHEVQTHLGSSGKPLGSMNLDELLKTVLPPAEEG
LVRQGSLTLPRDLSKKTVDEVWRDIQQDKNGNGTSTTTTHKQPTLGEITLEDLLLRAGVV
TETVVPQENVVNIASNGQWVEYHHQPQQQQGFMTYPVCEMQDMVMMGGLSDTPQAPGRKR
VAGEIVEKTVERRQKRMIKNRESAARSRARKQAYTHELEIKVSRLEEENEKLRRLKEVEK
ILPSEPPPDPKWKLRRTNSASL*
>13602983
CTGTGTCTTTATCTTCTCTCTCCTCTTCTTGAAAAACTGAACCTTTAATTCTTTCTTCAC
ATCTCCTTTAGCTTTCTGAAGCTGCTATCTTCAAGCTCAAAACCAAACCCTAGAAATAAA
ATGACGGCGACACAAATGCTGCTTCATCTTCTTCTTCTTCCACTTCTCTTCCTTCACTTC
AGCAACCAAGTCATGGCAGAAATCACCGACGAGTTAGCGACATTGATGGAGGTTAAAACA
GAGCTTGACCCAGAAGACAAACACTTAGCTTCATGGAGTGTTAATGGAGATCTGTGTAAA
GACTTTGAAGGTGTTGGTTGTGACTGGAAAGGACGAGTTTCTAACATATCTCTACAAGGG
AAAGGCTTATCTGGTAAAATCTCTCCGAACATTGGAAAGCTTAAACATTTGACGGGTTTG
TTCTTGCACTACAATGCTCTTGTTGGAGATATTCCTAGAGAACTTGGTAACTTGTCGGAA
CTTACGGATCTTTATCTCAATGTTAATAATCTCTCCGGTGAGATTCCTTCAAACATTGGG
AAGATGCAAGGCTTGCAAGTTTTGCAGCTCTGTTACAACAATTTGACTGGAAGCATTCCT
AGGGAGCTTAGTTCACTGAGGAAGCTGAGTGTTCTTGCTCTTCAATCTAACAAACTCACT
GGAGCTATACCTGCGAGTTTAGGAGATTTAAGTGCTCTAGAGCGGCTAGATTTGAGCTAC
AATCATTTGTTTGGTTCTGTACCAGGCAAGTTAGCTAGCCCTCCTCTGCTTCGAGTTCTC
GACATCCGCAACAACTCCCTCACTGGCAATGTACCTCCTGTACTGAAGAGATTAAATGAA
GGTTTTTCGTTTGAAAACAACTTGGGACTATGTGGAGCTGAGTTGTCGCCGTTGAAATCT
TGCAATGGCACAGCTCCTGAGGAACCTAAGCCATACGGTGCAACCGTGTTTGGTTTTCCA
TCCCGGGATATACCAGAATCAGCTAATCTACGGTCACCTTGTAATGGAACAAACTGTAAT
ACTCCTCCAAAATCTCACCAAGGTGGAATCCTTATTGGTTTGGTTGTCTCGACTATTGCA
TTGTCTGCTATTAGCATCCTCTTGTTCACGCATTATCGCAGACGCAAACAGAAGCTTTCA
ACCACTTATGAAATGTGGGACAACCGTCTCAACACCGTAGGAGGAGGTTTTAGGAAGAAC
AATGGTTCTCCATTGGCCAGTCTTGAATATACTAATGGTTGGGATCCACTTTCAGAGAAT
AGGAATCTCAGTGTCTTTGCTGAAGAAGTTATCCAGAGCTTTCGATTCAATCTTGAAGAG
GTTGAAACTGCTACACAGTATTTCTCAGAAGTGAATTTGCTTGGAAGAAGCAACTTCTCT
GCGACTTACAAAGGAATCTTGAGAGATGGTTCTGCTGTAGCGATCAAACGGTTTAGTAAA
ACAAGTTGCAAATCTGAAGAACCTGAGTTCTTGAAAGGACTCAACATGTTGGCGTCTCTG
AAACATGAAAACTTGTCAAAAGTTAGAGGCTTCTGCTGTTCAAGAGGCCGTGGAGAATGT
TTTCTCATCTATGATTTTGCTCCTAATGGAAATTTACTGAGCTATCTTGATCTCAAAGAT
GGTGATGCGCATGTTCTTGATTGGTCCACGAGAGTATCCATTGCTAAAGGCATCGCAAAA
GGGATTGGTTATCTACATTCATACAAAGGAAGGAAGCCAGCATTGGTTCACCAAAACATC
TGAGCGGAGAAAGTTCTAATCGAGCAGCGATAGAGTCCTCTACTCTCAAACTCAGGCCTC
CACACACTTCTCACAAACGAGATTGTCTTCTCTGCACTGAAAGACAGTGCAGCAATGGGC
TACCTCGCTCCAGAATACACCACAACCGGACGTTTCACCGAGAAAACCGATGTCTACGCT
TTCGGGATTCTTGTGTTTCAGATAATCTCCGGGAAACAGAAGGTTAGGCATCTTGTTAAG
CTTGGAACTGAAGCTTGTAGGTTCAATGATTACATAGATCCAAATCTTCAAGGAAGATTC
TTTGAATATGAAGCCACAAAGCTAGCTAGAATCGCGTGGCTATGCACTCATGAATCTCCC
ATTGAAAGACCATCCGTGGAAGCTGTAGTTCATGAGCTAGGTAACTGTAGTAGCTGTCTC
TGAGAACTCTCTCGAGGCAAATGTGTAATGTGTACCTTAGGAATCAATTAAGGAAGGGTT
TGTTAGTCACCTTTTCCTGTGTTTAGGGTTAAGAAAAAGGTGCCTTTTCCCTTCCCATTA
ACGTTTAAAAACTGTTCAACGTGTTGTACTTTATTGGTTTGTATGATTTGTGTTTATGTT
AAGCTCTTCAACC
>13602984
MTATQMLLHLLLLPLLFLHFSNQVMAEITDELATLMEVKTELDPEDKHLASWSVNGDLCK
DFEGVGCDWKGRVSNISLQGKGLSGKISPNIGKLKHLTGLFLHYNALVGDIPRELGNLSE
LTDLYLNVNNLSGEIPSNIGKMQGLQVLQLCYNNLTGSIPRELSSLRKLSVLALQSNKLT
GAIPASLGDLSALERLDLSYNHLFGSVPGKLASPPLLRVLDIRNNSLTGNVPPVLKRLNE
GFSFENNLGLCGAEFSPLKSCNGTAPEEPKPYGATVFGFPSRDIPESANLRSPCNGTNCN
TPPKSHQGAILIGLVVSTIALSAISILLFTHYRRRKQKLSTTYEMSDNRLNTVGGGFRKN
NGSPLASLEYTNGWDPLSDNRNLSVFAQEVTQSFRFNLEEVETATQYFSEVNLLGRSNFS
ATYKGILRDGSAVAIKRFSKTSCKSEEPEFLKGLNMLASLKHENLSKLRGFCCSRGRGEC
FLIYDFAPNGNLLSYLDLKDGDAHVLDWSTRVSIAKGTAKGIAYLHSYKGSKPALVHQNI
SAEKVLIDQRYSPLLSNSGLHTLLTNDIVFSALKDSAANGYLAPEYTTTGRFTEKTDVYA
FGILVFQTISGKQKVRHLVKLGTEACRFNDYIDPNLQGRFFEYEATKLARIAWLCTHESP
IERPSVEAVVHELGNCSSCL*
>13607852
GTTAGATGATCTGTCCATTTTTTTCTTTTTTCTTCTGTGTATAAATATATTTGAGCACAA
AGAAAAACTAATAACCTTCTGTTTTCAGCAAGTAGGGTCTTATAACCTTCAAAGAAATAT
TCCTTCAATTGAAAACCCATAAACCAAAATAGATATTACAAAAGGAAAGAGAGATATTTT
CAAGAACAACATAATTAGAAAAGCAGAAGCAGCAGTTAAGTGGTACTGAGATAAATGATA
TAGTTTCTCTTCAAGAACAGTTTCTCATTACCCACCTTCTCCTTTTTGCTGATCTATCGT
AATCTTGAGAACTCAGGATCAATGGAGGAAGGTGGGAGTAGTCACGACGCAGAGAGTAGC
AAGAAACTAGGGAGAGGGAAAATAGAGATAAAGAGGATAGAGAACACAACAAATCGTCAA
GTTACTTTCTGCAAACGACGCAATGGTCTTCTCAAGAAAGCTTATGAACTCTCTGTCTTG
TGTGATGCCGAAGTTGCCCTCGTCATCTTCTCCACTCGTGGCCGTCTCTATGAGTACGCC
AACAACAGTGTGAGGGGTACAATTGAAAGGTACAAGAAAGCTTGTTCCGATGCCGTCAAC
CCTCCTTCCGTCACCGAAGCTAATACTCAGTACTATCAGCAAGAAGCCTCTAAGCTTCGG
AGGCAGATTCGAGATATTCAGAATTCAAATAGGCATATTGTTGGGGAATCACTTGGTTCC
TTGAACTTCAAGGAACTCAAAAACCTAGAAGGACGTCTTGAAAAAGGAATCAGCCGTGTC
CGCTCCAAAAAGAATGAGCTGTTAGTGGCAGAGATAGAGTATATGCAGAAGAGGGAAATG
GAGTTGCAACACAATAACATGTACCTGCGAGCAAAGATAGCCGAAGGCGCCAGATTGAAT
CCGGACCAGCAGGAATCGAGTGTGATACAAGGGACGACAGTTTACGAATCCGGTGTATCT
TCTCATGACCAGTCGCAGCATTATAATCGGAACTATATTCCGGTGAACCTTCTTGAACCG
AATCAGCAATTCTCCGGCCAAGACCAACCTCCTCTTCAACTTGTGTAACTCAAAACATGA
TAACTTGTTTCTTCTCCTCATAACGATTAAGAGAGAGACGAGAGAGTTCATTTTATATTT
ATAACGCGACTGTGTATTCATAGTTTAGGTTCTAATAATGATAATAACAAAACTGTTGTT
TCTTTGCTT
>13607853
MEEGGSSHDAESSKKLGRGKIEIKRIENTTNRQVTFCKRRNGLLKKAYELSVLCDAEVAL
VIFSTRGRLYEYANNSVRGTIERYKKACSDAVNPPSVTEANTQYYQQEASKLRRQTRDIQ
NSNRHIVGESLGSLNFKELKNLEGRLEKGISRVRSKKNELLVAEIEYMQKREMELQHNNM
YLRAKIAEGARLNPDQQESSVIQGTTVYESGVSSHDQSQHYNRNYIPVNLLEPNQQFSGQ
DQPPLQLV*
>12677570
gtgacttaaaaagctcctaggcaccgaaatcgagacactctcgacaaccatgctctctga
cgccgggggtggttccgattgccggcgtcgggacttatcaactcccatcaaccttcatgt
gttctatataagctttatcttcattgaatcttcctccgttatctcaaatctctcaaaata
tctaaatctccttttttatgtgagcttcttcactgaaagttttctttgtgacggaagagt
atatcgatgctctattggctccgatctgactcagatcttggatgcatctctgtcttcgaa
cccaaaacaagaaaattcacaacagtccaacagctcctcttctcaaacatcagagcaaga
cttcatcaacttatcaaaaagctctagatctggactagcaccaacaccacctttggtttc
ttctcaccggttttcgttgatggcaggagtatctcttggaccatcagatgtgcttcttcc
gctgggaacgtcgacggcacacgacgagctcaaacgctggcaatggtcaccctatatgat
tcacagtcgcccatcattccaattcttcagaatgacggaggcgctttccttatcccgaca
acatcaaccctagggtttgattggatgagcctcatctttcattaaagcccaatatctccc
atgtggcccatatccaggtctgatttccctatcaatttgcatagtctctctttgctttgt
ttctcgacaaaggatggtcgtcgactccattcagctttaggaacaatgcatctagagggg
atttgcttattcaattatcgaaaccttatgattaaccaattggttcttaaccttgcatct
tcacaattgtttaaattaaaattttgaaacatgacatgtaagaagcaaatcttagttttt
aattcataaaaacttcttagatatgaagcaaatcttagttttt
>12677571
MLSDAGGGSDCRRRDLSTPINLHVFYISFIFIESSSVISNLSKYLNLLFYVSFFTESFLC
DGRVYRCSTGSDLTQILDASLSSNPKQENSQQSNSSSSQTSEQDFINLSKSSRSGLAPTP
PLVSSHRFSLMAGVSLGPSDVLLPLGTSTAHDELKRWQWSPYMTHSRPSFQFFRMTEALS
LSRQHQP*
>13504706
ACAACTCGATTACCAAAAAGCAGAGCCATCCAACCATAAAACTCAAAACACACAGATTCC
ACTGGCGTGTGCTCTCCTCACTTCACTCGTCCTTGAAACTTGAGCTTCAATTACAAAGAA
TGGCTGCTTCAAGTGCTGTCACCGCAAACTACGTCCTCAAGCCACCTCCATTCGCACTGG
ATGCTTTGGAGCCGCATATGAGCAAACAAACTCTGGAGTTTCACTGGGGAAAACATCACA
GAGCTTACGTGGACAACCTCAAGAAACAGGTTCTTGGAACCGAGCTTGAAGGCAAGCCCT
TAGAGCACATTATCCACAGCACTTACAACAATGGTGATCTCCTCCCTGCTTTCAACAACG
CTGCTCAGGCGTGGAACCACGAGTTCTTCTGGGAGTCAATGAAACCAGGTGGTGGAGGAA
AACCATCAGGAGAGCTTCTTGCTTTGCTTGAAAGAGATTTCACTTCTTATGAGAAGTTCT
ATGAAGAGTTCAATGCTGCTGCAGCCACTCAGTTTGGAGCTGGCTGGGCCTGGCTTGCTT
ATTCAAATGAAAAACTCAAAGTAGTGAAAACTCCCAATGCTGTGAATCCCCTTGTGCTCG
GCTCTTTCCCATTGCTTACCATTGATGTCTGGGAGCATGCTTACTACCTTGACTTCCAGA
ACCGAAGACCAGATTACATAAAGACATTCATGACCAATCTTGTGTCTTGGGAAGCTGTAA
GTGCCAGACTTGAGGCCGCCAAGGCTGCTTCTGCTTAAGCAAATTTCTGAACAATTTGAC
TTCAGTGACAGTGAGTTCTGCATCACCGAAGTCTCTTATAAAATATTGGTCGCTGTAATA
AGGACACAGCTCTCTTGTTGTGTATGTGTCACAGAGTTCTTCATTTTGCTTGTGTAATGA
ACAATTAAACATGCTCTTTTCTGAGTGTGTGTGCGTTTTGTGTGTGTCAAGTTTTTCATC
GTCTCCTTTATTAAACTCAAATTGGCACCTACCATCAGTAATTCATAGTTTGGCATGGCC
CATT
>13504708
MAASSAVTANYVLKPPPFALDALEPHMSKQTLEFHWGKHHRAYVDNLKKQVLGTELEGKP
LEHIIHSTYNNGDLLPAFNNAAQAWNHEFFWESMKPGGGGKPSGELLALLERDFTSYEKF
YEEFNAAAATQFGAGWAWLAYSNEKLKVVKTPNAVNPLVLGSFPLLTIDVWEHAYYLDFQ
NRRPDYIKTFMTNLVSWEAVSARLEAAKAASA*
>12728113
ATGAGTGACTCGGTGTCGATCTCGGTTCCGTATAGGAATTTGAGGAAGGAAATTGAACTT
GAGACGGTCACCAAGCATCGTCAAAACGAATCTGGTTCTTCGTCGTTCTCTGAATCTGCT
TCTCCTTCGAATCATTCTGATTCGGCTGATGGTGAATCTGTGTCGAAGAATTGTAGTTTA
GTGACGTTGGTTCTTAGTTGTACAGTTGCCGCTGGAGTTCAATTTGGTTGGGCATTGCAA
CTTTCTCTTCTTACTCCTTATATTCAGACCCTTGGAATATCGCATGCTTTTTCTTCGTTT
ATTTGGCTGTGCGGCCCAATTACAGGCCTTGTGGTCCAGCCTTTTGTTGGCATTTGGAGT
GATAAATGTACTTCAAAGTATGGAAGAAGACGACCATTTATTCTAGTTGGATCATTCATG
ATCTCAATAGCAGTGATAATAATCGGATTTTCTGCAGACATTGGGTATCTGTTAGGAGAT
TCAAAGGAACATTGCAGTAGTTTCAAAGGCACACGAACCAGGGCAGCTGTTGTGTTTATC
ATTGGGTTTTGGTTGTTGGATCTAGCAAACAATACAGTACAGGGACCTGCTCGTGCTCTT
CTAGCTGATCTATCAGGTCCTGATCAGCGGAATACTGCAAATGCTGTGTTCTGCTTGTGG
ATGGCTATTGGGAACATCCTTGGGTTTTCTGCCGGTGCTAGCGGAAAATGGCAAGAATGG
TTCCCTTTTTTAACTAGTAGAGCATGTTGTGCTGCATGTGGAAATCTCAAAGCAGCGTTT
CTTCTTGCAGTGGTCTTTCTCACTATAGTACTCTTGTACACAATGTATTTTGCTAAAGAG
ATTCCTTTTACAAGCAACAAGCCCACCCGCATACAAGATTCTGCACCTTTGTTGGATGAT
CTCCAGTCCAAAGGCCTTGAGCATTCAAAATTAAATAATGGTACTGCCAATGGAATCAAG
TATGAGAGAGTGGAACGTGATACGGATGAACAGTTTGGCAATTCAGAGAATGAGCATCAA
GATGAGACCTACGTTGATGGCCCTGGATCTGTTTTAGTGAATTTGCTAACTAGTTTAAGG
CATTTGCGACCGGCTATGCACTCAGTTCTTATCGTCATGGCTCTTACATGGTTATCCTGG
TTCCCCTTCTTTCTGTTCGATACAGATTGGATGGGAAGAGAAGTTTACCATGGGGATCCA
ACAGGAGATAGTTTGCATATGGAACTCTATGATCAAGGTGTACGTGAAGGTGCACTTGGT
TTGCTACTAAACTCTGTTGTTCTTGGGATCAGCTCATTTCTCATTGAACCAATGTGTCAG
CGGATGGGTGCTCGGGTTGTATGGGCTTTGAGCAATTTTACTGTATTTGCCTGCATGGCG
GGAACAGCTGTAATCAGCTTGATGTCTCTCAGTGATGACAAAAATGGAATTGAATACATA
ATGCGTGGAAACGAAACAACAAGAACCGCAGCCGTAATCGTTTTTGCACTCCTTGGTTTT
CCCCTAGCTATCACATACAGTGTCCCTTTCTCTGTCACAGCAGAAGTCACTGCTGATTCC
GGTGGCGGTCAAGGTTTGGCTATAGGAGTGTTGAATCTCGCAATCGTTATTCCCCAGATG
ATAGTATCAGTTGGAGCGGGTCCATGGGATCAATTGTTTGGAGGAGGAAACTTACCGGCG
TTTGTTTTGGCGTCTGTTGCTGCTTTCGCTGCTGGAGTTATTGCATTGCAAAGGCTTCCC
ACGCTATCGAGTTCTTTCAAGTCCACCGGTTTCCACATCGGCTAA
>12728114
MSDSVSISVPYRNLRKEIELETVTKHRQNESGSSSFSESASPSNHSDSADGESVSKNCSL
VTLVLSCTVAAGVQFGWALQLSLLTPYIQTLGISHAFSSFIWLCGPITGLVVQPFVGIWS
DKCTSKYGRRRPFILVGSFMISIAVTIIGFSADIGYLLGDSKEHCSTFKGTRTRAAVVFI
IGFWLLDLANNTVQGPARALLADLSGPDQRNTANAVFCLWMAIGNILGFSAGASGKWQEW
FPFLTSRACCAACGNLKAAFLLAVVFLTICTLVTIYFAKEIPFTSNKPTRIQDSAPLLDD
LQSKGLEHSKLNNGTANGIKYERVERDTDEQFGNSENEHQDETYVDGPGSVLVNLLTSLR
HLPPAMHSVLIVMALTWLSWFPFFLFDTDWMGREVYHGDPTGDSLHMELYDQGVREGALG
LLLNSVVLGISSFLIEPMCQRMGARVVWALSNFTVFACMAGTAVISLMSLSDDKNGIEYI
MRGNETTRTAAVIVFALLGFPLAITYSVPFSVTAEVTADSGGGQGLAIGVLNLAIVIPQM
IVSLGAGPWDQLFGGGNLPAFVLASVAAFAAGVIALQRLPTLSSSFKSTGFHIG*
>12705056
AAATACACCTAACTTGTTTAGTACACAACAGCAACATCAAACTCTAATAAACCCAAGTTG
GTGTATACTATAATGGTGATGGCTGGTGCTTCTTCTTTGGATGAGATCAGACAGGCTCAG
AGAGCTGATGGACCTGCAGGCATCTTGGCTATTGGCACTGCTAACCCTGAGAACCATGTG
CTTCAGGCGGAGTATCCTGACTACTACTTCCGCATCACCAACAGTGAACACATGACCGAC
CTCAAGGAGAAGTTCAAGCGCATGTGCGACAAGTCGACAATTCGGAAACGTCACATGCAT
CTGACGGAGGAATTCCTCAAGGAAAACCCACACATGTGTGCTTACATGGCTCCTTCTCTG
GACACCAGACAGGACATCGTGGTGGTCGAAGTCCCTAAGCTAGGCAAAGAAGCGGCAGTG
AAGGCCATCAAGGAGTGGGGCCAGCCCAAGTCAAAGATCACTCATGTCGTCTTCTGCACT
ACCTCCGGCGTCGACATGCCTGGTGGTGACTACCAGCTCACCAAGCTTCTTGGTCTGCGT
CCTTCCGTCAAGCGTCTCATGATGTACCAGCAAGGTTGCTTCGCCGGCGGTACTGTCCTC
CGTATCGCTAAGGATCTCGCCGAGAACAATCGTGGAGCACGTGTCCTCGTTGTCTGCTCT
GAGATCACAGCCGTTACCTTCCGTGGTCCCTCTGACACCCACCTTGACTCCCTCGTCGGT
CAGGCTCTTTTCAGTGATGGCGCCGCGGCACTCATTGTGGGGTCGGACCCTGACACATCT
GTCGGAGAGAAACCCATCTTTGAGATGGTGTCTGCCGCTCAGACCATCCTTCCAGACTCT
GATGGTGCCATAGACGGACATTTGAGGGAAGTTGGTGTGACCTTCCATCTCCTCAAGGAT
GTTCCCGGCCTCATCTCCAAGAACATTGTGAAGAGTCTAGACGAAGCGTTTAAACCTTTG
GGGATAAGTGACTGGAACTCCCTCTTCTGGATAGCCGACCCTGGAGGTCCAGCGATCCTA
GACCAGGTGGAGATAAAGCTAGGACTAAAGGAAGAGAAGATGAGGGCGACACGTCACGTG
TTGAGCGAGTATGGAAACATGTCGAGCGCGTGCGTTCTCTTCATACTAGACGAGATGAGG
AGGAAGTCAGCTAAGGATGGTGTGGCCACGACAGGAGAAGGGTTGGAGTGGGGTGTCTTG
TTTGGTTTCGGACCAGGTCTCACTGTTGAGACAGTCGTCTTGCACAGCGTTCCTCTCTAA
ACAGAACGCTTGCCTTCTATCTGCCTACCTACCTACGCAAAACTTTAATCCTGTCTTATG
TTTTATATAATATAATGATTATATGTTTACGCAATAATTAAGGAAGAATTCATTTGATGT
GATATGTGATATGTGCTGGACAGGTCTATTCGACTGTTTTTGTACTCTCTTTTTTGTGTC
TTTTTACAATATTAAATCTATGGGTCTTGAAT
>12705057
MVMAGASSLDETRQAQRADGPAGILAIGTANPENHVLQAEYPDYYFRITNSEHMTDLKEK
FKRMCDKSTIRKRHMHLTEEFLKENPHMCAYMAPSLDTRQDIVVVEVPKLGKEAAVKAIK
EWGQPKSKITHVVFCTTSGVDMPGADYQLTKLLGLRPSVKRLMMYQQGCFAGGTVLRIAK
DLAENNRGARVLVVCSEITAVTFRGPSDTHLDSLVGQALFSDGAAALIVGSDPDTSVGEK
PIFEMVSAAQTILPDSDGAIDGHLREVGLTFHLLKDVPGLISKNIVKSLDEAFKPLGISD
WNSLFWIAHPGGPAILDQVEIKLGLKEEKMRATRHVLSEYGNMSSACVLFILDEMRRKSA
KDGVATTGEGLEWGVLFGFGPGLTVETVVLHSVPL*
>12371852
AAATACACCTAACTTGTTTAGTACACAACAGCAACATCAAACTCTAATAAACCCAAGTTG
GTGTATACTATAATGGTGATGGCTGGTGCTTCTTCTTTGGATGAGATCAGACAGGCTCAG
AGAGCTGATGGACCTGCAGGCATCTTGGCTATTGGCACTGCTAACCCTGAGAACCATGTG
CTTCAGGCGGAGTATCCTGACTACTACTTCCGCATCACCAACAGTGAACACATGACCGAC
CTCAAGGAGAAGTTCAAGCGCATGTGCGACAAGTCGACAATTCGGAAACGTCACATGCAT
CTGACGGAGGAATTCCTCAAGGAAAACCCACACATGTGTGCTTACATGGCTCCTTCTCTG
GACACCAGACAGGACATCGTGGTGGTCGAAGTCCCTAAGCTAGGCAAAGAAGCGGCAGTG
AAGGCCATCAAGGAGTGGGGCCAGCCCAAGTCAAAGATCACTCATGTCGTCTTCTGCACT
ACCTCCGGCGTCGACATGCCTGGTGCTGACTACCAGCTCACCAAGCTTCTTGGTCTCCGT
CCTTCCGTCAAGCGTCTCATGATGTACCAGCAAGGTTGCTTCGCCGGCGGTACTGTCCTC
CGTATCGCTAAGGATCTCGCCGAGAACAATCGTGGAGCACGTGTCCTCGTTGTCTGCTCT
GAGATCACAGCCGTTACCTTCCGTGGTCCCTCTGACACCCACCTTGACTCCCTCGTCGGT
CAGGCTCTTTTCAGTGATGGCGCCGCCGCACTCATTGTGGGGTCGGACCCTGACACATCT
GTCGGAGAGAAACCCATCTTTGAGATGGTGTCTGCCGCTCAGACCATCCTTCCAGACTCT
GATGGTGCCATAGACGGACATTTGAGGGAAGTTGGTCTCACCTTCCATCTCCTCAAGGAT
GTTCCCGGCCTCATCTCCAAGAACATTGTGAAGAGTCTAGACGAAGCGTTTAAACCTTTG
GGGATAAGTGACTGGAACTCCCTCTTCTGGATAGCCCACCCTGGAGGTCCAGCGATCCTA
GACCAGGTGGAGATAAAGCTAGGACTAAAGGAAGAGAAGATGAGGGCGACACGTCACGTG
TTGAGCGAGTATGGAAACATGTCGAGCGCGTGCGTTCTCTTCATACTAGACGAGATGAGG
AGGAAGTCAGCTAAGGATGGTGTGGCCACGACAGGAGAAGGGTTGGAGTGGGGTGTCTTG
TTTGGTTTCGGACCAGGTCTCACTGTTGAGACAGTCGTCTTGCACAGCGTTCCTCTCTAA
ACAGAACGCTTGCCTTCTATCTGCCTACCTACCTACGCAAAACTTTAATCCTGTCTTATG
TTTTATATAATATAATCATTATATGTTTACGCAATAATTAAGGAAGAATTCATTTGATGT
GATATGTGATATGTGCTGGACAGGTCTATTCGACTGTTTTTGTACTCTCTTTTTTGTGTC
TTTTTACAATATTAAATCTATGGGTCTTGAATC
>12371853
MVMAGASSLDEIRQAQRADGPAGILAIGTANPENHVLQAEYPDYYFRITNSEHMTDLKEK
FKRNCDKSTIRKRHMHLTEEFLKENPHMCAYMAPSLDTRQDIVVVEVPKLGKEAAVKAIK
EWGQPKSKITHVVFCTTSGVDMPGADYQLTKLLGLRPSVKRLMMYQQGCFAGGTVLRIAK
DLAENNRGARVLVVCSEITAVTFRGPSDTHLDSLVGQALFSDGAAALIVGSDPDTSVGEK
PIFEMVSAAQTILPDSDGAIDGHLREVGLTFHLLKDVPGLISKNIVKSLDEAFKPLGISD
WNSLFWIAHPGGPAILDQVEIKLGLKEEKMRATRHVLSEYGNMSSAGVLFILDEMRRKSA
KDGVATTGEGLEWGVLFGFGPGLTVETVVLHSVPL*

TABLE 2

| CDNA_ID | EXPT_REP_ID | SHORT_NAME | DIFFERENTIAL (+/−) |
|---|---|---|---|
| 12323871 | 108434 | At_Root_Tips | + |
| 12323871 | 20000437 | At_Drought | + |
| 12323871 | 20000457 | At_42deg_Heat | − |
| 12323871 | 20000495 | At_Guard_Cells | + |
| 12323871 | 20000708 | At_Fis1_Siliques | − |
| 12323871 | 20001248 | At_Far-red-induction | + |
| 12323871 | 20001451 | At_Far-red-induction | + |
| 12370148 | 108434 | At_Root_Tips | − |
| 12370148 | 108454 | At_20uM_KNO3_H-to-L | + |
| 12370148 | 108461 | At_Germinating_Seeds | − |
| 12370148 | 108463 | At_Germinating_Seeds | + |
| 12370148 | 108470 | At_2mM_SA_CS3726- | + |
| | | Columbia | |
| 12370148 | 108481 | At_Shoot_Apices | + |
| 12370148 | 108501 | At_ap2_floral_buds | − |
| 12370148 | 108575 | At_Wounding | − |
| 12370148 | 108579 | At_4deg_Cold | − |
| 12370148 | 108584 | At_5mM_NaNP | + |
| 12370148 | 108588 | At_15mM_NH4NO3_L-to-H | + |
| 12370148 | 108606 | At_100uM_ABA | − |
| 12370148 | 108608 | At_100uM_ABA | − |
| 12370148 | 20000046 | At_CS237-vs-Columbia | − |
| 12370148 | 20000069 | At_100uM_ABA_Mutants | + |
| 12370148 | 20000111 | At_42deg_Heat | − |
| 12370148 | 20000166 | At_100uM_ABA | − |
| 12370148 | 20000171 | At_42deg_Heat | − |
| 12370148 | 20000173 | At_42deg_Heat | − |
| 12370148 | 20000180 | At_Germinating_Seeds | − |
| 12370148 | 20000184 | At_Shoots | − |
| 12370148 | 20000185 | At_Roots | − |
| 12370148 | 20000227 | At_Root-Tips-vs-Tops | − |
| 12370148 | 20000234 | At_Siliques | − |
| 12370148 | 20000235 | At_Siliques | − |
| 12370148 | 20000236 | At_Siliques | − |
| 12370148 | 20000264 | At_Open_Flower | − |
| 12370148 | 20000265 | At_Open_Flower | − |
| 12370148 | 20000268 | At_100mM_NaCl | − |
| 12370148 | 20000286 | At_Open_Flower | − |
| 12370148 | 20000288 | At_Drought | − |
| 12370148 | 20000308 | At_100mM_NaCl | − |
| 12370148 | 20000437 | At_Drought | − |
| 12370148 | 20000438 | At_Shoots | − |
| 12370148 | 20000439 | At_Roots | − |
| 12370148 | 20000441 | At_1uM_BR-BRZ | + |
| 12370148 | 20000443 | At_1uM_BR-BRZ | + |
| 12370148 | 20000460 | At_10%_PEG | + |
| 12370148 | 20000506 | At_Wounding | − |
| 12370148 | 20000527 | At_10%_PEG | + |
| 12370148 | 20000573 | At_100uM_ABA_Mutants | − |
| 12370148 | 20000574 | At_100uM_ABA_Mutants | − |
| 12370148 | 20000708 | At_Fis1_Siliques | − |
| 12370148 | 20001248 | At_Far-red-induction | + |
| 12370148 | 20001458 | At_50mM_NH4NO3_L-to-H | + |
| 12370148 | 20001557 | At_Drought_Soil_Dry | + |
| 12371508 | 108434 | At_Root_Tips | + |
| 12371508 | 108461 | At_Germinating_Seeds | + |
| 12371508 | 108463 | At_Germinating_Seeds | − |
| 12371508 | 108464 | At_Germinating_Seeds | − |
| 12371508 | 108474 | At_Drought_Flowers | − |
| 12371508 | 108561 | At_100uM_ABA | − |
| 12371508 | 108583 | At_5mM_H2O2 | − |
| 12371508 | 108590 | At_15mM_NH4NO3_L-to-H | + |
| 12371508 | 108668 | At_2mM_SA | − |
| 12371508 | 20000111 | At_42deg_Heat | − |
| 12371508 | 20000117 | At_100uM_ABA_Mutants | − |
| 12371508 | 20000144 | At_42deg_Heat | − |
| 12371508 | 20000173 | At_42deg_Heat | − |
| 12371508 | 20000180 | At_Germinating_Seeds | + |
| 12371508 | 20000184 | At_Shoots | + |
| 12371508 | 20000227 | At_Root-Tips-vs-Tops | + |
| 12371508 | 20000264 | At_Open_Flower | + |
| 12371508 | 20000265 | At_Open_Flower | + |
| 12371508 | 20000286 | At_Open_Flower | + |
| 12371508 | 20000437 | At_Drought | − |
| 12371508 | 20000445 | At_100uM_NAA | + |
| 12371508 | 20000458 | At_42deg_Heat | − |
| 12371508 | 20000495 | At_Guard_Cells | + |
| 12371508 | 20000794 | At_Petals | + |
| 12371508 | 20001248 | At_Far-red-induction | + |
| 12371508 | 20001450 | At_Far-red-induction | + |
| 12371508 | 20001451 | At_Far-red-induction | + |
| 12371508 | 20001555 | At_Drought_Soil_Dry | − |
| 12371852 | 108433 | At_rhl_Mutant2 | − |
| 12371852 | 108461 | At_Germinating_Seeds | + |
| 12371852 | 108462 | At_Germinating_Seeds | + |
| 12371852 | 108463 | At_Germinating_Seeds | + |
| 12371852 | 108464 | At_Germinating_Seeds | + |
| 12371852 | 108478 | At_Shoot_Apices | + |
| 12371852 | 108489 | At_50mM_NH4NO3_L-to- | − |
| | | H_Rosette | |
| 12371852 | 108561 | At_100uM_ABA | − |
| 12371852 | 108569 | At_0.001%_MeJA | + |
| 12371852 | 108575 | At_Wounding | + |
| 12371852 | 108577 | At_42deg_Heat | + |
| 12371852 | 20000069 | At_100uM_ABA_Mutants | − |
| 12371852 | 20000070 | At_100uM_ABA_Mutants | − |
| 12371852 | 20000071 | At_100uM_ABA_Mutants | − |
| 12371852 | 20000072 | At_100uM_ABA_Mutants | − |
| 12371852 | 20000086 | At_100uM_ABA_Mutants | − |
| 12371852 | 20000087 | At_100uM_ABA_Mutants | − |
| 12371852 | 20000088 | At_100uM_ABA_Mutants | − |
| 12371852 | 20000089 | At_2mM_SA_CS3726- | − |
| | | Columbia | |
| 12371852 | 20000090 | At_2mM_SA_CS3726- | − |
| | | Columbia | |
| 12371852 | 20000111 | At_42deg_Heat | + |
| 12371852 | 20000113 | At_42deg_Heat | + |
| 12371852 | 20000117 | At_100uM_ABA_Mutants | − |
| 12371852 | 20000173 | At_42deg_Heat | + |
| 12371852 | 20000179 | At_Germinating_Seeds | + |
| 12371852 | 20000180 | At_Germinating_Seeds | + |
| 12371852 | 20000184 | At_Shoots | + |
| 12371852 | 20000185 | At_Roots | + |
| 12371852 | 20000234 | At_Siliques | + |
| 12371852 | 20000236 | At_Siliques | + |
| 12371852 | 20000264 | At_Open_Flower | + |
| 12371852 | 20000265 | At_Open_Flower | + |
| 12371852 | 20000286 | At_Open_Flower | + |
| 12371852 | 20000308 | At_100mM_NaCl | + |
| 12371852 | 20000436 | At_Drought | + |
| 12371852 | 20000439 | At_Roots | + |
| 12371852 | 20000455 | At_100uM_ABA | + |
| 12371852 | 20000458 | At_42deg_Heat | + |
| 12371852 | 20000495 | At_Guard_Cells | − |
| 12371852 | 20000573 | At_100uM_ABA_Mutants | − |
| 12371852 | 20000708 | At_Fis1_Siliques | + |
| 12371852 | 20001248 | At_Far-red-induction | − |
| 12371852 | 20001450 | At_Far-red-induction | − |
| 12371852 | 20001451 | At_Far-red-induction | − |
| 12371852 | 20001503 | At_Far-red-enriched | + |
| 12371852 | 20001504 | At_Far-red-enriched | + |
| 12371852 | 20001555 | At_Drought_Soil_Dry | + |
| 12371852 | 20001556 | At_Drought_Soil_Dry | + |
| 12371852 | 20001557 | At_Drought_Soil_Dry | + |
| 12371852 | 20001558 | At_Drought_Soil_Dry | + |
| 12371852 | 20001559 | At_Drought_Soil_Dry | + |
| 12420894 | 108434 | At_Root_Tips | + |
| 12420894 | 108462 | At_Germinating_Seeds | − |
| 12420894 | 108463 | At_Germinating_Seeds | − |
| 12420894 | 20000111 | At_42deg_Heat | + |
| 12420894 | 20000184 | At_Shoots | + |
| 12420894 | 20000794 | At_Petals | − |
| 12420894 | 20001557 | At_Drought_Soil_Dry | + |
| 12420894 | 20001558 | At_Drought_Soil_Dry | + |
| 12560350 | 20000171 | At_42deg_Heat | − |
| 12560350 | 20000173 | At_42deg_Heat | − |
| 12560350 | 20000185 | At_Roots | − |
| 12560350 | 20000227 | At_Root-Tips-vs-Tops | − |
| 12560350 | 20000234 | At_Siliques | + |
| 12560350 | 20000235 | At_Siliques | + |
| 12560350 | 20000264 | At_Open_Flower | + |
| 12560350 | 20000265 | At_Open_Flower | + |
| 12560350 | 20000286 | At_Open_Flower | + |
| 12560350 | 20000458 | At_42deg_Heat | − |
| 12560350 | 20000573 | At_100uM_ABA_Mutants | − |
| 12560350 | 20000708 | At_Fis1_Siliques | + |
| 12560350 | 20000794 | At_Petals | + |
| 12560350 | 20001557 | At_Drought_Soil_Dry | + |
| 12560350 | 20001558 | At_Drought_Soil_Dry | + |
| 12673011 | 108434 | At_Root_Tips | + |
| 12673011 | 108462 | At_Germinating_Seeds | − |
| 12673011 | 108463 | At_Germinating_Seeds | − |
| 12673011 | 108488 | At_50mM_NH4NO3_L-to- | − |
| | | H_Rosette | |
| 12673011 | 108577 | At_42deg_Heat | − |
| 12673011 | 108606 | At_100uM_ABA | − |
| 12673011 | 108607 | At_100uM_ABA | − |
| 12673011 | 20000069 | At_100uM_ABA_Mutants | + |
| 12673011 | 20000070 | At_100uM_ABA_Mutants | + |
| 12673011 | 20000071 | At_100uM_ABA_Mutants | + |
| 12673011 | 20000072 | At_100uM_ABA_Mutants | + |
| 12673011 | 20000087 | At_100uM_ABA_Mutants | + |
| 12673011 | 20000088 | At_100uM_ABA_Mutants | + |
| 12673011 | 20000089 | At_2mM_SA_CS3726- | + |
| | | Columbia | |
| 12673011 | 20000090 | At_2mM_SA_CS3726- | + |
| | | Columbia | |
| 12673011 | 20000117 | At_100uM_ABA_Mutants | + |
| 12673011 | 20000213 | At_4deg_Cold | + |
| 12673011 | 20000438 | At_Shoots | − |
| 12673011 | 20001451 | At_Far-red-induction | + |
| 12673011 | 20001557 | At_Drought_Soil_Dry | + |
| 12677570 | 108434 | At_Root_Tips | + |
| 12677570 | 108463 | At_Germinating_Seeds | − |
| 12677570 | 108464 | At_Germinating_Seeds | − |
| 12677570 | 20000068 | At_CS3824_vs_Landsberg | + |
| 12677570 | 20000113 | At_42deg_Heat | − |
| 12679464 | 108434 | At_Root_Tips | − |
| 12679464 | 108464 | At_Germinating_Seeds | + |
| 12679464 | 108469 | At_2mM_SA_CS3726- | + |
| | | Columbia | |
| 12679464 | 108470 | At_2mM_SA_CS3726- | + |
| | | Columbia | |
| 12679464 | 108480 | At_Shoot_Apices | + |
| 12679464 | 108481 | At_Shoot_Apices | + |
| 12679464 | 108501 | At_ap2_floral_buds | − |
| 12679464 | 108568 | At_0.001%_MeJA | + |
| 12679464 | 108577 | At_42deg_Heat | − |
| 12679464 | 108584 | At_5mM_NaNP | + |
| 12679464 | 108585 | At_5mM_NaNP | + |
| 12679464 | 108588 | At_15mM_NH4NO3_L-to-H | + |
| 12679464 | 108594 | At_Ler-rhl_Root | + |
| 12679464 | 108595 | At_Ler-pi_Ovule | + |
| 12679464 | 108667 | At_2mM_SA | + |
| 12679464 | 20000046 | At_CS237-vs-Columbia | − |
| 12679464 | 20000070 | At_100uM_ABA_Mutants | + |
| 12679464 | 20000072 | At_100uM_ABA_Mutants | + |
| 12679464 | 20000086 | At_100uM_ABA_Mutants | + |
| 12679464 | 20000088 | At_100uM_ABA_Mutants | + |
| 12679464 | 20000111 | At_42deg_Heat | − |
| 12679464 | 20000117 | At_100uM_ABA_Mutants | + |
| 12679464 | 20000144 | At_42deg_Heat | − |
| 12679464 | 20000179 | At_Germinating_Seeds | − |
| 12679464 | 20000185 | At_Roots | − |
| 12679464 | 20000227 | At_Root-Tips-vs-Tops | − |
| 12679464 | 20000234 | At_Siliques | − |
| 12679464 | 20000235 | At_Siliques | − |
| 12679464 | 20000236 | At_Siliques | − |
| 12679464 | 20000264 | At_Open_Flower | − |
| 12679464 | 20000265 | At_Open_Flower | − |
| 12679464 | 20000286 | At_Open_Flower | − |
| 12679464 | 20000437 | At_Drought | − |
| 12679464 | 20000438 | At_Shoots | − |
| 12679464 | 20000439 | At_Roots | − |
| 12679464 | 20000441 | At_1uM_BR-BRZ | + |
| 12679464 | 20000443 | At_1uM_BR-BRZ | + |
| 12679464 | 20000460 | At_10%_PEG | + |
| 12679464 | 20000708 | At_Fis1_Siliques | − |
| 12679464 | 20000709 | At_15mM_NH4NO3_L-to-H | + |
| 12679464 | 20000794 | At_Petals | − |
| 12679464 | 20001556 | At_Drought_Soil_Dry | − |
| 12688873 | 108462 | At_Germinating_Seeds | + |
| 12688873 | 108463 | At_Germinating_Seeds | + |
| 12688873 | 108464 | At_Germinating_Seeds | + |
| 12688873 | 108488 | At_50mM_NH4NO3_L-to- | − |
| | | H_Rosette | |
| 12688873 | 108560 | At_100uM_ABA | + |
| 12688873 | 108572 | At_Drought | + |
| 12688873 | 108574 | At_Wounding | + |
| 12688873 | 108588 | At_15mM_NH4NO3_L-to-H | + |
| 12688873 | 108594 | At_Ler-rhl_Root | + |
| 12688873 | 108595 | At_Ler-pi_Ovule | + |
| 12688873 | 108606 | At_100uM_ABA | + |
| 12688873 | 108607 | At_100uM_ABA | + |
| 12688873 | 108608 | At_100uM_ABA | + |
| 12688873 | 108667 | At_2mM_SA | + |
| 12688873 | 20000070 | At_100uM_ABA_Mutants | + |
| 12688873 | 20000166 | At_100uM_ABA | + |
| 12688873 | 20000223 | At_CS6632_Shoots-Roots | + |
| 12688873 | 20000227 | At_Root-Tips-vs-Tops | − |
| 12688873 | 20000234 | At_Siliques | − |
| 12688873 | 20000264 | At_Open_Flower | − |
| 12688873 | 20000460 | At_10%_PEG | + |
| 12688873 | 20000506 | At_Wounding | − |
| 12688873 | 20000527 | At_10%_PEG | + |
| 12688873 | 20000573 | At_100uM_ABA_Mutants | − |
| 12688873 | 20000574 | At_100uM_ABA_Mutants | − |
| 12688873 | 20000794 | At_Petals | − |
| 12688873 | 20001247 | At_Far-red-induction | + |
| 12688873 | 20001248 | At_Far-red-induction | + |
| 12688873 | 20001450 | At_Far-red-induction | + |
| 12699286 | 20000087 | At_100uM_ABA_Mutants | − |
| 12699286 | 20000315 | At_14day_Shoots-Roots | + |
| 12704782 | 108469 | At_2mM_SA_CS3726- | + |
| | | Columbia | |
| 12704782 | 108481 | At_Shoot_Apices | + |
| 12704782 | 108489 | At_50mM_NH4NO3_L-to- | − |
| | | H_Rosette | |
| 12704782 | 108576 | At_42deg_Heat | + |
| 12704782 | 108577 | At_42deg_Heat | + |
| 12704782 | 108583 | At_5mM_H2O2 | + |
| 12704782 | 108584 | At_5mM_NaNP | + |
| 12704782 | 108585 | At_5mM_NaNP | + |
| 12704782 | 108589 | At_15mM_NH4NO3_L-to-H | − |
| 12704782 | 108595 | At_Ler-pi_Ovule | + |
| 12704782 | 108667 | At_2mM_SA | + |
| 12704782 | 108668 | At_2mM_SA | + |
| 12704782 | 20000069 | At_100uM_ABA_Mutants | + |
| 12704782 | 20000070 | At_100uM_ABA_Mutants | + |
| 12704782 | 20000090 | At_2mM_SA_CS3726- | + |
| | | Columbia | |
| 12704782 | 20000092 | At_42deg_Heat | − |
| 12704782 | 20000113 | At_42deg_Heat | + |
| 12704782 | 20000144 | At_42deg_Heat | + |
| 12704782 | 20000171 | At_42deg_Heat | + |
| 12704782 | 20000173 | At_42deg_Heat | + |
| 12704782 | 20000179 | At_Germinating_Seeds | + |
| 12704782 | 20000227 | At_Root-Tips-vs-Tops | + |
| 12704782 | 20000437 | At_Drought | + |
| 12704782 | 20000453 | At_100uM_ABA | + |
| 12704782 | 20000456 | At_100uM_BA | + |
| 12704782 | 20000457 | At_42deg_Heat | − |
| 12704782 | 20000458 | At_42deg_Heat | + |
| 12704782 | 20000506 | At_Wounding | + |
| 12704782 | 20000709 | At_15mM_NH4NO3_L-to-H | + |
| 12704782 | 20001248 | At_Far-red-induction | − |
| 12704782 | 20001450 | At_Far-red-induction | − |
| 12704782 | 20001555 | At_Drought_Soil_Dry | + |
| 12704782 | 20001556 | At_Drought_Soil_Dry | + |
| 12704782 | 20001560 | At_Drought_Soil_Dry | + |
| 12705056 | 108433 | At_rhl_Mutant2 | − |
| 12705056 | 108461 | At_Germinating_Seeds | + |
| 12705056 | 108462 | At_Germinating_Seeds | + |
| 12705056 | 108463 | At_Germinating_Seeds | + |
| 12705056 | 108464 | At_Germinating_Seeds | + |
| 12705056 | 108478 | At_Shoot_Apices | + |
| 12705056 | 108489 | At_50mM_NH4NO3_L-to- | − |
| | | H_Rosette | |
| 12705056 | 108561 | At_100uM_ABA | − |
| 12705056 | 108569 | At_0.001%_MeJA | + |
| 12705056 | 108575 | At_Wounding | + |
| 12705056 | 108577 | At_42deg_Heat | + |
| 12705056 | 20000069 | At_100uM_ABA_Mutants | − |
| 12705056 | 20000070 | At_100uM_ABA_Mutants | − |
| 12705056 | 20000071 | At_100uM_ABA_Mutants | − |
| 12705056 | 20000072 | At_100uM_ABA_Mutants | − |
| 12705056 | 20000086 | At_100uM_ABA_Mutants | − |
| 12705056 | 20000087 | At_100uM_ABA_Mutants | − |
| 12705056 | 20000088 | At_100uM_ABA_Mutants | − |
| 12705056 | 20000089 | At_2mM_SA_CS3726- | − |
| | | Columbia | |
| 12705056 | 20000090 | At_2mM_SA_CS3726- | − |
| | | Columbia | |
| 12705056 | 20000111 | At_42deg_Heat | + |
| 12705056 | 20000113 | At_42deg_Heat | + |
| 12705056 | 20000117 | At_100uM_ABA_Mutants | − |
| 12705056 | 20000173 | At_42deg_Heat | + |
| 12705056 | 20000179 | At_Germinating_Seeds | + |
| 12705056 | 20000180 | At_Germinating_Seeds | + |
| 12705056 | 20000184 | At_Shoots | + |
| 12705056 | 20000185 | At_Roots | + |
| 12705056 | 20000234 | At_Siliques | + |
| 12705056 | 20000236 | At_Siliques | + |
| 12705056 | 20000264 | At_Open_Flower | + |
| 12705056 | 20000265 | At_Open_Flower | + |
| 12705056 | 20000286 | At_Open_Flower | + |
| 12705056 | 20000308 | At_100mM_NaCl | + |
| 12705056 | 20000436 | At_Drought | + |
| 12705056 | 20000439 | At_Roots | + |
| 12705056 | 20000455 | At_100uM_ABA | + |
| 12705056 | 20000458 | At_42deg_Heat | + |
| 12705056 | 20000495 | At_Guard_Cells | − |
| 12705056 | 20000573 | At_100uM_ABA_Mutants | − |
| 12705056 | 20000708 | At_Fis1_Siliques | + |
| 12705056 | 20001248 | At_Far-red-induction | − |
| 12705056 | 20001450 | At_Far-red-induction | − |
| 12705056 | 20001451 | At_Far-red-induction | − |
| 12705056 | 20001503 | At_Far-red-enriched | + |
| 12705056 | 20001504 | At_Far-red-enriched | + |
| 12705056 | 20001555 | At_Drought_Soil_Dry | + |
| 12705056 | 20001556 | At_Drought_Soil_Dry | + |
| 12705056 | 20001557 | At_Drought_Soil_Dry | + |
| 12705056 | 20001558 | At_Drought_Soil_Dry | + |
| 12705056 | 20001559 | At_Drought_Soil_Dry | + |
| 12705120 | 108435 | At_stm_Mutants | − |
| 12705120 | 108461 | At_Germinating_Seeds | + |
| 12705120 | 108462 | At_Germinating_Seeds | + |
| 12705120 | 108463 | At_Germinating_Seeds | + |
| 12705120 | 108464 | At_Germinating_Seeds | + |
| 12705120 | 108488 | At_50mM_NH4NO3_L-to- | + |
| | | H_Rosette | |
| 12705120 | 108595 | At_Ler-pi_Ovule | − |
| 12705120 | 20000069 | At_100uM_ABA_Mutants | − |
| 12705120 | 20000070 | At_100uM_ABA_Mutants | − |
| 12705120 | 20000071 | At_100uM_ABA_Mutants | − |
| 12705120 | 20000072 | At_100uM_ABA_Mutants | − |
| 12705120 | 20000086 | At_100uM_ABA_Mutants | − |
| 12705120 | 20000087 | At_100uM_ABA_Mutants | − |
| 12705120 | 20000088 | At_100uM_ABA_Mutants | − |
| 12705120 | 20000090 | At_2mM_SA_CS3726- | − |
| | | Columbia | |
| 12705120 | 20000117 | At_100uM_ABA_Mutants | − |
| 12705120 | 20000180 | At_Germinating_Seeds | + |
| 12705120 | 20000185 | At_Roots | − |
| 12705120 | 20000234 | At_Siliques | − |
| 12705120 | 20000264 | At_Open_Flower | − |
| 12705120 | 20000438 | At_Shoots | + |
| 12705120 | 20000439 | At_Roots | − |
| 12705120 | 20000495 | At_Guard_Cells | − |
| 12705120 | 20000496 | At_Guard_Cells | − |
| 12705120 | 20000794 | At_Petals | − |
| 12705120 | 20001451 | At_Far-red-induction | − |
| 12705120 | 20001557 | At_Drought_Soil_Dry | − |
| 12705120 | 20001558 | At_Drought_Soil_Dry | − |
| 12705120 | 20001560 | At_Drought_Soil_Dry | − |
| 12712671 | 20000314 | At_14day_Shoots-Roots | − |
| 12712671 | 20000327 | At_42deg_Heat | + |
| 12712671 | 20000328 | At_42deg_Heat | + |
| 12712671 | 20000355 | At_Siliques | − |
| 12719868 | 108434 | At_Root_Tips | − |
| 12719868 | 108454 | At_20uM_KNO3_H-to-L | + |
| 12719868 | 108463 | At_Germinating_Seeds | + |
| 12719868 | 108464 | At_Germinating_Seeds | + |
| 12719868 | 108481 | At_Shoot_Apices | + |
| 12719868 | 108575 | At_Wounding | − |
| 12719868 | 108579 | At_4deg_Cold | + |
| 12719868 | 108584 | At_5mM_NaNP | + |
| 12719868 | 108588 | At_15mM_NH4NO3_L-to-H | + |
| 12719868 | 108595 | At_Ler-pi_Ovule | + |
| 12719868 | 20000046 | At_CS237-vs-Columbia | − |
| 12719868 | 20000069 | At_100uM_ABA_Mutants | + |
| 12719868 | 20000144 | At_42deg_Heat | − |
| 12719868 | 20000171 | At_42deg_Heat | − |
| 12719868 | 20000173 | At_42deg_Heat | − |
| 12719868 | 20000179 | At_Germinating_Seeds | − |
| 12719868 | 20000213 | At_4deg_Cold | + |
| 12719868 | 20000234 | At_Siliques | − |
| 12719868 | 20000235 | At_Siliques | − |
| 12719868 | 20000236 | At_Siliques | − |
| 12719868 | 20000264 | At_Open_Flower | − |
| 12719868 | 20000265 | At_Open_Flower | − |
| 12719868 | 20000286 | At_Open_Flower | − |
| 12719868 | 20000326 | At_Pollen | − |
| 12719868 | 20000438 | At_Shoots | − |
| 12719868 | 20000441 | At_1uM_BR-BRZ | + |
| 12719868 | 20000443 | At_1uM_BR-BRZ | + |
| 12719868 | 20000458 | At_42deg_Heat | − |
| 12719868 | 20000460 | At_10%_PEG | + |
| 12719868 | 20000506 | At_Wounding | − |
| 12719868 | 20000527 | At_10%_PEG | + |
| 12719868 | 20000573 | At_100uM_ABA_Mutants | − |
| 12719868 | 20000574 | At_100uM_ABA_Mutants | − |
| 12719868 | 20000578 | At_CS3726_YF | − |
| 12719868 | 20000606 | At_CS8548_Mutant | − |
| 12719868 | 20000708 | At_Fis1_Siliques | − |
| 12719868 | 20000794 | At_Petals | − |
| 13504706 | 108433 | At_rhl_Mutant2 | + |
| 13504706 | 108461 | At_Germinating_Seeds | + |
| 13504706 | 108462 | At_Germinating_Seeds | + |
| 13504706 | 108463 | At_Germinating_Seeds | + |
| 13504706 | 108464 | At_Germinating_Seeds | + |
| 13504706 | 108470 | At_2mM_SA_CS3726- | − |
| | | Columbia | |
| 13504706 | 108474 | At_Drought_Flowers | + |
| 13504706 | 108480 | At_Shoot_Apices | + |
| 13504706 | 108481 | At_Shoot_Apices | + |
| 13504706 | 108501 | At_ap2_floral_buds | + |
| 13504706 | 108573 | At_Drought | + |
| 13504706 | 108595 | At_Ler-pi_Ovule | − |
| 13504706 | 20000066 | At_CS3071_vs_Columbia | − |
| 13504706 | 20000068 | At_CS3824_vs_Landsberg | + |
| 13504706 | 20000088 | At_100uM_ABA_Mutants | − |
| 13504706 | 20000184 | At_Shoots | + |
| 13504706 | 20000185 | At_Roots | + |
| 13504706 | 20000234 | At_Siliques | + |
| 13504706 | 20000235 | At_Siliques | + |
| 13504706 | 20000236 | At_Siliques | + |
| 13504706 | 20000264 | At_Open_Flower | + |
| 13504706 | 20000265 | At_Open_Flower | + |
| 13504706 | 20000286 | At_Open_Flower | + |
| 13504706 | 20000438 | At_Shoots | + |
| 13504706 | 20000439 | At_Roots | + |
| 13504706 | 20000441 | At_1uM_BR-BRZ | + |
| 13504706 | 20000443 | At_1uM_BR-BRZ | + |
| 13504706 | 20000458 | At_42deg_Heat | + |
| 13504706 | 20000460 | At_10%_PEG | − |
| 13504706 | 20000527 | At_10%_PEG | − |
| 13504706 | 20000573 | At_100uM_ABA_Mutants | − |
| 13504706 | 20000574 | At_100uM_ABA_Mutants | − |
| 13504706 | 20000576 | At_100uM_ABA_Mutants | − |
| 13504706 | 20000708 | At_Fis1_Siliques | − |
| 13504706 | 20001557 | At_Drought_Soil_Dry | − |
| 13504706 | 20001560 | At_Drought_Soil_Dry | − |
| 13601536 | 108461 | At_Germinating_Seeds | + |
| 13601536 | 108576 | At_42deg_Heat | − |
| 13601536 | 108577 | At_42deg_Heat | − |
| 13601536 | 108583 | At_5mM_H2O2 | − |
| 13601536 | 108595 | At_Ler-pi_Ovule | − |
| 13602983 | 20000264 | At_Open_Flower | + |
| 13602983 | 20000265 | At_Open_Flower | + |
| 13602983 | 20000286 | At_Open_Flower | + |
| 13602983 | 20000437 | At_Drought | − |
| 13602983 | 20000438 | At_Shoots | + |
| 13602983 | 20000458 | At_42deg_Heat | − |
| 13602983 | 20000495 | At_Guard_Cells | + |
| 13603142 | 108434 | At_Root_Tips | + |
| 13603142 | 108461 | At_Germinating_Seeds | − |
| 13603142 | 108462 | At_Germinating_Seeds | − |
| 13603142 | 108463 | At_Germinating_Seeds | − |
| 13603142 | 108464 | At_Germinating_Seeds | − |
| 13603142 | 108573 | At_Drought | − |
| 13603142 | 20000227 | At_Root-Tips-vs-Tops | + |
| 13603142 | 20000234 | At_Siliques | + |
| 13603142 | 20000458 | At_42deg_Heat | + |
| 13603142 | 20001557 | At_Drought_Soil_Dry | + |
| 13603142 | 20001558 | At_Drought_Soil_Dry | + |
| 13603142 | 20001560 | At_Drought_Soil_Dry | + |
| 13603177 | 108461 | At_Germinating_Seeds | − |
| 13603177 | 108462 | At_Germinating_Seeds | + |
| 13603177 | 108463 | At_Germinating_Seeds | + |
| 13603177 | 108464 | At_Germinating_Seeds | + |
| 13603177 | 108488 | At_50mM_NH4NO3_L-to- | − |
| | | H_Rosette | |
| 13603177 | 108560 | At_100uM_ABA | + |
| 13603177 | 108569 | At_0.001%_MeJA | + |
| 13603177 | 108573 | At_Drought | + |
| 13603177 | 108590 | At_15mM_NH4NO3_L-to-H | − |
| 13603177 | 108591 | At_15mM_NH4NO3_L-to-H | − |
| 13603177 | 108594 | At_Ler-rhl_Root | − |
| 13603177 | 108595 | At_Ler-pi_Ovule | + |
| 13603177 | 108609 | At_100uM_ABA | + |
| 13603177 | 20000069 | At_100uM_ABA_Mutants | + |
| 13603177 | 20000070 | At_100uM_ABA_Mutants | + |
| 13603177 | 20000071 | At_100uM_ABA_Mutants | + |
| 13603177 | 20000072 | At_100uM_ABA_Mutants | + |
| 13603177 | 20000086 | At_100uM_ABA_Mutants | + |
| 13603177 | 20000087 | At_100uM_ABA_Mutants | + |
| 13603177 | 20000088 | At_100uM_ABA_Mutants | + |
| 13603177 | 20000112 | At_42deg_Heat | + |
| 13603177 | 20000113 | At_42deg_Heat | + |
| 13603177 | 20000117 | At_100uM_ABA_Mutants | + |
| 13603177 | 20000306 | At_Germinating_Seeds | − |
| 13603177 | 20000307 | At_Germinating_Seeds | − |
| 13603177 | 20000329 | At_4deg_Cold | + |
| 13603177 | 20000332 | At_4deg_Cold | + |
| 13603177 | 20000355 | At_Siliques | − |
| 13607229 | 20000708 | At_Fis1_Siliques | − |
| 13607852 | 108434 | At_Root_Tips | − |
| 13607852 | 108501 | At_ap2_floral_buds | + |
| 13607852 | 20000184 | At_Shoots | − |
| 13607852 | 20000234 | At_Siliques | + |
| 13607852 | 20000235 | At_Siliques | + |
| 13607852 | 20000264 | At_Open_Flower | + |
| 13607852 | 20000265 | At_Open_Flower | + |
| 13607852 | 20000286 | At_Open_Flower | + |
| 13607852 | 20000437 | At_Drought | + |
| 13607852 | 20000708 | At_Fis1_Siliques | + |
| 13607852 | 20001451 | At_Far-red-induction | + |
| 13608279 | 108434 | At_Root_Tips | − |
| 13608279 | 108435 | At_stm_Mutants | − |
| 13608279 | 108461 | At_Germinating_Seeds | + |
| 13608279 | 108462 | At_Germinating_Seeds | + |
| 13608279 | 108463 | At_Germinating_Seeds | + |
| 13608279 | 108464 | At_Germinating_Seeds | + |
| 13608279 | 108573 | At_Drought | + |
| 13608279 | 20000306 | At_Germinating_Seeds | + |
| 13608279 | 20000307 | At_Germinating_Seeds | + |
| 13608279 | 20000328 | At_42deg_Heat | − |
| 13616623 | 20000306 | At_Germinating_Seeds | + |
| 13616623 | 20000307 | At_Germinating_Seeds | + |
| 13616623 | 20000314 | At_14day_Shoots-Roots | − |
| 13616623 | 20000315 | At_14day_Shoots-Roots | − |
| 13616623 | 20000327 | At_42deg_Heat | − |
| 13616623 | 20000329 | At_4deg_Cold | + |
| 13616623 | 20000332 | At_4deg_Cold | + |
| 13616623 | 20000346 | At_2mM_SA | + |
| 13616623 | 20000347 | At_2mM_SA | + |
| 13616623 | 20000352 | At_Drought | + |
| 13616623 | 20000355 | At_Siliques | − |
| 13616623 | 20000440 | At_Closed_Bud | − |
| 13616623 | 20000634 | At_10%_PEG | + |
| 13618061 | 20000185 | At_Roots | − |
| 13618061 | 20000264 | At_Open_Flower | + |
| 13618061 | 20000265 | At_Open_Flower | + |
| 13618061 | 20000286 | At_Open_Flower | + |
| 13618061 | 20000438 | At_Shoots | − |
| 13618061 | 20000439 | At_Roots | − |
| 13618061 | 20000794 | At_Petals | + |
| 13619634 | 20000326 | At_Pollen | − |
| 13619634 | 20000437 | At_Drought | − |
| 13619634 | 20000794 | At_Petals | − |
| 13619634 | 20001451 | At_Far-red-induction | − |
| 13619634 | 20001557 | At_Drought_Soil_Dry | + |
| 13619634 | 20001558 | At_Drought_Soil_Dry | + |
| 13619634 | 20001560 | At_Drought_Soil_Dry | + |
| 12688873 | 20000460 | At_10%_PEG | + |
| 12688873 | 20000527 | At_10%_PEG | + |
| 13619728 | 20000527 | At_10%_PEG | − |
| 12688873 | 20000070 | At_100uM_ABA_Mutants | + |
| 12688873 | 20000573 | At_100uM_ABA_Mutants | − |
| 12688873 | 20000574 | At_100uM_ABA_Mutants | − |
| 12688873 | 20000166 | At_100uM_ABA | + |
| 12688873 | 108560 | At_100uM_ABA | + |
| 12688873 | 108606 | At_100uM_ABA | + |
| 12688873 | 108607 | At_100uM_ABA | + |
| 12688873 | 108608 | At_100uM_ABA | + |
| 13619728 | 108608 | At_100uM_ABA | − |
| 13619728 | 20000709 | At_15mM_NH4NO3_L-to-H | + |
| 12688873 | 108588 | At_15mM_NH4NO3_L-to-H | + |
| 13619728 | 108455 | At_20uM_KNO3_H-to-L | + |
| 13619728 | 20000089 | At_2mM_SA_CS3726- | + |
| | | Columbia | |
| 12707591 | 20000090 | At_2mM_SA_CS3726- | − |
| | | Columbia | |
| 13619728 | 108469 | At_2mM_SA_CS3726- | + |
| | | Columbia | |
| 13619728 | 108470 | At_2mM_SA_CS3726- | + |
| | | Columbia | |
| 12688873 | 108667 | At_2mM_SA | + |
| 13619728 | 108667 | At_2mM_SA | + |
| 13619728 | 108668 | At_2mM_SA | + |
| 12385295 | 20000113 | At_42deg_Heat | − |
| 13619728 | 20000173 | At_42deg_Heat | − |
| 13619728 | 20000458 | At_42deg_Heat | − |
| 12385295 | 108577 | At_42deg_Heat | − |
| 13619728 | 108579 | At_4deg_Cold | + |
| 12688873 | 108488 | At_50mM_NH4NO3_L-to- | − |
| | | H_Rosette | |
| 13619728 | 108489 | At_50mM_NH4NO3_L-to- | − |
| | | H_Rosette | |
| 13619728 | 108584 | At_5mM_NaNP | + |
| 13619728 | 108585 | At_5mM_NaNP | + |
| 12688873 | 20000223 | At_CS6632_Shoots-Roots | + |
| 13619728 | 108473 | At_Drought_Flowers | − |
| 13617271 | 20001554 | At_Drought_Soil_Dry | + |
| 13617271 | 20001555 | At_Drought_Soil_Dry | + |
| 12688873 | 108572 | At_Drought | + |
| 12707591 | 108573 | At_Drought | + |
| 13619728 | 108572 | At_Drought | − |
| 12688873 | 20001247 | At_Far-red-induction | + |
| 12688873 | 20001248 | At_Far-red-induction | + |
| 12688873 | 20001450 | At_Far-red-induction | + |
| 13619728 | 20001450 | At_Far-red-induction | − |
| 13619728 | 20001451 | At_Far-red-induction | − |
| 12385295 | 20000180 | At_Germinating_Seeds | + |
| 12385295 | 108461 | At_Germinating_Seeds | + |
| 12688873 | 108462 | At_Germinating_Seeds | + |
| 12688873 | 108463 | At_Germinating_Seeds | + |
| 12688873 | 108464 | At_Germinating_Seeds | + |
| 12707591 | 108462 | At_Germinating_Seeds | + |
| 12707591 | 108463 | At_Germinating_Seeds | + |
| 12707591 | 108464 | At_Germinating_Seeds | + |
| 13619728 | 108462 | At_Germinating_Seeds | + |
| 13619728 | 108463 | At_Germinating_Seeds | + |
| 13619728 | 108464 | At_Germinating_Seeds | + |
| 12688873 | 108595 | At_Ler-pi_Ovule | + |
| 13619728 | 108595 | At_Ler-pi_Ovule | + |
| 12707591 | 108595 | At_Ler-pi_Ovule | − |
| 12688873 | 108594 | At_Ler-rhl_Root | + |
| 12385295 | 20000264 | At_Open_Flower | + |
| 12385295 | 20000265 | At_Open_Flower | + |
| 12385295 | 20000286 | At_Open_Flower | + |
| 12688873 | 20000264 | At_Open_Flower | − |
| 12688873 | 20000794 | At_Petals | − |
| 12385295 | 20000326 | At_Pollen | + |
| 12385295 | 108434 | At_Root_Tips | + |
| 12707591 | 108434 | At_Root_Tips | − |
| 13619728 | 108434 | At_Root_Tips | − |
| 13619728 | 20000185 | At_Roots | + |
| 13617271 | 20000185 | At_Roots | − |
| 13617271 | 20000439 | At_Roots | − |
| 12688873 | 20000227 | At_Root-Tips-vs-Tops | − |
| 13619728 | 108478 | At_Shoot_Apices | + |
| 13617271 | 20000184 | At_Shoots | − |
| 13617271 | 20000438 | At_Shoots | − |
| 12385295 | 20000234 | At_Siliques | + |
| 12688873 | 20000234 | At_Siliques | − |
| 12688873 | 108574 | At_Wounding | + |
| 13619728 | 108574 | At_Wounding | + |
| 12688873 | 20000506 | At_Wounding | − |
| 13604752 | 20000171 | At_42deg_Heat | − |
| 13604752 | 20000173 | At_42deg_Heat | − |
| 13604752 | 20000184 | At_Shoots | − |
| 13604752 | 20000438 | At_Shoots | − |
| 13604752 | 20000458 | At_42deg_Heat | − |
| 13604752 | 20000180 | At_Germinating_Seeds | + |

TABLE 3

| Utility Section | Expt_Rep_ID | Short_Name | Parameter | Value |
|---|---|---|---|---|
| Viability | 107881 | At_Herbicide_v2_cDNA_P | Timepoint (hr) | 4 |
| | 107881 | At_Herbicide_v2_cDNA_P | Treatment | Glean vs. No Treatment |
| | 107891 | At_Herbicide_v2_cDNA_P | Timepoint (hr) | 12 |
| | 107891 | At_Herbicide_v2_cDNA_P | Treatment | Trimec vs. No Treatment |
| Root | 108429 | At_Tissue_Specific_Expression_cDNA_P | Probe Amount | 50 |
| | 108429 | At_Tissue_Specific_Expression_cDNA_P | Probe Method | operon |
| | 108429 | At_Tissue_Specific_Expression_cDNA_P | Tissue | Green Flower vs. Whole Plant |
| Root | 108434 | At_Root_Tips_cDNA_P | Tissue | Root Tips |
| Shoot Meristem | 108435 | At_stm_Mutants_cDNA_P | Plant Line | wt Landsburg vs stm |
| | 108435 | At_stm_Mutants_cDNA_P | Tissue | Shoot Apical Meristem Region |
| Reproductive and Seed & Fruit | 108437 | At_Tissue_Specific_Expression_cDNA_P | Probe Amount | 33 |
| Development | 108437 | At_Tissue_Specific_Expression_cDNA_P | Probe Method | operon |
| | 108437 | At_Tissue_Specific_Expression_cDNA_P | Tissue | <5 mm Siliques vs. Whole Plant |
| Reproductive and Seed & Fruit | 108438 | At_Tissue_Specific_Expression_cDNA_P | Probe Amount | 33 |
| Development | 108438 | At_Tissue_Specific_Expression_cDNA_P | Probe Method | operon |
| | 108438 | At_Tissue_Specific_Expression_cDNA_P | Tissue | 5 wk Siliques vs. Whole Plant |
| Root | 108439 | At_Tissue_Specific_Expression_cDNA_P | Probe Amount | 33 |
| | 108439 | At_Tissue_Specific_Expression_cDNA_P | Probe Method | operon |
| | 108439 | At_Tissue_Specific_Expression_cDNA_P | Tissue | Roots (2 wk) vs. Whole Plant |
| Imbibition & Germination | 108461 | At_Germinating_Seeds_cDNA_P | Age | 1 vs. 0 |
| | 108461 | At_Germinating_Seeds_cDNA_P | Tissue | Germinating Seeds |
| Imbibition & Germination | 108462 | At_Germinating_Seeds_cDNA_P | Age | 2 vs. 0 |
| | 108462 | At_Germinating_Seeds_cDNA_P | Tissue | Greminating Seeds |
| Early Seedling Phase | 108463 | At_Germinating_Seeds_cDNA_P | Age | 3 vs. 0 |
| | 108463 | At_Germinating_Seeds_cDNA_P | Tissue | Germinating Seeds |
| Early Seedling Phase | 108464 | At_Germinating_Seeds_cDNA_P | Age | 4 vs. 0 |
| | 108464 | At_Germinating_Seeds_cDNA_P | Tissue | Germinating Seeds |
| Viability | 108465 | At_Herbicide_v3_1_cDNA_P | Timepoint (hr) | 12 |
| | 108465 | At_Herbicide_v3_1_cDNA_P | Treatment | Roundup vs. No Treatment |
| Drought and Reproductive | 108473 | At_Drought_Flowers_cDNA_P | Timepoint (hr) | 7 d |
| | 108473 | At_Drought_Flowers_cDNA_P | Tissue | Flowers |
| | 108473 | At_Drought_Flowers_cDNA_P | Treatment | Drought vs. No Drought |
| Shoot Meristem | 108480 | At_Shoot_Apices_cDNA_P | Plant Line | Ws-2 |
| | 108480 | At_Shoot_Apices_cDNA_P | Treatment | 1 uM BR vs. No Treatment |
| Shoot Meristem | 108481 | At_Shoot_Apices_cDNA_P | Plant Line | Ws-2 |
| | 108481 | At_Shoot_Apices_cDNA_P | Treatment | 1 uM BRZ vs. No Treatment |
| Leaves | 108488 | At_50 mM_NH4NO3_L-to-H_Rosette_{—} | Timepoint (hr) | 2 |
| | | cDNA_P | | |
| Heat | 108523 | Zm_42deg_Heat_P | Temperature | Heat (42 deg C.) |
| | 108523 | Zm_42deg_Heat_P | Timepoint (hr) | 6 |
| | 108523 | Zm_42deg_Heat_P | Tissue | Aerial |
| Imbibition & Germination | 108528 | Zm_Imbibed_Seeds_P | Age | 5 vs. 2 |
| | 108528 | Zm_Imbibed_Seeds_P | Tissue | Aerial vs. Embryo |
| | 108528 | Zm_Imbibed_Seeds_P | Treatment | Imbibition |
| Imbibition & Germination | 108530 | Zm_Imbibed_Seeds_P | Age | 6 vs. 2 |
| | 108530 | Zm_Imbibed_Seeds_P | Tissue | Aerial vs. Embryo |
| | 108530 | Zm_Imbibed_Seeds_P | Treatment | Imbibition |
| Imbibition & Germination, | 108543 | Zm_Imbibed_Embryo_Endosperm_P | Age | 2 |
| Reproductive | 108543 | Zm_Imbibed_Embryo_Endosperm_P | Tissue | Embryo vs. Whole Plant |
| | 108543 | Zm_Imbibed_Embryo_Endosperm_P | Treatment | Imbibed |
| Imbibition & Germination | 108546 | Zm_Imbibed_Seeds_P | Age | 3 vs. 2 |
| | 108546 | Zm_Imbibed_Seeds_P | Tissue | Roots vs. Embryo |
| | 108546 | Zm_Imbibed_Seeds_P | Treatment | Imbibition |
| Jasmonate | 108569 | At_0.001%_MeJA_cDNA_P | Timepoint (hr) | 6 |
| | 108569 | At_0.001%_MeJA_cDNA_P | Tissue | Aerial |
| | 108569 | At_0.001%_MeJA_cDNA_P | Treatment | 0.001% MeJA vs. No Treatment |
| Heat | 108577 | At_42deg_Heat_cDNA_P | Temperature | 42 vs. 22 |
| | 108577 | At_42deg_Heat_cDNA_P | Timepoint (hr) | 6 |
| | 108577 | At_42deg_Heat_cDNA_P | Tissue | Aerial |
| Cold | 108579 | At_4deg_Cold_cDNA_P | Temperature | 4 vs. 22 |
| | 108579 | At_4deg_Cold_cDNA_P | Timepoint (hr) | 6 |
| | 108579 | At_4deg_Cold_cDNA_P | Tissue | Aerial |
| Root and Root Hairs | 108594 | At_Ler-rhl_Root_cDNA_P | Plant Line | Ler_rhl |
| | 108594 | At_Ler-rhl_Root_cDNA_P | Tissue | Root |
| ABA, Drought, Germination | 108614 | At_100uM_ABA_Mutants_cDNA_P | Plant Line | CS24 |
| | 108614 | At_100uM_ABA_Mutants_cDNA_P | Timepoint (hr) | 6 |
| | 108614 | At_100uM_ABA_Mutants_cDNA_P | Tissue | Aerial |
| | 108614 | At_100uM_ABA_Mutants_cDNA_P | Treatment | 100 uM ABA vs. No Treatment |
| ABA, Drought, Germination | 108622 | At_100uM_ABA_Mutants_cDNA_P | Plant Line | CS22 |
| | 108622 | At_100uM_ABA_Mutants_cDNA_P | Timepoint (hr) | 6 |
| | 108622 | At_100uM_ABA_Mutants_cDNA_P | Tissue | Aerial |
| | 108622 | At_100uM_ABA_Mutants_cDNA_P | Treatment | 100 uM ABA vs. No Treatment |
| Viability | 108629 | At_Herbicide_v3_1_cDNA_P | Timepoint (hr) | 1 |
| | 108629 | At_Herbicide_v3_1_cDNA_P | Treatment | Glean vs. No Treatment |
| Viability | 108630 | At_Herbicide_v3_1_cDNA_P | Timepoint (hr) | 1 |
| | 108630 | At_Herbicide_v3_1_cDNA_P | Treatment | Trimec vs. No Treatment |
| Salicylic Acid | 108668 | At_2mM_SA_cDNA_P | Plant Line | WS |
| | 108668 | At_2mM_SA_cDNA_P | Timepoint (hr) | 6 |
| | 108668 | At_2mM_SA_cDNA_P | Treatment | 2 mM SA vs. No Treatment |
| Reproductive and Seed & Fruit | 108687 | Zm_Embryos-Flowers_P | Tissue | Embryo |
| Development | 108688 | Zm_Embryos-Flowers_P | Tissue | Immature Flowers |
| ABA, Drought, Germination | 20000069 | At_100uM_ABA_Mutants_cDNA_P | Plant Line | CS23 |
| | 20000069 | At_100uM_ABA_Mutants_cDNA_P | Timepoint (hr) | 6 |
| | 20000069 | At_100uM_ABA_Mutants_cDNA_P | Tissue | Aerial |
| | 20000069 | At_100uM_ABA_Mutants_cDNA_P | Treatment | 100 uM ABA vs. No Treatment |
| ABA, Drought, Germination | 20000070 | At_100uM_ABA_Mutants_cDNA_P | Plant Line | CS24 |
| | 20000070 | At_100uM_ABA_Mutants_cDNA_P | Timepoint (hr) | 6 |
| | 20000070 | At_100uM_ABA_Mutants_cDNA_P | Tissue | Aerial |
| | 20000070 | At_100uM_ABA_Mutants_cDNA_P | Treatment | 100 uM ABA vs. No Treatment |
| ABA, Drought, Germination | 20000071 | At_100uM_ABA_Mutants_cDNA_P | Plant Line | CS8104 |
| | 20000071 | At_100uM_ABA_Mutants_cDNA_P | Timepoint (hr) | 6 |
| | 20000071 | At_100uM_ABA_Mutants_cDNA_P | Tissue | Aerial |
| | 20000071 | At_100uM_ABA_Mutants_cDNA_P | Treatment | 100 uM ABA vs. No Treatment |
| ABA, Drought, Germination | 20000072 | At_100uM_ABA_Mutants_cDNA_P | Plant Line | CS8105 |
| | 20000072 | At_100uM_ABA_Mutants_cDNA_P | Timepoint (hr) | 6 |
| | 20000072 | At_100uM_ABA_Mutants_cDNA_P | Tissue | Aerial |
| | 20000072 | At_100uM_ABA_Mutants_cDNA_P | Treatment | 100 uM ABA vs. No Treatment |
| ABA, Drought, Germination | 20000086 | At_100uM_ABA_Mutants_cDNA_P | Plant Line | CS22 |
| | 20000086 | At_100uM_ABA_Mutants_cDNA_P | Timepoint (hr) | 6 |
| | 20000086 | At_100uM_ABA_Mutants_cDNA_P | Tissue | aeriel |
| | 20000086 | At_100uM_ABA_Mutants_cDNA_P | Treatment | 100 uM ABA vs. No Treatment |
| ABA, Drought, Germination | 20000087 | At_100uM_ABA_Mutants_cDNA_P | Plant Line | WS |
| | 20000087 | At_100uM_ABA_Mutants_cDNA_P | Timepoint (hr) | 6 |
| | 20000087 | At_100uM_ABA_Mutants_cDNA_P | Tissue | aeriel |
| | 20000087 | At_100uM_ABA_Mutants_cDNA_P | Treatment | 100 uM ABA vs. No Treatment |
| ABA, Drought, Germination | 20000088 | At_100uM_ABA_Mutants_cDNA_P | Plant Line | Landsberg |
| | 20000088 | At_100uM_ABA_Mutants_cDNA_P | Timepoint (hr) | 6 |
| | 20000088 | At_100uM_ABA_Mutants_cDNA_P | Tissue | aeriel |
| | 20000088 | At_100uM_ABA_Mutants_cDNA_P | Treatment | 100 uM ABA vs. No Treatment |
| Salicylic Acid | 20000090 | At_2mM_SA_CS3726-Columbia_cDNA_P | Plant Line | Columbia |
| | 20000090 | At_2mM_SA_CS3726-Columbia_cDNA_P | Timepoint (hr) | 6 |
| | 20000090 | At_2mM_SA_CS3726-Columbia_cDNA_P | Tissue | Aerial |
| | 20000090 | At_2mM_SA_CS3726-Columbia_cDNA_P | Treatment | 2 mM SA vs. No Treatment |
| Heat | 20000111 | At_42deg_Heat_cDNA_P | Temperature | 42 vs. 23 |
| | 20000111 | At_42deg_Heat_cDNA_P | Timepoint (hr) | 6 |
| | 20000111 | At_42deg_Heat_cDNA_P | Tissue | Aerial |
| Heat | 20000113 | At_42deg_Heat_cDNA_P | Temperature | 42 vs. 23 |
| | 20000113 | At_42deg_Heat_cDNA_P | Timepoint (hr) | 8 |
| | 20000113 | At_42deg_Heat_cDNA_P | Tissue | Aerial |
| ABA, Drought, Germination | 20000117 | At_100uM_ABA_Mutants_cDNA_P | Plant Line | columbia |
| | 20000117 | At_100uM_ABA_Mutants_cDNA_P | Timepoint (hr) | 6 |
| | 20000117 | At_100uM_ABA_Mutants_cDNA_P | Tissue | aerial |
| | 20000117 | At_100uM_ABA_Mutants_cDNA_P | Treatment | 100 uM ABA vs. No Treatment |
| Heat | 20000171 | At_42deg_Heat_P | Probe Method | mRNA vs. mRNA |
| | 20000171 | At_42deg_Heat_P | Temperature | 42 vs. 22 |
| | 20000171 | At_42deg_Heat_P | Timepoint (hr) | 1 |
| | 20000171 | At_42deg_Heat_P | Tissue | Aerial |
| Heat | 20000173 | At_42deg_Heat_P | Probe Method | mRNA vs. mRNA |
| | 20000173 | At_42deg_Heat_P | Temperature | 42 vs. 22 |
| | 20000173 | At_42deg_Heat_P | Timepoint (hr) | 6 |
| | 20000173 | At_42deg_Heat_P | Tissue | Aerial |
| Early Seedling Phase | 20000179 | At_Germinating_Seeds_P | Age | 6 vs. 0 |
| | 20000179 | At_Germinating_Seeds_P | Tissue | Germinating Seeds |
| Early Seedling Phase | 20000180 | At_Germinating_Seeds_P | Age | 24 vs. 0 |
| | 20000180 | At_Germinating_Seeds_P | Tissue | Germinating Seeds |
| Salicylic Acid | 20000182 | At_2mM_SA_P | Timepoint (hr) | 6 |
| | 20000182 | At_2mM_SA_P | Tissue | Aerial |
| | 20000182 | At_2mM_SA_P | Treatment | 2 mM SA vs. No Treatment |
| Leaves, Shoot Meristem | 20000184 | At_Shoots_P | Age | 7 |
| | 20000184 | At_Shoots_P | Tissue | Shoots vs. Whole Plant |
| Root | 20000185 | At_Roots_P | Age | 7 |
| | 20000185 | At_Roots_P | Tissue | Roots vs. Whole Plant |
| Cold | 20000213 | At_4deg_Cold_P | Temperature | 4 vs. 22 |
| | 20000213 | At_4deg_Cold_P | Timepoint (hr) | 2 |
| Seed and Fruit Development | 20000234 | At_Siliques_P | Tissue | <5 mm Siliques vs. Whole Plant |
| Seed and Fruit Development | 20000235 | At_Siliques_YF_6-05-02_P | Tissue | 5-10 mm Siliques vs. Whole Plant |
| Seed and Fruit Development | 20000236 | At_Siliques_P | Tissue | >10 mm Siliques vs. Whole Plant |
| Reproductive and Seed & Fruit | 20000264 | At_Open_Flower_P | Tissue | Open Flower vs. Whole Plant |
| Development | | | | |
| Reproductive and Seed & Fruit | 20000265 | At_Open_Flower_P | Tissue | Closed Bud vs. Whole Plant |
| Development | | | | |
| Reproductive and Seed & Fruit | 20000286 | At_Open_Flower_P | Tissue | Half Open vs. Whole Plant |
| Development | | | | |
| Drought | 20000437 | At_Drought_P | Timepoint (hr) | 24 |
| | 20000437 | At_Drought_P | Tissue | Whole Plant |
| | 20000437 | At_Drought_P | Treatment | Drought vs. No Drought |
| Leaves, Shoot Meristem | 20000438 | At_Shoots_P | Age | 14 |
| | 20000438 | At_Shoots_P | Tissue | Shoots vs. Whole Plant |
| Roots | 20000439 | At_Roots_P | Age | 14 |
| | 20000439 | At_Roots_P | Tissue | Roots vs. Whole Plant |
| Brassinolide | 20000441 | At_1uM_BR-BRZ_P | Tissue | Shoot Apices |
| | 20000441 | At_1uM_BR-BRZ_P | Treatment | 1 uM BR vs. No Treatment |
| | 20000443 | At_1uM_BR-BRZ_P | Tissue | Shoot Apices |
| | 20000443 | At_1uM_BR-BRZ_P | Treatment | 1 uM BRZ vs. No Treatment |
| Salicylic Acid | 20000478 | Zm_5mM_SA_P | Age | 8 |
| | 20000478 | Zm_5mM_SA_P | Plant Line | Hybrid |
| | 20000478 | Zm_5mM_SA_P | Timepoint (hr) | 72 |
| | 20000478 | Zm_5mM_SA_P | Tissue | Aerial |
| | 20000478 | Zm_5mM_SA_P | Treatment | 5 mM SA vs. No Treatment |
| Reproductive and Seed & Fruit | 20000493 | Zm_Hybrid_Seed_Dev_P | DAP | 20 vs. 12 |
| Development | 20000493 | Zm_Hybrid_Seed_Dev_P | Plant Line | Hybrid |
| | 20000493 | Zm_Hybrid_Seed_Dev_P | Tissue | Endosperm vs. Unfert Floret |
| Guard Cells | 20000495 | At_Guard_Cells_P | Harvest Date | Aug. 2, 2002 |
| | 20000495 | At_Guard_Cells_P | Organism | A. thaliana |
| | 20000495 | At_Guard_Cells_P | Tissue | Guard Cells vs. Leaves |
| PEG | 20000527 | At_10%_PEG_P | Age | 20 |
| | 20000527 | At_10%_PEG_P | Tissue | Aerial |
| | 20000527 | At_10%_PEG_P | Treatment | 10% PEG vs. No Treatment |
| ABA, Drought, Germination | 20000573 | At_100uM_ABA_Mutants_P | Organism | A. thaliana |
| | 20000573 | At_100uM_ABA_Mutants_P | Plant Line | CS22 vs. Ler wt |
| | 20000573 | At_100uM_ABA_Mutants_P | Timepoint (hr) | N/A |
| | 20000573 | At_100uM_ABA_Mutants_P | Tissue | Whole Plant |
| | 20000573 | At_100uM_ABA_Mutants_P | Treatment | None |
| Viability | 20000629 | Zm_Herbicide-Treatments_P | Timepoint (hr) | 12 |
| | 20000629 | Zm_Herbicide-Treatments_P | Tissue | Aerial |
| | 20000629 | Zm_Herbicide-Treatments_P | Treatment | Trimec vs. No Treatment |
| Drought | 20000638 | At_Drought_cDNA_P | Timepoint (hr) | 144 |
| | 20000638 | At_Drought_cDNA_P | Tissue | sdf |
| Reproductive | 20000794 | At_Petals_P | Age | 23-25 days |
| | 20000794 | At_Petals_P | Tissue | Petals vs. Whole plant |
| Shade | 20001247 | At_Far-red-induction_P | Age | 7 |
| | 20001247 | At_Far-red-induction_P | Light | Far Red vs. White |
| | 20001247 | At_Far-red-induction_P | Plant Line | Columbia |
| | 20001247 | At_Far-red-induction_P | Timepoint (hr) | 1 |
| Shade | 20001248 | At_Far-red-induction_P | Age | 7 |
| | 20001248 | At_Far-red-induction_P | Light | Far Red vs. White |
| | 20001248 | At_Far-red-induction_P | Plant Line | Columbia |
| | 20001248 | At_Far-red-induction_P | Timepoint (hr) | 4 |
| Shade | 20001450 | At_Far-red-induction_P | Age | 7 |
| | 20001450 | At_Far-red-induction_P | Light | Far Red vs. White |
| | 20001450 | At_Far-red-induction_P | Plant Line | Columbia |
| | 20001450 | At_Far-red-induction_P | Timepoint (hr) | 8 |
| Shade | 20001451 | At_Far-red-induction_P | Age | 7 |
| | 20001451 | At_Far-red-induction_P | Light | Far Red vs. White |
| | 20001451 | At_Far-red-induction_P | Plant Line | Columbia |
| | 20001451 | At_Far-red-induction_P | Timepoint (hr) | 24 |
| Nitrogen | 20001459 | At_50mM_NH4NO3_L-to-H_P | Timepoint (hr) | 4 |
| | 20001459 | At_50mM_NH4NO3_L-to-H_P | Tissue | Siliques |
| | 20001459 | At_50mM_NH4NO3_L-to-H_P | Treatment | 50 mM NH4NO3 vs. 100 mM |
| | | | | Manitol |
| Viability | 20000530 | Zm_2-4D_YF_8-26-02_P | Organism | Zea Mays |
| | 20000530 | Zm_2-4D_YF_8-26-02_P | Timepoint (hr) | 48 |
| | 20000530 | Zm_2-4D_YF_8-26-02_P | Tissue | Aerial |
| | 20000530 | Zm_2-4D_YF_8-26-02_P | Treatment | 2,4-D vs. No Treatment |
| Guard Cells | 20000570 | At_Guard_Cells_JD_9-9-02_cDNA_P | Harvest Date | Jul. 19, 2002 |
| | 20000570 | At_Guard_Cells_JD_9-9-02_cDNA_P | Organism | Canola |
| | 20000570 | At_Guard_Cells_JD_9-9-02_cDNA_P | Tissue | Guard Cells vs. Leaves |
| Nitric Oxide Responsive | | At_5mM NaNP; Zm_5mMNO | | |
| Reproductive, fruit and seed | | At_ap2_floral_buds | | |
| development | | | | |
| Reproductive | | At_Ler-pi_Ovule | | |
| Root and Root Hairs | | At_rhl_Mutants | | |
| Wounding | | At_Wounding | | |
| Methyl Jasmonate | | Zm_0.001%MeJA | | |
| Shoot Meristem | | Zm_Meristem | | |

1. An isolated nucleic acid molecule comprising:
a) a nucleic acid having a nucleotide sequence which encodes an amino acid sequence exhibiting at least 85% sequence identity to an amino acid sequence in TABLE 1;
b) a nucleic acid which is a complement of a nucleotide sequence according to paragraph (a);
c) a nucleic acid which is the reverse of the nucleotide sequence according to subparagraph (a), such that the reverse nucleotide sequence has a sequence order which is the reverse of the sequence order of the nucleotide sequence according to subparagraph (a); or
d) a nucleic acid capable of hybridizing to a nucleic acid according to any one of paragraphs (a)-(c), under conditions that permit formation of a nucleic acid duplex at a temperature from about 40° C. and 48° C. below the melting temperature of the nucleic acid duplex.

2. The isolated nucleic acid molecule according to claim 1, which has the nucleotide sequence according to any sequence in TABLE 1.

3. The isolated nucleic acid molecule according to claim 1, wherein said amino acid sequence comprises any polypeptide sequence in TABLE 1.

4. A vector construct comprising:
a) a first nucleic acid having a regulatory sequence capable of causing transcription and/or translation in a plant; and
b) a second nucleic acid having the sequence of the isolated nucleic acid molecule according to any one of claims 1-3;
wherein said first and second nucleic acids are operably linked and wherein said second nucleic acid is heterologous to any element in said vector construct.

5. The vector construct according to claim 4, wherein said first nucleic acid is native to said second nucleic acid.

6. The vector construct according to claim 4, wherein said first nucleic acid is heterologous to said second nucleic acid.

7. A host cell comprising an isolated nucleic acid molecule according to any one of claims 1-3 wherein said nucleic acid molecule is flanked by exogenous sequence.

8. A host cell comprising a vector construct according to claim 4.

9. An isolated polypeptide comprising an amino acid sequence exhibiting at least 85% sequence identity of an amino acid sequence of Table 1.

10. A method of introducing an isolated nucleic acid into a host cell comprising:
a) providing an isolated nucleic acid molecule according to any one of claims 1-3; and
b) contacting said isolated nucleic with said host cell under conditions that permit insertion of said nucleic acid into said host cell.

11. A method of transforming a host cell which comprises contacting a host cell with a vector construct according to claim 4.

12. A method for detecting a nucleic acid in a sample which comprises:
a) providing an isolated nucleic acid molecule according to any one of claims 1-3;
b) contacting said isolated nucleic acid molecule with a sample under conditions which permit a comparison of the sequence of said isolated nucleic acid molecule with the sequence of DNA in said sample; and
c) analyzing the result of said comparison.

13. A plant, plant cell, plant material or seed of a plant which comprises a nucleic acid molecule according to any one of claims 1-3 which is exogenous or heterologous to said plant or plant cell.

14. A plant, plant cell, plant material or seed of a plant which comprises a vector construct according to claim 4.

15. A plant which has been regenerated from a plant cell or seed according to claim 13.

16. A plant which has been regenerated from a plant cell or seed according to claim 14.
